(19) United States
(12) Patent Application Publication
(10) Pub. No.: US 2024/0182444 A1
(43) Pub. Date: Jun. 6, 2024

(54) ANTI-VIRAL COMPOUNDS

(71) Applicants: Aligos Therapeutics, Inc., South San Francisco, CA (US); Katholieke Universiteit Leuven, Leuven (BE)

(72) Inventors: Dorothée Alice Marie-Eve Bardiot, Leuven (BE); Sandro Boland, Leuven (BE); Arnaud Didier Marie Marchand, Leuven (BE); Koen Vandyck, Paal (BE); David McGowan, Brussels (BE); Leonid Beigelman, San Mateo, CA (US)

(21) Appl. No.: 18/487,809

(22) Filed: Oct. 16, 2023

Related U.S. Application Data
(60) Provisional application No. 63/379,825, filed on Oct. 17, 2022.

(51) Int. Cl. C07D 401/12; A61P 31/14; C07D 403/12; C07D 471/04; C07D 491/04

(52) U.S. Cl. CPC C07D 401/12; A61P 31/14; C07D 403/12; C07D 471/04; C07D 491/04

(57) ABSTRACT

Provided herein are compounds of Formula (I), or pharmaceutically acceptable salts thereof, pharmaceutical compositions that include a compound described herein (including pharmaceutically acceptable salts of a compound described herein) and methods of synthesizing the same. Also provided herein are methods of treating diseases and/or conditions with a compound of Formula (I), or a pharmaceutically acceptable salt thereof.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference in their entireties under 37 CFR 1.57, and Rules 4.18 and 20.6, including U.S. Provisional Application No. 63/379,825, filed Oct. 17, 2022.

### BACKGROUND

### Field

The present application relates to the fields of chemistry, biochemistry and medicine. Disclosed herein are compounds of Formula (I), or pharmaceutically acceptable salt thereof, pharmaceutical compositions that include a compound described herein (including pharmaceutically acceptable salts of a compound described herein) and methods of synthesizing the same. Also disclosed herein are methods of treating diseases and/or conditions with a compound of Formula (I), or a pharmaceutically acceptable salt thereof.

### Description

A positive-sense single-stranded RNA virus ((+)ssRNA virus) is a virus that uses positive sense, single stranded, RNA as its genetic material. Positive-sense single-stranded RNA viruses can be enveloped or non-enveloped. Coronaviridae, Picornaviridae and Norviruses are each a (+)ssRNA virus. Each of the aforementioned viruses are known to infect mammals, including humans.

### SUMMARY

Some embodiments disclosed herein relate to a compound of Formula (I), or a pharmaceutically acceptable salt thereof.

Some embodiments disclosed herein relate to a pharmaceutical composition that can contain an effective amount of a compound of Formula (I), or a pharmaceutically acceptable salt thereof.

Some embodiments described herein relate to a method of treating a coronavirus infection that can include administering to a subject identified as suffering from the coronavirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of treating a coronavirus infection.

Some embodiments disclosed herein relate to a method of inhibiting replication of a coronavirus that can include contacting a cell infected with the coronavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of inhibiting the replication a coronavirus.

Some embodiments described herein relate to a method of treating a picornavirus infection that can include administering to a subject identified as suffering from the picornavirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of treating a picornavirus infection.

Some embodiments disclosed herein relate to a method of inhibiting replication of a picornavirus that can include contacting a cell infected with the picornavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of inhibiting the replication a picornavirus.

Some embodiments described herein relate to a method of treating a norovirus infection that can include administering to a subject identified as suffering from the norovirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of treating a norovirus infection.

Some embodiments disclosed herein relate to a method of inhibiting replication of a norovirus that can include contacting a cell infected with the norovirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to a compound, or a pharmaceutically acceptable salt thereof, as described herein, or a pharmaceutical composition that includes an effective amount of a compound, or a pharmaceutically acceptable salt thereof, as described herein for the use of inhibiting the replication a norovirus.

These are other embodiments are described in greater detail below.

### DETAILED DESCRIPTION

Coronaviridae viruses are a family of enveloped, positive-stranded, single-stranded, spherical RNA viruses. Coronaviruses are named for the crown-like spikes on their surface. The Coronaviridae family includes two sub-families, Coronavirus and Torovirus. The Coronavirus genus has a helical nucleocapsid, and Torovirus genus has a tubular nucleocapsid. The Coronaviridae family of viruses includes Middle East respiratory syndrome coronavirus (MERS-CoV), SARS and SARS-CoV-2.

Coronavirus disease 2019 (COVID-19) (also referred to as novel coronavirus pneumonia or 2019-nCoV acute respiratory disease) is an infectious disease caused by the virus severe acute respiratory syndrome coronavirus 2 (SARS-CoV-2) (also referred to as novel coronavirus 2019, or 2019-nCoV). The disease was first identified in December 2019 and spread globally, causing a pandemic. Symptoms of COVID-19 include fever, cough, shortness of breath, fatigue, headache, loss of smell, nasal congestion, sore throat, coughing up sputum, pain in muscles or joints, chills, nausea, vomiting, and diarrhea. In severe cases, symptoms can include difficulty waking, confusion, blueish face or lips, coughing up blood, decreased white blood cell count, and kidney failure. Complications can include pneumonia, viral sepsis, acute respiratory distress syndrome, and kidney failure.

COVID-19 is especially threatening to public health. The virus is highly contagious, and studies currently indicate that it can be spread by asymptomatic carriers or by those who are pre-symptomatic. Likewise, the early stage of the disease is slow-progressing enough that carriers do not often realize they are infected, leading them to expose numerous others to the virus. The combination of COVID-19's ease of transmission, its high rate of hospitalization of victims, and its death rate make the virus a substantial public health risk, especially for countries without a healthcare system equipped to provide supportive care to pandemic-level numbers of patients. There is not yet a vaccine or specific antiviral treatment for COVID-19 and accordingly, there is a pressing need for treatments or cures.

SARS-CoV-2 is not the only coronavirus that causes disease. It is a β-coronavirus, a genus of coronaviruses that includes other human pathogens, including SARS-CoV (the causative agent of SARS), MERS-CoV (the causative agent of MERS), and HCoV-OC43 (a causative agent of the common cold). The infectivity of these viruses, and the severity of the diseases they cause, varies widely. β-coronavirus can also manifest as zoonotic infections, spread to and from humans and animals. Additionally, non-human species such as camels, bats, tigers, non-human primates, and rabbits can be susceptible to β-coronavirus. Accordingly, there is a pressing need for treatments or cures to multiple coronaviruses.

The present disclosure provides molecules useful against coronaviruses, and especially SARS-CoV-2, the causative agent of COVID-19 in humans. Accordingly, the present disclosure fulfills the need in the art for compounds that can be safely and effectively treat or prevent coronavirus infections in humans.

Picornaviruses are a family of positive strand RNA, nonenveloped viruses. A picornavirus has 60 identical subunits (vertices) which contain five protomers. Each protomer is made up of one copy of four proteins, named VP1, VP2, VP3 and VP4. There are several genera of picornaviruses, including, Enterovirus, Aphthovirus, Cardiovirus and Hepatovirus. Enteroviruses known to infect human include, but are not limited to, Rhinovirus A, Rhinovirus B, Rhinovirus C, Coxsackievirus A, Coxsackievirus B and Poliovirus. There is no specific treatment for a picornavirus infection.

Noroviruses are single-stranded positive-sense RNA, non-enveloped viruses belonging to the Caliciviridae family. Noroviruses are often spread by the fecal-oral route, and are a common cause of gastroenteritis. Infected subjects can experience nausea, non-bloody diarrhea, vomiting and/or abdominal pain. Those suffering from a norovirus infection can become severely dehydrated and require medical attention. As with a picornavirus infection, there is no specific treatment for a norovirus infection. Accordingly, there is a need for compounds that effectively treat or prevent a picornavirus and/or a norovirus infection.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more of the indicated substituents. If no substituents are indicated, it is meant that the indicated “optionally substituted” or “substituted” group may be substituted with one or more group(s) (such as 1, 2 or 3) individually and independently selected from deuterium, alkyl, alkenyl, alkynyl, cycloalkyl, cycloalkenyl, aryl, heteroaryl, heterocyclyl, aryl(alkyl), heteroaryl(alkyl), heterocyclyl(alkyl), hydroxy, alkoxy, acyl, cyano, halogen, thiocarbonyl, 0-carbamyl, N-carbamyl, O-thiocarbamyl, N-thiocarbamyl, C-amido, N-amido, S-sulfonamido, N-sulfonamido, C-carboxy, O-carboxy, C-amido(alkyl), isocyanato, thiocyanato, nitro, azido, silyl, sulfenyl, sulfinyl, sulfonyl, haloalkyl, haloalkoxy, trihalomethanesulfonyl, trihalomethanesulfonamido, an amino, a mono-substituted amine and a di-substituted amine.

As used herein, “Cₐ to C_{b}” or “C_{a-b}” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heterocyclyl group. That is, the alkyl, alkenyl, alkynyl, ring of the cycloalkyl, ring of the cycloalkenyl, ring of the aryl, ring of the heteroaryl or ring of the heterocyclyl can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heterocyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. The length of an alkenyl can vary. For example, the alkenyl can be a C₂₋₄ alkenyl, C₂₋₆ alkenyl or C₂₋₈ alkenyl. Examples of alkenyl groups include allenyl, vinylmethyl and ethenyl. An alkenyl group may be unsubstituted or substituted.

As used herein, “alkynyl” refers to an alkyl group that contains in the straight or branched hydrocarbon chain one or more triple bonds. The length of an alkynyl can vary. For example, the alkynyl can be a C₂₋₄ alkynyl, C₂₋₆ alkynyl or C₂₋₈ alkynyl. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused- or spiro-fashion. Cycloalkyl groups can contain 3 to 10 atoms in the ring(s). 3 to 8 atoms in the ring(s) or 3 to 6 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused- or spiro-fashion. A cycloalkenyl can contain 3 to 10 atoms in the ring(s) or 3 to 8 atoms in the ring(s). A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic, bicyclic and tricyclic aromatic ring system (a ring system with fully delocalized pi-electron system) that contain(s) one or more heteroatoms (for example, 1 to 5 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” refers to a monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The number of atoms in the ring(s) of a heterocyclyl group can vary. For example, the heterocyclyl group can contain 4 to 14 atoms in the ring(s), 5 to 10 atoms in the ring(s) or 5 to 6 atoms in the ring(s). The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heterocyclyl may be quaternized. Heterocyclyl groups may be unsubstituted or substituted. Examples of such “heterocyclyl groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and 3,4-methylenedioxyphenyl).

As used herein, “cycloalkyl(alkyl)” refers to a cycloalkyl group connected, as a substituent, via a lower alkylene group. The lower alkylene and cycloalkyl group of a cycloalkyl(alkyl) may be substituted or unsubstituted. A cycloalkyl(alkyl) group may be unsubstituted or substituted.

As used herein, “aryl(alkyl)” refers to an aryl group connected, as a substituent, via a lower alkylene group. The lower alkylene and aryl group of an aryl(alkyl) may be substituted or unsubstituted. Examples include but are not limited to benzyl, 2-phenyl(alkyl), 3-phenyl(alkyl), and naphthyl(alkyl).

As used herein, “heteroaryl(alkyl)” refer to a heteroaryl group connected, as a substituent, via a lower alkylene group. The lower alkylene and heteroaryl group of heteroaryl(alkyl) may be substituted or unsubstituted. Examples include but are not limited to 2-thienyl(alkyl), 3-thienyl(alkyl), furyl(alkyl), thienyl(alkyl), pyrrolyl(alkyl), pyridyl(alkyl), isoxazolyl(alkyl), imidazolyl(alkyl), and their benzo-fused analogs.

A “heterocyclyl(alkyl)” refer to a heterocyclic group connected, as a substituent, via a lower alkylene group. The lower alkylene and heterocyclyl of a heterocyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

“Lower alkylene groups” are straight-chained —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—) and butylene (—CH₂CH₂CH₂CH₂—). A lower alkylene group can be substituted by replacing one or more hydrogen of the lower alkylene group with a substituent(s) listed under the definition of “substituted.” Further, when a lower alkylene group is substituted, the lower alkylene can be substituted by replacing both hydrogens on the same carbon with a cycloalkyl group (e.g.,

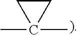

As used herein, “alkoxy” refers to the formula —OR wherein R is an alkyl, an alkenyl, an alkynyl, a cycloalkyl, a cycloalkenyl, an aryl, a heteroaryl, a heterocyclyl, a cycloalkyl(alkyl), an aryl(alkyl), a heteroaryl(alkyl) or a heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzyloxy. In some instances, an alkoxy can be —OR, wherein R is an unsubstituted C₁₋₄ alkyl. An alkoxy may be substituted or unsubstituted.

As used herein, “acyl” refers to a hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl) connected, as substituents, via a carbonyl group. Examples include formyl, acetyl, propanoyl, benzoyl and acryl. An acyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, 1-chloro-2-fluoromethyl and 2-fluoroisobutyl. A haloalkyl may be substituted or unsubstituted.

As used herein, “haloalkoxy” refers to a O-alkyl group and O-monocyclic cycloalkyl group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy, 2-fluoroisobutoxy, chloro-substituted cyclopropyl, fluoro-substituted cyclopropyl, chloro-substituted cyclobutyl and fluoro-substituted cyclobutyl. In some instances, a haloalkoxy can be —OR, wherein R is a C₁₋₄ alkyl substituted by 1, 2 or 3 halogens. A haloalkoxy may be substituted or unsubstituted.

A “sulfenyl” group refers to an “—SR” group in which R can be hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “—S(═O)₂R” group in which R can be the same as defined with respect to sulfenyl. A sulfonyl may be substituted or unsubstituted.

An “O-carboxy” group refers to a “RC(═O)O—” group in which R can be hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CS(═O)₂—” group wherein each X is a halogen.

A “trihalomethanesulfonamido” group refers to an “X₃CS(═O)₂N(R_{A})—” group wherein each X is a halogen, and R_{A} is hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl).

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—SCN” group.

An “isothiocyanato” group refers to an “—NCS” group.

A “mercapto” group refers to an “—SH” group.

A “carbonyl” group refers to a —C(═O)— group.

An “S-sulfonamido” group refers to a “—S(═O)₂N(R_{A}R_{B})” group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An S-sulfonamido may be substituted or unsubstituted.

An “N-sulfonamido” group refers to a “RS(═O)₂N(R_{A})—” group in which R and R_{A} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An N-sulfonamido may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An N-carbamyl may be substituted or unsubstituted.

An “O-thiocarbamyl” group refers to a “—OC(═S)N(R_{A}R_{B})” group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). An N-amido may be substituted or unsubstituted.

A “mono-substituted amine” refers to a “—NHR_{A}” in which R_{A} can be independently an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A mono-substituted amine may be substituted or unsubstituted. In some instances, a mono-substituted amine can be —NHR_{A}, wherein R_{A} can be an unsubstituted C₁₋₆ alkyl or an unsubstituted or a substituted benzyl.

A “di-substituted amine” refers to a “—NR_{A}R_{B}” in which R_{A} and R_{B} can be independently can be independently an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A mono-substituted amine may be substituted or unsubstituted. In some instances, a mono-substituted amine can be —NR_{A}R_{B}, wherein R_{A} and R_{B} can be independently an unsubstituted C₁₋₆ alkyl or an unsubstituted or a substituted benzyl.

A “ketoamide” group refers to a —C(═O)—C(═O)N(R_{A}R_{B}) group in which R_{A} and R_{B} can be independently hydrogen, an alkyl, an alkenyl, an alkynyl, a cycloalkyl, a cycloalkenyl, an aryl, a heteroaryl, a heterocyclyl, an aryl(alkyl), a heteroaryl(alkyl) or a heterocyclyl(alkyl). A ketoamide may be substituted or unsubstituted.

The term “halogen atom” or “halogen” as used herein, means any one of the radio-stable atoms of column 7 of the Periodic Table of the Elements, such as, fluorine, chlorine, bromine and iodine.

As used herein, the term “fused” refers to two rings which have two atoms and one bond in common. As used herein, the term “spiro” refers to two rings which have one atom in common and the two rings are not linked by a bridge.

Where the numbers of substituents are not specified (e.g., haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof. In addition, the term “comprising” is to be interpreted synonymously with the phrases “having at least” or “including at least”. When used in the context of a compound or composition, the term “comprising” means that the compound or composition includes at least the recited features or components but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality.

It is understood that, in any compound described herein having one or more chiral centers, if an absolute stereochemistry is not expressly indicated, then each center may independently be of (R)-configuration or (S)-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Where a range of values is provided, it is understood that the upper and lower limit, and each intervening value between the upper and lower limit of the range is encompassed within the embodiments.

### Compounds

Some embodiments disclosed herein relate to a compound of Formula (I), or a pharmaceutically acceptable salt thereof:

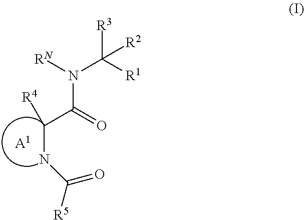

wherein: R^{N} can be hydrogen, deuterium or an unsubstituted or a substituted C₁₋₆ alkyl; Ring A¹ can be an unsubstituted or a substituted

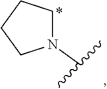 [SMILES: *N1CCCC1]

an unsubstituted or a substituted

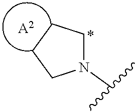

an unsubstituted or a substituted

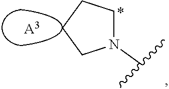

an unsubstituted or a substituted

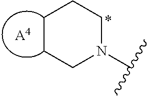

or an unsubstituted or a substituted

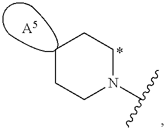

wherein the N is the nitrogen of Ring A¹ shown in Formula (I) and the carbon indicated with an asterisk is the carbon to which R⁴ is connected; Ring A² and Ring A⁴ can be an unsubstituted or a substituted 3- to 10-membered ring system that optionally includes 1 to 3 heteroatoms selected from O (oxygen), S (sulfur) and N (nitrogen), and wherein Ring A² and Ring A⁴ can be optionally substituted with one or more moieties independently selected from ═O, ═CH₂, deuterium, halogen, hydroxy, an unsubstituted C₁₋₄ alkyl, an unsubstituted C₁₋₄ haloalkyl, an unsubstituted C₂₋₄ alkenyl and an unsubstituted or a substituted C₃₋₆ monocyclic cycloalkyl; Ring A³ and Ring A⁵ can be an unsubstituted or a substituted monocyclic C₃₋₆ cycloalkyl; R¹ can be selected from cyano, an unsubstituted or a substituted C₂₋₅ alkynyl, an unsubstituted or a substituted acyl, an unsubstituted or a substituted ketoamide, —CH(OH)—(S(═O)₂—OH), —CH(OH)—(S(═O)₂—O—), —CH(OH)((P═O)(OR⁶)₂) and —C(═O)CH₂—O—((P═O)(OR⁷)₂); each R⁶ and each R⁷ can be independently hydrogen, an unsubstituted C₁₋₆ alkyl, an unsubstituted C₂₋₆ alkenyl, an unsubstituted C₁₋₆ haloalkyl, an unsubstituted or a substituted aryl or an unsubstituted or a substituted aryl(C₁₋₄ alkyl); R² can be hydrogen, deuterium, halogen or an unsubstituted C₁₋₄ alkyl; and R³ can be an unsubstituted or a substituted monocyclic nitrogen-containing heteroaryl, an unsubstituted or a substituted bicyclic nitrogen-containing heteroaryl, an unsubstituted or a substituted bicyclic nitrogen-containing heterocyclyl or an unsubstituted or a substituted naphthyl; or R² and R³ can be taken together along with the carbon to which they are attached to form an unsubstituted or a substituted bicyclic, nitrogen-containing heterocyclyl; R⁴ can be hydrogen, deuterium or halogen; R⁵ can be

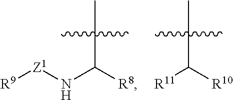

or R¹²; Z¹ can be —C(═O)— or —S(═O)₂—; R⁸ and R¹⁰ can be independently selected from an unsubstituted or a substituted C₁₋₆ alkyl, an unsubstituted or a substituted C₂₋₆ alkenyl, an unsubstituted or a substituted C₂₋₆ alkynyl, an unsubstituted or a substituted monocyclic C₃₋₆ cycloalkyl, an unsubstituted or a substituted bicyclic C₅₋₈ cycloalkyl, an unsubstituted or a substituted monocyclic 4- to 6-membered heterocyclyl and an unsubstituted monocyclic C₃₋₆ cycloalkyl(CH₂)—, wherein when the C₁₋₆ alkyl is substituted, the C₁₋₆ alkyl can be substituted 1, 2, 3 or 4 times with a substituent independently selected from halogen, cyano, an unsubstituted or a substituted monocyclic C₃₋₆ cycloalkyl, an unsubstituted C₁₋₄ alkoxy and an unsubstituted C₁₋₄ haloalkoxy, or the C₁₋₆ alkyl can be substituted 1 to 13 times with deuterium; wherein when the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl are substituted, the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl can be substituted 1, 2, 3 or 4 times with a substituent independently selected from halogen, an unsubstituted C₁₋₄ alkyl, an unsubstituted C₂₋₄ alkenyl, an unsubstituted C₂₋₄ alkynyl, an unsubstituted C₁₋₄ haloalkyl, an unsubstituted or a substituted monocyclic C₃₋₆ cycloalkyl and an unsubstituted C₁₋₄ alkoxy; and R⁹ can be selected from an unsubstituted or a substituted C₁₋₆ alkyl, an unsubstituted or a substituted C₁₋₆ haloalkyl, an unsubstituted or a substituted monocyclic C₃₋₆ cycloalkyl, an unsubstituted or a substituted bicyclic C₅₋₆ cycloalkyl, an unsubstituted or a substituted monocyclic heteroaryl, an unsubstituted or a substituted monocyclic heterocyclyl, an unsubstituted or a substituted alkoxy and —NR¹⁷R¹⁸, wherein the substituted C₁₋₆ alkyl can be substituted 1 or 2 times with an unsubstituted C₁₋₄ alkoxy, wherein the substituted monocyclic C₃₋₆ cycloalkyl can be substituted 1, 2, 3 or 4 times with a substituent independently selected from halogen, an unsubstituted C₁₋₄ alkyl, an unsubstituted C₂₋₄ alkenyl, an unsubstituted C₁₋₄ alkoxy, an unsubstituted C₁₋₄ haloalkyl and an unsubstituted monocyclic C₃₋₆ cycloalkyl, and wherein the substituted C₁₋₆ haloalkyl can be substituted 1 or 2 times with an unsubstituted C₁₋₄ alkoxy; R¹¹ can be an unsubstituted or a substituted monocyclic 4- to 6-membered heterocyclyl, —(NH)ₘ— (an unsubstituted or a substituted 5- to 6-membered monocyclic heteroaryl), —O-(an unsubstituted or a substituted C₁₋₆ alkyl), —O-(an unsubstituted or a substituted C₃₋₈ cycloalkyl) or —O—(C₁₋₄ alkyl)-(an unsubstituted or a substituted C₃₋₈ cycloalkyl), wherein m can be 0 or 1; R¹² can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl), an unsubstituted or a substituted heteroaryl(alkyl), an unsubstituted or a substituted heterocyclyl(alkyl), an unsubstituted or a substituted C-carboxy, —OR¹³, —NR¹⁴R¹⁵ or —C(═O)—NR^{16A}R^{16B}; R¹³ can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl); R¹⁴ and R¹⁵ can be independently selected from hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl); or R¹⁴ and R¹⁵ can be taken together with the nitrogen to which R¹⁴ and R¹⁵ are attached to form an unsubstituted or a substituted 3- to 8-membered heterocyclyl; R^{16A} can be hydrogen or an unsubstituted C₁₋₃ alkyl; R^{16B} can be an unsubstituted or a substituted aryl, an unsubstituted or a substituted heteroaryl or an unsubstituted or a substituted 3- to 8-membered monocyclic heterocyclyl; and R¹⁷ and R¹⁸ can be independently selected from hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted 3-8 membered heterocyclyl, an unsubstituted or a substituted aryl, an unsubstituted or a substituted heteroaryl, an unsubstituted or a substituted aryl(alkyl) and an unsubstituted or a substituted heteroaryl(alkyl); or R¹⁷ and R¹⁸ can be taken together along with the nitrogen to which they are connected to form an unsubstituted or a substituted 3-8 membered heterocyclyl.

The substituent R¹ can be various moieties. In some embodiments, R¹ can be an unsubstituted ketoamide. In some embodiments, R¹ can be a substituted ketoamide. The ketoamide can have the structure —C(═O)—C(═O)NR^{y1}R^{z1}. In some embodiments, R¹ can be an acyl, for example, R¹ can be —C(═O)H, —C(═O)(an unsubstituted C₁₋₄ alkyl), —C(═O)(an unsubstituted or a substituted benzyl), —C(═O)(an unsubstituted or a substituted monocyclic heteroaryl) or —C(═O)(an unsubstituted or a substituted bicyclic heteroaryl). In some embodiments, R¹ can be a substituted acyl. The acyl for R¹ can have the structure —C(═O)R^{y2}. When the acyl is substituted, the possible groups that can be present on the acyl include hydroxy, a substituted or an unsubstituted alkoxy (such as —O-(an unsubstituted C₁₋₄ alkyl), —O-(an unsubstituted C₃₋₆ cycloalkyl), a substituted or an unsubstituted phenoxy or a substituted or an unsubstituted benzyloxy) or —O—(C═O)-(an unsubstituted C₁₋₆ alkyl).

R^{y1}, R^{y2} and R^{z1} can be a variety of groups. In some embodiments, R^{y1}, R^{y2} and R^{z1} can be independently selected from hydrogen, C₁₋₈ alkyl, C₂₋₈ alkenyl, C₂₋₈ alkynyl, C₃₋₈ cycloalkyl (for example, a monocyclic C₃₋₈ cycloalkyl), C₃₋₈ cycloalkenyl (such as a monocyclic C₃₋₈ cycloalkenyl), aryl (such as phenyl or naphthyl), heteroaryl (including a monocyclic or a bicyclic heteroaryl), heterocyclyl (for example, a monocyclic or a bicyclic heterocyclyl), aryl(alkyl) (such as benzyl), heteroaryl(alkyl) (including a monocyclic heteroaryl(CH₂)— and a monocyclic (heteroaryl(CH₂CH₂)—) or heterocyclyl(alkyl) (such as a monocyclic heterocyclyl(CH₂)— and a monocyclic heterocyclyl(CH₂CH₂)—), wherein each of the aforementioned R^{y1}, R^{y2} and R^{z1} groups can be unsubstituted or substituted. In some embodiments, R^{y1}, R^{y2} and R^{z1} can be independently selected from H, C₁₋₈ alkyl, an unsubstituted C₁₋₄ haloalkyl (including —CF₃, —CClF₂, —CHF₂, —C(CH₃)F₂, —CH₂F, —CH(CH₃)F, —CH₂CF₃, —CH(CH₃)CF₃, —CH₂CH₂CF₃, —CH₂CH(CH₃)CF₃, —CF₂CF₃, —CH₂CH₂F and —CH₂CH₂CH₂F), —C₁₋₄ alkyl(OH) (including —CH₂OH, —CH₂CH₂OH and —CH(CH₃)OH), —C₁₋₄ alkyl(C₁₋₄ alkoxy) (such as —CH₂O (an unsubstituted C₁₋₄ alkyl) and —CH₂CH₂O (an unsubstituted C₁₋₄ alkyl)), —C₁₋₄ alkyl-O-(a monocyclic C₃₋₆ cycloalkyl) (such as —CH₂O (a monocyclic C₃₋₆ cycloalkyl), —CH₂CH₂O (a monocyclic C₃₋₆ cycloalkyl)), —C₁₋₄ alkyl-O-(phenyl) (for example, —CH₂O (phenyl) and —CH₂CH₂O (phenyl)), —C₁₋₄ alkyl-O-(5- to 6-membered monocyclic heteroaryl) (such as —CH₂O (5- to 6-membered monocyclic heteroaryl) and —CH₂CH₂O (5- to 6-membered monocyclic heteroaryl)), —C₁₋₄ alkyl-O-(5- to 6-membered monocyclic heterocyclyl) (for example, —CH₂O (5- to 6-membered monocyclic heterocyclyl) and —CH₂CH₂O (5- to 6-membered monocyclic heterocyclyl)), —C₁₋₄ alkyl-O-(a monocyclic C₃₋₆ cycloalkyl(C₁₋₄ alkyl) (such as —C₁₋₄ alkyl-O—CH₂-(monocyclic C₃₋₆ cycloalkyl) and —C₁₋₄ alkyl-O—CH₂CH₂-(monocyclic C₃₋₆ cycloalkyl)), —C₁₋₄ alkyl-O-(benzyl) (for example, —CH₂O (benzyl) and —CH₂CH₂O (benzyl)), —C₁₋₄ alkyl-O-(5- to 6-membered monocyclic heteroaryl(C₁₋₄ alkyl), —C₁₋₄ alkyl-O-(5- to 6-membered monocyclic heterocyclyl(C₁₋₄ alkyl), —C₁₋₄ alkyl-O(C═O)(an unsubstituted C₁₋₆ alkyl) (for example, —CH₂O(C═O)(an unsubstituted C₁₋₆ alkyl)), a monocyclic C₃₋₈ cycloalkyl (such as cyclopropyl, cyclobutyl, cyclopentyl, cyclohexyl, cycloheptyl and cyclooctyl), a monocyclic heteroaryl (such as imidazole, 1,3,4-oxadiazole and pyridinyl), a monocyclic heterocyclyl (for example, tetrahydrofuran and tetrahydropyran), a bicyclic heteroaryl (for example, benzothiazole, benzoimidazole and benzooxazole), a bicyclic heterocyclyl, a monocyclic C₃₋₆ cycloalkyl(alkyl), aryl(alkyl) (such as benzyl), heteroaryl(alkyl) (for example, a monocyclic heteroaryl-(CH₂)—, such as pyridinyl-(CH₂)—) and heterocyclyl(alkyl) (for example, a monocyclic heterocyclyl-(CH₂)—), wherein each of the aforementioned R^{y1}, R^{y2} and R^{z1} groups can be unsubstituted or substituted.

In some embodiments, R¹ can be —C(═O)R^{y2}, wherein R^{y2} can be —C₁₋₄ alkyl(OH) (such as —CH₂OH). In some embodiments, R¹ can be —C(═O)—C(═O)NR^{y1}R^{z1}; wherein R^{y1} can be H; and R^{z1} can be any of the moieties listed for R^{z1} in the previous paragraph. In some embodiments, R¹ can be —C(═O)—C(═O)NR^{y1}R^{z1}; wherein R^{y1} can be H; and R^{z1} can be a monocyclic C₃₋₈ cycloalkyl (for example, cyclopropyl, cyclobutyl, cyclopentyl, cyclohexyl, cycloheptyl and cyclooctyl).

Prodrug-type and phosphate-containing moieties can be present at R¹. In some embodiments, R¹ can be —CH(OH)—(S(═O)₂—OH) or —CH(OH)—(S(═O)₂—O—). Those skilled in the art understand that when R¹ is —CH(OH)—(S(═O)₂—O—), the negative charge can be balanced with a positive ion and form a salt. For example, R¹ can be —CH(OH)—(S(═O)₂—O—)(Na⁺). In other embodiments, R¹ can be —CH(OH)((P═O)(OR⁶)₂), wherein each R⁶ can be independently hydrogen, an unsubstituted C₁₋₆ alkyl, an unsubstituted C₂₋₆ alkenyl, an unsubstituted C₁₋₆ haloalkyl, an unsubstituted or a substituted aryl or an unsubstituted or a substituted aryl(C₁₋₄ alkyl). In still other embodiments, R¹ can be —C(═O)CH₂—O—((P═O)(OR⁷)₂), wherein each R⁷ can be independently hydrogen, an unsubstituted C₁₋₆ alkyl, an unsubstituted C₂₋₆ alkenyl, an unsubstituted C₁₋₆ haloalkyl, an unsubstituted or a substituted aryl or an unsubstituted or a substituted aryl(C₁₋₄ alkyl). Other examples of R⁶ and R⁷ groups include methyl, ethyl, n-propyl, iso-propyl, n-butyl, iso-butyl, sec-butyl, tert-butyl, pentyl (straight-chained and branched), hexyl (straight-chained and branched), ethenyl, propenyl, butenyl, pentenyl, hexenyl, chloromethyl, fluoromethyl, difluoromethyl, dichloromethyl, trifluoromethyl, trichloromethyl, an unsubstituted or a substituted phenyl and an unsubstituted or a substituted benzyl.

In some embodiments, R¹ can be cyano. In other embodiments, R¹ can be an unsubstituted C₂₋₅ alkynyl. In still other embodiments, R¹ can be a substituted C₂₋₅ alkynyl. The C₂₋₅ alkynyl can have various structures. For example, the C₂₋₅ alkynyl can have the structure —(CH₂)₁—C₂₋₄ alkynyl or —(CH₂)₂—C₂₋₃ alkynyl. In some embodiments, R¹ can be an unsubstituted C₂ alkynyl (—CH—CH).

Ring A¹ can be a variety of monocyclic or multicyclic rings. When Ring A¹ is a multicyclic ring moiety, the rings can be connected in a fused and/or spiro-fashion. In some embodiments,

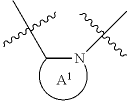

can be unsubstituted. In some embodiments,

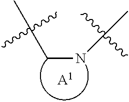

can be substituted. For example,

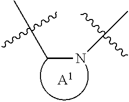

can be substituted with one or more moieties (such as 1, 2 or 3 moieties) independently selected from the group consisting of ═O, ═CH₂, deuterium, halogen, hydroxy, an unsubstituted C₁₋₄ alkyl, an unsubstituted C₁₋₄ haloalkyl, an unsubstituted C₂₋₄ alkenyl, an unsubstituted or a substituted C₃₋₆ monocyclic cycloalkyl and an unsubstituted or a substituted phenyl.

In some embodiments, Ring A¹ can be an unsubstituted or a substituted

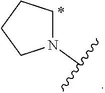 [SMILES: *N1CCCC1]

In other embodiments, Ring A¹ can be an unsubstituted or a substituted

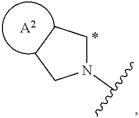

wherein Ring A² can be an unsubstituted or a substituted 3- to 10-membered ring system that optionally includes 1 to 3 heteroatoms selected from O (oxygen), S (sulfur) and N (nitrogen), and wherein Ring A² can be optionally substituted with one or more moieties (such as 1, 2 or 3 moieties) independently selected from ═O, ═CH₂, deuterium, halogen, hydroxy, an unsubstituted C₁₋₄ alkyl, an unsubstituted C₁₋₄ haloalkyl, an unsubstituted C₂₋₄ alkenyl, an unsubstituted or a substituted C₃₋₆ monocyclic cycloalkyl and an unsubstituted or a substituted phenyl; and wherein the N is the nitrogen of Ring A¹ shown in Formula (I) and the carbon indicated with an asterisk is the carbon to which R⁴ is connected.

Examples of

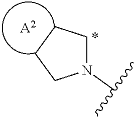

moieties are provided herein. In some embodiments, Ring A¹ can be an unsubstituted or a substituted

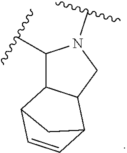 [SMILES: *C1C2C3C=CC(C3)C2CN1*]

In other embodiments, Ring A¹ can be an unsubstituted or a substituted

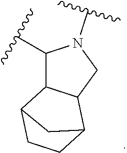 [SMILES: *C1C2C3CCC(C3)C2CN1*]

In still other embodiments, Ring A¹ can be an unsubstituted or a substituted

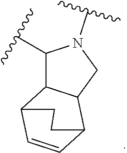 [SMILES: *C1C2C3C=CC(CC3)C2CN1*]

In yet still other embodiments, Ring A¹ can be an unsubstituted or a substituted

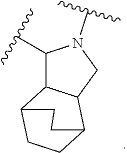 [SMILES: *C1C2C3CCC(CC3)C2CN1*]

In some embodiments, Ring A¹ can be an unsubstituted or a substituted

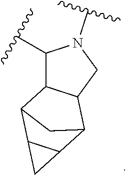 [SMILES: *C1C2C3CC(C4CC43)C2CN1*]

In other embodiments, Ring A¹ can be an unsubstituted or a substituted

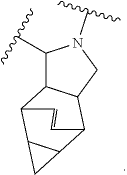 [SMILES: *C1C2C3C=CC(C4CC34)C2CN1*]

In still other embodiments, Ring A¹ can be an unsubstituted or a substituted

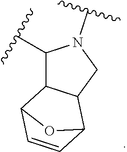 [SMILES: *C1C2C3C=CC(O3)C2CN1*]

In yet still other embodiments, Ring A¹ can be an unsubstituted or a substituted

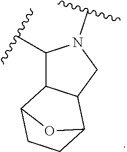 [SMILES: *C1C2C3CCC(O3)C2CN1*]

Those skilled in the art understand that the nitrogen shown in each of the ring structures for Ring A¹ corresponds to the ring nitrogen shown in Formula (I), and the carbon adjacent to the ring nitrogen with the

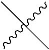

corresponds to the carbon to which R⁴ is attached. For example, those skilled in the art understand that when Ring A¹ is

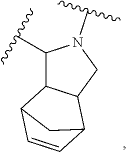 [SMILES: *C1C2C3C=CC(C3)C2CN1*]

then a compound of Formula (I), or a pharmaceutically acceptable salt thereof, can have the following structure:

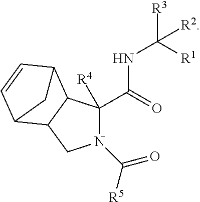

In some embodiments, Ring A¹ can be an unsubstituted or a substituted

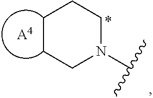

wherein Ring A⁴ can be an unsubstituted or a substituted 3- to 10-membered ring system that optionally includes 1 to 3 heteroatoms selected O (oxygen), S (sulfur) and N (nitrogen), and wherein Ring A⁴ can be optionally substituted with one or more moieties (such as 1, 2 or 3 moieties) independently selected from ═O, ═CH₂, deuterium, halogen, hydroxy, an unsubstituted C₁₋₄ alkyl, an unsubstituted C₁₋₄ haloalkyl, an unsubstituted C₂₋₄ alkenyl, an unsubstituted or a substituted C₃₋₆ monocyclic cycloalkyl and an unsubstituted or a substituted phenyl. The examples of Ring A² provided in the previous paragraph can also be examples of Ring A⁴. For example, Ring A² can be an unsubstituted or a substituted version of the following:

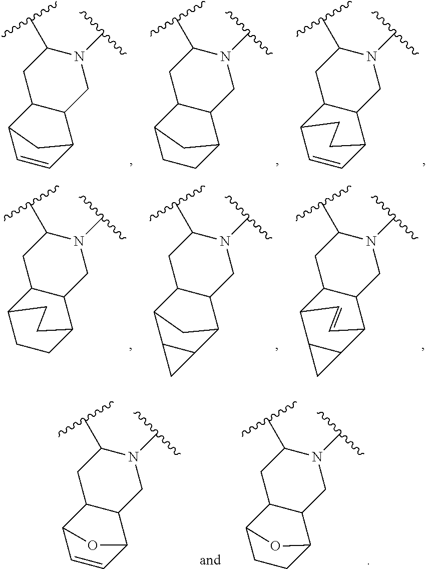

Those skilled in the art understand that the nitrogen shown in each of the ring structures for Ring A¹ corresponds to the ring nitrogen shown in Formula (I), and the carbon adjacent to the ring nitrogen with the

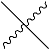

corresponds to the carbon to which R⁴ is attached.

As provided herein, Ring A¹ can be rings connected in a spiro-fashion. In some embodiments, Ring A¹ can be an unsubstituted or a substituted

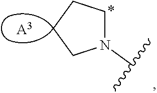

wherein Ring A³ can be an unsubstituted or a substituted monocyclic C₃₋₆ cycloalkyl. In other embodiments, Ring A¹ can be an unsubstituted or a substituted

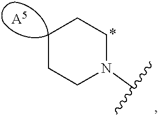

wherein Ring A⁵ can be an unsubstituted or a substituted monocyclic C₃₋₆ cycloalkyl. Examples of monocyclic C₃₋₆ cycloalkyls include cyclopropyl, cyclobutyl, cyclopentyl and cyclohexyl. In some embodiments, Ring A¹ can be selected from:

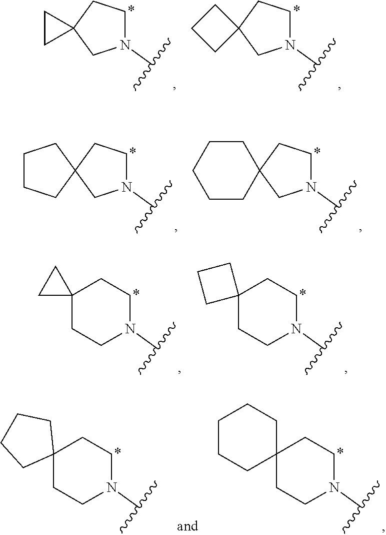

wherein each can be unsubstituted or substituted.

As provided herein, Ring A¹ can be substituted with one or more moieties (such as 1, 2 or 3 moieties) independently selected from ═O, ═CH₂, deuterium, halogen, hydroxy, an unsubstituted C₁₋₄ alkyl, an unsubstituted C₁₋₄ haloalkyl, an unsubstituted C₂₋₄ alkenyl, an unsubstituted or a substituted C₃₋₆ monocyclic cycloalkyl and an unsubstituted or a substituted phenyl. Example of suitable substituents that can be present in Ring A¹ include halogen (such as F or Cl), an unsubstituted C₁₋₄ alkyl (for example, methyl, ethyl, n-propyl, iso-propyl, n-butyl, iso-butyl, sec-butyl and tert-butyl), an unsubstituted C₁₋₄ haloalkyl (including CF₃, —CClF₂, —CHF₂, —C(CH₃)F₂, —CH₂F, —CH(CH₃)F, —CH₂CF₃, —CH(CH₃)CF₃, —CH₂CH₂CF₃, —CH₂CH(CH₃)CF₃, —CF₂CF₃, —CH₂CH₂F and —CH₂CH₂CH₂F), an unsubstituted C₂₋₄ alkenyl (such as ethenyl, propenyl and butenyl), an unsubstituted or a substituted C₃₋₆ monocyclic cycloalkyl (for example, cyclopropyl, cyclobutyl, cyclopentyl and cyclohexyl) and an unsubstituted or a substituted phenyl. When Ring A¹ is substituted by an unsubstituted or a substituted C₃₋₆ monocyclic cycloalkyl, the unsubstituted or a substituted C₃₋₆ monocyclic cycloalkyl can replace one hydrogen. In some embodiments, an unsubstituted or a substituted C₃₋₆ monocyclic cycloalkyl can replace two hydrogens of Ring A¹ such that the unsubstituted or a substituted C₃₋₆ monocyclic cycloalkyl is connected to Ring A in a spiro-fashion. Examples of an unsubstituted or a substituted C₃₋₆ monocyclic cycloalkyl replacing two hydrogen of Ring A¹ includes the following:

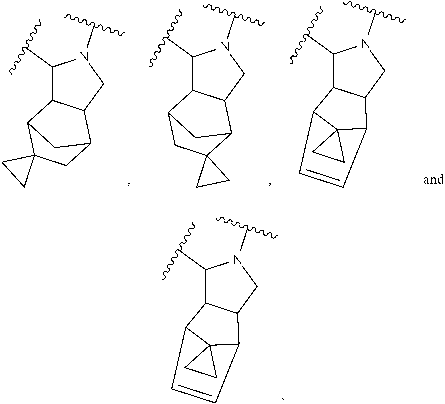 [SMILES: *C1C2C(CN1*)C1CC2C2(CC2)C1 ; *C1C2C3CC(C2CN1*)C1(CC1)C3 ; *C1C2C(CN1*)C1C=CC2C12CC2 ; *C1C2C(CN1*)C1C=CC2C12CC2]

wherein each can be unsubstituted or substituted as described herein. In some embodiments, when Ring A¹ is substituted by a substituted C₃₋₆ monocyclic and/or a substituted phenyl, the C₃₋₆ monocyclic and/or phenyl can be substituted by 1, 2 or 3 moieties selected from halogen and unsubstituted C₁₋₄ alkyl.

In some embodiments,

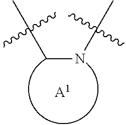

can be selected from:

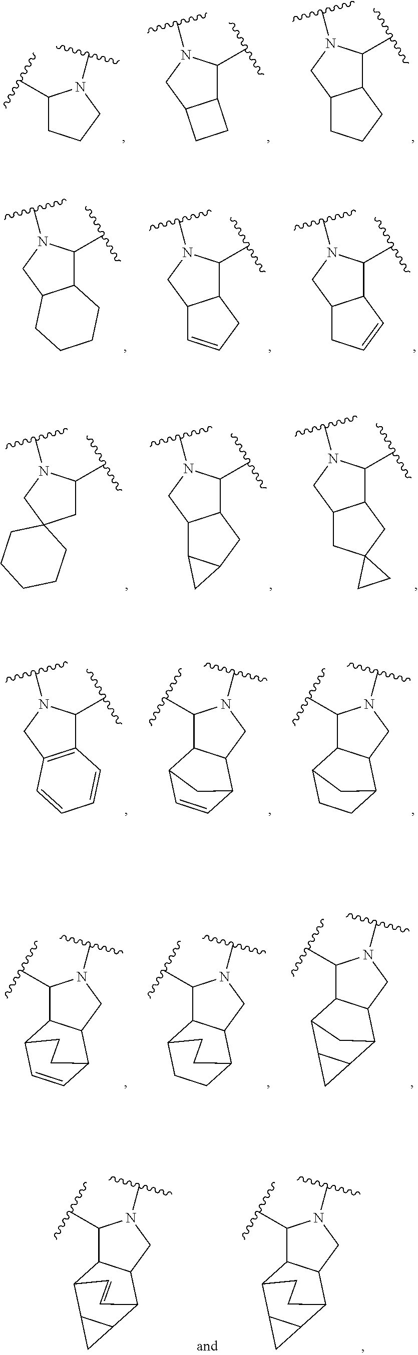

wherein each of the moieties can be unsubstituted or substituted. In some embodiments,

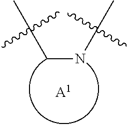

can be selected from:

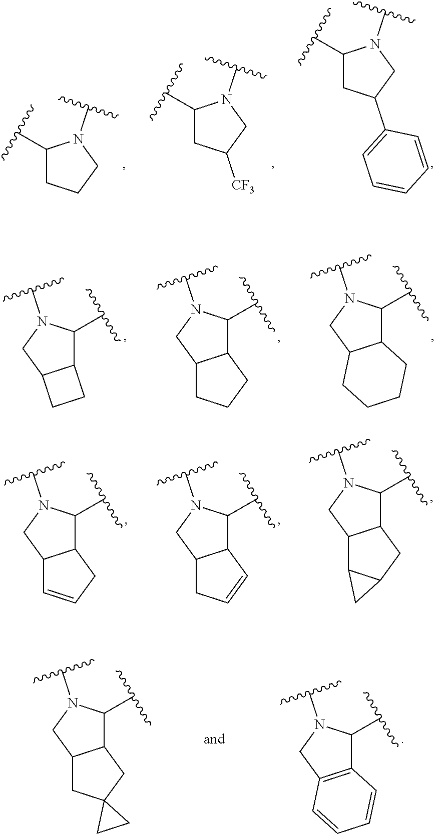

In other embodiments,

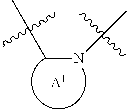

can be selected from:

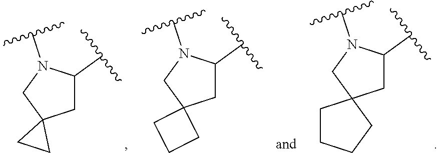 [SMILES: *C1CC2(CC2)CN1* ; *C1CC2(CCC2)CN1* ; *C1CC2(CCCC2)CN1*]

In still other embodiments

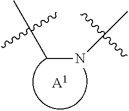

can be

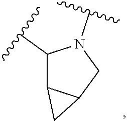 [SMILES: *C1C2CC2CN1*]

wherein

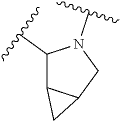 [SMILES: *C1C2CC2CN1*]

can be unsubstituted or substituted (such as

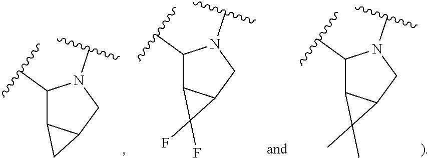

Examples of Ring A¹ include, but are not limited to, the following:

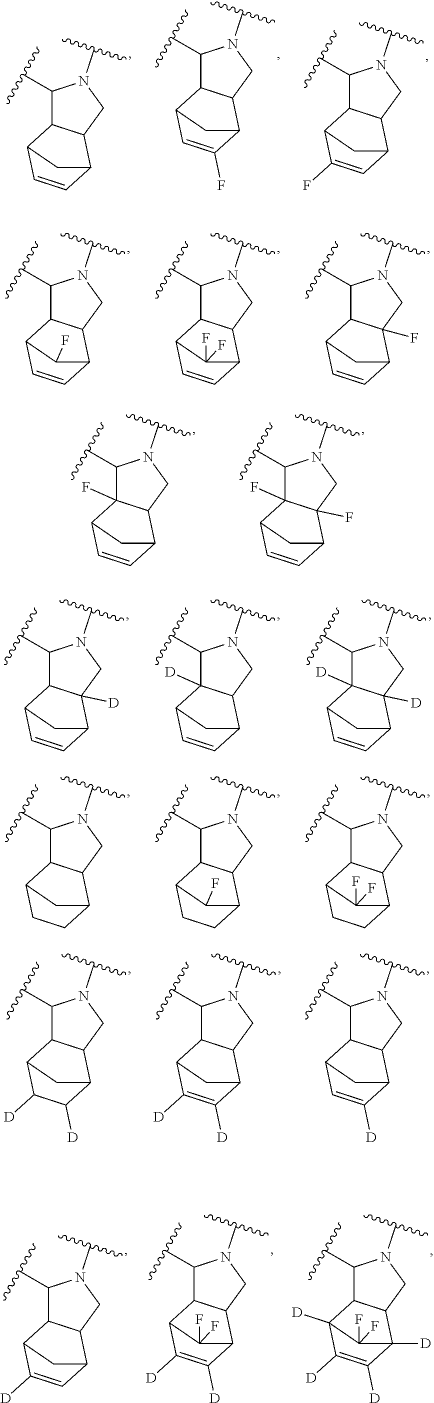

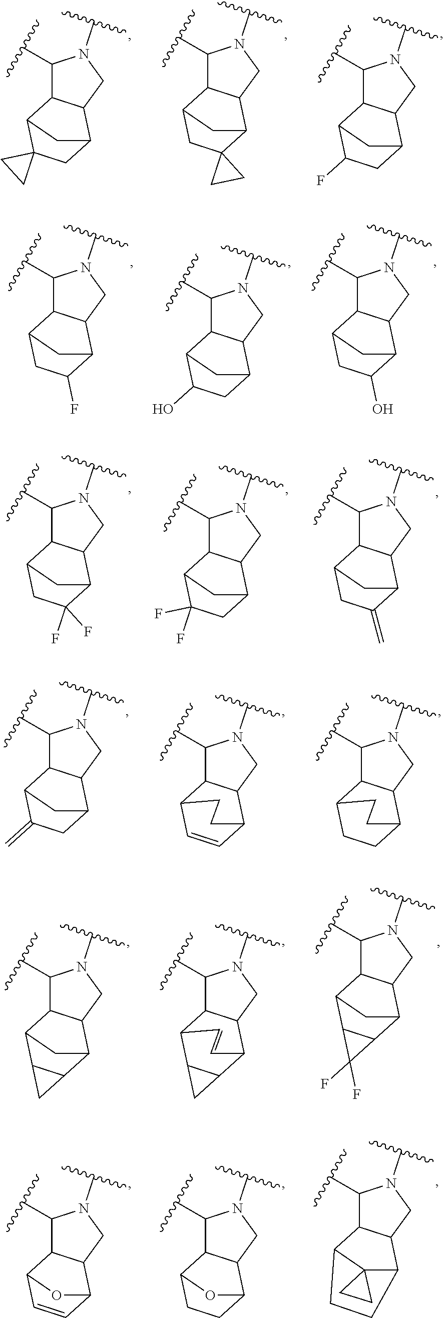

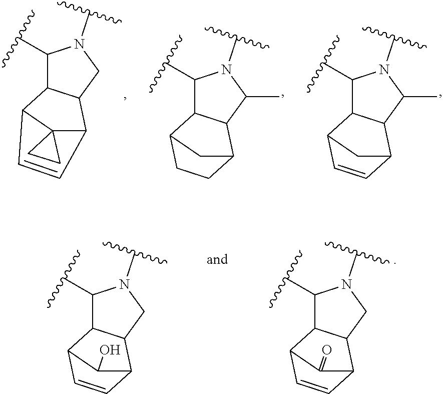 [SMILES: *C1C2C(CN1*)C1C=CC2C12CC2 ; *C1C2C3CCC(C3)C2C(C)N1* ; *C1C2C3C=CC(C3)C2C(C)N1* ; *C1C2C3C=CC(C3O)C2CN1* ; *C1C2C3C=CC(C3=O)C2CN1*]

Additional examples of Ring A¹ are

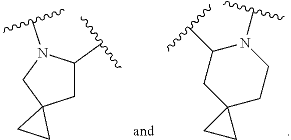 [SMILES: *C1CC2(CC2)CN1* ; *C1CC2(CCN1*)CC2]

In some embodiments, R⁴ can be hydrogen. In other embodiments, R⁴ can be deuterium. In still other embodiments, R⁴ can be halogen (such as fluoro or chloro).

In some embodiments, R² can be hydrogen. In other embodiments, R² can be deuterium. In still other embodiments, R² can be halogen (for example, fluoro or chloro). In yet still other embodiments, R² can be an unsubstituted C₁₋₄ alkyl, such as methyl, ethyl, n-propyl, isopropyl, n-butyl, iso-butyl, sec-butyl and tert-butyl. In some embodiments, R² can be methyl.

When R² is any one of embodiments of the previous paragraph, R³ can be an unsubstituted or a substituted monocyclic nitrogen-containing heteroaryl, an unsubstituted or a substituted bicyclic nitrogen-containing heteroaryl, an unsubstituted or a substituted bicyclic nitrogen-containing heterocyclyl or an unsubstituted or a substituted naphthyl. In some embodiments, R³ can be an unsubstituted or a substituted monocyclic nitrogen-containing heteroaryl. For example, R³ can be an unsubstituted or a substituted 5- or 6-membered, monocyclic nitrogen-containing heteroaryl, such as pyrrole, pyrazole, 1,2,3-triazole, 1,2,4-triazole, tetrazole, pyridine, pyrimidine and pyrazine, wherein each of the aforementioned may be unsubstituted or substituted. In some embodiments, R³ can be an unsubstituted or a substituted bicyclic nitrogen-containing heteroaryl. As an example, R³ can be an unsubstituted or a substituted 9- or 10-membered, bicyclic nitrogen-containing heteroaryl. In some embodiments, R³ can be an unsubstituted or a substituted 6,5 bicyclic nitrogen-containing heteroaryl, an unsubstituted or a substituted 5,6 bicyclic nitrogen-containing heteroaryl or an unsubstituted or a substituted 6,6 bicyclic nitrogen-containing heteroaryl. Exemplary bicyclic nitrogen-containing heteroaryls include imidazo[1,2-a]pyridine, quinoline, isoquinoline, 1,6-naphthyridine, 1,7-naphthyridine, 2,6-naphthyridine, 2,7-naphthyridine, phthalazine, cinnoline, a pyrazolopyridine (such as pyrazolo[3,4-b]pyridine, pyrazolo[3,4-c]pyridine, pyrazolo[4,3-c]pyridine, pyrazolo[4,3-b]pyridine and pyrazolo[1,5-a]pyridine), [1,2,4]triazolo[1,5-a]pyrazine, a pyrrolopyridine (for example, pyrrolo[1,2-a]pyrazine, pyrrolo[1,2-a]pyrimidine and pyrrolo[1,2-c]pyrimidine), a pyrrolopyridine, 2H-indazole, [1,2,4]triazolo[4,3-a]pyridine and an imidazopyridine (such as imidazo[1,2-a]pyridine, imidazo[1,5-a]pyridine, imidazo[4,5-b]pyridine and imidazo[4,5-c]pyridine) (including unsubstituted or substituted versions thereof). In still other embodiments, R³ can be an unsubstituted or a substituted bicyclic nitrogen-containing heterocyclyl. For example, the unsubstituted or substituted bicyclic nitrogen-containing heterocyclyl can be an unsubstituted or a substituted 9- or 10-membered, bicyclic nitrogen-containing heterocyclyl. In some embodiments, R³ can be an unsubstituted or a substituted 6,5 bicyclic nitrogen-containing heterocyclyl, an unsubstituted or a substituted 5,6 bicyclic nitrogen-containing heterocyclyl or an unsubstituted or a substituted 6,6 bicyclic nitrogen-containing heterocyclyl. Exemplary bicyclic nitrogen-containing heterocyclyl include 6,7-dihydro-5H-cyclopenta[c]pyridine, 5,6,7,8-tetrahydroisoquinoline, 6,7-dihydro-5H-cyclopenta[c]pyridazine and 5,6,7,8-tetrahydrocinnoline. The bicyclic nitrogen-containing heteroaryl and/or bicyclic nitrogen-containing heterocyclyl can include a nitrogen at a connection point between the two rings. In yet still other embodiments, R³ can be an unsubstituted or a substituted naphthyl. Example of R³ groups include, but are not limited to, the following:

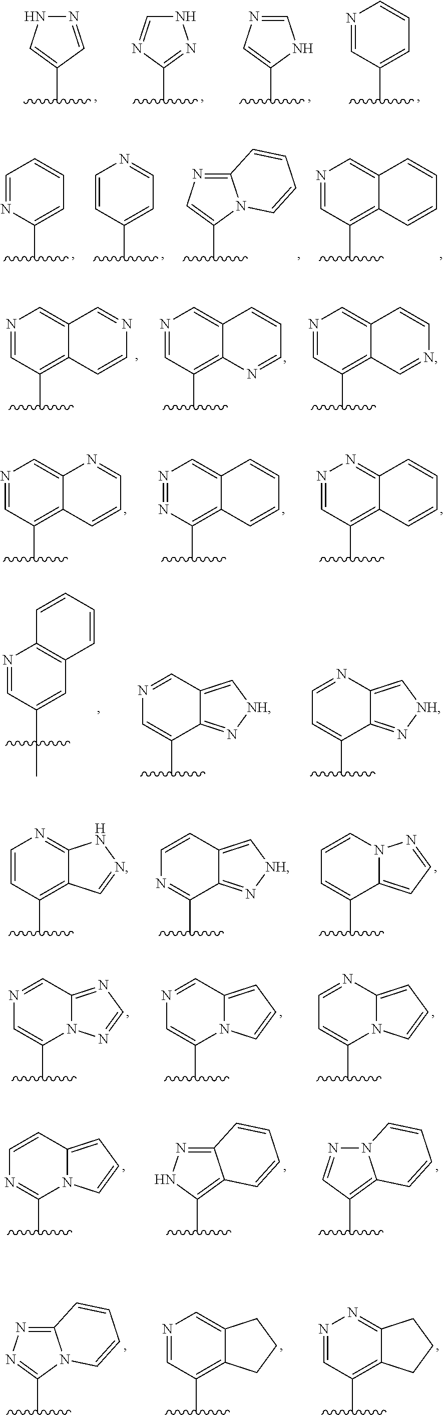

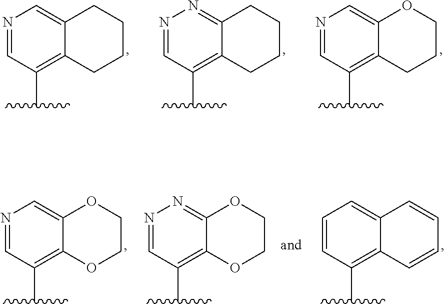

wherein each of the moieties is unsubstituted or substituted.

In some embodiments, R² and R³ can be taken together along with the carbon to which they are attached to form an unsubstituted or a substituted bicyclic, nitrogen-containing heterocyclyl. In some embodiments, R² and R³ can be taken together along with the carbon to which they are attached to form an unsubstituted 9- or 10-membered, bicyclic, nitrogen-containing heterocyclyl. In other embodiments, R² and R³ can be taken together along with the carbon to which they are attached to form a substituted 9- or 10-membered, bicyclic, nitrogen-containing heterocyclyl. A limiting list of examples of an unsubstituted or a substituted bicyclic, nitrogen-containing heterocyclyls include 6,7-dihydro-5H-cyclopenta[c]pyridine (for example,

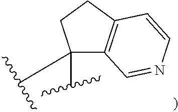 [SMILES: *C1(*)CCc2ccncc21]

and 5,6,7,8-tetrahydroisoquinoline (for example,

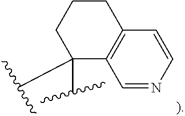 [SMILES: *C1(*)CCCc2ccncc21]

When R³ is substituted, R³ can be substituted with a variety of substituents including those provided for “optionally substituted.” The bicyclic, nitrogen-containing heterocyclyl that is formed by taking R² and R³ together along with the carbon to which they are attached can be also substituted by a variety of substituents including those provided for “optionally substituted.” The number of substituents that can be present can vary. In some embodiments, R³ can be substituted with 1, 2, 3 or 4 substituents. In some embodiments, the bicyclic, nitrogen-containing heterocyclyl that is formed by taking R² and R³ together along with the carbon to which they are attached can be substituents with 1, 2, 3 or 4 substituents. Examples of substituents that can be present on a substituted R³ and/or a substituted bicyclic, nitrogen-containing heterocyclyl that is formed by taking R² and R³ together along with the carbon to which they are attached include halogen (chloro and fluoro), an unsubstituted C₁₋₄ alkyl (for example, methyl, ethyl, n-propyl, isopropyl, n-butyl, iso-butyl, sec-butyl and tert-butyl), an unsubstituted C₁₋₄ alkoxy (such as methoxy, ethoxy, n-propoxy, isopropoxy, n-butoxy, iso-butoxy, sec-butoxy and tert-butoxy), an unsubstituted C₁₋₄ haloalkyl (for example, —CF₃, —CClF₂, —CHF₂, —C(CH₃)F₂, —CH₂F, —CH(CH₃)F, —CH₂CF₃, —CH(CH₃)CF₃, —CH₂CH₂CF₃, —CH₂CH(CH₃)CF₃, —CF₂CF₃, —CH₂CH₂F and —CH₂CH₂CH₂F) and an unsubstituted phenyl.

Non-limiting examples of R³ groups include, but are not limited to, the following:

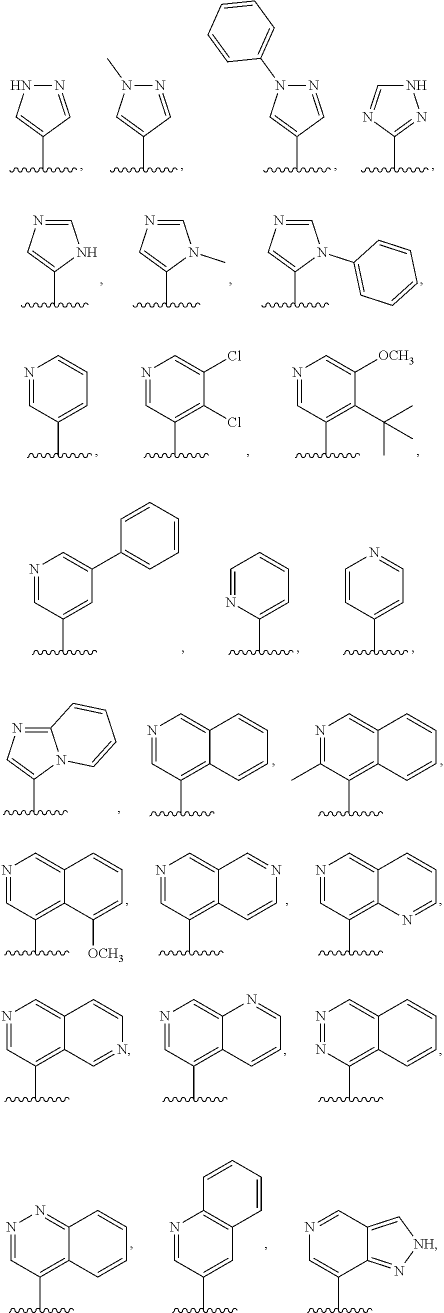

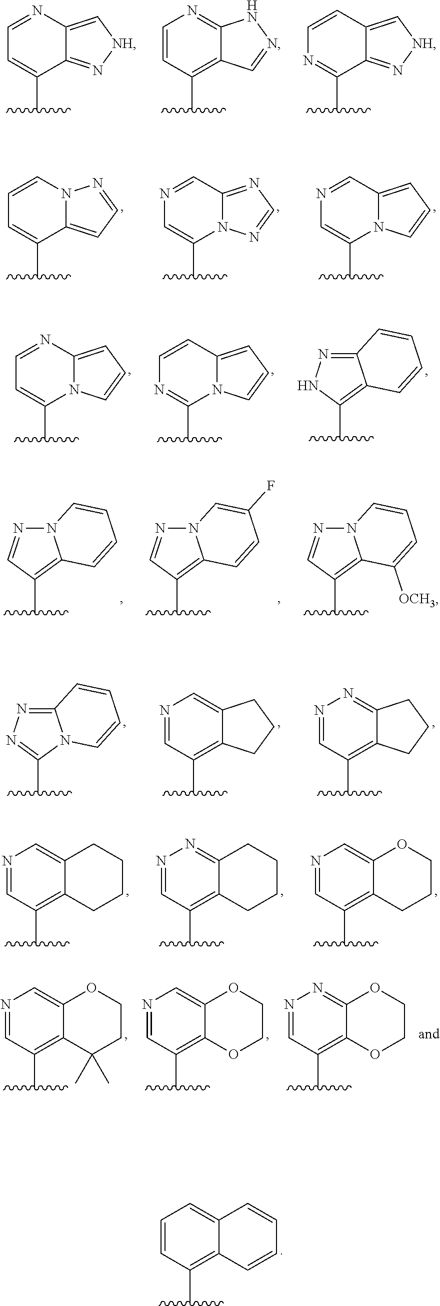

In some embodiments, R^{N} can be hydrogen. In other embodiments, R^{N} can be deuterium. In still other embodiments, R^{N} can be an unsubstituted C₁₋₆ alkyl. For example, R^{N} can be methyl, ethyl, n-propyl, iso-propyl, n-butyl, iso-butyl, sec-butyl, tert-butyl, pentyl (straight-chained and/or branched) or hexyl (straight-chained and/or branched). In some embodiments, R^{N} can be methyl. In yet still other embodiments, R^{N} can be a substituted C₁₋₆ alkyl. When R^{N} is a substituted C₁₋₆ alkyl, the C₁₋₆ alkyl can be substituted one or more times (such as 1, 2, 3, 4, 5 or 6 times) with substituent(s) independently selected from those provided for “optionally substituted.” In some embodiments, R^{N} can be a C₁₋₆ alkyl substituted 1, 2, 3, 4, 5 or 6 times with a halogen (such as chloro and/or fluoro).

As provided herein, R⁵ can be

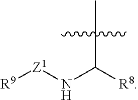

In some embodiments, R⁵ can be

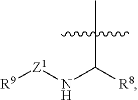

wherein Z¹ is —C(═O)— such that R⁵ can be

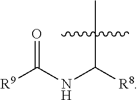

In some embodiments, R⁵ can be

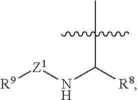

wherein Z¹ is —S(═O)₂— such that R⁵ can be

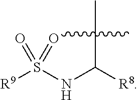

In some embodiments, R⁹ can be an unsubstituted C₁₋₆ haloalkyl. For example, R⁹ can be —CF₃, —CClF₂, —CHF₂, —C(CH₃)F₂, —CH₂F, —CH(CH₃)F, —CH₂CF₃, —CH(CH₃)CF₃, —CH₂CH₂CF₃, —CH₂CH(CH₃)CF₃, —CF₂CF₃, —CH₂CH₂F and —CH₂CH₂CH₂F. In some embodiments, R⁹ can be —CF₃. In other embodiments, R⁹ can be a substituted C₁₋₆ haloalkyl where the C₁₋₆ haloalkyl can be substituted 1 or 2 times with an unsubstituted C₁₋₄ alkoxy. When the C₁₋₆ haloalkyl is substituted with 1 or 2 unsubstituted C₁₋₄ alkoxys, one or more hydrogens of the C₁₋₆ haloalkyl (for example, 1, 2 or 3 hydrogens) can be replaced with an unsubstituted C₁₋₄ alkoxy (such as methoxy, ethoxy, n-propoxy, isopropoxy, n-butoxy, isobutoxy, sec-butoxy and tert-butoxy). Exemplary C₁₋₆ haloalkyls substituted with an unsubstituted C₁₋₄ alkoxy include —C(OCH₃)F₂, —CH(OCH₃)F, —C(OCH₃)(CH₃)F, —CH(OCH₃)CF₃, —C(OCH₃)(CH₃)CF₃, —CH₂CH(OCH₃)CF₃, —CH₂C(OCH₃)(CH₃)CF₃, —CH₂CH(OCH₃)F and —CH₂CH₂CH(OCH₃)F. In still other embodiments, R⁹ can be an unsubstituted C₁₋₆ alkyl, such as methyl, ethyl, n-propyl, iso-propyl, n-butyl, iso-butyl, sec-butyl, tert-butyl, pentyl (straight-chained or branched) and hexyl (straight-chained or branched). In yet still other embodiments, R⁹ can be a C₁₋₆ alkyl substituted 1 or 2 times with an unsubstituted C₁₋₄ alkoxy. When the C₁₋₆ alkyl is substituted with an unsubstituted C₁₋₄ alkoxy, a hydrogen of the C₁₋₆ alkyl can be replaced with an unsubstituted C₁₋₄ alkoxy such as those described herein. A non-limiting list of C₁₋₆ alkyls substituted 1 or 2 times with an unsubstituted C₁₋₄ alkoxy include —CH₂(OCH₃), —CH(OCH₃)₂, —CH(CH₃)(OCH₃) and —C(CH₃)₂(OCH₃). In some embodiments, R⁹ can be an unsubstituted or a substituted monocyclic heteroaryl. A variety of an unsubstituted or a substituted monocyclic heteroaryls can be present for R⁹. For example, the heteroaryl can be a 5- or 6-membered heteroaryl that includes 1, 2 or 3 heteroatoms selected from nitrogen (N), oxygen (O) and sulfur (S). Exemplary heteroaryls for an unsubstituted or a substituted monocyclic heteroaryl include, but are not limited to, furane, isoxazole, isothiazole, pyrrole, pyrazole, oxazole, thiazole, 1,2,3-triazole, 1,2,4-triazole, imidazole, 1,3,4-oxadiazole, 1,3,4-thiadiazole, pyridine, pyridazine, pyrimidine and pyrazine. In yet still other embodiments, R⁹ can be an unsubstituted or a substituted monocyclic heterocyclyl. A non-limiting list of monocyclic heterocyclyls for R⁹ include azetidine, oxetane, thietane, tetrahydrofuran, tetrahydrothiophene, pyrrolidine, oxazolidin-2-one, imidazolidin-2-one, tetrahydropyran, tetrahydrothiopyran, piperidine, piperazine, morpholine and thiomorpholine. Various substituents can be present on a substituted heteroaryl and/or a substituted heterocyclyl of R⁹. For example, the heteroaryl can be substituted 1, 2 or 3 times with a moiety selected from halogen, an unsubstituted C₁₋₆ alkyl, an unsubstituted C₁₋₆ haloalkyl and an unsubstituted C₁₋₆ alkoxy. Suitable halogens, unsubstituted C₁₋₆ alkyls, unsubstituted C₁₋₆ haloalkyls and unsubstituted C₁₋₆ alkoxys are described herein.

In some embodiments, R⁹ can be an unsubstituted monocyclic C₃₋₆ cycloalkyl, such as cyclopropyl, cyclobutyl, cyclopentyl and cyclohexyl. In other embodiments, R⁹ can be a halogen-substituted monocyclic C₃₋₆ cycloalkyl. In still other embodiments, R⁹ can be a monocyclic C₃₋₆ cycloalkyl substituted with an unsubstituted C₁₋₄ alkyl. In yet still other embodiments, R⁹ can be a monocyclic C₃₋₆ cycloalkyl substituted with an unsubstituted C₂₋₄ alkenyl. In yet still other embodiments, R⁹ can be a monocyclic C₃₋₆ cycloalkyl substituted with an unsubstituted C₁₋₄ alkoxy. In other embodiments, R⁹ can be a monocyclic C₃₋₆ cycloalkyl substituted with an unsubstituted C₁₋₄ haloalkyl. In still other embodiments, R⁹ can be a monocyclic C₃₋₆ cycloalkyl substituted with an unsubstituted monocyclic C₃₋₆ cycloalkyl, such as cyclopropyl, cyclobutyl, cyclopentyl or cyclohexyl. In some embodiments, R⁹ can be an unsubstituted bicyclic C₅₋₆ cycloalkyl. In other embodiments, R⁹ can be a substituted bicyclic C₅₋₆ cycloalkyl. The two rings of a bicyclic C₅₋₆ cycloalkyl can be connected in a spiro-fashion or a fused-fashion. In some embodiments, R⁹ can be a halogen-substituted bicyclic C₅₋₆ cycloalkyl. In still other embodiments, R⁹ can be a bicyclic C₅₋₆ cycloalkyl substituted with an unsubstituted C₁₋₄ alkyl. In some embodiments, R⁹ can be a bicyclic C₅₋₆ cycloalkyl substituted with an unsubstituted C₂₋₄ alkenyl. In yet still other embodiments, R⁹ can be a bicyclic C₅₋₆ cycloalkyl substituted with an unsubstituted C₁₋₄ alkoxy. In other embodiments, R⁹ can be a bicyclic C₅₋₆ cycloalkyl substituted with an unsubstituted C₁₋₄ haloalkyl. In still other embodiments, R⁹ can be a bicyclic C₅₋₆ cycloalkyl substituted with an unsubstituted monocyclic C₃₋₆ cycloalkyl (including cyclopropyl, cyclobutyl, cyclopentyl and cyclohexyl). A non-liming list of bicyclic C₅₋₆ cycloalkyls include spiro[2.2]pentane, spiro[2.3]hexane, bicyclo[1.1.1]pentane and bicyclo[2.1.1]hexane.

For R⁹, suitable halogen-substituted monocyclic C₃₋₆ cycloalkyls include halogen-substituted cyclopropyl, halogen-substituted cyclobutyl, halogen-substituted cyclopentyl and halogen-substituted cyclohexyl. Additional monocyclic C₃₋₆ cycloalkyls include cyclopropyl substituted with an unsubstituted C₁₋₄ alkyl, an unsubstituted C₂₋₄ alkenyl, an unsubstituted C₁₋₄ alkoxy, an unsubstituted C₁₋₄ haloalkyl and/or an unsubstituted monocyclic C₃₋₆ cycloalkyl, cyclobutyl substituted with an unsubstituted C₁₋₄ alkyl, an unsubstituted C₁₋₄ alkoxy, an unsubstituted C₂₋₄ alkenyl, an unsubstituted C₁₋₄ haloalkyl and/or an unsubstituted monocyclic C₃₋₆ cycloalkyl, cyclopentyl substituted with an unsubstituted C₁₋₄ alkyl, an unsubstituted C₁₋₄ alkoxy, an unsubstituted C₂₋₄ alkenyl, an unsubstituted C₁₋₄ haloalkyl and/or an unsubstituted monocyclic C₃₋₆ cycloalkyl and cyclohexyl substituted with an unsubstituted C₁₋₄ alkyl, an unsubstituted C₁₋₄ alkoxy, an unsubstituted C₂₋₄ alkenyl, an unsubstituted C₁₋₄ haloalkyl and/or an unsubstituted monocyclic C₃₋₆ cycloalkyl. The number halogens on a halogen-substituted monocyclic C₃₋₆ cycloalkyl and/or a bicyclic C₅₋₆ cycloalkyl, the number of unsubstituted C₁₋₄ alkyls on a monocyclic C₃₋₆ cycloalkyl and/or a bicyclic C₅₋₆ cycloalkyl, the number of unsubstituted C₁₋₄ alkoxys on a monocyclic C₃₋₆ cycloalkyl and/or a bicyclic C₅₋₆ cycloalkyl, the number of unsubstituted C₂₋₄ alkenyls on a monocyclic C₃₋₆ cycloalkyl and/or a bicyclic C₅₋₆ cycloalkyl, the number of unsubstituted C₁₋₄ haloalkyls on a monocyclic C₃₋₆ cycloalkyl and/or a bicyclic C₅₋₆ cycloalkyl and the number of unsubstituted monocyclic C₃₋₆ cycloalkyls on a monocyclic C₃₋₆ cycloalkyl and/or a bicyclic C₅₋₆ cycloalkyl can vary. For example, 1, 2, 3 or 4 halogens can be present on a halogen-substituted monocyclic C₃₋₆ cycloalkyl, 1, 2, 3 or 4 unsubstituted C₁₋₄ alkyls can be present on a monocyclic C₃₋₆ cycloalkyl substituted with an unsubstituted C₁₋₄ alkyl, 1, 2, 3 or 4 unsubstituted C₁₋₄ alkoxys can be present on a monocyclic C₃₋₆ cycloalkyl substituted with an unsubstituted C₁₋₄ alkoxy, 1, 2, 3 or 4 unsubstituted C₂₋₄ alkenyls can be present on a monocyclic C₃₋₆ cycloalkyl substituted with an unsubstituted C₂₋₄ alkenyl, 1, 2, 3 or 4 unsubstituted C₁₋₄ haloalkyls can be present on a monocyclic C₃₋₆ cycloalkyl substituted with an unsubstituted C₁₋₄ haloalkyl, 1 or 2 unsubstituted monocyclic C₃₋₆ cycloalkyls can be present on a monocyclic C₃₋₆ cycloalkyl, 1, 2, 3 or 4 halogens can be present on a halogen-substituted bicyclic C₅₋₆ cycloalkyl, 1, 2, 3 or 4 unsubstituted C₁₋₄ alkyls can be present on a bicyclic C₅₋₆ cycloalkyl substituted with an unsubstituted C₁₋₄ alkyl, 1, 2, 3 or 4 unsubstituted C₁₋₄ alkoxys can be present on a bicyclic C₅₋₆ cycloalkyl substituted with an unsubstituted C₂₋₄ alkoxy, 1, 2, 3 or 4 unsubstituted C₂₋₄ alkenyls can be present on a bicyclic C₅₋₆ cycloalkyl substituted with an unsubstituted C₂₋₄ alkenyl, 1, 2, 3 or 4 unsubstituted C₁₋₄ haloalkyls can be present on a bicyclic C₅₋₆ cycloalkyl substituted with an unsubstituted C₁₋₄ haloalkyl and 1 or 2 unsubstituted monocyclic C₃₋₆ cycloalkyls can be present on a bicyclic C₅₋₆ cycloalkyl. In some embodiments, a monocyclic C₃₋₆ cycloalkyl can be substituted with 1 or more substituents (such as 1, 2, 3 or 4 substituents) selected from halogen, an unsubstituted C₁₋₄ alkyl, an unsubstituted C₁₋₄ alkoxy, an unsubstituted C₂₋₄ alkenyl, and an unsubstituted C₁₋₄ haloalkyl. In other embodiments, a bicyclic C₅₋₆ cycloalkyl can be substituted with 1 or more substituents (such as 1, 2, 3 or 4 substituents) selected from halogen, an unsubstituted C₁₋₄ alkyl, an unsubstituted C₁₋₄ alkoxy an unsubstituted C₂₋₄ alkenyl, and an unsubstituted C₁₋₄ haloalkyl. Suitable halogens that can be present on a substituted monocyclic C₃₋₆ cycloalkyl include, but are not limited to, fluoro (F) and chloro (Cl). Examples of unsubstituted C₁₋₄ haloalkyls include, but are not limited to, —CF₃, —CClF₂, —CHF₂, —C(CH₃)F₂, —CH₂F, —CH(CH₃)F, —CH₂CF₃, —CH(CH₃)CF₃, —CH₂CH₂CF₃, —CH₂CH(CH₃)CF₃, —CF₂CF₃, —CH₂CH₂F and —CH₂CH₂CH₂F.

In some embodiments, R⁹ can be an unsubstituted alkoxy. In other embodiments, R⁹ can be a substituted alkoxy. Various alkoxys can be present for R⁹. For example, —O-(hydrocarbon) (such as —O—(C₁₋₈ alkyl)), —O-(monocyclic C₃₋₈ cycloalkyl), —O-(bicyclic C₅₋₈ cycloalkyl), —O-(phenyl), —O-(bicyclic aryl), —O-(monocyclic heteroaryl), —O-(bicyclic heteroaryl), —O-(monocyclic heterocyclyl) and —O-(bicyclic heterocyclyl). A non-limiting list of examples of C₁₋₆ alkoxys are methoxy, ethoxy, n-propoxy, iso-propoxy, n-butoxy, iso-butoxy, sec-butoxy, tert-butoxy, pentoxy (straight-chained or branched), hexoxy (straight-chained or branched), —O-cyclopropyl, —O-cyclobutyl, —O-cyclopentyl, —O— cyclohexyl and —O-(bicyclo[1.1.1]pentyl). In some embodiments, R⁹ can be —O-(an unsubstituted or a substituted C₁₋₈ alkyl). In some embodiments, R⁹ can be —O-(an unsubstituted C₁₋₄ alkyl). A variety of substituents can be present on a substituted alkoxy for R⁹. Examples of suitable substituents are those provided for “optionally substituted.” In some embodiments, 1, 2, 3 or 4 substituents can be present on a substituted alkoxy. For example, a substituted alkoxy can be substituted 1, 2, 3 or 4 times with substituents independently selected from halogen (for example, F or Cl), hydroxy, an unsubstituted C₁₋₄ alkyl and an unsubstituted C₁₋₄ haloalkyl.

In some embodiments, R⁹ can be an amino or an amine, such as —NR¹⁷R¹⁸, wherein R¹⁷ and R¹⁸ can be independently selected from hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted 3-8 membered heterocyclyl, an unsubstituted or a substituted aryl, an unsubstituted or a substituted heteroaryl, an unsubstituted or a substituted aryl(alkyl) and an unsubstituted or a substituted heteroaryl(alkyl). In other embodiments, R⁹ can be —NR¹⁷R¹⁸, wherein R¹⁷ and R¹⁸ are taken together along with the nitrogen to which they are connected to form an unsubstituted or a substituted 3-8 membered heterocyclyl.

In some embodiments, R¹⁷ and/or R¹⁸ can be an unsubstituted C₁₋₈ alkyl. In other embodiments, R¹⁷ and/or R¹⁸ can be a substituted C₁₋₈ alkyl. In still other embodiments, R¹⁷ and/or R¹⁸ can be an unsubstituted C₂₋₈ alkenyl. In yet still other embodiments, R¹⁷ and/or R¹⁸ can be a substituted C₂₋₈ alkenyl. In some embodiments, R¹⁷ and/or R¹⁸ can be an unsubstituted C₂₋₈ alkynyl. In other embodiments, R¹⁷ and/or R¹⁸ can be a substituted C₂₋₈ alkynyl. In still other embodiments, R¹⁷ and/or R¹⁸ can be an unsubstituted C₃₋₆ cycloalkyl, for example an unsubstituted monocyclic C₃₋₈ cycloalkyl. In yet still other embodiments, R¹⁷ and/or R¹⁸ can be a substituted C₃₋₈ cycloalkyl, for example a substituted monocyclic C₃₋₆ cycloalkyl. Various cyclic moieties can be present for R¹⁷ and/or R¹⁸. In some embodiments, R¹⁷ and/or R¹⁸ can be an unsubstituted aryl. In other embodiments, R¹⁷ and/or R¹⁸ can be a substituted aryl. In still other embodiments, R¹⁷ and/or R¹⁸ can be an unsubstituted heteroaryl. In yet still other embodiments, R¹⁷ and/or R¹⁸ can be a substituted heteroaryl. In some embodiments, R¹⁷ and/or R¹⁸ can be an unsubstituted 3- to 8-membered monocyclic heterocyclyl. In other embodiments, R¹⁷ and/or R¹⁸ can be a substituted 3- to 8-membered monocyclic heterocyclyl. In still other embodiments, R¹⁷ and/or R¹⁸ can be an unsubstituted aryl(alkyl). In yet still other embodiments, R¹⁷ and/or R¹⁸ can be a substituted aryl(alkyl). In some embodiments, R¹⁷ and/or R¹⁸ can be an unsubstituted heteroaryl(alkyl). In other embodiments, R¹⁷ and/or R¹⁸ can be a substituted heteroaryl(alkyl). The aryl, heteroaryl and heterocyclyl can be monocyclic or bicyclic, and include 1, 2, 3, 4 or 5 heteroatoms independently selected from O (oxygen), S (sulfur) and N (nitrogen). When R¹⁷ and/or R¹⁸ is aryl(alkyl) or heteroaryl(alkyl), the alkyl linker can be 1, 2 or 3 alkylene groups, such as —CH₂—, —CH₂CH₂— and —CH₂CH₂CH₂—. In some embodiments, R⁹ can be —NHR¹⁸, wherein R¹⁸ can be as provided herein. For example, In some embodiments, R⁹ can be —NHR¹⁸, wherein R¹⁸ can be an unsubstituted C₁₋₈ alkyl.

In some embodiments, R⁵ can be

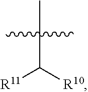

wherein R¹⁰ can be independently selected from an unsubstituted or a substituted C₁₋₆ alkyl, an unsubstituted or a substituted C₂₋₆ alkenyl, an unsubstituted or a substituted C₂₋₆ alkynyl, an unsubstituted or a substituted monocyclic C₃₋₆ cycloalkyl, an unsubstituted or a substituted bicyclic C₅₋₈ cycloalkyl and an unsubstituted or a substituted monocyclic 4- to 6-membered heterocyclyl, wherein when the C₁₋₆ alkyl is substituted, the C₁₋₆ alkyl can be substituted 1, 2, 3 or 4 times with a substituent independently selected from halogen, cyano, an unsubstituted or a substituted monocyclic C₃₋₆ cycloalkyl, an unsubstituted C₁₋₄ alkoxy and an unsubstituted C₁₋₄ haloalkoxy, wherein when the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl are substituted, the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl can be substituted 1, 2, 3 or 4 times with a substituent independently selected from halogen, an unsubstituted C₁₋₄ alkyl, an unsubstituted C₂₋₄ alkenyl, an unsubstituted C₂₋₄ alkynyl, an unsubstituted C₁₋₄ haloalkyl and an unsubstituted C₁₋₄ alkoxy; and R¹¹ can be —(NH)ₘ-(an unsubstituted or a substituted 5- to 6-membered monocyclic heteroaryl), wherein m can be 0 or 1. In some embodiments, R¹¹ can be an unsubstituted or a substituted monocyclic 4- to 6-membered heterocyclyl. Examples of heterocyclyls for R¹¹ include an unsubstituted or a substituted 4- to 6-membered monocyclic heterocyclyls that include 1, 2 or 3 heteroatoms independently selected from N (nitrogen), O (oxygen) and S (sulfur). A non-limiting list of heterocyclyl for R¹¹ include the following: azetidine, oxetane, thietane, tetrahydrofuran, tetrahydrothiophene, pyrrolidine, oxazolidin-2-one, imidazolidin-2-one, tetrahydropyran, tetrahydrothiopyran, piperidine, piperazine, morpholine and thiomorpholine. In other embodiments, m can be 0; and R¹¹ can be an unsubstituted 5- to 6-membered monocyclic heteroaryl. In other embodiments, m can be 0; and R¹¹ can be a substituted 5- to 6-membered monocyclic heteroaryl. In still other embodiment, m can be 1; and R¹¹ can be an —(NH)-(an unsubstituted 5- to 6-membered monocyclic heteroaryl). In other embodiments, m can be 1; and R¹¹ can be a —(NH)-(a substituted 5- to 6-membered monocyclic heteroaryl). An example of a 5- to 6-membered monocyclic heteroaryl that can be present for R¹¹ include a 5- to 6-membered monocyclic heteroaryl that includes 1, 2 or 3 heteroatoms independently selected from N (nitrogen), O (oxygen) and S (sulfur). Examples of suitable 5- to 6-membered monocyclic heteroaryls include, but are not limited to, furane, isoxazole, isothiazole, pyrrole, pyrazole, oxazole, thiazole, 1,2,3-triazole, 1,2,4-triazole, imidazole, 1,3,4-oxadiazole, 1,3,4-thiadiazole, pyridine, pyridazine, pyrimidine, pyridazine and pyrazine. In still other embodiments, R¹¹ can be —O-(an unsubstituted or a substituted C₁₋₆ alkyl). In yet still other embodiments, R¹¹ can be —O-(an unsubstituted or a substituted C₃₋₈ cycloalkyl). In some embodiments, R¹¹ can be —O—(C₁₋₄ alkyl)-(an unsubstituted or a substituted C₃₋₈ cycloalkyl). The cycloalkyl of —O-(an unsubstituted or a substituted C₃₋₈ cycloalkyl) and —O—(C₁₋₄ alkyl)-(an unsubstituted or a substituted C₃₋₈ cycloalkyl) can be a monocyclic C₃₋₆ cycloalkyl or a bicyclic C₅₋₈ cycloalkyl. The C₁₋₄ alkyl of —O—(C₁₋₄ alkyl)-(an unsubstituted or a substituted C₃₋₈ cycloalkyl) can be —CH₂—, —CH₂CH₂—, —CH₂CH₂CH₂— or —CH₂CH₂CH₂CH₂—.

As described herein, R¹¹ can be substituted. Exemplary groups that can be present on R¹¹ include halogen, an unsubstituted C₁₋₄ alkyl, an unsubstituted monocyclic C₃₋₆ cycloalkyl, an unsubstituted C₁₋₄ alkoxy and an unsubstituted C₁₋₄ haloalkyl (such as those described herein).

The R⁸ and R¹⁰ moieties can be a substituted or an unsubstituted version of a C₁₋₆ alkyl, a C₂₋₆ alkenyl, a C₂₋₆ alkynyl, a monocyclic C₃₋₆ cycloalkyl, a bicyclic C₅₋₈ cycloalkyl, a monocyclic 4- to 6-membered heterocyclyl and an unsubstituted monocyclic C₃₋₆ cycloalkyl(CH₂)—. In some embodiments, R⁸ and/or R¹⁰ can be an unsubstituted C₁₋₆ alkyl. In other embodiments, R⁸ and/or R¹⁰ can be a substituted C₁₋₆ alkyl. Exemplary C₁₋₆ alkyls include methyl, ethyl, n-propyl, isopropyl, n-butyl, isobutyl, sec-butyl, tert-butyl, pentyl (straight-chained and branched) and hexyl (straight-chained and branched). In some embodiments, R⁸ and/or R¹⁰ can be an unsubstituted C₂₋₆ alkenyl. In other embodiments, R⁸ and/or R¹⁰ can be a substituted C₂₋₆ alkenyl. In still other embodiments, R⁸ and/or R¹⁰ can be an unsubstituted C₂₋₆ alkynyl. In yet still other embodiments, R⁸ and/or R¹⁰ can be a substituted C₂₋₆ alkynyl.

Cyclic moieties, including monocyclic and bicyclic moieties, can also be present for R⁸ and/or R¹⁰. In some embodiments, R⁸ and/or R¹⁰ can be an unsubstituted monocyclic C₃₋₆ cycloalkyl. In some embodiments, R⁸ and/or R¹⁰ can be a substituted monocyclic C₃₋₆ cycloalkyl. For example, R⁸ and/or R¹⁰ can be a substituted or an unsubstituted cyclopropyl, a substituted or an unsubstituted cyclobutyl, a substituted or an unsubstituted cyclopentyl or a substituted or an unsubstituted cyclohexyl. In some embodiments, R⁸ and/or R¹⁰ can be an unsubstituted bicyclic C₅₋₈ cycloalkyl. In other embodiments, R⁸ and/or R¹⁰ can be an unsubstituted bicyclic C₅₋₈ cycloalkyl. The two rings of the bicyclic C₅₋₈ cycloalkyl can joined in a fused or a spiro-fashion. Examples of rings connected in a fused and a spiro-fashion are provided herein. In some embodiments, R⁸ and/or R¹⁰ can be an unsubstituted or a substituted bicyclo[1.1.1]pentyl. In still other embodiments, R⁸ and/or R¹⁰ can be an unsubstituted monocyclic 4- to 6-membered heterocyclyl. In yet still other embodiments, R⁸ and/or R¹⁰ can be an unsubstituted monocyclic 4- to 6-membered heterocyclyl. The number of heteroatoms present in a monocyclic 4- to 6-membered heterocyclyl for R⁸ and/or R¹⁰ can vary. Suitable heteroatoms include, but are not limited to, O (oxygen), S (sulfur) and N (nitrogen). Examples of monocyclic 4- to 6-membered heterocyclyls are azetidine, oxetane, thietane, tetrahydrofuran, tetrahydrothiophene, pyrrolidine, oxazolidin-2-one, imidazolidin-2-one, tetrahydropyran, tetrahydrothiopyran, piperidine, piperazine, morpholine and thiomorpholine (including unsubstituted or substituted versions of each of the aforementioned). In some embodiments, R⁸ and/or R¹⁰ can be an unsubstituted monocyclic C₃₋₆ cycloalkyl(CH₂)—. Various monocyclic C₃₋₆ cycloalkyl are described herein. As examples, R⁸ and/or R¹⁰ can be selected from cyclopropyl(CH₂)—, cyclobutyl(CH₂)—, cyclopentyl(CH₂)— and cyclohexyl(CH₂)—.

As described herein, R⁸ and/or R¹⁰ can be substituted. In some embodiments, when R⁸ and/or R¹⁰ is a C₁₋₆ alkyl that is substituted, the C₁₋₆ alkyl can be substituted 1, 2, 3 or 4 times with a substituent independently selected from halogen, cyano, an unsubstituted or a substituted monocyclic C₃₋₆ cycloalkyl, an unsubstituted C₁₋₄ alkoxy and an unsubstituted C₁₋₄ haloalkoxy. In some embodiments, R⁸ and/or R¹⁰ can be a C₁₋₆ alkyl that is substituted 1 to 13 times with deuterium. In some embodiments, R⁸ and/or R¹⁰ can be a C₁₋₆ alkyl that is substituted 1 to 9 times with deuterium, 1 to 6 times with deuterium, 1 to 5 times with deuterium or 1 to 3 times with deuterium. Each halogen can be independently F (fluoro) or C₁ (chloro). Exemplary unsubstituted and substituted monocyclic C₃₋₆ cycloalkyls that can be present on a substituted C₁₋₆ alkyl for R⁸ and/or R¹⁰ include cyclopropyl, cyclobutyl, cyclopentyl, cyclohexyl and halogen-substituted monocyclic C₃₋₆ cycloalkyls. Suitable unsubstituted C₁₋₄ alkoxys that can be substituted on a C₁₋₆ alkyl of R⁸ and/or R¹⁰ include methoxy, ethoxy, n-propoxy, isopropoxy, n-butoxy, isobutoxy, sec-butoxy and tert-butoxy. Examples of an unsubstituted C₁₋₄ haloalkoxy can be substituted on a C₁₋₆ alkyl of R⁸ and/or R¹⁰ include —OCl₃, —OCF₃, —OCH₂C₁, —OCH₂F, —OCHCl₂ and —OCHF₂. In some embodiments, when R⁸ and/or R¹⁰ is a substituted C₂₋₆ alkenyl, a substituted C₂₋₆ alkynyl, a substituted monocyclic C₃₋₆ cycloalkyl, a substituted bicyclic C₅₋₈ cycloalkyl or a substituted monocyclic 4- to 6-membered heterocyclyl, each of the aforementioned can be substituted 1, 2, 3 or 4 times with a substituents independently selected from halogen, an unsubstituted C₁₋₄ alkyl, an unsubstituted C₂₋₄ alkenyl, an unsubstituted C₂₋₄ alkynyl, an unsubstituted C₁₋₄ haloalkyl, an unsubstituted or a substituted monocyclic C₃₋₆ cycloalkyl and an unsubstituted C₁₋₄ alkoxy. Examples of unsubstituted C₁₋₄ alkyls, an unsubstituted C₂₋₄ alkenyl and an unsubstituted C₂₋₄ alkynyl that can be substituted on a substituted C₂₋₆ alkenyl, a substituted C₂₋₆ alkynyl, a substituted monocyclic C₃₋₆ cycloalkyl, a substituted bicyclic C₅₋₈ cycloalkyl or a substituted monocyclic 4- to 6-membered heterocyclyl include methyl, ethyl, n-propyl, isopropyl, n-butyl, iso-butyl, sec-butyl, tert-butyl, ethenyl, propenyl, butenyl, ethynyl, propynyl and butynyl. Suitable halogens and unsubstituted C₁₋₄ alkoxys that can be present on a substituted C₂₋₆ alkenyl, a substituted C₂₋₆ alkynyl, a substituted monocyclic C₃₋₆ cycloalkyl, a substituted bicyclic C₅₋₈ cycloalkyl or a substituted monocyclic 4- to 6-membered heterocyclyl are described herein, such as in the previous paragraph. Non-limiting list of unsubstituted and substituted monocyclic C₃₋₆ cycloalkyls include cyclopropyl, cyclobutyl, cyclopentyl, cyclohexyl and halogen-substituted monocyclic C₃₋₆ cycloalkyls. Examples of unsubstituted C₁₋₆ haloalkyls that can be present on a substituted C₂₋₆ alkenyl, a substituted C₂₋₆ alkynyl, a substituted monocyclic C₃₋₆ cycloalkyl, a substituted bicyclic C₅₋₈ cycloalkyl or a substituted monocyclic 4- to 6-membered heterocyclyl include, but are not limited to, —CF₃, —CClF₂, —CHF₂, —C(CH₃)F₂, —CH₂F, —CH(CH₃)F, —CH₂CF₃, —CH(CH₃)CF₃, —CH₂CH₂CF₃, —CH₂CH(CH₃)CF₃, —CF₂CF₃, —CH₂CH₂F and —CH₂CH₂CH₂F.

In some embodiments, R⁵ can be R¹². As described herein, R¹² can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted monocyclic C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl), an unsubstituted or a substituted heteroaryl(alkyl), an unsubstituted or a substituted heterocyclyl(alkyl), an unsubstituted or a substituted C-carboxy, —OR¹³, —NR¹⁴R¹⁵ or —C(═O)—NR^{16A}R^{16B}. In some embodiments, R¹² can be an unsubstituted C₁₋₈ alkyl. In other embodiments, R¹² can be a substituted C₁₋₈ alkyl. In still other embodiments, R¹² can be an unsubstituted C₂₋₈ alkenyl. In yet still other embodiments, R¹² can be a substituted C₂₋₈ alkenyl. In some embodiments, R¹² can be an unsubstituted C₂₋₈ alkynyl. In other embodiments, R¹² can be a substituted C₂₋₈ alkynyl. In still other embodiments, R¹² can be an unsubstituted C₃₋₈ cycloalkyl. In yet still other embodiments, R¹² can be a substituted C₃₋₈ cycloalkyl. For example, R¹² can be an unsubstituted or a substituted monocyclic C₃₋₈ cycloalkyl.

A variety of cyclic moieties can be present for R¹². In some embodiments, R¹² can be an unsubstituted C₃₋₈ cycloalkyl. In other embodiments, R¹² can be a substituted C₃₋₈ cycloalkyl. For example, R¹² can be an unsubstituted or a substituted monocyclic C₃₋₈ cycloalkyl. In some embodiments, R¹² can be an unsubstituted aryl. In other embodiments, R¹² can be a substituted aryl. As an example, R¹² can be an unsubstituted or a substituted phenyl. In still other embodiments, R¹² can be an unsubstituted heteroaryl. In yet still other embodiments, R¹² can be a substituted heteroaryl. In some embodiments, R¹² can be an unsubstituted 3- to 8-membered monocyclic heterocyclyl. In other embodiments, R¹² can be a substituted 3- to 8-membered monocyclic heterocyclyl. In still other embodiments, R¹² can be an unsubstituted aryl(alkyl). For example, R¹² can be an unsubstituted or a substituted benzyl. In yet still other embodiments, R¹² can be a substituted aryl(alkyl). In some embodiments, R¹² can be an unsubstituted heteroaryl(alkyl). In other embodiments, R¹² can be a substituted heteroaryl(alkyl). In some embodiments, R¹² can be an unsubstituted heterocyclyl(alkyl). In other embodiments, R¹² can be a substituted heterocyclyl(alkyl). The aryl, heteroaryl and heterocyclyl, including that those of an aryl(alkyl), heteroaryl(alkyl) and heterocyclyl(alkyl)), can be monocyclic or bicyclic (unless stated otherwise), and include 1, 2, 3, 4 or 5 heteroatoms independently selected from O (oxygen), S (sulfur) and N (nitrogen). Exemplary heteroaryls for R¹² include, but are not limited to, furane, isoxazole, isothiazole, pyrrole, pyrazole, oxazole, thiazole, 1,2,3-triazole, 1,2,4-triazole, imidazole, 1,3,4-oxadiazole, 1,3,4-thiadiazole, pyridine, pyridazine, pyrimidine, pyridazine and pyrazine. Examples of heterocyclyls for R¹² include azetidine, oxetane, thietane, tetrahydrofuran, tetrahydrothiophene, pyrrolidine, oxazolidin-2-one, imidazolidin-2-one, tetrahydropyran, tetrahydrothiopyran, piperidine, piperazine, morpholine and thiomorpholine. When R¹² is aryl(alkyl) or heteroaryl(alkyl), the alkyl linker can be 1, 2 or 3 alkylene groups, such as —CH₂—, —CH₂CH₂— and —CH₂CH₂CH₂—. The alkyl linker of an aryl(alkyl), a heteroaryl(alkyl) and a heterocyclyl(alkyl) can be also substituted. Possible substituents that take the place of one or more of the hydrogens (such as 1, 2, 3 or 4 hydrogens) include, but are not limited to, halogen and hydroxy, or two hydrogen can be replaced with a spiro-connected monocyclic C₃₋₄ cycloalkyl (for example,

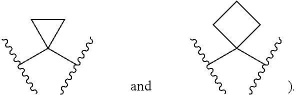 [SMILES: *C1(*)CC1 ; *C1(*)CCC1]

In some embodiments, R¹² can be an unsubstituted C-carboxy. In other embodiments, R¹² can be a substituted C-carboxy. In still other embodiments, R¹² can be an alkoxy. For example, in some embodiments, R¹² can be —OR¹³, wherein R¹³ can be an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl), an unsubstituted or a substituted heteroaryl(alkyl) or an unsubstituted or a substituted heterocyclyl(alkyl). In still other embodiments, R¹² can be amino, mono-substituted amine or a di-substituted amine. In some embodiments, the amine can be —NR¹⁴R¹⁵, wherein R¹⁴ and R¹⁵ can be independently selected from hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl). In other embodiments, the amine can be —NR¹⁴R¹⁵, wherein R¹⁴ and R¹⁵ can be taken together with the nitrogen to which R¹⁴ and R¹⁵ are attached to form an unsubstituted or a substituted 3- to 8-membered heterocyclyl. In yet still other embodiments, R¹² can be C-amido. In some embodiments, R¹² can be —C(═O)—NR^{16A}R^{16B}, wherein R^{16A} can be hydrogen or an unsubstituted C₁₋₃ alkyl; and R^{16B} can be an unsubstituted or a substituted aryl, an unsubstituted or a substituted heteroaryl or an unsubstituted or a substituted 3- to 8-membered monocyclic heterocyclyl.

As provided herein, R¹² can be substituted. For example, R¹² can be substituted 1, 2, 3 or 4 times with a substituent independently selected from halogen, hydroxy, an unsubstituted C₁₋₄ alkyl, an unsubstituted monocyclic C₃₋₆ cycloalkyl, an unsubstituted phenyl, a substituted phenyl, an unsubstituted 5- or 6-membered heteroaryl and a substituted 5- or 6-membered heteroaryl (such as a phenyl and/or 5- or 6-membered heteroaryl can be substituted 1, 2, 3, 4 or 5 times with a substituent selected from halogen (for example, F, Cl and Br), an unsubstituted C₁₋₄ alkyl (for example, methyl, ethyl, n-propyl, iso-propyl, n-butyl, iso-butyl, sec-butyl and tert-butyl), an unsubstituted C₁₋₄ alkoxy (such as methoxy, ethoxy, n-propoxy, iso-propoxy, n-butoxy, iso-butoxy, sec-butoxy and tert-butoxy), an unsubstituted C₁₋₄ haloalkyl (such as —CF₃, —CClF₂, —CHF₂, —C(CH₃)F₂, —CH₂F, —CH(CH₃)F, —CH₂CF₃, —CH(CH₃)CF₃, —CH₂CH₂CF₃, —CH₂CH(CH₃)CF₃, —CF₂CF₃, —CH₂CH₂F and —CH₂CH₂CH₂F), an unsubstituted —O(an unsubstituted C₁₋₄ haloalkyl) (for example, —OCF₃, —OCHF₂, —OC(CH₃)F₂, —OCH₂F, —OCH(CH₃)F, —OCH₂CF₃, —OCH₂CH₂F and —OCH₂CH₂CH₂F) and —S(═O)₂ (an unsubstituted C₁₋₄ alkyl). The alkyl linker of an aryl(alkyl), a heteroaryl(alkyl) and a heterocyclyl(alkyl) can be also substituted. Possible substituents that take the place of one or more of the hydrogens (such as 1, 2, 3 or 4 hydrogens) include, but are not limited to, halogen and hydroxy.

Various examples of R¹² groups include the following:

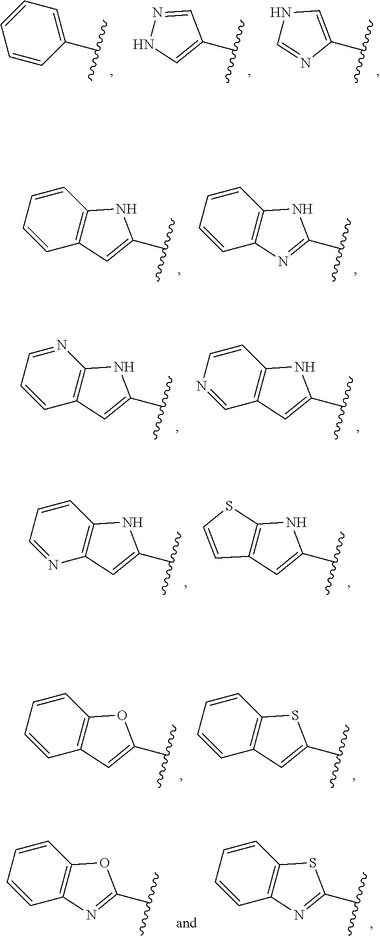

wherein each is unsubstituted or substituted. For example, R¹² can be substituted 1, 2, 3, or more than 3 times with a substituent independently selected from halogen, (such as F), an unsubstituted C₁₋₄ alkyl, an unsubstituted C₁₋₄ haloalkyl, hydroxy, an unsubstituted C₁₋₄ alkoxy and an unsubstituted or a substituted phenyl (for example, an unsubstituted phenyl or a phenyl substituted with 1, 2 or 3 substituents independently selected from halogen, an unsubstituted C₁₋₄ alkyl, an unsubstituted C₁₋₄ haloalkyl and an unsubstituted C₁₋₄ alkoxy). Further examples of R¹² include the following:

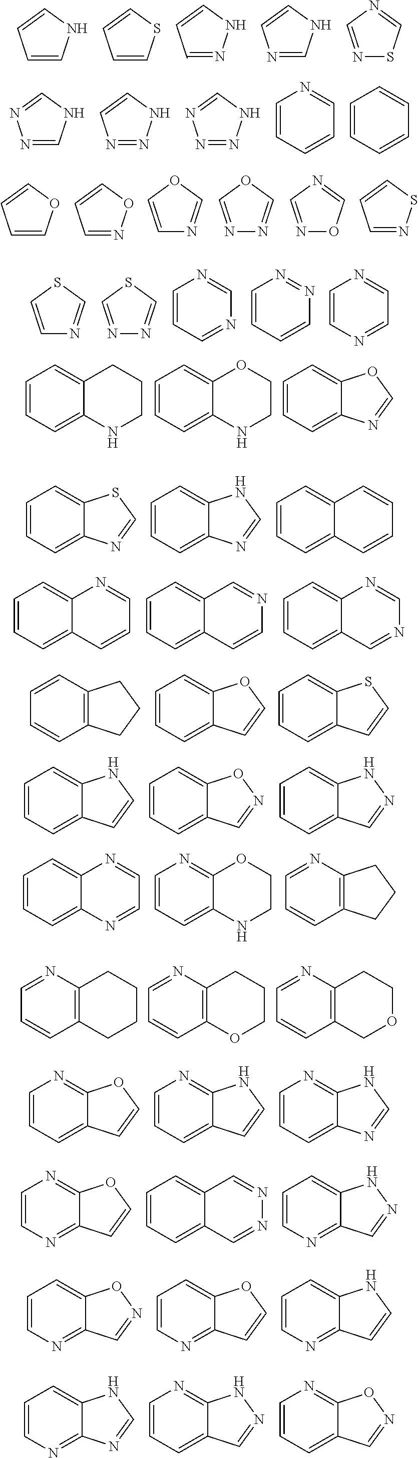

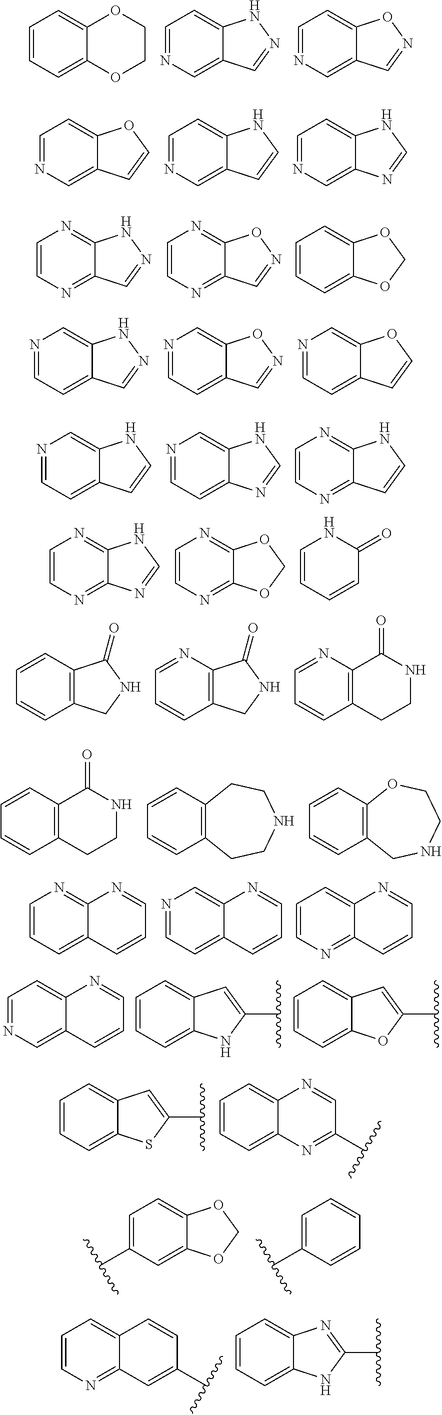

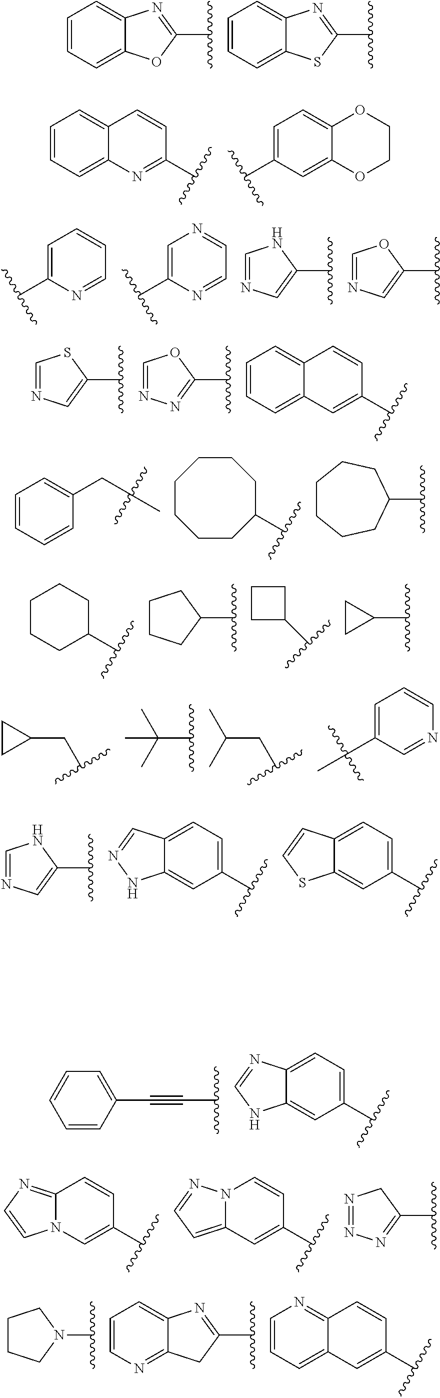

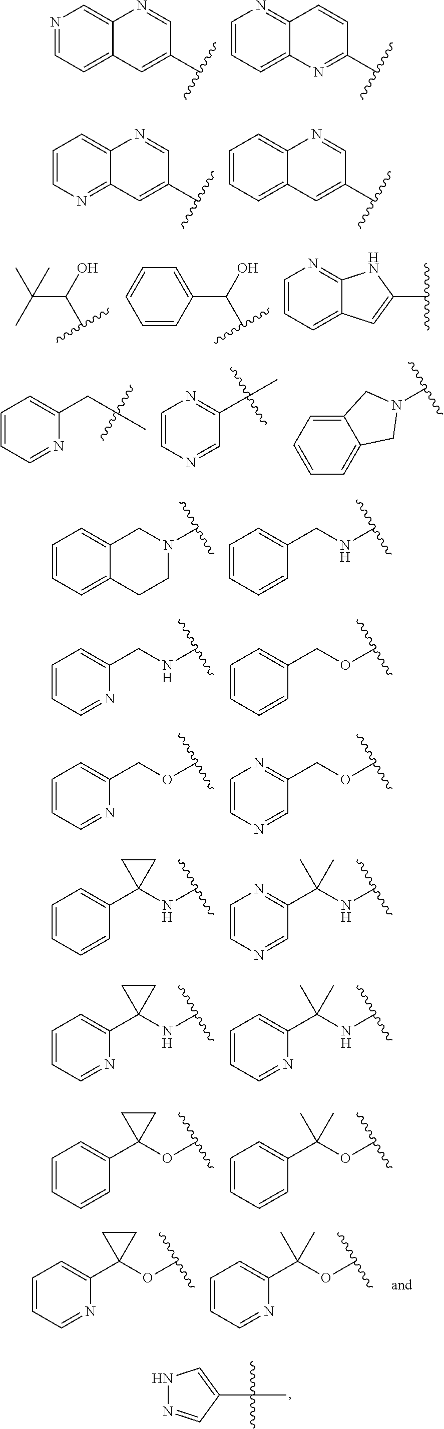

and wherein each is unsubstituted or substituted.

Exemplary R⁵ groups include the following:

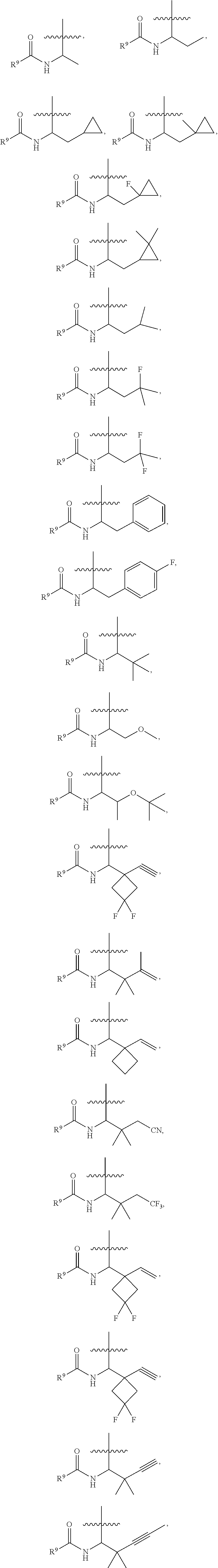

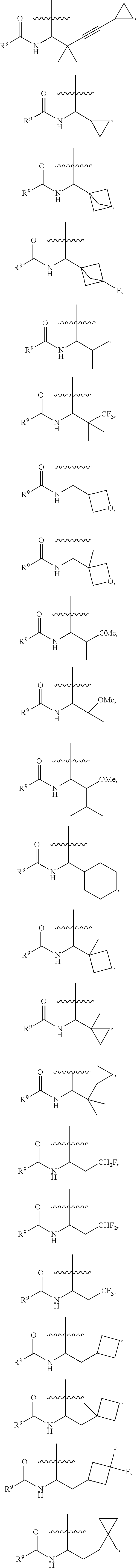

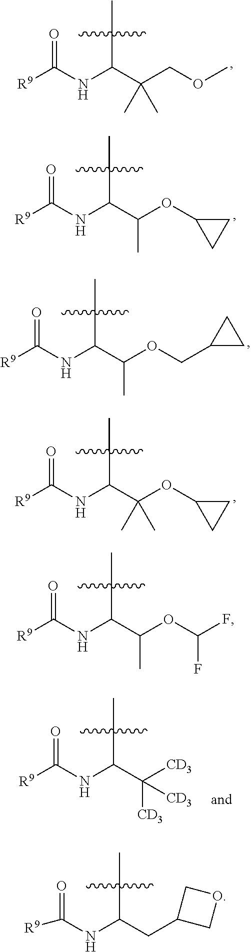

Some examples of R⁵ groups include the following:

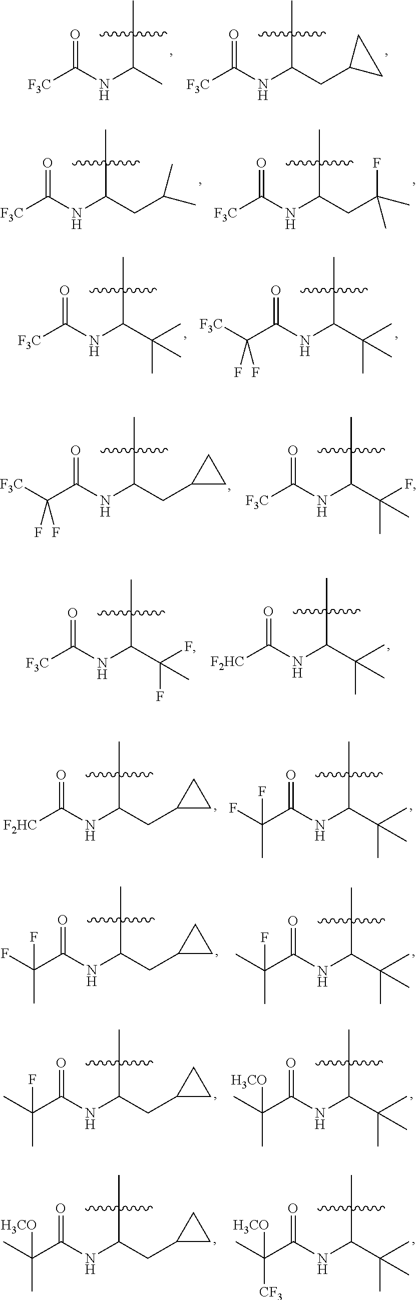

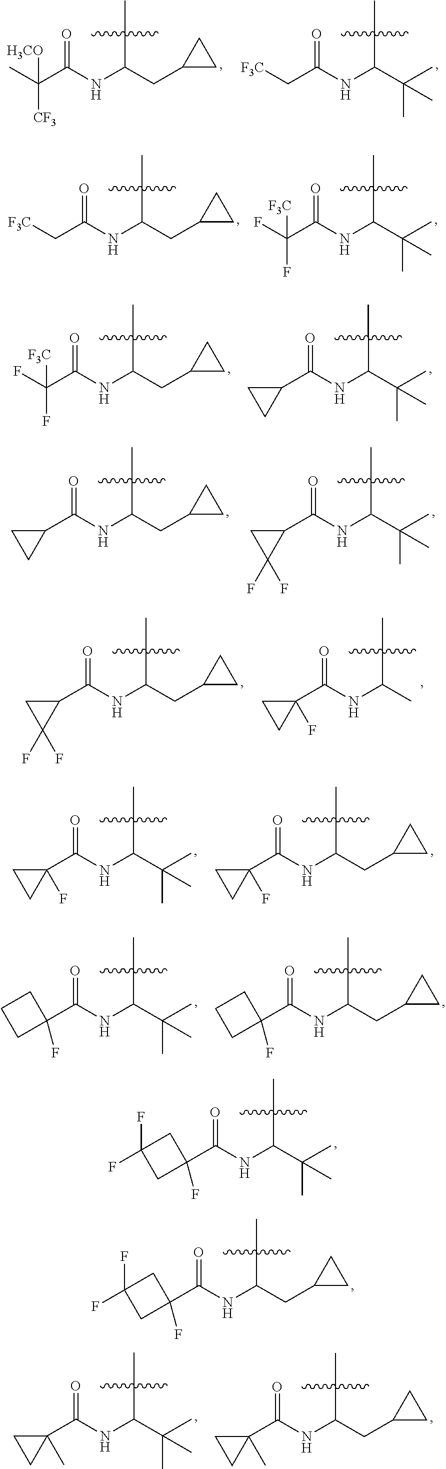

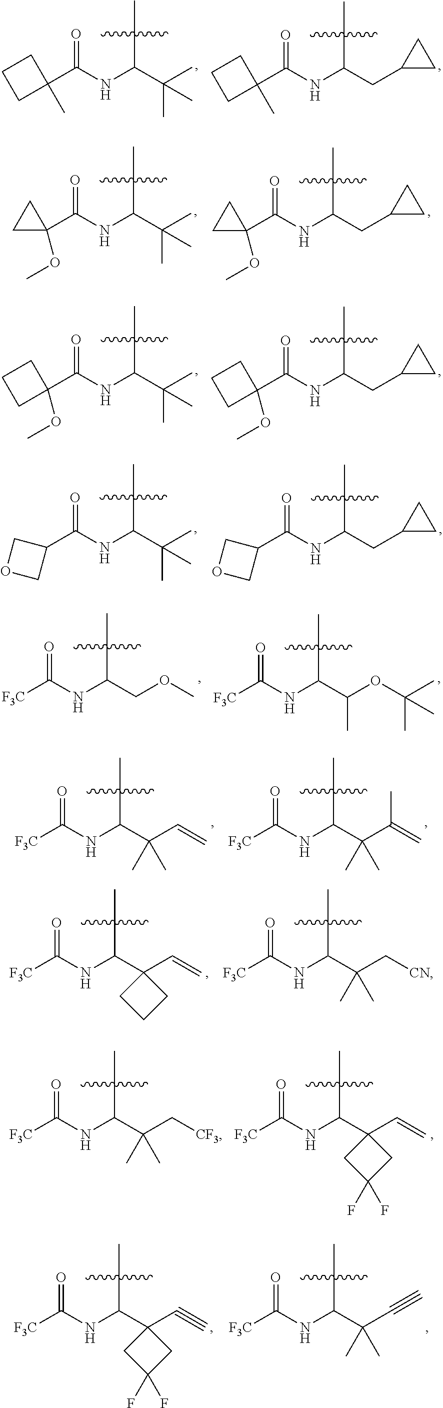

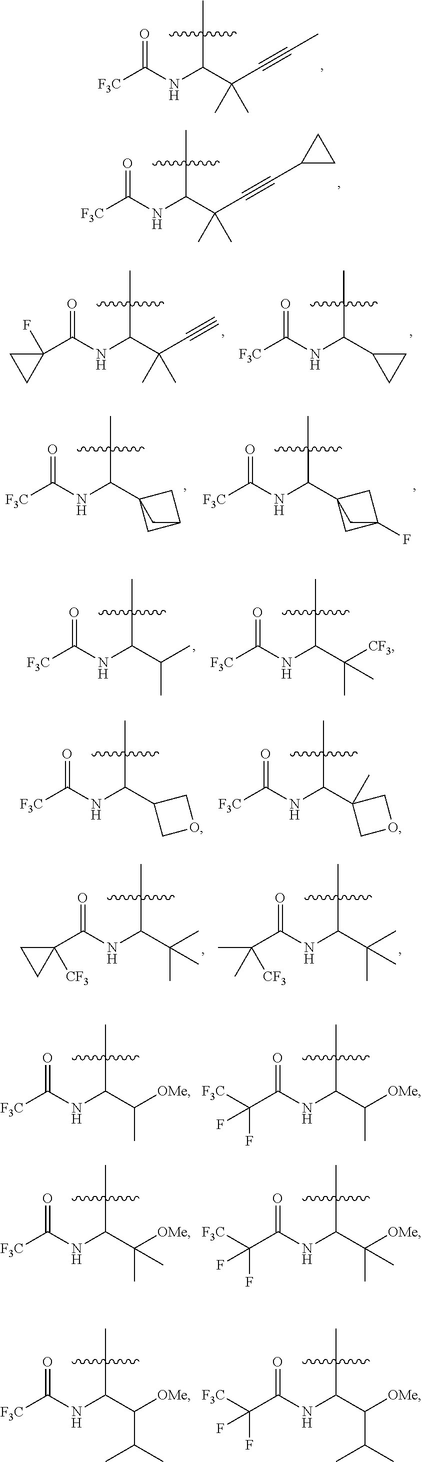

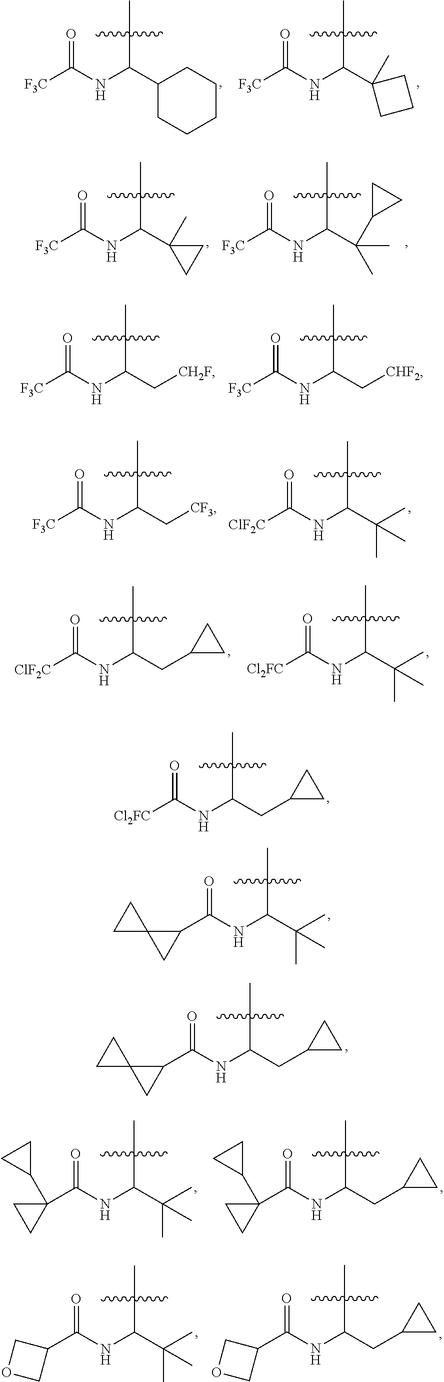

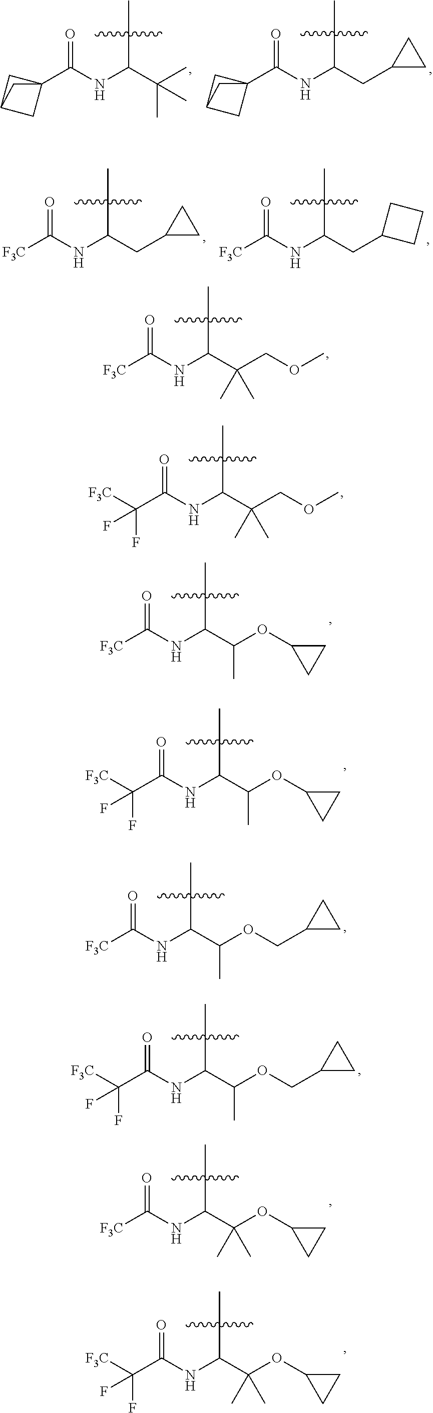

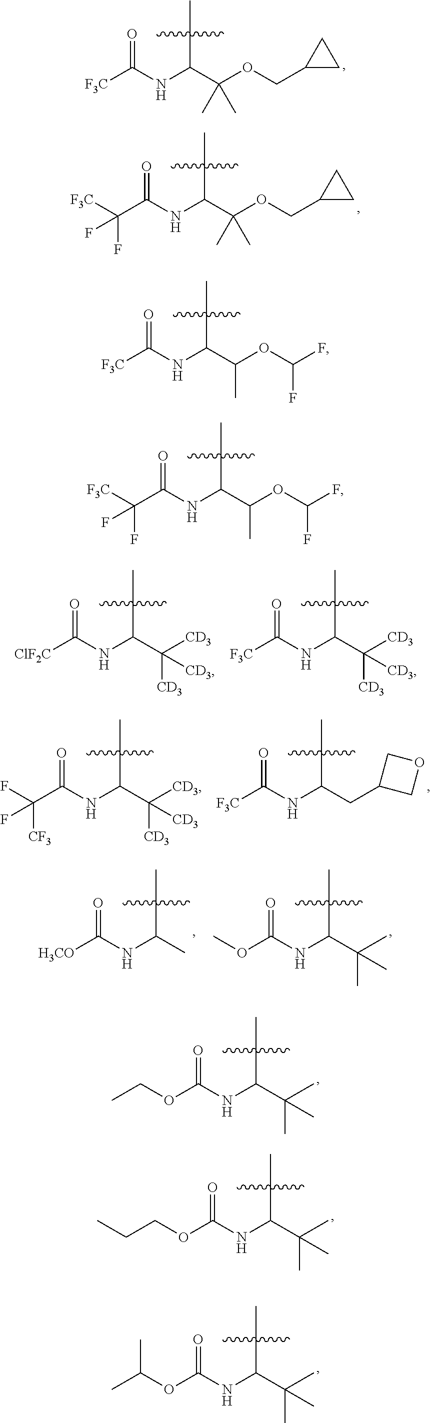

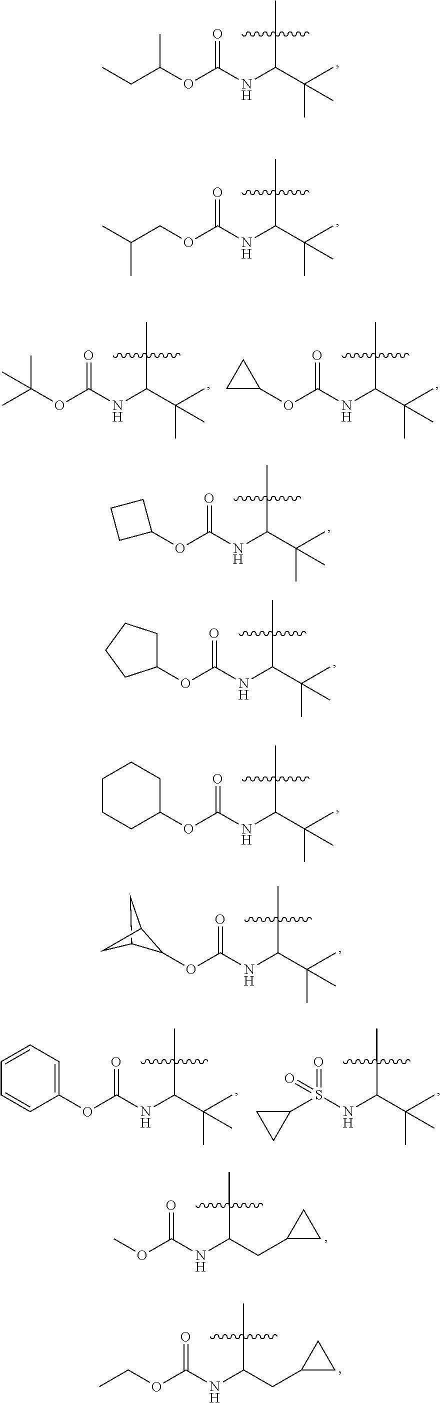

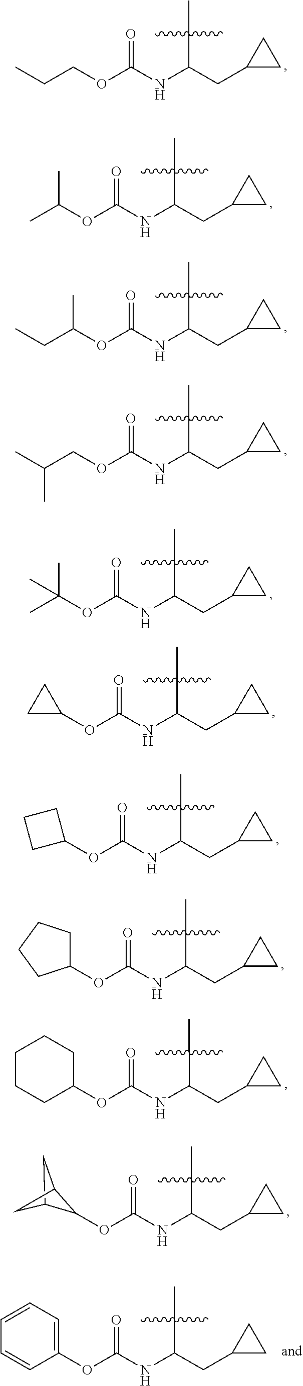

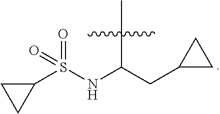 [SMILES: *C(CC1CC1)NS(=O)(=O)C1CC1]

Further examples of R⁵ groups include:

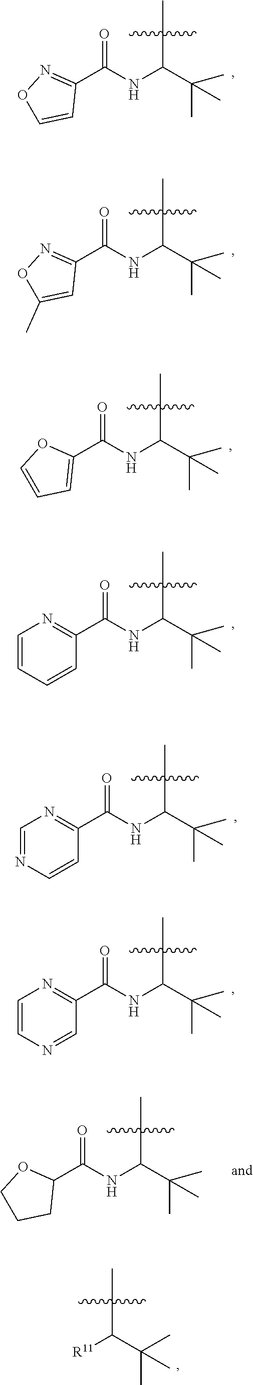

wherein each moiety is unsubstituted or substituted.

In some embodiments, a compound of Formula (I). or a pharmaceutically acceptable salt thereof, can be where when R¹² is

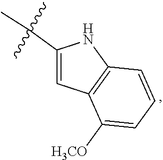 [SMILES: *c1cc2c(OC)cccc2[nH]1]

then R² and R³ cannot be taken together along with the carbon to which they are attached to form an unsubstituted

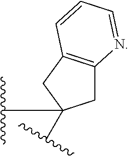 [SMILES: *C1(*)Cc2cccnc2C1]

In some embodiments, Ring A¹ cannot be an unsubstituted or a substituted

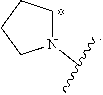 [SMILES: *N1CCCC1]

In other embodiments, Ring A¹ cannot be an unsubstituted or a substituted

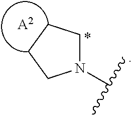

In still other embodiments, Ring A¹ cannot be an unsubstituted or a substituted

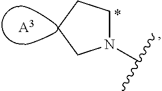

for example, Ring A¹ cannot be an unsubstituted or a substituted

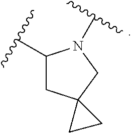 [SMILES: *C1CC2(CC2)CN1*]

In some embodiments, Ring A¹ cannot be an unsubstituted or a substituted

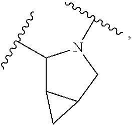 [SMILES: *C1C2CC2CN1*]

such as

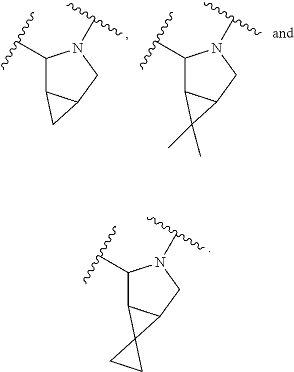

In some embodiments, Ring A¹ cannot be an unsubstituted

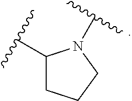 [SMILES: *C1CCCN1*]

In other embodiments, Ring A¹ cannot be a substituted

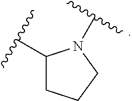 [SMILES: *C1CCCN1*]

such as

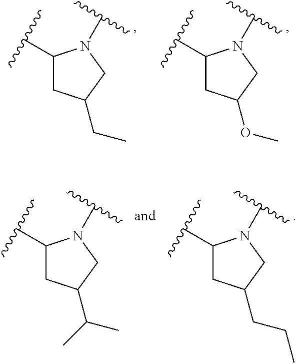

In some embodiments, Ring A¹ cannot be an unsubstituted or a substituted or

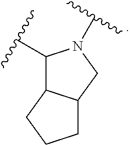 [SMILES: *C1C2CCCC2CN1*]

In some embodiment, R^{N} cannot be a C₁₋₆ alkyl, such as methyl. In some embodiments, R¹² cannot be a ketoamide. In some embodiments, R¹² cannot be an unsubstituted or a substituted heteroaryl. For example, in some embodiments, R¹² cannot be an unsubstituted or a substituted indolyl. In some embodiments, R¹² cannot be an unsubstituted, halogen-substituted indolyl or alkoxy-substituted indolyl. In some embodiments, R¹² cannot be an unsubstituted or a substituted

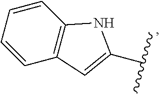 [SMILES: *c1cc2ccccc2[nH]1]

for example,

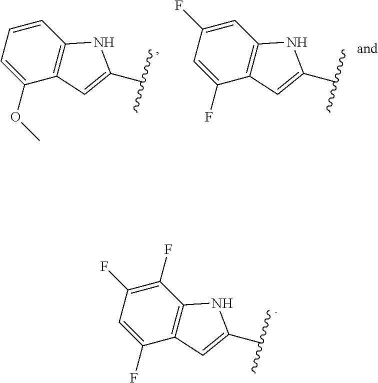 [SMILES: *c1cc2c(OC)cccc2[nH]1 ; *c1cc2c(F)cc(F)cc2[nH]1 ; *c1cc2c(F)cc(F)c(F)c2[nH]1]

In some embodiments, R¹² cannot be an unsubstituted or a substituted heterocyclyl. In some embodiments, R¹² cannot be an unsubstituted or a substituted bicyclic heterocyclyl, such as an unsubstituted or a substituted 5,6,7,8-tetrahydroquinoline, an unsubstituted or a substituted 5,6,7,8-tetrahydroisoquinoline, an unsubstituted or a substituted 5,6,7,8-tetrahydroisoquinolin-1(2H)-one or an unsubstituted or a substituted 2,5,6,7-tetrahydro-1H-cyclopenta[c]pyridin-1-one. In some embodiments, R¹² cannot be selected from

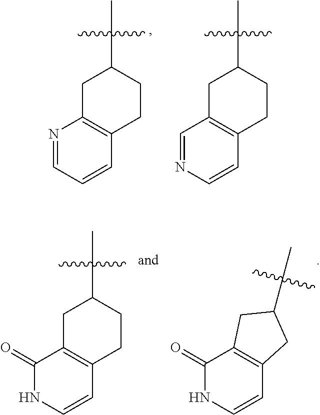

In some embodiments, R¹² cannot be one or more of the following:

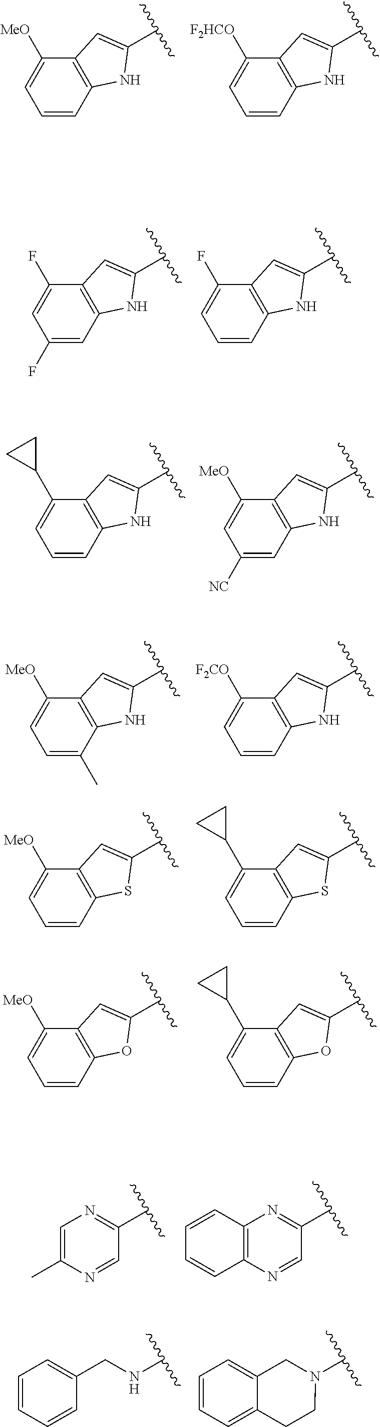

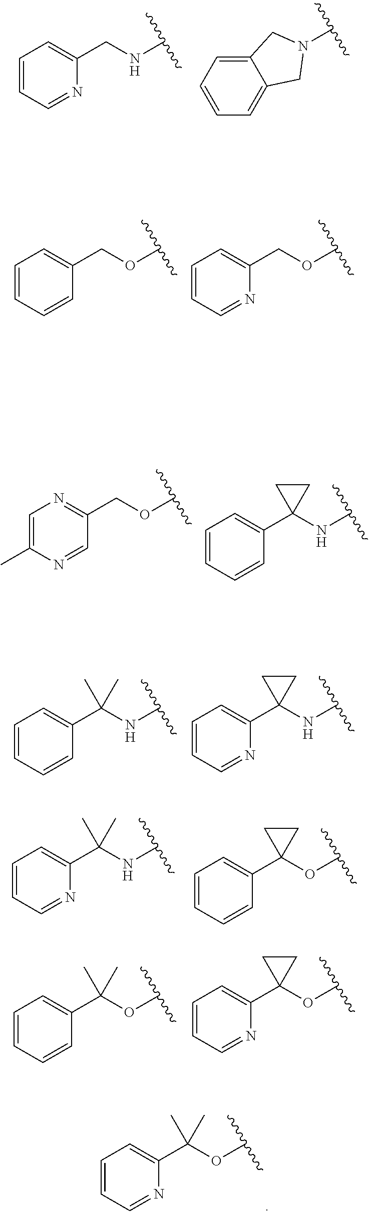

In some embodiments, R¹² cannot be one or more of the following:

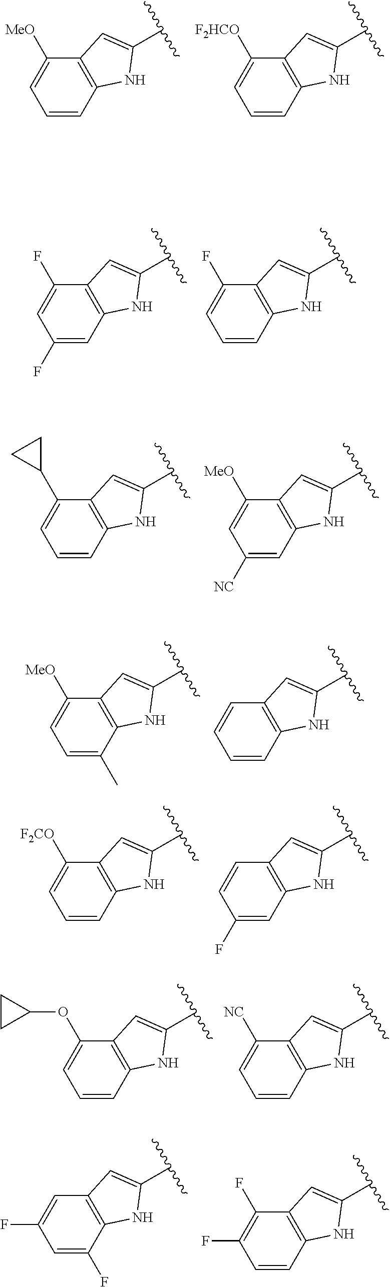

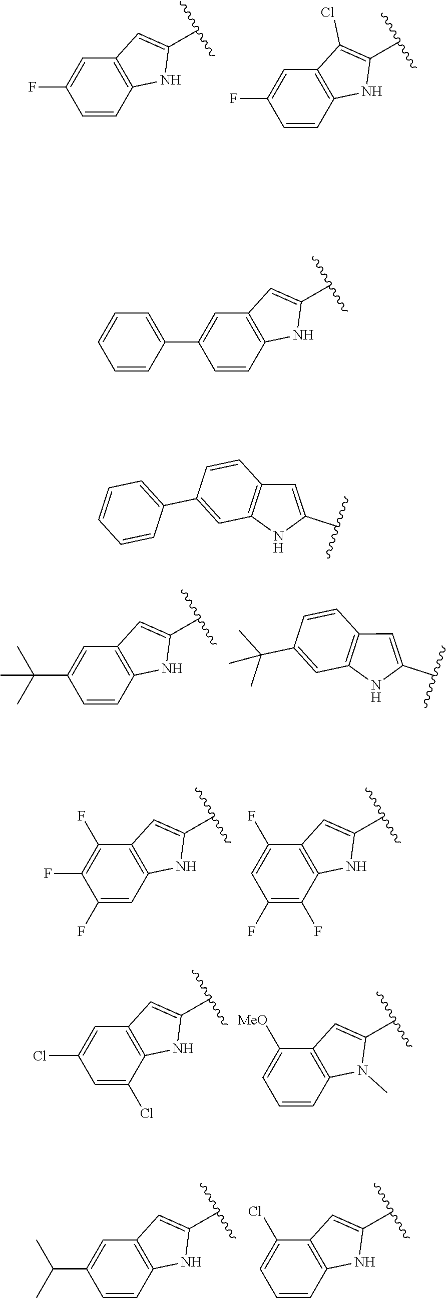

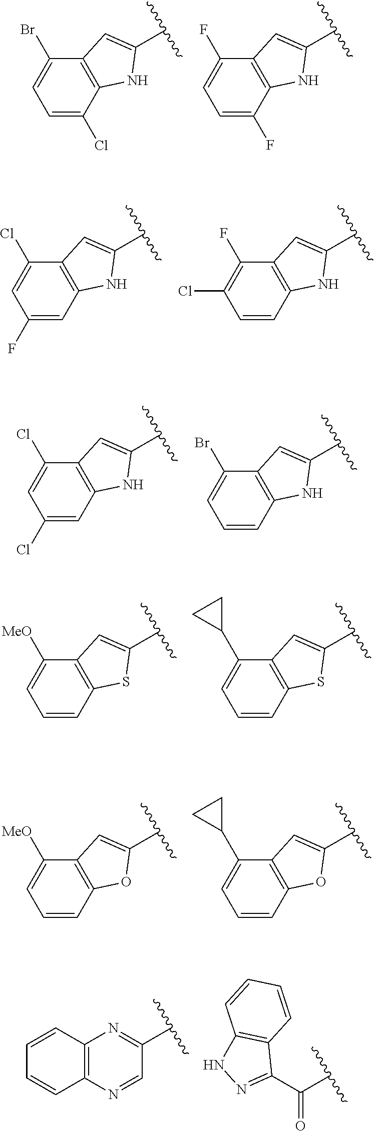

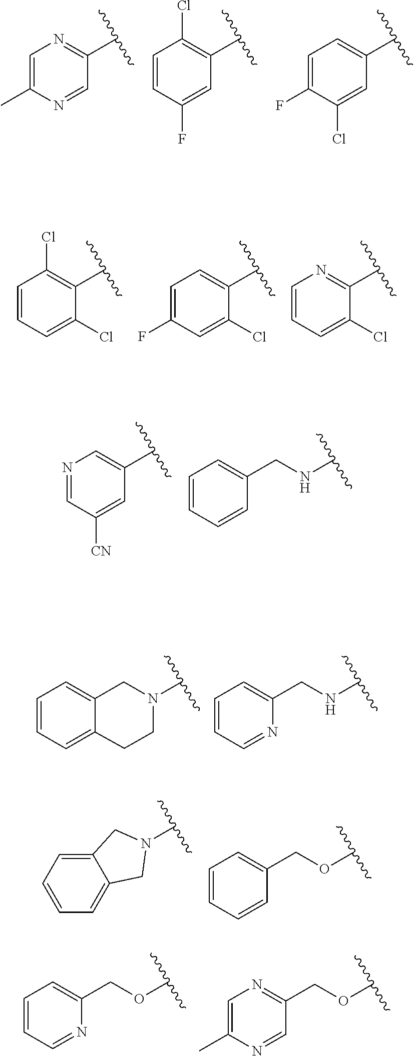

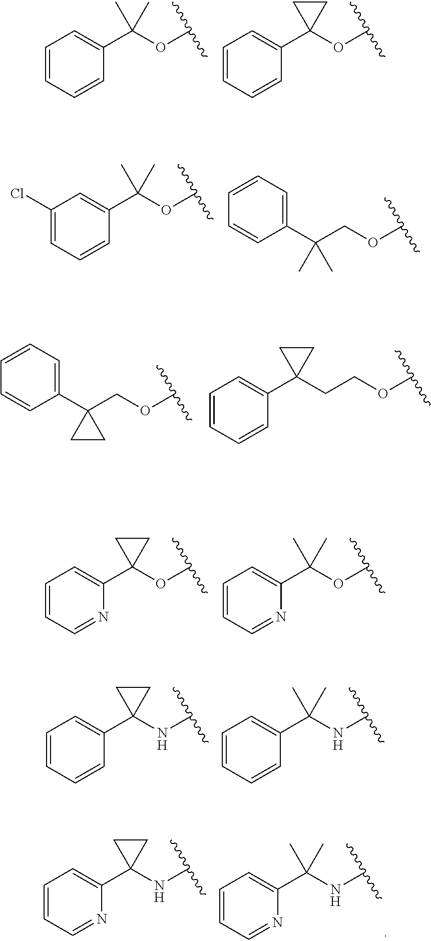

In some embodiments, a compound of Formula (I), or a pharmaceutically acceptable salt thereof, cannot be a compound (including salts thereof) provided in WO 2023/180189. In some embodiments, a compound of Formula (I), or a pharmaceutically acceptable salt thereof, cannot be a compound (including salts thereof) provided in WO 2023/011443.

Examples of compounds of Formula (I), include the following:

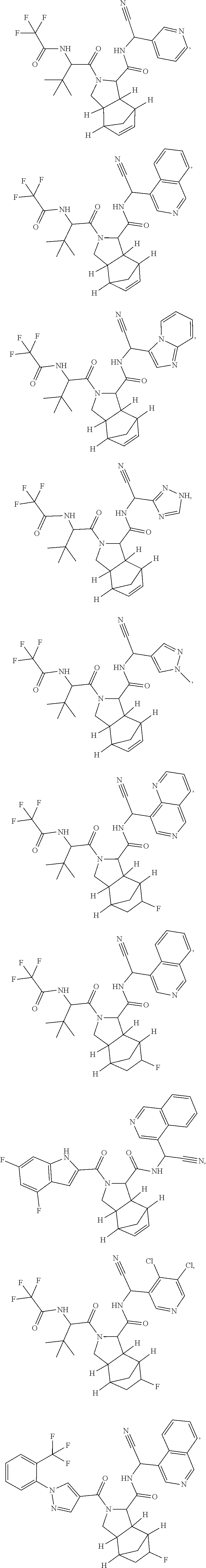

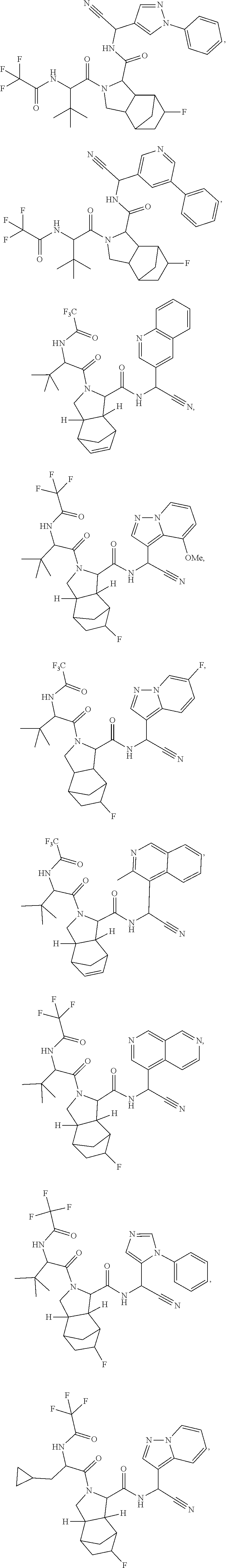

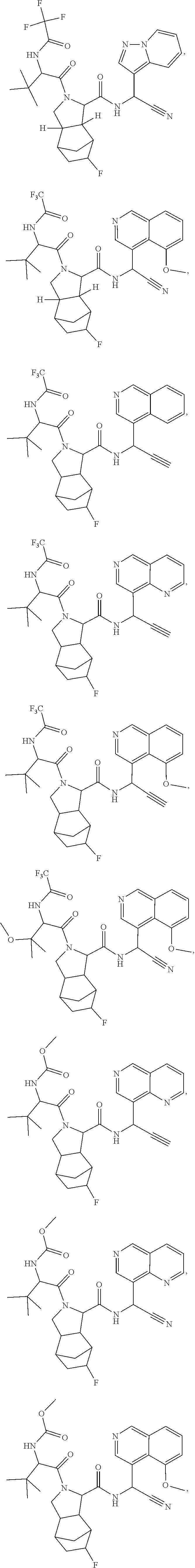

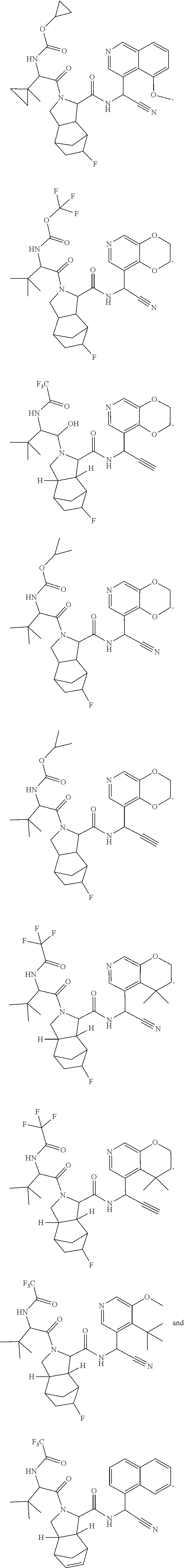

or a pharmaceutically acceptable salt of any of the foregoing.

Further examples of compounds of Formula (I), include the following:

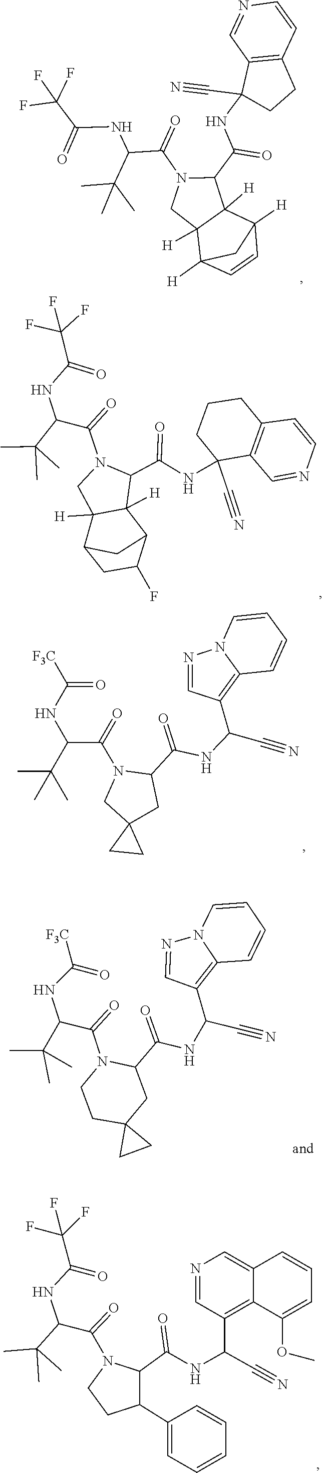 [SMILES: CC(C)(C)C(NC(=O)C(F)(F)F)C(=O)N1CC2C3C=CC(C3)C2C1C(=O)NC1(C#N)CCc2ccncc21 ; CC(C)(C)C(NC(=O)C(F)(F)F)C(=O)N1CC2C3CC(F)C(C3)C2C1C(=O)NC1(C#N)CCCc2ccncc21 ; CC(C)(C)C(NC(=O)C(F)(F)F)C(=O)N1CC2(CC2)CC1C(=O)NC(C#N)c1cnn2ccccc12 ; CC(C)(C)C(NC(=O)C(F)(F)F)C(=O)N1CCC2(CC2)CC1C(=O)NC(C#N)c1cnn2ccccc12 ; COc1cccc2cncc(C(C#N)NC(=O)C3C(c4ccccc4)CCN3C(=O)C(NC(=O)C(F)(F)F)C(C)(C)C)c12]

or a pharmaceutically acceptable salt of any of the foregoing.

Some additional examples of Formula (I) include:

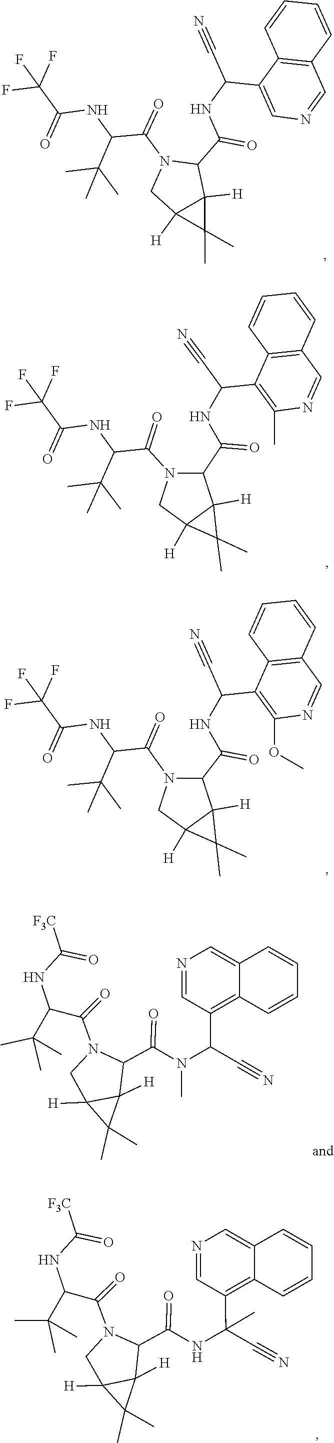 [SMILES: CC(C)(C)C(NC(=O)C(F)(F)F)C(=O)N1CC2C(C1C(=O)NC(C#N)c1cncc3ccccc13)C2(C)C ; Cc1ncc2ccccc2c1C(C#N)NC(=O)C1C2C(CN1C(=O)C(NC(=O)C(F)(F)F)C(C)(C)C)C2(C)C ; COc1ncc2ccccc2c1C(C#N)NC(=O)C1C2C(CN1C(=O)C(NC(=O)C(F)(F)F)C(C)(C)C)C2(C)C ; CN(C(=O)C1C2C(CN1C(=O)C(NC(=O)C(F)(F)F)C(C)(C)C)C2(C)C)C(C#N)c1cncc2ccccc12 ; CC(C#N)(NC(=O)C1C2C(CN1C(=O)C(NC(=O)C(F)(F)F)C(C)(C)C)C2(C)C)c1cncc2ccccc12]

or a pharmaceutically acceptable salt of any of the foregoing.

Additional examples of compounds of Formula (I), include the following:

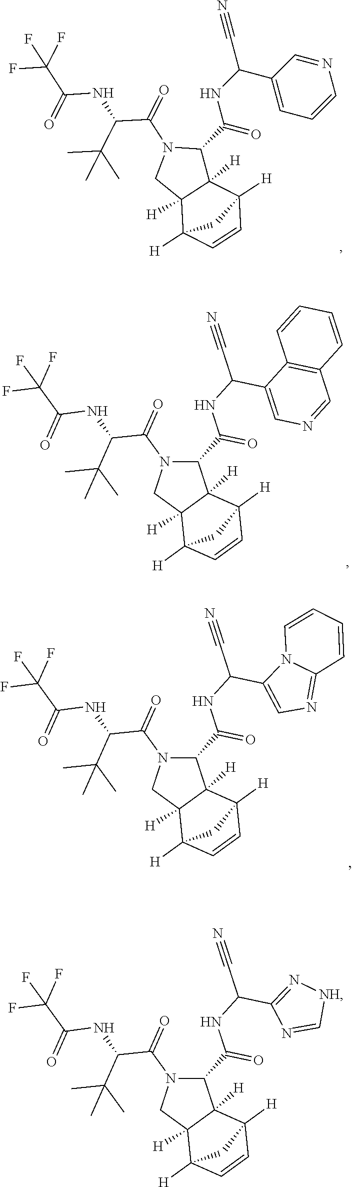 [SMILES: CC(C)(C)[C@H](NC(=O)C(F)(F)F)C(=O)N1C[C@H]2[C@@H]([C@H]1C(=O)NC(C#N)c1cccnc1)[C@H]1C=C[C@@H]2C1 ; CC(C)(C)[C@H](NC(=O)C(F)(F)F)C(=O)N1C[C@H]2[C@@H]([C@H]1C(=O)NC(C#N)c1cncc3ccccc13)[C@H]1C=C[C@@H]2C1 ; CC(C)(C)[C@H](NC(=O)C(F)(F)F)C(=O)N1C[C@H]2[C@@H]([C@H]1C(=O)NC(C#N)c1cnc3ccccn13)[C@H]1C=C[C@@H]2C1 ; CC(C)(C)[C@H](NC(=O)C(F)(F)F)C(=O)N1C[C@H]2[C@@H]([C@H]1C(=O)NC(C#N)c1nc[nH]n1)[C@H]1C=C[C@@H]2C1]

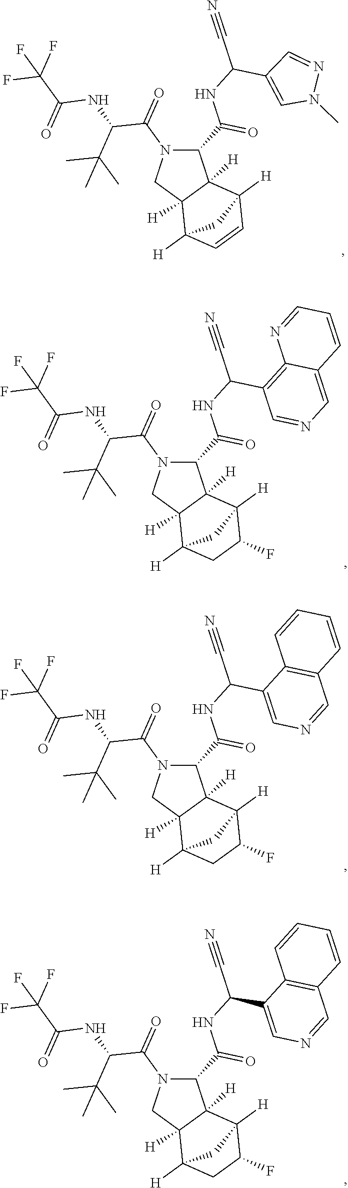 [SMILES: Cn1cc(C(C#N)NC(=O)[C@@H]2[C@@H]3[C@H](CN2C(=O)[C@@H](NC(=O)C(F)(F)F)C(C)(C)C)[C@@H]2C=C[C@H]3C2)cn1 ; CC(C)(C)[C@H](NC(=O)C(F)(F)F)C(=O)N1C[C@@H]2[C@H]3C[C@@H]([C@@H]2[C@H]1C(=O)NC(C#N)c1cncc2cccnc12)[C@H](F)C3 ; CC(C)(C)[C@H](NC(=O)C(F)(F)F)C(=O)N1C[C@@H]2[C@H]3C[C@@H]([C@@H]2[C@H]1C(=O)NC(C#N)c1cncc2ccccc12)[C@H](F)C3 ; CC(C)(C)[C@H](NC(=O)C(F)(F)F)C(=O)N1C[C@@H]2[C@H]3C[C@@H]([C@@H]2[C@H]1C(=O)N[C@@H](C#N)c1cncc2ccccc12)[C@H](F)C3]

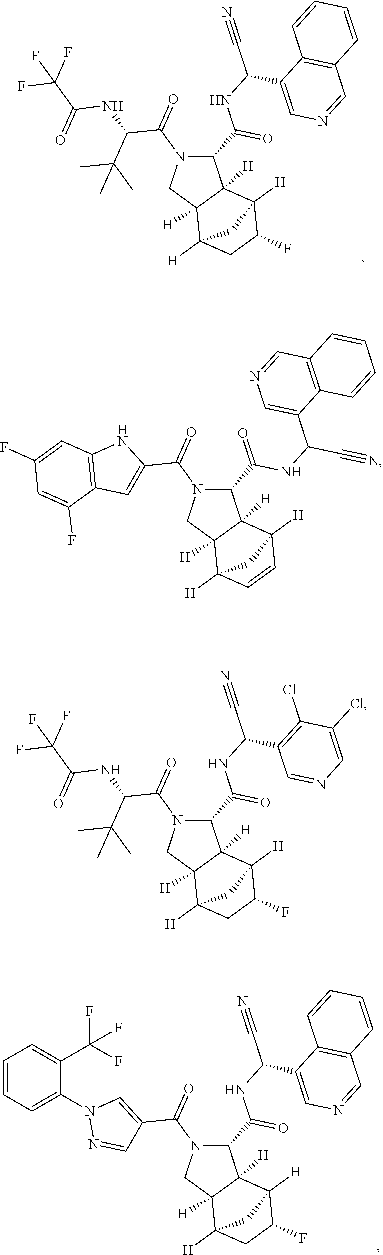 [SMILES: CC(C)(C)[C@H](NC(=O)C(F)(F)F)C(=O)N1C[C@@H]2[C@H]3C[C@@H]([C@@H]2[C@H]1C(=O)N[C@H](C#N)c1cncc2ccccc12)[C@H](F)C3 ; N#CC(NC(=O)[C@@H]1[C@@H]2[C@H](CN1C(=O)c1cc3c(F)cc(F)cc3[nH]1)[C@@H]1C=C[C@H]2C1)c1cncc2ccccc12 ; CC(C)(C)[C@H](NC(=O)C(F)(F)F)C(=O)N1C[C@@H]2[C@H]3C[C@@H]([C@@H]2[C@H]1C(=O)N[C@H](C#N)c1cncc(Cl)c1Cl)[C@H](F)C3 ; N#C[C@@H](NC(=O)[C@@H]1[C@@H]2[C@H](CN1C(=O)c1cnn(-c3ccccc3C(F)(F)F)c1)[C@H]1C[C@@H]2[C@H](F)C1)c1cncc2ccccc12]

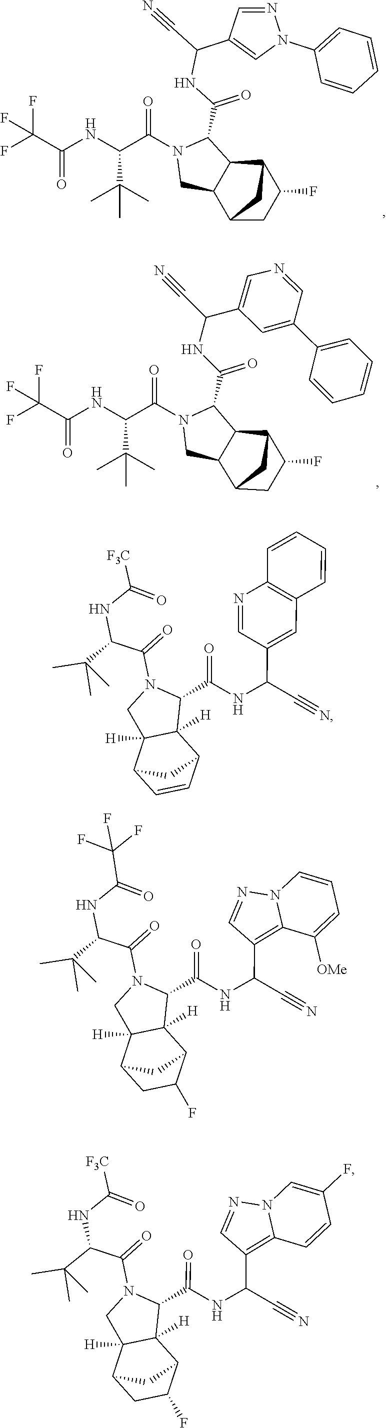

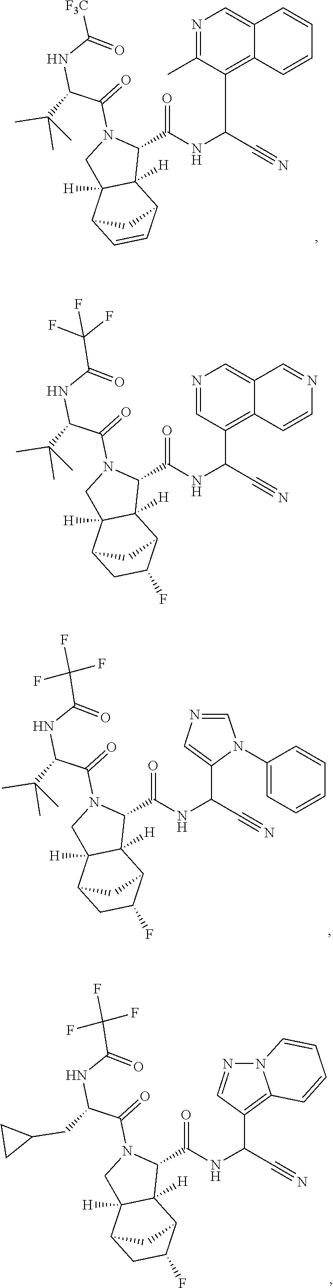 [SMILES: Cc1ncc2ccccc2c1C(C#N)NC(=O)[C@@H]1[C@@H]2[C@H](CN1C(=O)[C@@H](NC(=O)C(F)(F)F)C(C)(C)C)[C@@H]1C=C[C@H]2C1 ; CC(C)(C)[C@H](NC(=O)C(F)(F)F)C(=O)N1C[C@@H]2[C@H]3C[C@@H]([C@@H]2[C@H]1C(=O)NC(C#N)c1cncc2cnccc12)[C@H](F)C3 ; CC(C)(C)[C@H](NC(=O)C(F)(F)F)C(=O)N1C[C@@H]2[C@H]3C[C@@H]([C@@H]2[C@H]1C(=O)NC(C#N)c1cncn1-c1ccccc1)[C@H](F)C3 ; N#CC(NC(=O)[C@@H]1[C@@H]2[C@H](CN1C(=O)[C@H](CC1CC1)NC(=O)C(F)(F)F)[C@H]1C[C@@H]2[C@H](F)C1)c1cnn2ccccc12]

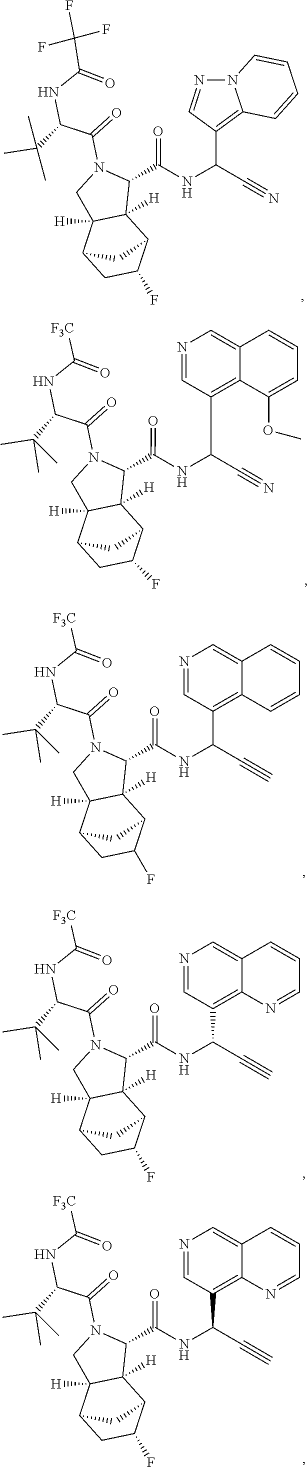

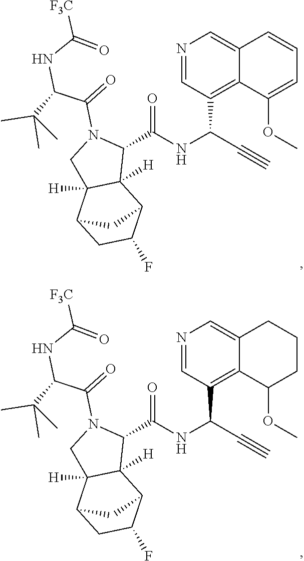

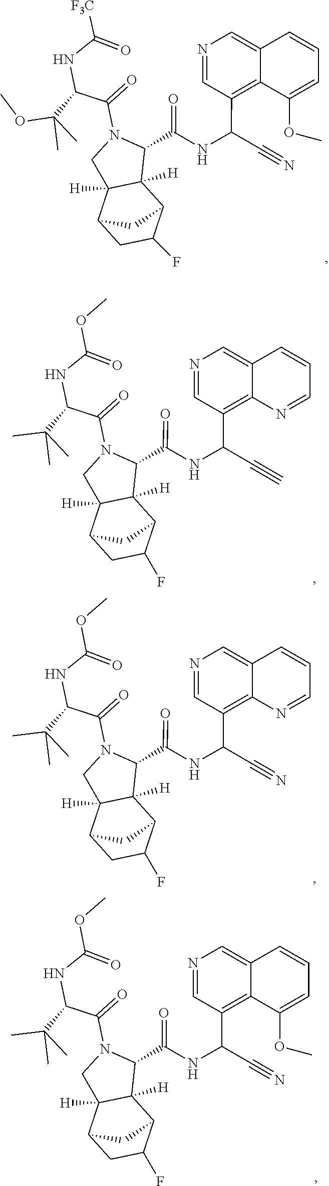

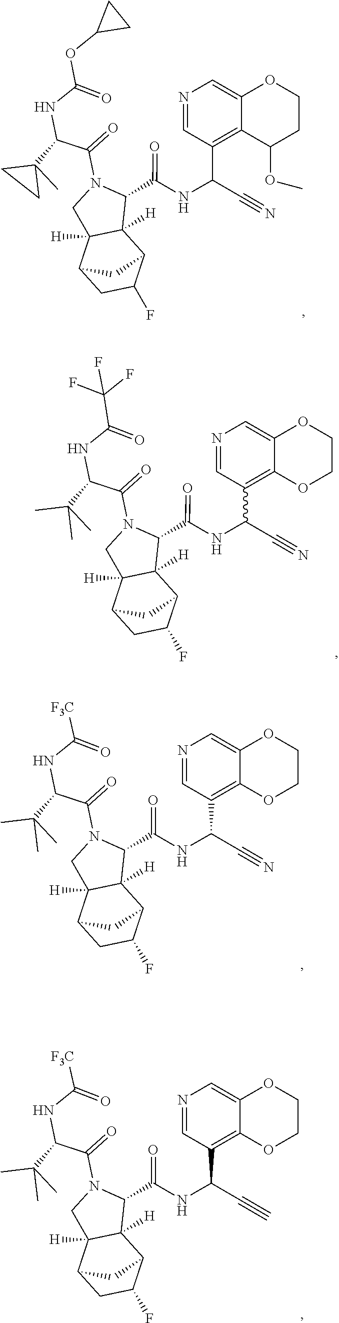

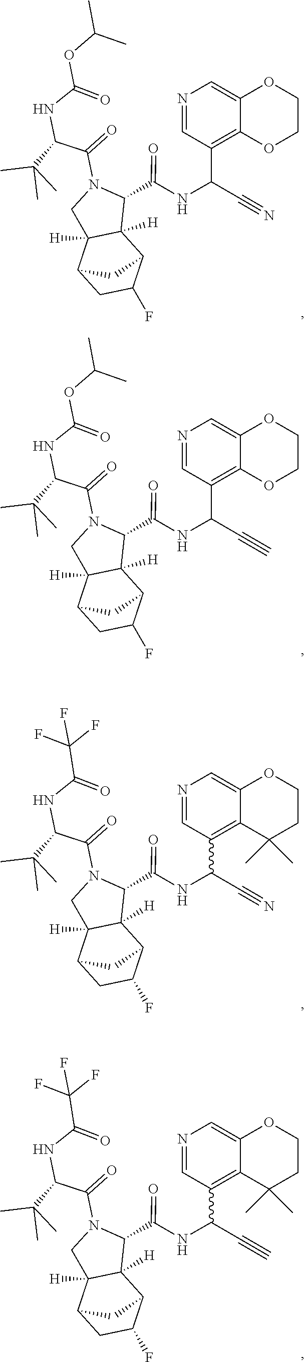

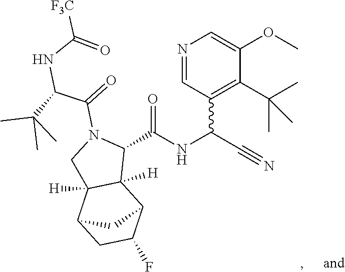 [SMILES: COc1cncc(C(C#N)NC(=O)[C@@H]2[C@@H]3[C@H](CN2C(=O)[C@@H](NC(=O)C(F)(F)F)C(C)(C)C)[C@H]2C[C@@H]3[C@H](F)C2)c1C(C)(C)C]

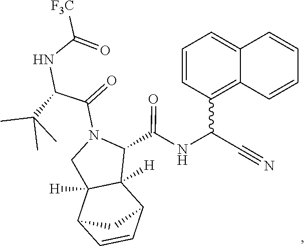 [SMILES: CC(C)(C)[C@H](NC(=O)C(F)(F)F)C(=O)N1C[C@H]2[C@@H]([C@H]1C(=O)NC(C#N)c1cccc3ccccc13)[C@H]1C=C[C@@H]2C1]

or a pharmaceutically acceptable salt of any of the foregoing.

Further examples of compounds of Formula (I), include the following:

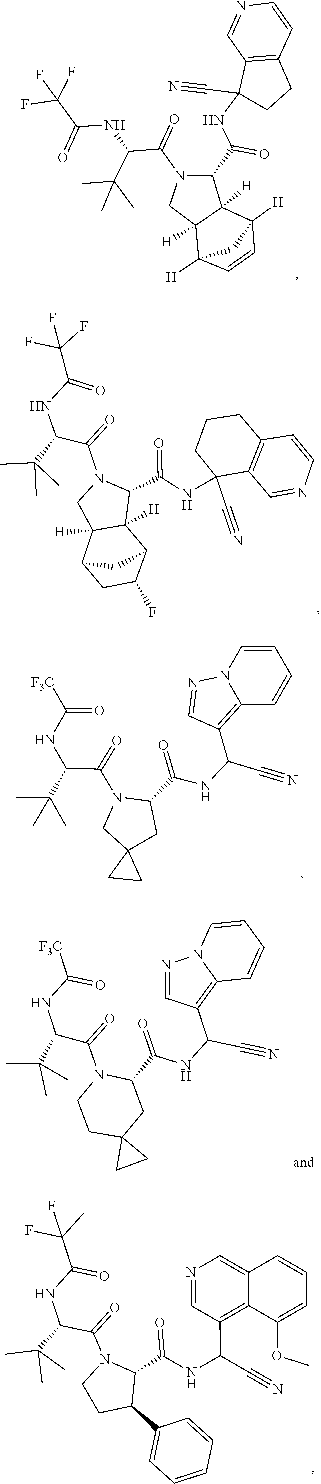 [SMILES: CC(C)(C)[C@H](NC(=O)C(F)(F)F)C(=O)N1C[C@H]2[C@@H]([C@H]1C(=O)NC1(C#N)CCc3ccncc31)[C@H]1C=C[C@@H]2C1 ; CC(C)(C)[C@H](NC(=O)C(F)(F)F)C(=O)N1C[C@@H]2[C@H]3C[C@@H]([C@@H]2[C@H]1C(=O)NC1(C#N)CCCc2ccncc21)[C@H](F)C3 ; CC(C)(C)[C@H](NC(=O)C(F)(F)F)C(=O)N1CC2(CC2)C[C@H]1C(=O)NC(C#N)c1cnn2ccccc12 ; CC(C)(C)[C@H](NC(=O)C(F)(F)F)C(=O)N1CCC2(CC2)C[C@H]1C(=O)NC(C#N)c1cnn2ccccc12 ; COc1cccc2cncc(C(C#N)NC(=O)[C@@H]3[C@@H](c4ccccc4)CCN3C(=O)[C@@H](NC(=O)C(C)(F)F)C(C)(C)C)c12]

or a pharmaceutically acceptable salt of any of the foregoing.

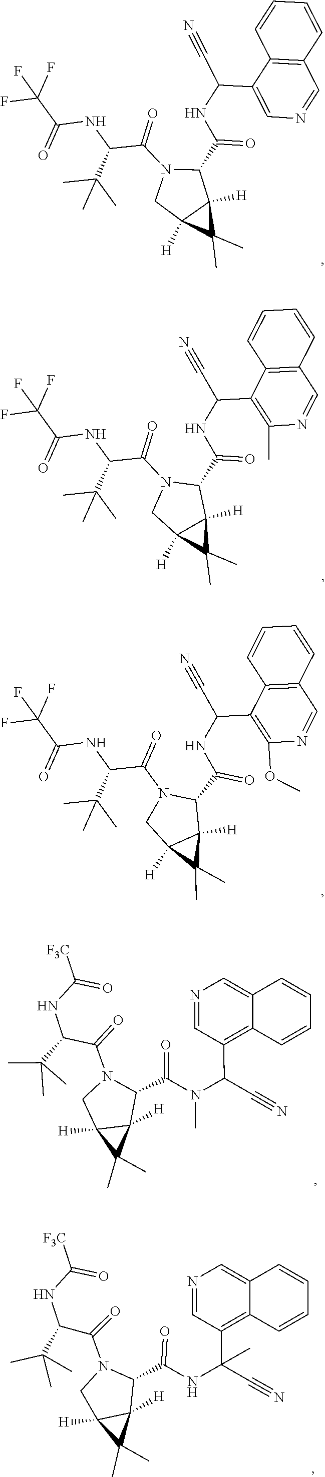 [SMILES: CC(C)(C)[C@H](NC(=O)C(F)(F)F)C(=O)N1C[C@H]2[C@@H]([C@H]1C(=O)NC(C#N)c1cncc3ccccc13)C2(C)C ; Cc1ncc2ccccc2c1C(C#N)NC(=O)[C@@H]1[C@@H]2[C@H](CN1C(=O)[C@@H](NC(=O)C(F)(F)F)C(C)(C)C)C2(C)C ; COc1ncc2ccccc2c1C(C#N)NC(=O)[C@@H]1[C@@H]2[C@H](CN1C(=O)[C@@H](NC(=O)C(F)(F)F)C(C)(C)C)C2(C)C ; CN(C(=O)[C@@H]1[C@@H]2[C@H](CN1C(=O)[C@@H](NC(=O)C(F)(F)F)C(C)(C)C)C2(C)C)C(C#N)c1cncc2ccccc12 ; CC(C#N)(NC(=O)[C@@H]1[C@@H]2[C@H](CN1C(=O)[C@@H](NC(=O)C(F)(F)F)C(C)(C)C)C2(C)C)c1cncc2ccccc12]

Further examples of compounds of Formula (I), include the following:

a pharmaceutically acceptable salt of any of the foregoing.

### Synthesis

Compounds of Formula (I) along with those described herein may be prepared in various ways. General synthetic routes for preparing compounds of Formula (I) are shown and described herein along with some examples of starting materials used to synthesize compounds described herein. Additionally, for the purpose of the general synthetic routes, the structures depicted are appropriately protected, as known by one skilled in the art and the generic structures are meant to include these protecting groups. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

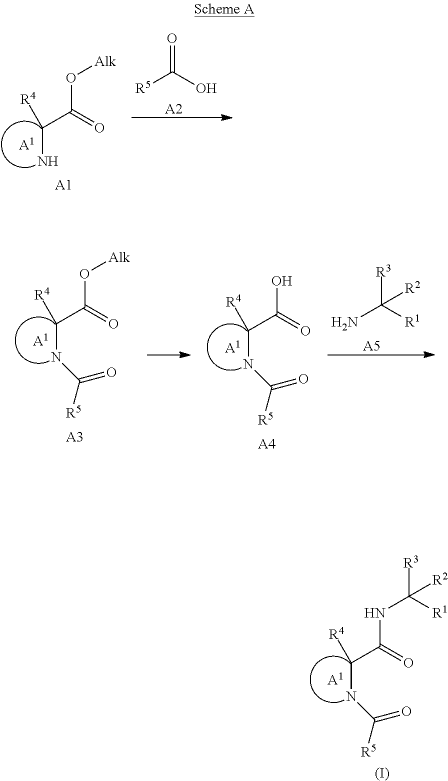

Scheme A describes the synthesis of compounds of general Formula (I). An amino ester of general Formula (A1) (Alk represents alkyl) with an acid of general Formula (A2), either by activating the carboxylic acid by converting it to an acid chloride, followed by reaction with the amino acid in the presence of a base, or by activation of the acid with a coupling reagent (such as HATU) followed by coupling with the amino ester in the presence of a base (such as DIPEA), resulting in a compound of general Formula (A3). The ester functionality of general Formula (A3) can be hydrolyzed, for example, under basic conditions if -OAlk is -Ome, using LiOH in CH₃OH, providing a compound of general Formula (A4). Further coupling of the carboxylic acid of general Formula (A4) with an amine of general Formula (A5) can provide a compound of Formula (I). For the purpose of the generic synthesis, R¹ may be a latent functionality, converted to a functionality as described herein for R¹.

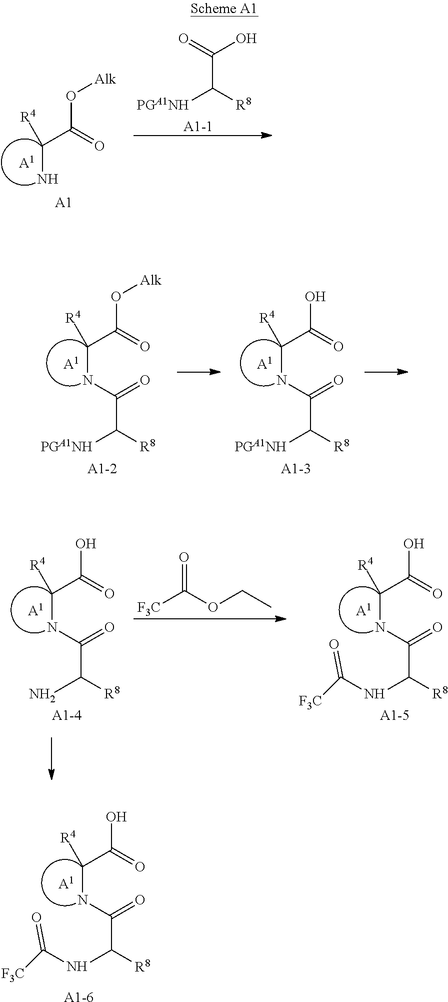

Alternatively, as described in Scheme A1, a sub-group of amino acids of general Formula (A1-5) can be prepared as described in Scheme A1. A protected (PG^{A1}) amino acid of general Formula (A1-1) can be coupled with an aminoester of general Formula (A1) under known amide formation conditions, for example, HATU and iPr₂Net. The ester of a compound of Formula (A1-2) can be deprotected, for example, by using LiOH in THF/H₂O, resulting in the acid of general Formula (A1-3). The protecting group PG^{A1} can be removed, for example, by treatment with TFA in case PG^{A1} is Boc, resulting in a compound of general Formula (A1-4). This compound can be converted to a compound of general Formula (A1-5), for example, by treatment with ethyl 2,2,2-trifluoroacetate in the presence of triethylamine or alternatively a compound of general Formula (A1-6), for example, by treatment of a compound of general Formula (A1-4) with an alkyl trihaloacetate, such as ethyl 2,2-dichloro-2-fluoroacetate, methyl 2-chloro-2,2-difluoroacetate or ethyl 2-chloro-2,2-difluoroacetate; in the presence of a base (for example, triethylamine and optionally an additive, such as N-methylimidazole), or an alkyl 2,2,3,3,3-pentafluoropropanoate (for example, methyl or ethyl 2,2,3,3,3-pentafluoropropanoate) in the presence of a base, for example, triethylamine and an additive, for example, N-methylimidazole (NMI).

General methodology for the synthesis of amino acids of general Formula (A1-1), or precursors that could be converted to an amino acid of general Formula (A1-1) using methods and materials known to one skilled in the art, are described in the literature, and include the following examples:

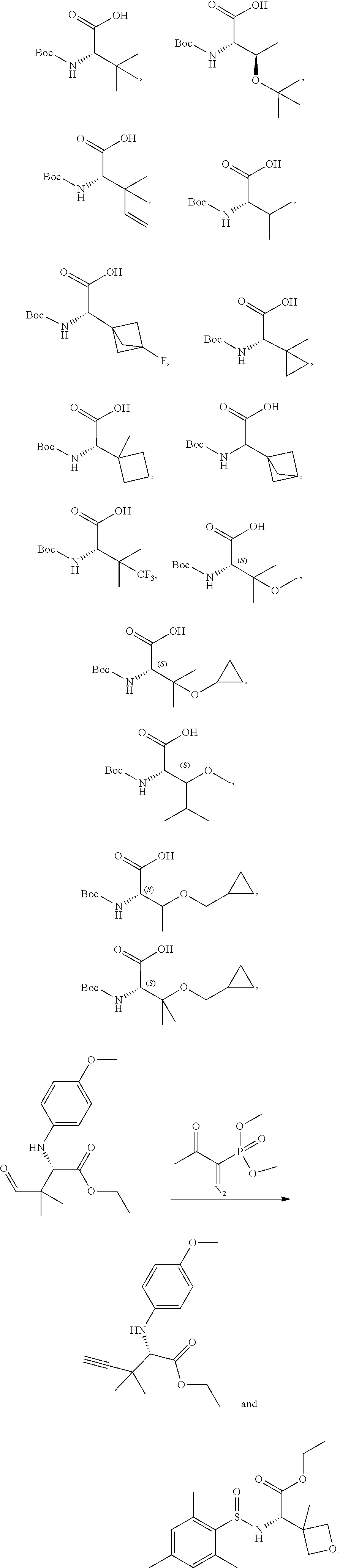

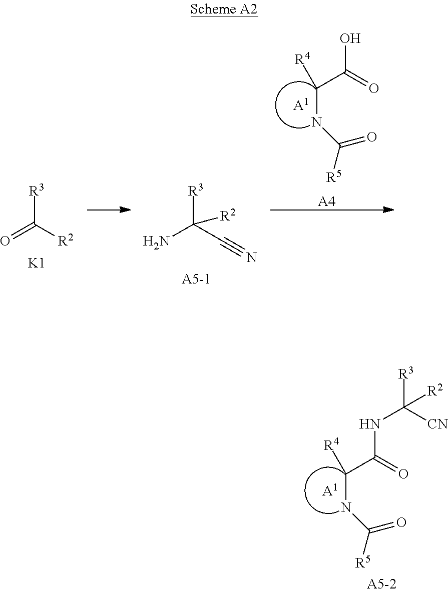

In the event that R¹ is CN, the synthesis of the compounds of Formula (A5-1) may be obtained in the following way as shown in Scheme A2. An aldehyde or ketone of the Formula (K-1) can be reacted with NH₃, KCN (or alternatively TMSCN), in an alcoholic solvent (e.g., methanol) to afford the aminonitrile compounds of Formula (A5-1). Enantiomeric mixtures may be used in subsequent steps, or alternatively, separated by chiral HPLC or supercritical fluid chromatography (SFC). Compounds of the Formula (A5-1) can be reacted with a carboxylic acid of the Formula (A4) using standard amino acid coupling conditions to afford compounds of the Formula (A5-2).

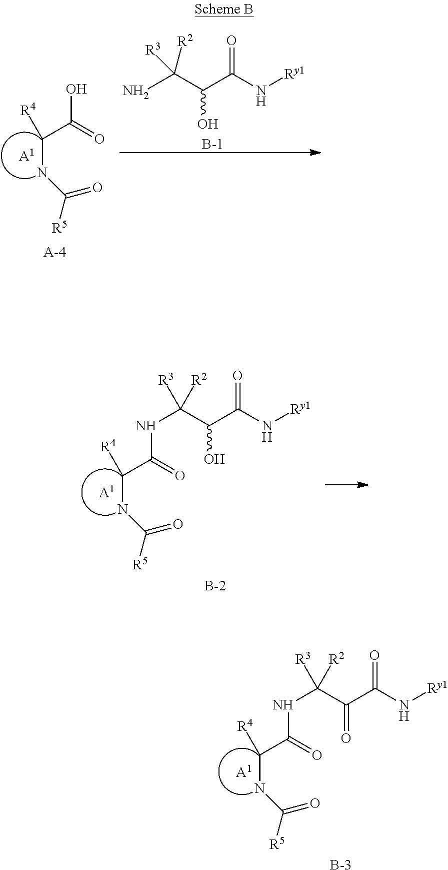

In Scheme B, a carboxylic acid of general Formula (A-4) can be coupled with an amino acid of general Formula (B-1), for example, under the influence of a coupling reagent (such as T3P) and a base (for example, DIPEA). The obtained compound of general Formula (B-2) can be oxidized, providing in a compound of general Formula (B-3). In Scheme B, R^{y1} can be part of the ketoamide described herein with respect to R¹.

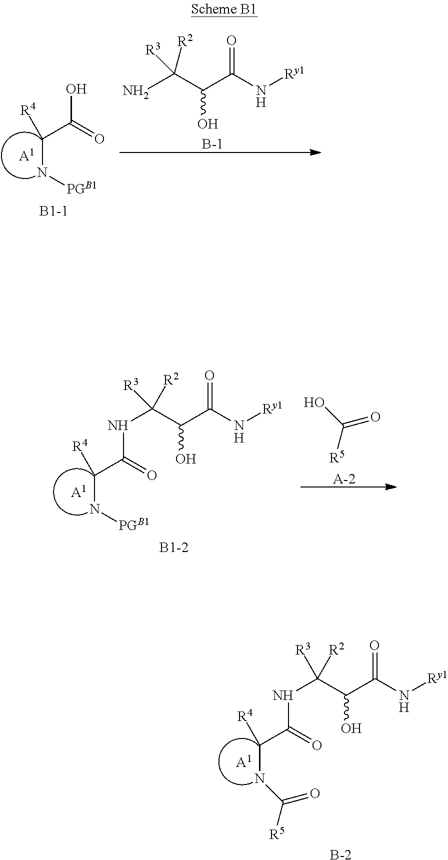

Alternatively, as depicted in Scheme B1, an amino acid of general Formula (B1-1) (with PG^{B1}, a protecting group of the nitrogen, for example, -Boc) can be coupled with a compound of general Formula (B-1), similar as described for the conversion of a compound of general Formula (A-4) to a compound of general Formula (B-2). The protecting group can be removed, for example, by treatment with an acid in case of PG^{B1} being Boc, followed by coupling with a compound of general Formula (A-2), resulting in the formation of a compound of general Formula (B-2).

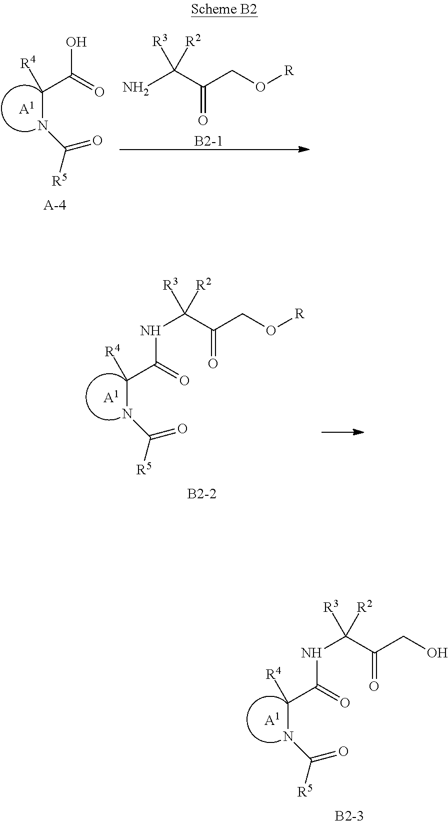

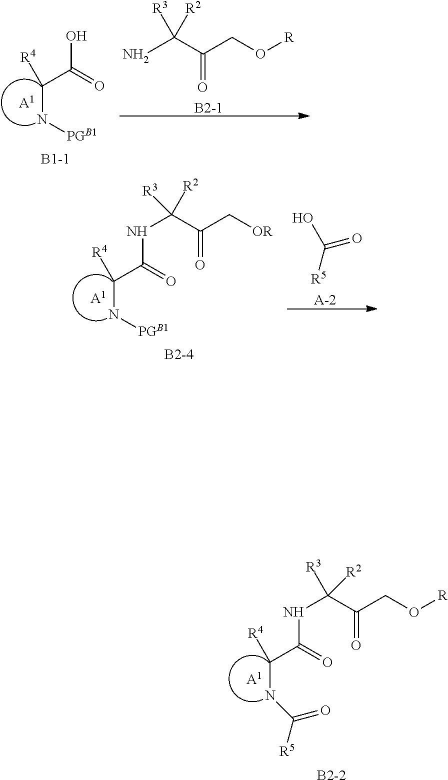

As described herein, R¹ can be a substituted acyl, where the possible groups that can be present on the acyl include hydroxy, a substituted or an unsubstituted alkoxy (for example, —O-(an unsubstituted C₁₋₄ alkyl) and —O-(an unsubstituted C₃₋₆ cycloalkyl)), an unsubstituted C₁₋₄ alkyl (such as a heteroaryl substituted with an unsubstituted C₁₋₄ alkyl), a substituted or an unsubstituted phenoxy or a substituted or an unsubstituted benzyloxy). In Scheme B2, R can represent any of the aforementioned moieties that can be present on a substituted acyl for R¹. Compounds of general Formulae (B2-2) and (B2-3) can be prepared as described in Scheme B2. An amino-ketone compound of general Formula (B2-1) can be coupled to a carboxylic acid of general Formula (A-4) or (B1-1) under typical amide coupling conditions. A compound of general Formula (B2-2) can be optionally further converted in a hydroxyketone of general Formula (B2-3), for example, in case where R represents a benzyl group, by catalytic hydrogenolysis. The PG^{B1} of a compound of general Formula (B2-4) can be deprotected (for example, in the case wherein PG^{B1} is a Boc-group, by treatment with HCl in Et₂O). The amine can then be coupled with a carboxylic acid of general Formula (A-2) under typical amide bond formation conditions, to provide a compound of general Formula (B2-2).

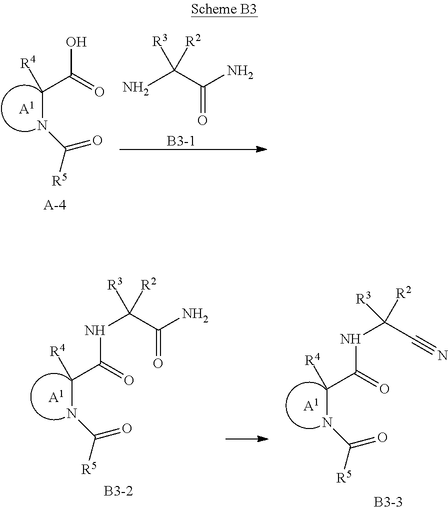

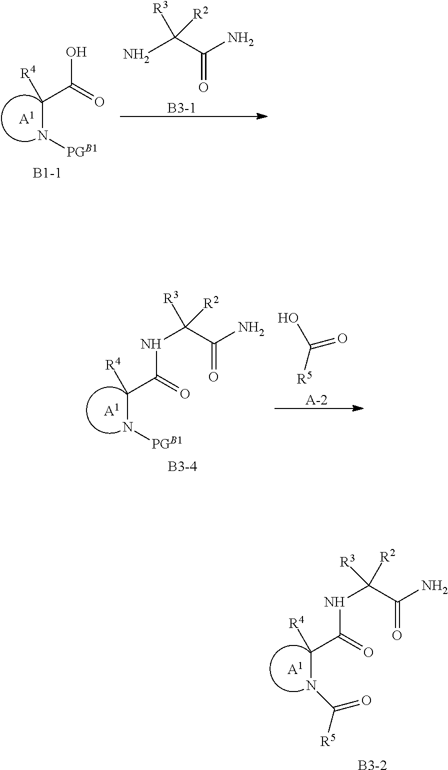

The preparation of compounds of Formula (B3-3) are shown in Scheme B3. Substituted amides of the Formula (B3-1) may be coupled to carboxylic acids of Formula (A-4) using standard amino acid coupling conditions to afford amide compounds of Formula (B3-2). Dehydration of the primary amide, employing one of several conditions described in the literature (for example, trifluoroacetic anhydride (TFAA) and pyridine in CH₂Cl₂, or by application of the Burgess reagent), can afford compounds of Formula (B3-3).

The preparation of compounds of Formula (B3-2) are also shown in Scheme B3. Substituted amides of the Formula (B3-1) may be coupled to carboxylic acids of Formula (B1-1) using standard amino acid coupling conditions to afford compounds of Formula (B3-4). Deprotection of the protecting group PG^{B1} (for example using HCl when PG^{B1} is a Boc group), followed by the coupling of the carboxylic acids of Formula (A-2) using standard coupling conditions, can afford compounds of the Formula (B3-2).

The preparation of compounds of Formula (B3-1) are also described in the literature and include but are not limited to the following references: Shi et al., “Direct Synthesis of α-Amino Nitriles from Sulfonamides via Base-Mediated C-H Cyanation” Org. Lett. (2021) 23(10):4018-4022, and Corey et al, “Enantioselective Synthesis of α-Amino Nitriles from N-Benzhydryl Imines and HCN with a Chiral Bicyclic Guanidine as Catalyst” Org. Lett. (1999) 1(1):157-160. A highly efficient, direct C(sp3)-H cyanation under mild photocatalytic conditions offers functional group tolerance. Complex natural products and bioactive compounds have been efficiently cyanated. Kim et al., “Direct C(sp³)-H Cyanation Enabled by a Highly Active Decatungstate Photocatalyst” Org. Lett. (2021) 23(14): 5501-5505. An N-heterocyclic carbene as a nucleophilic organocatalyst can allow for the cyanation of ketones and ketimines with TMSCN in good yields under mild reaction conditions. Fukuda et al. “Construction of Tetrasubstituted Carbon by an Organocatalyst: Cyanation Reaction of Ketones and Ketimines Catalyzed by a Nucleophilic-N-Heterocyclic Carbene” Synthesis, (2006) 16:2649-2652.

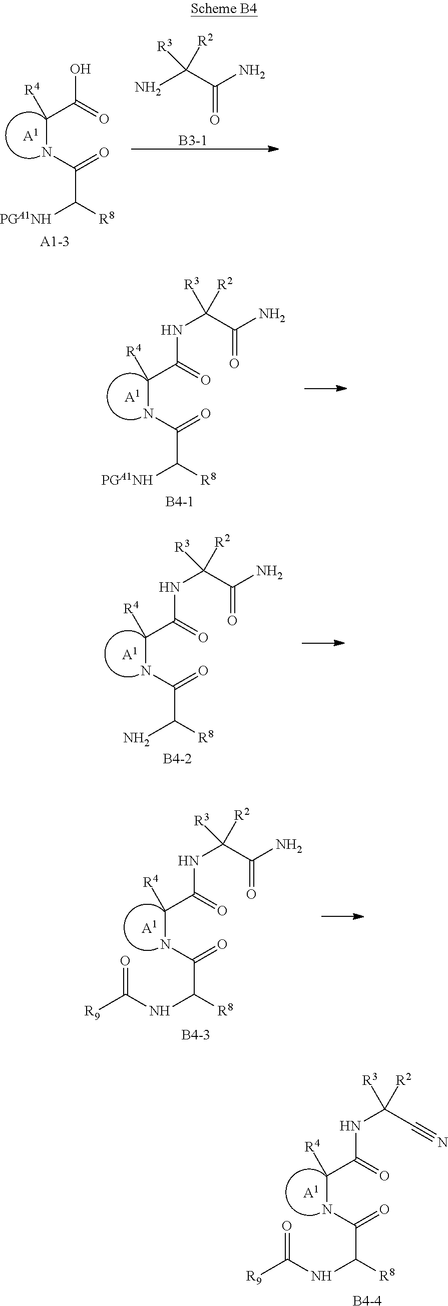

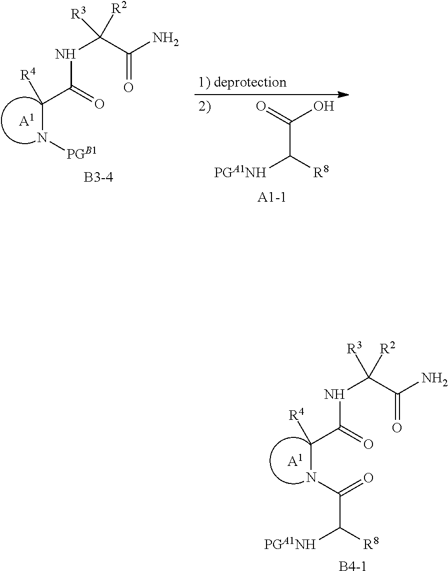

In Scheme B4, a compound of general Formula (A1-3) can be coupled with amine of Formula (B3-1) to obtain a compound of general Formula (B4-1), where PG^{A1} can be a protecting group which can be removed, for example, in case PG^{A1} is Boc, for example, by treatment with HCl or TFA. The compound of general Formula (B4-2) can then be converted in a compound of general Formula (B4-3), for example, by treatment with an alkyl trihaloacetate (such as ethyl 2,2-dichloro-2-fluoroacetate, methyl 2-chloro-2,2-difluoroacetate ethyl 2-chloro-2,2-difluoroacetate or ethyl 2,2,2-trifluoroacetate) in the presence of a base (for example, triethylamine and optionally an additive, such as N-methylimidazole) or an alkyl 2,2,3,3,3-pentafluoropropanoate (such as methyl or ethyl 2,2,3,3,3-pentafluoropropanoate) in the presence of a base (for example, triethylamine and optionally an additive, such as N-methylimidazole) or a carboxylic acid in the presence of a coupling reagent, such as EDC or HATU, and a base (for example, Net₃) The compound of general Formula (B4-3) can be converted to a compound of general Formula (B4-4) in a similar way as outlined for the conversion of a compound of Formula (B3-2) to a compound of Formula (B3-3). Alternatively, a compound of Formula (B4-2) can be converted to a compound of Formula (B4-4), for example, by treatment with T3P and pyridine in the presence of potassium 2,2,3,3,3-pentafluoropropanoate when —R⁹ is —CF₂CF₃. In addition, a compound of general Formula (B4-1) can be obtained by deprotection of PG^{B1} of a compound of general Formula (B3-4), followed by coupling with a compound of general Formula (A1-1).

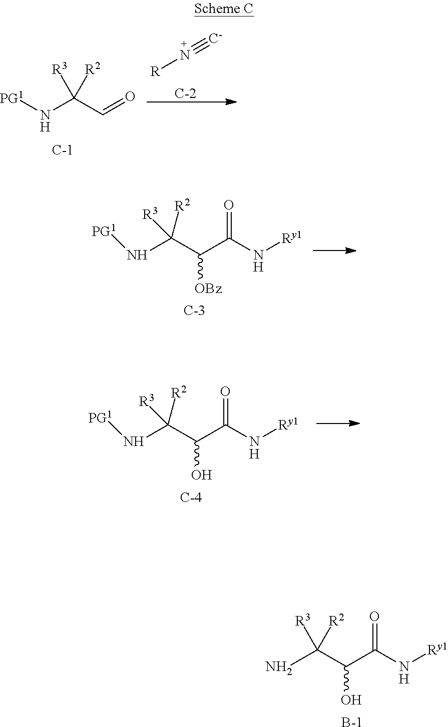

A compound of general Formula (B-1) can be prepared as outlined in Scheme C. An aldehyde of general Formula (C-1) (PG¹ can be a nitrogen protecting group, for example -Boc) and an isonitrile of general Formula (C-2), in the presence of a carboxylic acid (for example, benzoic acid), can be condensed in a Passerini-like reaction towards a compound of general Formula (C-3). After hydrolysis, a compound of general Formula (C-4) can be obtained. The PG¹ can be removed, for example, by treatment with HCl when PG¹ is Boc to afford a compound of Formula (B-1).

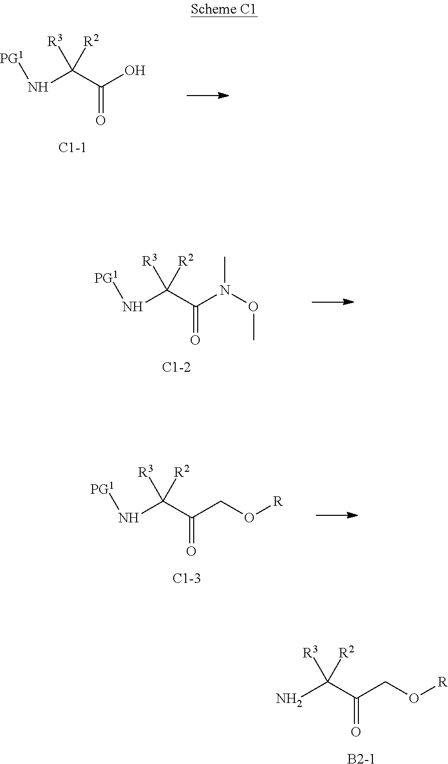

An amino ketone of general Formula (B2-1), can be prepared as outlined in Scheme C1. A protected amino acid of general Formula (C1-1) can be converted to its corresponding N-methoxy-N-methyl amide (Weinreb amide) under typical amide coupling conditions. Addition of an organometallic reagent to the Weinreb amide, followed by work-up, can result in a ketone of general Formula (C1-3). An example, wherein R can be benzyl, is the formation of an organometallic reagent by mixing Mg, HgCl₂ and benzylchloromethyl ether, followed by addition to a Weinreb amide of general Formula (C1-2), followed by work-up with saturated NH₄Cl. The protecting group (PG¹) can be removed (for example, when PG¹ is Boc, the protecting group can be removed using HCl) resulting in the formation of an amino ketone of general Formula (B2-1). When HCl is used for the deprotection, a compound of general Formula (B2-1) can be obtained as an HCl salt.

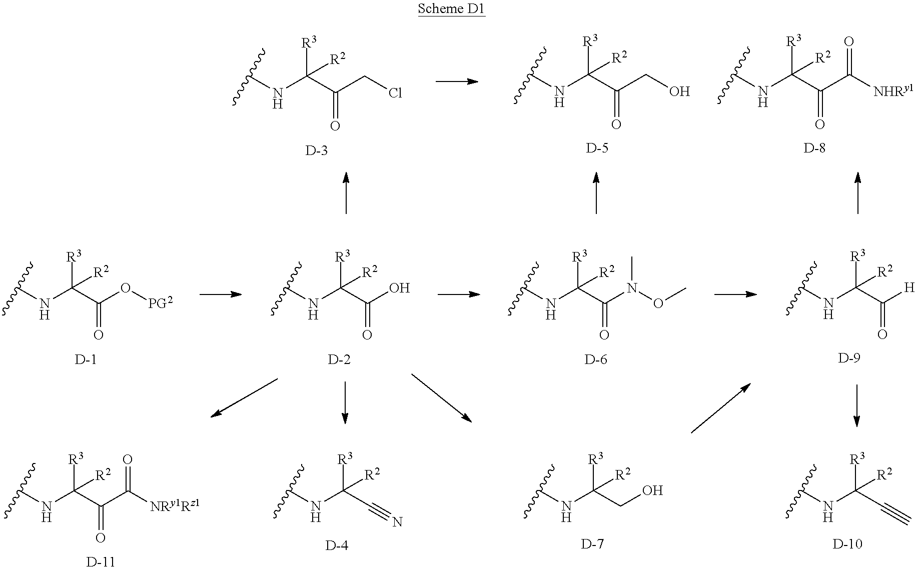

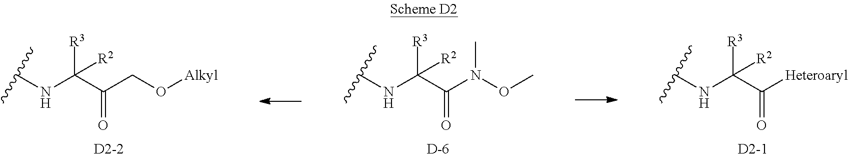

Other conversions for R¹ described herein are shown in Schemes D1 and D2. In Schemes D1 and D2, PG² represents an appropriate protecting group, and R^{z1} and R^{y1} are part of the ketoamide described herein with respect to R¹.

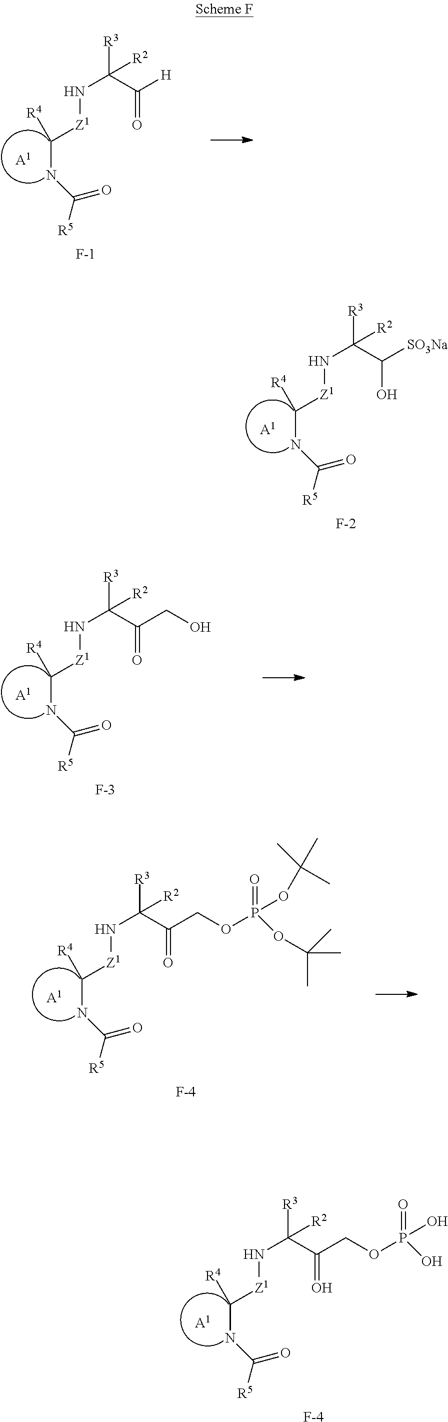

Compounds of Formula (I) can include a prodrug moiety. A method for including a prodrug moiety is depicted in Scheme F. For example, an aldehyde of general Formula (F-1) can be transformed into the corresponding bisulfite adduct of general Formula (F-2), by treatment with NaHSO₃. A hydroxyketone of general Formula (F-3), can be transformed to the corresponding phosphate of general Formula (F-5), for example, by treatment with di-tert-butyl N,N-dipropan-2-ylphosphoramidite and tetrazole followed by oxidation with H₂O₂, that can provide a compound of general Formula (F-4). A compound of general Formula (F-4) can be deprotected (for example by treatment with TFA) to provide a compound of general Formula (F-5).

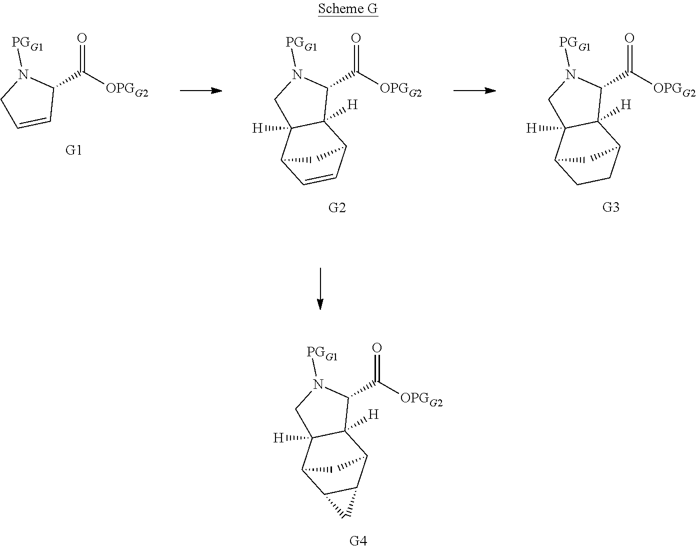

As shown in Scheme G, the synthesis of an amino ester of general Formula (G2) can be accomplished via a Diels-Alder reaction, such as described in Arakawa et al., Chemical & Pharmaceutical Bulletin (2003) 51(8):1015-1020 (-PG_{G1} can be -Bz and -PG_{G2} can be —CH₃). Also described herein in the synthesis of intermediates, where -PG_{G1} is -Boc and -PG_{G2} is -t-Butyl or Me. A compound of general Formula (G2) can be deprotected using methods known to those skilled in the art and depending on the protecting group used for PG_{G1} and PG_{G2}. Alternatively, a compound of general Formula (G2) can be converted to a compound of general Formula (G3), by hydrogenation of the double bond, or to a compound of general Formula (G4), by cyclopropanation of the double bond. The cyclopropanation can, for example, be performed by application of a Simmons-smith cyclopropanation, by treatment with CH₂N₂ in the presence of Pd(OAc)₂, or other methods described known to those skilled in the art. Alternatively, deuterated intermediates can be used.

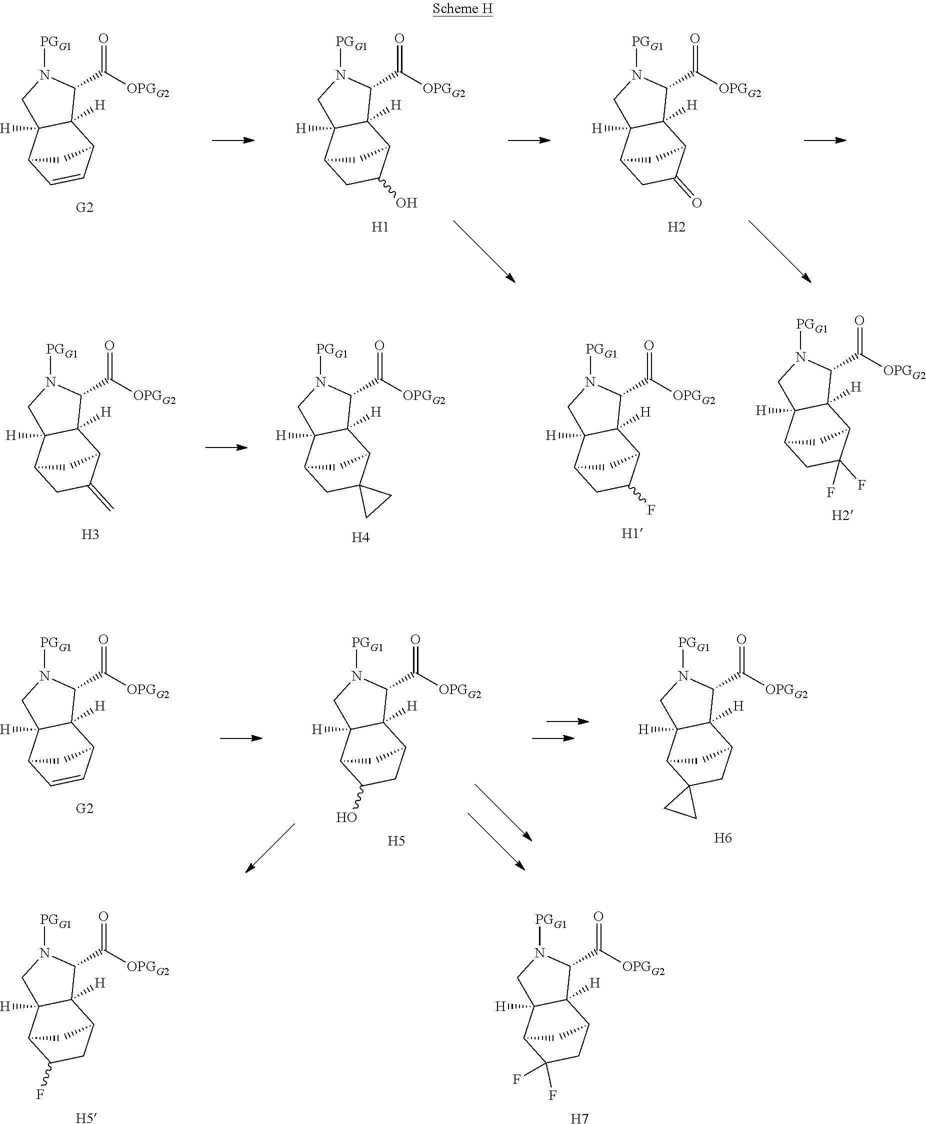

Other intermediates are described in Scheme H. The intermediate of general Formula (G2), can be selectively hydroxylated, for example, by hydrosilylation with trichlorosilane in the presence of a chiral Pd-catalyst, followed by SiCl₃/OH exchange (for example, Breuning et al, Beilstein Journal of Organic Chemistry (2009) 5(81):1-5). Oxidation of the alcohol of general Formula (H1) can provide a ketone of general Formula (H2). The ketone of general Formula (H2) can be converted to an alkene of general Formula (H3), for example, by using a Wittig or a Tebbe reagent. Transformation of the double bond towards the cyclopropyl can be done by treatment with CH₂N₂ in the presence of Pd(Oac)₂, or other methods described in the literature and known to those skilled in the art, and can result in a compound of general Formula (H4). A similar approach can be done with the isomer of a compound of general Formula (H1), a compound of general Formula (H5) can be obtained by using an enantiomeric chiral Pd-catalyst. A compound of general Formula (H5) can then be converted to a compound of general Formula (H6), similar as outlined for the conversion of a compound of general Formula (H1) to a compound of general Formula (H4). Alternatively, the ketone of compound of general Formula (H2) can be converted to a compound of general Formula (H2′) by fluorination, for example by application of the DAST (diethylaminosulfur trifluoride) reagent. The isomeric compound of general Formula (H7) can be obtained starting from a related isomer. The alcohols of general Formulae (H1) and (H5) can be converted to the related fluoro derivatives of general Formulae (H1′) and (H5′), by treatment with a fluorination reagent like DAST.

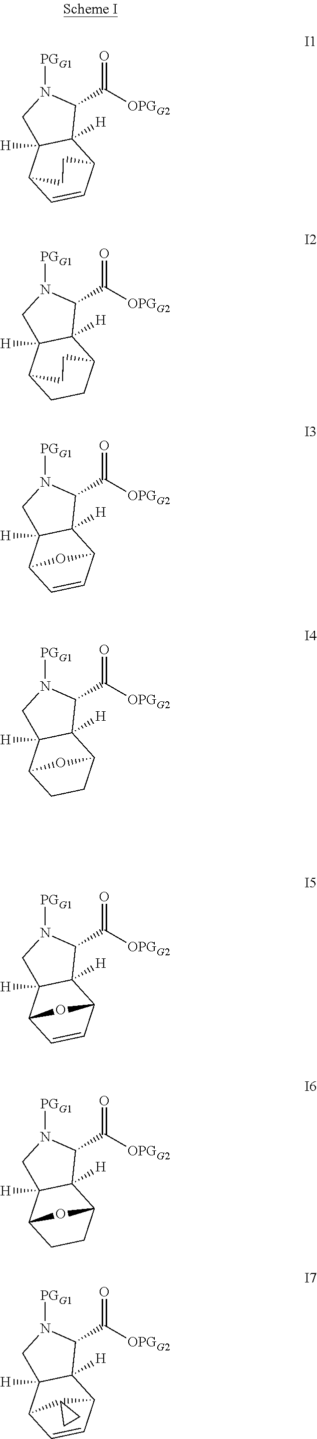

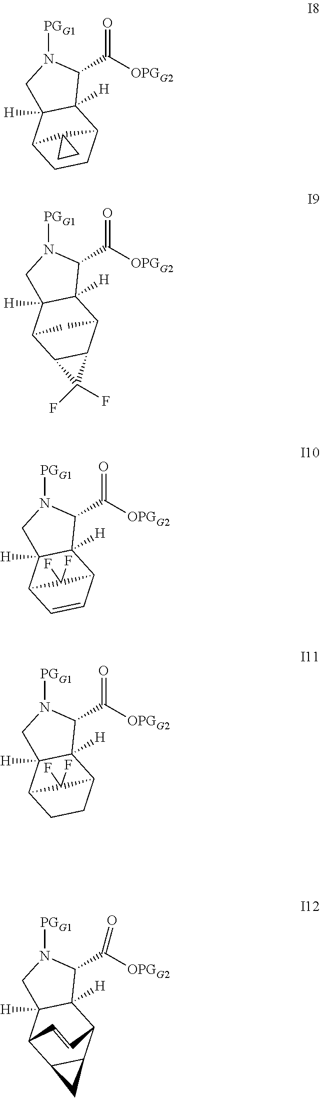

Other compounds of general Formulae (I1), (I2) (Johnson et al., Synthetic Communications (2011) 41(18):2769-2793), (I3), (I4), (I5), (I6), (I7), (I8), (I9), (I10), (I11) and (I12) as depicted in Scheme I can be obtained by methods described in literature (for example, de Graaff et al., Org. Biomol. Chem. (2015) 13:10108-10112; and Johnson et al., Synthetic Communications (2011) 41(18):2769-2793) and/or by applying methodologies as described herein. Compounds general Formulae (I1), (I2), (I3), (I4), (I5), (I6), (I7), (I8), (I9), (I10), (I11) and (I12) can be used to obtain compounds of Formula (I), along with pharmaceutically acceptable salts, using similar methods as described herein.

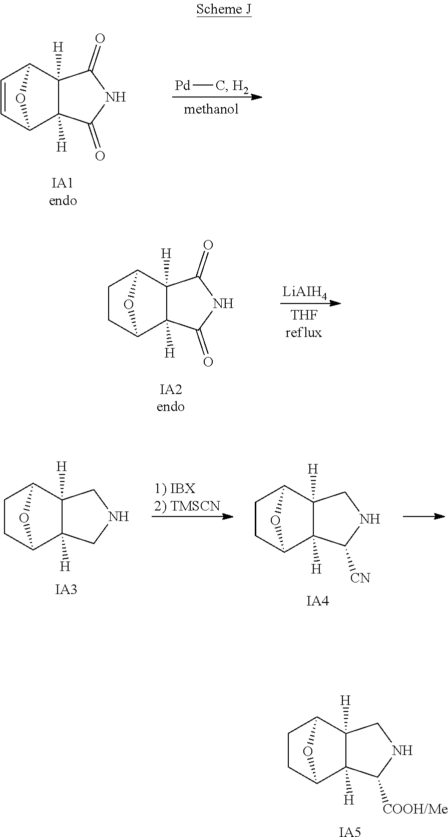

As an example, as depicted in Scheme J, a compound of Formula (IA1) (Rulíšek et al., J. Org. Chem. (2005) 70(16):6295-6302) can be hydrogenated to a compound of Formula (IA2). After reduction of a compound of Formula (IA2), (for example, with LiAlH₄ (Johnson et al., Synthetic Communications (2011) 41(18):2769-2793), can result in a compound of Formula (IA3). A compound of Formula (IA3) can be oxidized using IBX (de Graaff et al., Org. Biomol. Chem. (2015) 13:10108-10112) followed by introduction of nitrile (Liu et al., Org. Process Res. Dev. (2016) 20(2):320-324) to provide a compound of Formula (IA4). The nitrile can next be converted to a carboxylic acid or ester of a compound of Formula (IA5). In the above scheme, racemic material can be obtained upon nitrile introduction from a compound of Formula (IA3) to a compound of Formula (IA4). Alternatively, a chiral method can be used to provide enantioenriched compound(s).

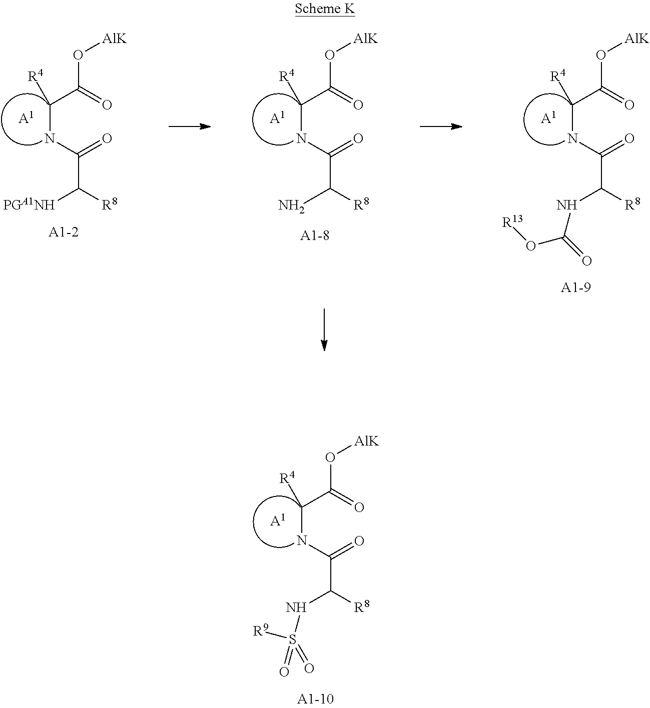

Scheme K describes the transformation of compounds of Formula (A1-2) to compounds of Formulae (A1-9) and (A1-10). Compounds of Formula (A1-2) can be deprotected, by liberating the amine group. The amine group of a compound of Formula (A1-8) can be reacted with a chloroformate, in the presence of a base to form the carbamate of Formula (A1-9). Alternatively, the amine group of a compound of Formula (A1-8) can be reacted with a sulfonyl chloride, in the presence of a base, to afford sulfonamides of the Formula (A1-10).

### Pharmaceutical Compositions

Some embodiments described herein relate to a pharmaceutical composition, that can include an effective amount of a compound described herein (e.g., a compound, or a pharmaceutically acceptable salt thereof, as described herein) and a pharmaceutically acceptable carrier, excipient or combination thereof. A pharmaceutical composition described herein is suitable for human and/or veterinary applications.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

Proper formulation is dependent upon the route of administration chosen. Techniques for formulation and administration of the compounds described herein are known to those skilled in the art. Multiple techniques of administering a compound exist in the art including, but not limited to, oral, rectal, topical, aerosol, injection, inhalation and parenteral delivery, including intramuscular, subcutaneous, intravenous, intramedullary injections, intrathecal, direct intraventricular, intraperitoneal, intranasal and intraocular injections. Pharmaceutical compositions will generally be tailored to the specific intended route of administration.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes may be targeted to and taken up selectively by the organ.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. As described herein, compounds used in a pharmaceutical composition may be provided as salts with pharmaceutically compatible counterions.

### Methods of Use

Some embodiments described herein relate to a method of treating a coronavirus infection that can include administering to a subject identified as suffering from the coronavirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a coronavirus infection. Still other embodiments described herein relate to the use of a compound, or a pharmaceutically acceptable salt thereof, as described herein or a pharmaceutical composition that includes a compound, or a pharmaceutically acceptable salt thereof, as described herein for treating a coronavirus infection.

Some embodiments disclosed herein relate to a method of treating a coronavirus infection that can include contacting a cell infected with the coronavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a coronavirus infection. Still other embodiments described herein relate to the use of a compound, or a pharmaceutically acceptable salt thereof, as described herein described herein, or a pharmaceutical composition that includes an effective amount of a compound, or a pharmaceutically acceptable salt thereof, as described herein for treating a coronavirus infection.

Some embodiments disclosed herein relate to a method of inhibiting replication of a coronavirus that can include contacting a cell infected with the coronavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for inhibiting replication of a coronavirus. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein, for inhibiting replication of a coronavirus.

In some embodiments, the coronavirus can be an α-coronavirus or a β-coronavirus. A compound described herein may be effective against one or more variants of a coronavirus. Examples of variants include, but are not limited, to alpha-variant (B.1.1.7), beta-variant (B.1.351), gamma variant (P.1) and delta-variant (B.1.617.2). In some embodiments, the coronavirus can be selected from CoV 229E, CoV NL63, CoV OC43, CoV HKU1, Middle East Respiratory Syndrome (MERS)-CoV, Severe Acute Respiratory Syndrome (SARS)-CoV, and SARS-CoV-2.

Some embodiments described herein relate to a method of treating a picornavirus infection that can include administering to a subject identified as suffering from the picornavirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a picornavirus infection. Still other embodiments described herein relate to the use of a compound, or a pharmaceutically acceptable salt thereof, as described herein or a pharmaceutical composition that includes a compound, or a pharmaceutically acceptable salt thereof, as described herein for treating a picornavirus infection.

Some embodiments disclosed herein relate to a method of treating a picornavirus infection that can include contacting a cell infected with the picornavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a picornavirus infection. Still other embodiments described herein relate to the use of a compound, or a pharmaceutically acceptable salt thereof, as described herein described herein, or a pharmaceutical composition that includes an effective amount of a compound, or a pharmaceutically acceptable salt thereof, as described herein for treating a picornavirus infection.

Some embodiments disclosed herein relate to a method of inhibiting replication of a picornavirus that can include contacting a cell infected with the picornavirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for inhibiting replication of a picornavirus. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein, for inhibiting replication of a picornavirus.

In some embodiments, the picornavirus can be a rhinovirus, including rhinovirus A, B and/or C. In some embodiments, a compound described herein, including a compound of Formula (I), or a pharmaceutically acceptable salt thereof, can be used to treat one or serotypes of a rhinovirus.

Some embodiments described herein relate to a method of treating a norovirus infection that can include administering to a subject identified as suffering from the norovirus infection an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a norovirus infection. Still other embodiments described herein relate to the use of a compound, or a pharmaceutically acceptable salt thereof, as described herein or a pharmaceutical composition that includes a compound, or a pharmaceutically acceptable salt thereof, as described herein for treating a norovirus infection.

Some embodiments disclosed herein relate to a method of treating a norovirus infection that can include contacting a cell infected with the norovirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a norovirus infection. Still other embodiments described herein relate to the use of a compound, or a pharmaceutically acceptable salt thereof, as described herein described herein, or a pharmaceutical composition that includes an effective amount of a compound, or a pharmaceutically acceptable salt thereof, as described herein for treating a norovirus infection.

Some embodiments disclosed herein relate to a method of inhibiting replication of a norovirus that can include contacting a cell infected with the norovirus with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for inhibiting replication of a norovirus. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein, for inhibiting replication of a norovirus.

Some embodiments disclosed herein relate to a method of treating a respiratory condition that is developed because of a coronavirus and/or a picornavirus infection that can include administering to a subject suffering from the respiratory condition and/or contacting a cell infected with the coronavirus and/or the picornavirus in a subject suffering from the respiratory condition with an effective amount of a compound, or a pharmaceutically acceptable salt thereof, as described herein, or a pharmaceutical composition that includes an effective amount of a compound, or a pharmaceutically acceptable salt thereof, as described herein. Other embodiments described herein relate to using a compound, or a pharmaceutically acceptable salt thereof, as described herein in the manufacture of a medicament for treating a respiratory condition due to a coronavirus infection and/or a picornavirus infection with an effective amount of the compound, or a pharmaceutically acceptable salt thereof. Still other embodiments described herein relate to the use of a compound, or a pharmaceutically acceptable salt thereof, as described herein, or a pharmaceutical composition that includes an effective amount of a compound, or a pharmaceutically acceptable salt thereof, as described herein for treating a respiratory condition due to a coronavirus infection and/or a picornavirus infection.

A subject infected with a coronavirus can be asymptotic. A coronavirus infection can manifest itself via one or more symptoms. Examples of symptoms include, but are not limited to, coughing, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, haemoptysis, conjunctival congestion, sputum production, chest tightness and/or palpitations. A coronavirus infection can cause complications. A non-limiting list of complications include, but are not limited to, sinusitis, otitis media, pneumonia, acute respiratory distress syndrome, disseminated intravascular coagulation, pericarditis and/or kidney failure.

As with a coronavirus, a subject infected with a picornavirus can be asymptotic. Alternatively, a subject can exhibit one or more of symptoms. Examples of symptoms of a picornavirus infection include, but are not limited to, aseptic meningitis, rash, conjunctivitis, runny nose a headache a cough a fever a sore throat, chest and/or abdominal pain and paralysis. As provided herein, subjects infected with a norovirus can exhibit one or more the symptoms including, but not limited to, nausea, non-bloody diarrhea, vomiting and abdominal pain. An example of a complication that can be attributed to a norovirus infection is dehydration, including severe dehydration.

Various indicators for determining the effectiveness of a method for treating a coronavirus, picornavirus and/or norovirus infection are also known to those skilled in the art. Examples of suitable indicators include, but are not limited to, a reduction in viral load indicated by reduction in coronavirus (or load) (e.g., reduction <10⁵ copies/mL in serum), a reduction in plasma viral load, a reduction in viral replication, a reduction in time to seroconversion (virus undetectable in patient serum), an increase in the rate of sustained viral response to therapy a reduction of morbidity or mortality in clinical outcomes, reduction in the need for a ventilator and/or total time on a ventilator, reduction in hospitalization rates and/or reduction in time in an ICU (intensive care unit) and/or hospital.

As used herein, the terms “treat,” “treating,” “treatment,” “therapeutic,” and “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the subject's overall feeling of well-being or appearance.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, camels, non-human primates, such as monkeys, chimpanzees, and apes, and, in particular, humans. In some embodiments, the subject can be human, for example, a human subject that is 60 years old or older.

The term “effective amount” is used to indicate an amount of an active compound, or pharmaceutical agent, that elicits the biological or medicinal response indicated. For example, an effective amount of compound can be the amount needed to alleviate or ameliorate symptoms of disease or prolong the survival of the subject being treated. This response may occur in a tissue, system, animal or human and includes alleviation of the signs or symptoms of the disease being treated. Determination of an effective amount is well within the capability of those skilled in the art, in view of the disclosure provided herein. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

In some embodiments, the subject can be asymptomatic, for example, the subject can be infected with coronavirus but does not exhibit any symptoms of the viral infection. In some embodiments, the subject can be have a pre-existing condition, such as asthma, hypertension, immunocompromised subjects (such as subjects with cancer, HIV and/or genetic immune deficiencies, bone marrow transplant subjects, solid organ transplant subjects, subjects who have had stem cells for cancer treatment and/or subjects who use oral or intravenous corticosteroids or other medicines called immunosuppressants), liver disease, subjects at risk for severe illness, chronic kidney disease being treated with dialysis, chronic lung disease, diabetes, hemoglobin disorders, serious heart conditions (for example, heart failure, coronary artery disease, congenital heart disease, cardiomyopathies, and pulmonary hypertension), severe obesity (such as subjects with a body mass index (BMI) of 40 or above) and people who live in a nursing home or long-term care facility. Additional examples and/or further information is provided by the CDC (https://www.cdc.gov/coronavirus/2019-ncov/need-extra-precautions/groups-at-higher-risk.html).

A compound described herein, including a compound of Formula (I), or a pharmaceutically acceptable salt thereof, can be administered after a subject is infected with a coronavirus. In addition and/or alternatively, a compound described herein, including a compound of Formula (I), or a pharmaceutically acceptable salt thereof, can be administered prophylactically.

Examples of agents that have been used to treat a coronavirus infection include Remdesivir. However, there can be drawbacks associated with compounds being used to treat a coronavirus including, but not limited to, one or more adverse side effects, the need for subcutaneous administration and/or high cost. Potential advantages of a compound of Formula (I), or a pharmaceutically acceptable salt thereof, can be less adverse side effects, delay in the onset of an adverse side effect and/or reduction in the severity of an adverse side effect.

A coronavirus infection can be treated by inhibiting certain mechanisms. In some embodiments, a compound described herein (such as a compound of Formula (I), or a pharmaceutically acceptable salt thereof) can be selective for a coronavirus protease. For example, a compound of Formula (I), or a pharmaceutically acceptable salt thereof, can be selective for a coronavirus protease compared to a host protease, for example, one or more host proteases selected from Cathepsin L, Cathepsin B, Cathepsin D, Cathepsin K, Leukocyte Elastase, Chymotrypsin, Trypsin, Thrombin, Pepsin, Caspase 2, Elastase and Calpain. In some embodiments, the selectivity for a coronavirus protease over a host protease (such as those described herein) can be >2-fold. In some embodiments, the selectivity for a coronavirus protease over a host protease (such as those described herein) can be >10-fold. In some embodiments, the selectivity for a coronavirus protease over a host protease (such as those described herein) can be >100-fold.

Studies have shown that the entry of SARS-CoV-2 into the target cells is a process that can be mediated by multiple proteases including cysteine cathepsins L and/or transmembrane protease serine 2 (TMPRSS2) (Shang et al., PNAS (2020) 117:11727, and Hoffmann et al., Cell (2020) 181:271-280). The cathepsin L inhibitor K117777, which lacks an inhibitory effect on the 3Clpro, can result in potent inhibition of SARS-CoV-2 in VeroE6, A549-ACE2 and/or HeLa-ACE2 (Mellott et al., bioRxiv (2020) 2020.2010.2023.347534). It has also been shown that the potent antiviral effect of K117777 is abolished when TMPRSS2 was expressed In A549-ACE2 (Steuten et al., bioRxiv (2020) 2020.2011.2021.392753). Off target activity of 3cLpro inhibitors, for example, on cathepsin L, may lead to an inaccurate assessment of the 3cLpro component of a compound's cellular potency. As an example, a compound described herein (such as a compound of Formula (I), or a pharmaceutically acceptable salt thereof) can have greater selectivity for a coronavirus protease over a host protease, such as cathepsin L. The selectivity can be determined by those skilled in the art, for example, using IC₅₀ and/or Ki values. In some embodiments, a compound described herein does not significantly inhibit cathepsin L (for example, IC₅₀≥10000 nM or >3.3 μM), but inhibits a coronavirus protease (for example, SARS-Cov-2 3Clpro).

A drawback with anti-viral treatment can be the development of resistance, including cross-resistance. Resistance can be a cause for treatment failure. The term “resistance” as used herein refers to a viral strain displaying a delayed, lessened and/or null response to an anti-viral agent. In some embodiments, a compound, or a pharmaceutically acceptable salt thereof, as described herein can be provided to a subject infected with a coronavirus strain that is resistant to one or more other anti-viral agents. In some embodiments, development of coronavirus resistant strains is delayed when a subject is treated with a compound, or a pharmaceutically acceptable salt thereof, as described herein compared to the development of a coronavirus resistant strain when treated with one or more other anti-viral agents.

### Combination Therapies

In some embodiments, a compound, or a pharmaceutically acceptable salt thereof, as described herein can be used in combination with one or more additional agent(s) for treating and/or inhibiting replication a coronavirus. Additional agents include, but are not limited to, an ACE inhibitor, an anticoagulant, an anti-inflammatory, an ARB, an ASO, a Covid-19 convalescent plasma, an entry inhibitor, an H₂ pump antagonist, an H-conducting channel, an HIV protease inhibitor, an HMG-CoA reductase inhibitor, an immune globulin, an immunosuppressant, an immunotherapeutic agent, a monoclonal antibody, a neuraminidase inhibitor, a nucleoside inhibitor, a nucleoside analog inhibitor, a polymerase inhibitor, a protease inhibitor, an siRNA, a statin, a tissue plasminogen activator, an antibiotic, an antimicrobial and a vaccine. Examples of additional agents include Ascorbic acid, Anakin, Azithromycin, Baloxavir, Baricitinib, Chloroquine Phosphate, Colchicine, a corticosteroid, Epoprostenol, Famotidine, Favipiravir, an IGIV, an interferon (for example, recombinant interferon alpha 2b, IFN-α and/or PEG-IFN-α-2a), an IVIG, Ivermectin, γ-globulin, lopinavir, Methylprednisolone, Molnupiravir (MK-4482 or EIDD-2801), Niclosamide, Nitazoxanide, Nitric oxide, Oseltamivir, Peramivir, RANTES, ribavirin, Remdesivir, Ruxolitinib, Sarilumab, Siltuximab, Sirolimus, a statin, Tacrolimus, Tocilizumab, Umifenovir, Zanamivir, Casirivimab, imdevimab, bamlanivimab, etesevimab and AT-527 (Good et al., Antimicrobial Agents and Chemotherapy (2021) 65(4):e02479-20)

In some embodiments, a compound, or a pharmaceutically acceptable salt thereof, as described herein can be administered with one or more additional agent(s) together in a single pharmaceutical composition. In some embodiments, a compound, or a pharmaceutically acceptable salt thereof, can be administered with one or more additional agent(s) as two or more separate pharmaceutical compositions. Further, the order of administration of a compound, or a pharmaceutically acceptable salt thereof, as described herein with one or more additional agent(s) can vary.

### EXAMPLES

Additional embodiments are disclosed in further detail in the following examples, which are not in any way intended to limit the scope of the claims.

### Compounds

Compounds of Formula (I), along with pharmaceutically acceptable salts thereof, can be prepared in various ways, including those synthetic schemes shown and described herein, are provided below. Those skilled in the art will be able to recognize modifications of the disclosed syntheses and to devise routes based on the disclosures herein; all such modifications and alternate routes are within the scope of the claims.

### Synthesis of Intermediates

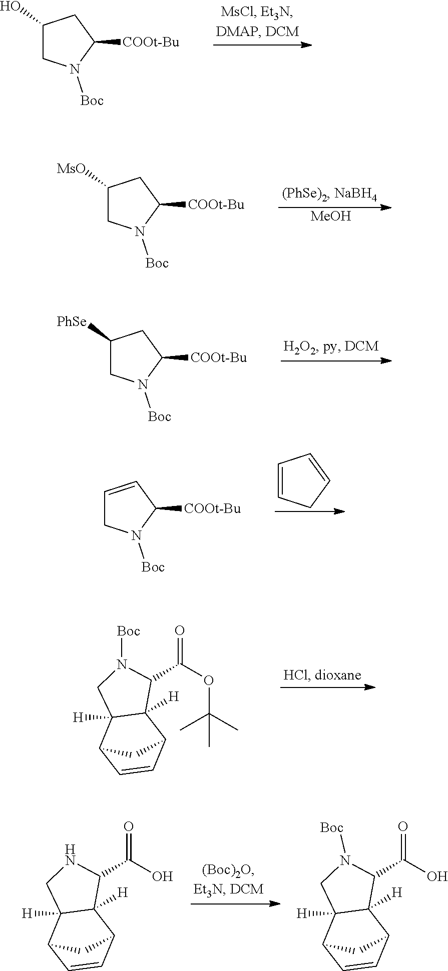

To a solution of 1,2-di-tert-butyl (2S,4R)-4-hydroxypyrrolidine-1,2-dicarboxylate (15 g, 52.2 mmol) in DCM (250 mL) was added triethylamine (9.51 g, 93.9 mmol) and DMAP (1.91 g, 15.7 mmol). MsCl (8.97 g, 78.3 mmol) was added dropwise at 0° C. The mixture was stirred at room temperature (rt) for 2 h, and the reaction was quenched with water (100 mL). The solution was extracted with DCM (3×150 mL). The organic layers were combined, washed with brine (100 mL), dried over anhydrous sodium sulfate, the solids were removed by filtration and the filtrate was concentrated under reduced pressure. The residue was chromatographed on a silica gel column with EA:PE (1:10) to provide 1,2-di-tert-butyl (2S,4R)-4-(methanesulfonyloxy)pyrrolidine-1,2-dicarboxylate (17.8 g, 89%) as a colorless oil. LC-MS (ESI, m/z): 366 [M+H]⁺.

To a solution of 1,2-di-t-butyl (2S,4R)-4-(methanesulfonyloxy)pyrrolidine-1,2-dicarboxylate (17.8 μg, 48.7 mmol) in CH₃OH (400 mL) was added (phenyldiselanyl)benzene (9.12 g, 29.2 mmol). NaBH₄ (2.4 g, 63.3 mmol) was added at 0° C. in several portions. The mixture was refluxed overnight and then concentrated under reduced pressure. Water (100 mL) was added, and the mixture was extracted with EA (3×150 mL). The organic layers were combined, washed with brine (100 mL), dried over anhydrous sodium sulfate, filtered and concentrated under reduced pressure. The residue was chromatographed on a silica gel column with EA:PE (1:5) to provide 1,2-di-tert-butyl (2S,4S)-4-(phenylselanyl)pyrrolidine-1,2-dicarboxylate (7.5 g, 32%) as a colorless oil. LC-MS (ESI, m/z): 428 [M+H]⁺.

To a solution of 1,2-di-t-butyl (2S,4S)-4-(phenylselanyl)pyrrolidine-1,2-dicarboxylate (7.5 g, 17.6 mmol) in DCM (100 mL) was added pyridine (2.4 mL, 30.5 mmol) and 30% aqueous H₂O₂ (5.6 mL, 71.6 mmol). The mixture was stirred at rt for 12 h, and the reaction was quenched with water (20 mL). The solution was extracted with DCM (3×150 mL). The organic layers were combined, washed with 1 M citric acid (80 mL), sat. aq. Na₂SO₃ (100 mL) and brine (100 mL), dried over anhydrous sodium sulfate, filtered and concentrated under reduced pressure. The residue was chromatographed on a silica gel column with EA:PE (1:9) to provide 1,2-di-t-butyl (2S)-2,5-dihydropyrrole-1,2-dicarboxylate (2.8 g, 53%) as a colorless oil. ¹H NMR (300 MHz, DMSO-d₆) δ 6.02-6.09 (m, 1H), 5.76-5.83 (m, 1H), 4.72-4.78 (m, 1H), 4.05-4.09 (m, 2H), 1.17-1.42 (m, 18H). LC-MS (ESI, m/z): 270 [M+H]⁺.

A solution of 1,2-di-tert-butyl (2S)-2,5-dihydropyrrole-1,2-dicarboxylate (2.8 g, 10.4 mmol) in dicyclopentadiene (60 mL) was stirred at 170° C. for 48 h under nitrogen and then reconstituted in DCM (200 mL). After removal of the solvent, the residue was chromatographed on a silica gel column with EA:PE (1:9) to provide the product (2.5 g, crude) as a yellow oil. The crude oil was chromatographed on a C18 column with H₂O:CH₃CN (2:1) to provide di-t-butyl (1S,3aR,4S,7R,7aS)-1,3,3a,4,7,7a-hexahydro-2H-4,7-methanoisoindole-1,2-dicarboxylate (690 mg, 19%) as a white solid. ¹H NMR (300 MHz, DMSO-d₆) δ 6.14-6.21 (m, 2H), 3.55-3.60 (m, 1H), 3.23-3.27 (m, 1H), 2.95-3.02 (m, 2H), 2.74-2.87 (m, 3H), 1.24-1.48 (m, 20H). LC-MS (ESI, m/z): 270 [M+H]⁺.

To a solution of di-t-butyl (1S,3aR,4S,7R,7aS)-1,3,3a,4,7,7a-hexahydro-2H-4,7-methanoisoindole-1,2-dicarboxylate (690 mg, 2.1 mmol) in dioxane (10 mL) was added HCl (10 mL, 9M, aq.). The mixture was stirred at rt overnight and then concentrated under reduced pressure to provide (1S,3aR,4S,7R,7aS)-2,3,3a,4,7,7a-hexahydro-1H-4,7-methanoisoindole-1-carboxylic acid (320 mg, crude) as a black solid. LC-MS (ESI, m/z): 180 [M+H]⁺.

To a solution of (1S,3aR,4S,7R,7aS)-2,3,3a,4,7,7a-hexahydro-1H-4,7-methanoisoindole-1-carboxylic acid (320 mg, 1.79 mmol) in DCM (8 mL) was added di-t-butyl dicarbonate (429 mg, 1.97 mmol) and triethylamine (542 mg, 5.34 mmol). The mixture was stirred at rt for 3 h and then concentrated under reduced pressure to provide (1S,3aR,4S,7R,7aS)-2-(tert-butoxycarbonyl)-2,3,3a,4,7,7a-hexahydro-1H-4,7-methanoisoindole-1-carboxylic acid (430 mg, crude) as a brown solid. LC-MS (ESI, m/z): 280 [M+H]⁺.

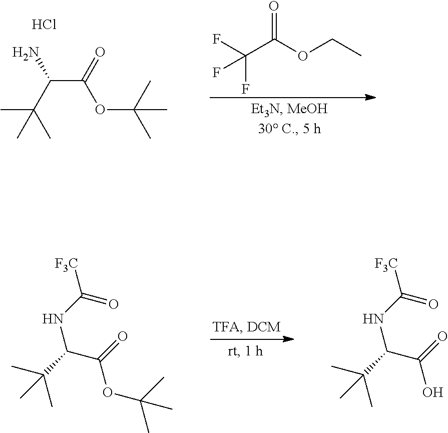

To a stirred mixture of tert-butyl (2S)-2-amino-3,3-dimethylbutanoate hydrochloride (6.00 g, 26.8 mmol) and ethyl 2,2,2-trifluoroacetate (7.62 g, 53.6 mmol) in CH₃OH (100 mL) was added triethylamine (5.43 g, 53.7 mmol) at 0° C. The mixture was stirred for 5 h at 30° C. and then concentrated under reduced pressure to afford the crude product. The crude product was diluted with DCM (150 mL) and made into a slurry with 100˜200 silica gel mesh (15 g), and the slurry was loaded to a column chromatography after removing the DCM. The sample was purified by column chromatography (Column size 6×24 cm, column volume: 600 mL, silica gel size (100˜200 mesh) quantity: 330 g) and eluted with CH₃OH:DCM (0%˜10% over 30 min). The collected fractions: 0% CH₃OH:DCM fractions were chosen as the pure fractions. and those fractions were combined and concentrated under reduced pressure to provide t-butyl (2S)-3,3-dimethyl-2-(2,2,2-trifluoroacetamido)butanoate (7.20 g, 90%) as a white solid. ¹H NMR (300 MHz, CDCl₃) δ 6.78-6.90 (m, 1H), 4.32-4.38 (m, 1H), 1.50 (s, 9H), 1.01 (s, 9H). LC-MS (ESI, m/z): 282 [M−H]⁻.

To a mixture of t-butyl (2S)-3,3-dimethyl-2-(2,2,2-trifluoroacetamido)butanoate (1.03 g, 3.64 mmol) in DCM (5 mL) was added trifluoroacetic acid (5 mL). The mixture was stirred for 1 h at rt and then concentrated under reduced pressure to (2S)-3,3-dimethyl-2-(2,2,2-trifluoroacetamido)butanoic acid (826 mg, crude) as a yellow oil. LC-MS (ESI, m/z): 226 [M−H]⁻.

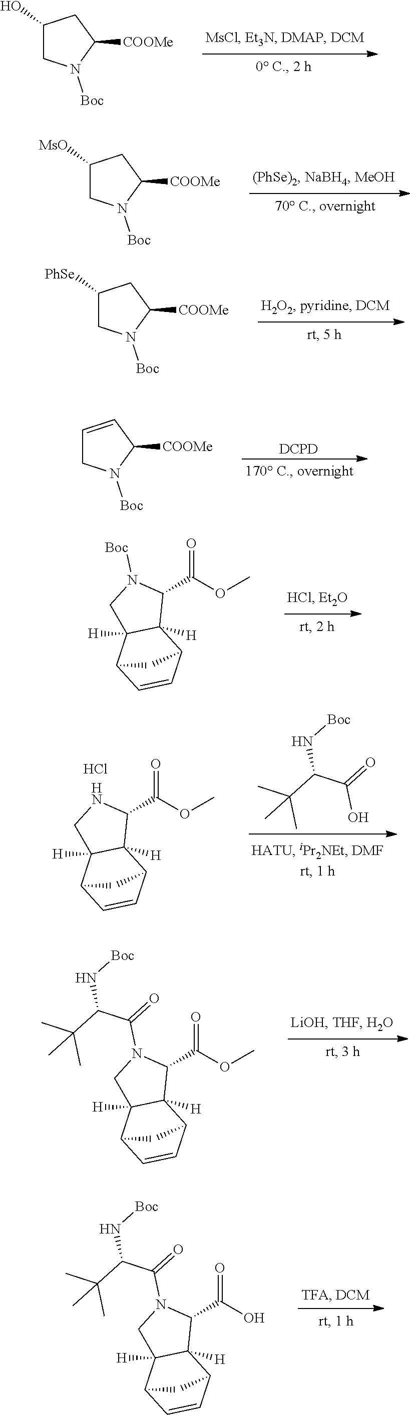

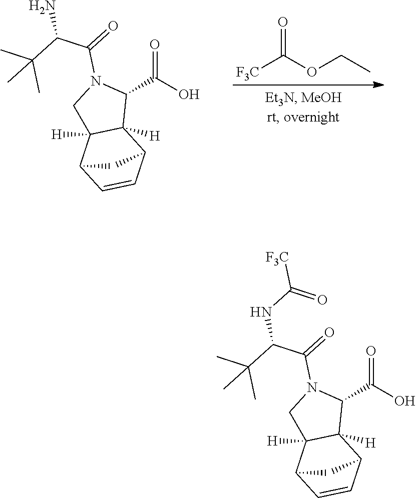

To a mixture of 1-t-butyl 2-methyl (2S,4R)-4-hydroxypyrrolidine-1,2-dicarboxylate (30.0 g, 122 mmol), triethylamine (22.3 g, 220 mmol) and N,N-dimethylpyridin-4-amine (4.48 g, 36.7 mmol) in DCM (500 mL) was added dropwise methanesulfonyl chloride (21.0 g, 183 mmol) at 0° C. The mixture was stirred for 2 h at 0° C. The reaction was quenched with water (500 mL). The mixture was extracted with DCM (3×500 mL). The organic layers were combined, washed with brine (2×500 mL), dried over anhydrous sodium sulfate, the solids were removed by filtration and the filtrate was concentrated under reduced pressure to afford the crude product. The crude product was chromatographed on a silica gel column with EA:PE (1:1) to provide 1-t-butyl 2-methyl (2S,4R)-4-(methanesulfonyloxy)pyrrolidine-1,2-dicarboxylate (35.0 g, 85%) as a light yellow solid. LC-MS (ESI, m/z): 224 [M+H-Boc]⁺.

To a mixture of 1-t-butyl 2-methyl (2S,4R)-4-(methanesulfonyloxy)pyrrolidine-1,2-dicarboxylate (25.0 g, 77.3 mmol) and diphenyl diselenide (24.1 g, 77.3 mmol) in CH₃OH (600 mL) was added sodium borohydride (3.80 g, 100 mmol) at 0° C. The mixture was stirred overnight at 70° C. and then concentrated under reduced pressure to remove the CH₃OH. Water (600 mL) was added, and the mixture was extracted with EA (3×600 mL). The organic layers were combined, washed with brine (600 mL), dried over anhydrous sodium sulfate, the solids were removed by filtration and the filtrate was concentrated under reduced pressure to afford the crude product. The crude product was chromatographed on a silica gel column with EA:PE (1:8) to provide 1-tert-butyl 2-methyl (2S,4S)-4-(phenylselanyl)pyrrolidine-1,2-dicarboxylate (26.8 g, 84%) as a yellow oil. LC-MS (ESI, m/z): 286 [M−100+H]⁺.

To a mixture of 1-t-butyl 2-methyl (2S,4S)-4-(phenylselanyl)pyrrolidine-1,2-dicarboxylate (26.8 g, 69.7 mmol) and pyridine (9.38 g, 118 mmol) in DCM (300 mL) was added hydrogen peroxide (31.6 mL, 279 mmol, 30% in water). The mixture was stirred for 5 h at rt. The reaction was quenched with water (500 mL). The mixture was extracted with DCM (3×400 mL). The organic layers were combined, washed with citric acid (500 mL, 1 M), saturated aqueous sodium sulfite (500 mL), washed with brine (500 mL), dried over anhydrous sodium sulfate, the solids were removed by filtration and the filtrate was concentrated under reduced pressure to afford the crude product. The crude product was chromatographed on a silica gel column with EA:PE (1:5) to provide 1-t-butyl 2-methyl (2S)-2,5-dihydropyrrole-1,2-dicarboxylate (10.5 g, 62%) as a yellow oil. ¹H NMR (300 MHz, CDCl₃) δ 5.91-5.04 (m, 1H), 5.67-5.79 (m, 1H), 4.92-5.09 (m, 1H), 4.17-4.35 (m, 2H), 3.71-3.79 (m, 3H), 1.42-1.52 (m, 9H). LC-MS (ESI, m/z): 128 [M+H-Boc]⁺.

A mixture of 1-t-butyl 2-methyl (2S)-2,5-dihydropyrrole-1,2-dicarboxylate (3.68 g, 16.2 mmol) in dicyclopentadiene (40 mL) was stirred overnight at 170° C. The mixture was diluted with DCM (500 mL) and made into a slurry with 100-200 silica gel mesh (50 g). The mixture was loaded to a column. After removed the DCM under reduced pressure, the sample was purified by column chromatography (Column size 6×24 cm, column volume: 600 mL, silica gel size (100˜200 mesh) quantity: 330 g) and eluted with EA:PE (0%-50% over 30 min). The collected fractions: 19%-25% EA:PE were combined and concentrated under reduced pressure to afford the crude product (2.5 g). The crude product was purified by C18 column with CH₃CN:Water (0.05% TFA). The compound fraction was concentrated under reduced pressure to provide 4-t-butyl 3-methyl (1R,2S,3S,6R,7S)-4-azatricyclo[5.2.1.0\hat{ }{2,6}]dec-8-ene-3,4-dicarboxylate (1.70 g, 32%) as a yellow oil. ¹H NMR (300 MHz, CDCl₃) δ 6.12-6.30 (m, 2H), 3.78-3.98 (m, 1H), 3.72 (s, 3H), 3.38-3.51 (m, 1H), 3.04-3.21 (m, 2H), 2.77-2.96 (m, 3H), 1.50-1.58 (m, 1H), 1.32-1.46 (m, 10H). LC-MS (ESI, m/z): 194 [M+H-Boc]⁺.

A mixture of 4-t-butyl 3-methyl (1R,2S,3S,6R,7S)-4-azatricyclo[5.2.1.0\hat{ }{2,6}]dec-8-ene-3,4-dicarboxylate (500 mg, 1.70 mmol) in hydrogen chloride (10 mL, 2 M in Et₂O) was stirred for 2 h at rt. The mixture was concentrated under reduced pressure to afford methyl (1R,2S,3S,6R,7S)-4-azatricyclo[5.2.1.0\hat{ }{2,6}]dec-8-ene-3-carboxylate hydrochloride (391 mg, crude) as a yellow oil. LC-MS (ESI, m/z): 194 [M+H]⁺.

To a mixture of methyl (1R,2S,3S,6R,7S)-4-azatricyclo[5.2.1.0\hat{ }{2,6}]dec-8-ene-3-carboxylate hydrochloride (391 mg, 1.70 mmol), (2S)-2-[(tert-butoxycarbonyl)amino]-3,3-dimethylbutanoic acid (394 mg, 1.70 mmol) and o-(7-azabenzotriazol-1-yl)-N,N,″,″-tetramethyluroniumhexafluorophosphate (777 mg, 2.04 mmol) in DMF (10 mL) was added N-ethyl-N-isopropylpropan-2-amine (1.32 g, 10.2 mmol) at 0° C. The mixture was stirred for 1 h at rt. The reaction was quenched with water (20 mL). The mixture was extracted with EA (3×20 mL). The organic layers were combined, washed with brine (2×20 mL), dried over anhydrous sodium sulfate, the solids were removed by filtration and the filtrate was concentrated under reduced pressure to afford the crude product. The crude product was chromatographed on a silica gel column with EA:PE (8:92) to provide methyl (1R,2S,3S,6R,7S)-4-[(2S)-2-[(tert-butoxycarbonyl)amino]-3,3-dimethylbutanoyl]-4-azatricyclo[5.2.1.0\hat{ }{2,6}]dec-8-ene-3-carboxylate (490 mg, 69%) as an off-white semi-solid. ¹H NMR (300 MHz, CDCl₃) δ 6.03-6.27 (m, 2H), 5.11-5.26 (m, 1H), 4.18-4.36 (m, 2H), 3.72-3.78 (m, 3H), 3.54-3.70 (m, 2H), 2.97-3.14 (m, 2H), 2.86-2.94 (m, 2H), 1.49-1.54 (m, 1H), 1.41-1.48 (m, 9H), 1.33-1.39 (m, 1H), 0.92-1.00 (m, 9H). LC-MS (ESI, m/z): 407 [M+H]⁺.

To a mixture of methyl (1R,2S,3S,6R,7S)-4-[(2S)-2-[(tert-butoxycarbonyl)amino]-3,3-dimethylbutanoyl]-4-azatricyclo[5.2.1.0\hat{ }{2,6}]dec-8-ene-3-carboxylate (490 mg, 1.205 mmol) in THF (5 mL)/water (5 mL) was added lithium hydroxide (144 mg, 6.03 mmol). The mixture was stirred for 3 h at rt. The mixture was concentrated under reduced pressure to removed then THF and the pH was adjusted to 5 with HCl (2 M). The mixture was extracted with EA (3×10 mL). The organic layers were combined, washed with brine (2×10 mL), dried over anhydrous sodium sulfate, filtered and concentrated under reduced pressure to provide (1R,2S,3S,6R,7S)-4-[(2S)-2-[(tert-butoxycarbonyl)amino]-3,3-dimethylbutanoyl]-4-azatricyclo[5.2.1.0\hat{ }{2,6}]dec-8-ene-3-carboxylic acid (465 mg, 97%) as a white solid. ¹H NMR (300 MHz, CDCl₃) δ 6.04-6.30 (m, 2H), 5.21-5.29 (m, 1H), 4.22-4.32 (m, 2H), 3.52-3.79 (m, 2H), 3.06-3.24 (m, 2H), 2.91-3.04 (m, 2H), 1.51-1.56 (m, 1H), 1.37-1.47 (m, 10H), 0.95-1.00 (m, 9H). LC-MS (ESI, m/z): 393 [M+H]⁺.

To a mixture of (1R,2S,3S,6R,7S)-4-[(2S)-2-[(t-butoxycarbonyl)amino]-3,3-dimethylbutanoyl]-4-azatricyclo[5.2.1.0\hat{ }{2,6}]dec-8-ene-3-carboxylic acid (465 mg, 1.18 mmol) in DCM (15 mL) was added trifluoroacetic acid (5 mL). The mixture was stirred for 1 h at rt and then concentrated under reduced pressure to afford (1R,2S,3S,6R,7S)-4-[(2S)-2-amino-3,3-dimethylbutanoyl]-4-azatricyclo[5.2.1.0\hat{ }{2,6}]dec-8-ene-3-carboxylic acid (346 mg, crude) as a dark blue semi-solid. LC-MS (ESI, m/z): 293 [M+H]⁺.

To a mixture of (1R,2S,3S,6R,7S)-4-[(2S)-2-amino-3,3-dimethylbutanoyl]-4-azatricyclo[5.2.1.0\hat{ }{2,6}]dec-8-ene-3-carboxylic acid (346 mg, 1.18 mmol) in CH₃OH (10 mL) was added triethylamine (1.44 g, 14.2 mmol) and ethyl 2,2,2-trifluoroacetate (1.01 g, 7.10 mmol). The mixture was stirred overnight at rt and concentrated under reduced pressure to remove then CH₃OH. The crude product was purified by C18 column with CH₃CN:Water (0.05% TFA). The compound fraction was concentrated under reduced pressure to provide (1R,2S,3S,6R,7S)-4-[(2S)-3,3-dimethyl-2-(2,2,2-trifluoroacetamido)butanoyl]-4-azatricyclo[5.2.1.0\hat{ }{2,6}]dec-8-ene-3-carboxylic acid (310 mg, 65%) as a yellow solid. ¹H NMR (300 MHz, DMSO-d₆) δ 12.22-13.12 (m, 1H), 8.96-9.49 (m, 1H), 5.88-6.24 (m, 2H), 4.24-4.60 (m, 1H), 3.94-4.05 (m, 1H), 3.43-3.58 (m, 2H), 2.67-3.04 (m, 4H), 1.30-1.44 (m, 2H), 0.76-1.05 (m, 9H). LC-MS (ESI, m/z): 389 [M+H]⁺.

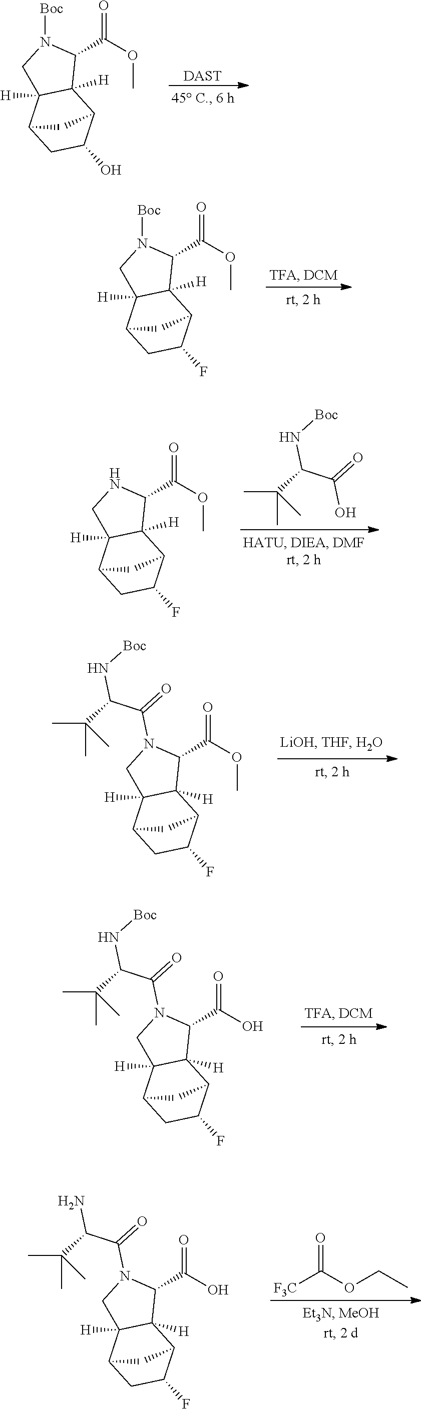

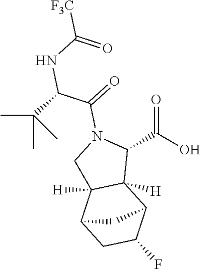 [SMILES: CC(C)(C)[C@H](NC(=O)C(F)(F)F)C(=O)N1C[C@@H]2[C@H]3C[C@@H]([C@@H]2[C@H]1C(=O)O)[C@H](F)C3]

A mixture of 4-t-butyl 3-methyl (1S,2R,3S,6R,7S,9R)-9-hydroxy-4-azatricyclo[5.2.1.0\hat{ }{2,6}]decane-3,4-dicarboxylate (500 mg, 1.60 mmol) in diethylaminosulfur trifluoride (10 mL) was stirred for 6 h at 45° C. The mixture was diluted with DCM (80 mL), and the reaction quenched with sat. NaHCO₃ (50 mL) at 0° C. The mixture was extracted with DCM (3×80 mL). The organic layers were combined, washed with brine (2×50 mL), dried over anhydrous sodium sulfate, the solids were removed by filtration and the filtrate was concentrated under reduced pressure. The residue was purified by silica gel column chromatography with PE:EA (7:3) to afford 4-t-butyl 3-methyl (1S,2R,3S,6R,7S,9R)-9-fluoro-4-azatricyclo[5.2.1.0\hat{ }{2,6}]decane-3,4-dicarboxylate (210 mg, 42%) as a yellow oil. LC-MS (ESI, m/z): 258 [M−56+H]⁺.

To a stirred mixture of 4-t-butyl 3-methyl (1S,2R,3S,6R,7S,9R)-9-fluoro-4-azatricyclo[5.2.1.0\hat{ }{2,6}]decane-3,4-dicarboxylate (210 mg, 0.670 mmol) in dichloromethane (DCM) (3 mL) was added trifluoroacetic acid (1 mL) at rt. The mixture was stirred for 2 h at rt and then concentrated under reduced pressure to provide product methyl (1S,2R,3S,6R,7S,9R)-9-fluoro-4-azatricyclo[5.2.1.0\hat{ }{2,6}]decane-3-carboxylate (155 mg, crude) as a yellow oil. LC-MS (ESI, m/z): 213 [M+H]⁺.

To a mixture of (2S)-2-[(t-butoxycarbonyl)amino]-3,3-dimethylbutanoic acid (155 mg, 0.671 mmol) in N,N-dimethylformamide (2 mL) was added HATU (305 mg, 0.805 mmol) and N-ethyl-N-isopropylpropan-2-amine (520 mg, 4.026 mmol) at 0° C. The mixture was stirred for 20 min at 0° C., and then methyl (1S,2R,3S,6R,7S,9R)-9-fluoro-4-azatricyclo[5.2.1.0\hat{ }{2,6}]decane-3-carboxylate (143 mg, 0.671 mmol) was added at 0° C. The mixture was stirred for 2 h at rt and then purified by a C18 column with CH₃CN/Water (0.05% TFA). The desired fraction was concentrated under reduced pressure to provide methyl (1S,2R,3S,6R,7S,9R)-4-[(2S)-2-[(tert-butoxycarbonyl)amino]-3,3-dimethylbutanoyl]-9-fluoro-4-azatricyclo[5.2.1.0\hat{ }{2,6}]decane-3-carboxylate (190 mg, 66%) as a yellow solid. LC-MS (ESI, m/z): 427 [M+H]⁺.

To a stirred methyl (1S,2R,3S,6R,7S,9R)-4-[(2S)-2-[(t-butoxycarbonyl)amino]-3,3-dimethylbutanoyl]-9-fluoro-4-azatricyclo[5.2.1.0\hat{ }{2,6}]decane-3-carboxylate (190 mg, 0.445 mmol) in THF (2 mL) and water (2 mL) was added LiOH (53.3 mg, 2.22 mmol) at rt. The mixture was stirred for 2 h and acidified to pH=3 with hydrochloric acid (2M). The mixture was extracted with EA (3×50 mL). The organic layers were combined, washed with brine (2×20 mL) and dried over anhydrous sodium sulfate. The mixture was concentrated under reduced pressure to afford (1S,2R,3S,6R,7S,9R)-4-[(2S)-2-[(t-butoxycarbonyl)amino]-3,3-dimethylbutanoyl]-9-fluoro-4-azatricyclo[5.2.1.0\hat{ }{2,6}]decane-3-carboxylic acid (180 mg, crude) as an orange solid. LC-MS (ESI, m/z): 413 [M+H]⁺.

To a stirred mixture of (1S,2R,3S,6R,7S,9S)-4-[(2S)-2-[(tert-butoxycarbonyl)amino]-3,3-dimethylbutanoyl]-9-fluoro-4-azatricyclo[5.2.1.0\hat{ }{2,6}]decane-3-carboxylic acid (160 mg, 0.388 mmol) in DCM (2 mL) was added trifluoroacetic acid (0.6 mL) at rt. The mixture was stirred for 2 h at rt and then concentrated under reduced pressure to afford (1S,2R,3S,6R,7S,9S)-4-[(2S)-2-amino-3,3-dimethylbutanoyl]-9-fluoro-4-azatricyclo[5.2.1.0\hat{ }{2,6}]decane-3-carboxylic acid (121 mg, crude) as a yellow oil. LC-MS (ESI, m/z): 313 [M+H]⁺.

To a stirred mixture of (1S,2R,3S,6R,7S,9R)-4-[(2S)-2-amino-3,3-dimethylbutanoyl]-9-fluoro-4-azatricyclo[5.2.1.0\hat{ }{2,6}]decane-3-carboxylic acid (121 mg, 0.387 mmol) and trimethylamine (470 mg, 4.64 mmol) in CH₃OH (1 mL) was added ethyl 2,2,2-trifluoroacetate (550 mg, 3.870 mmol). The mixture was stirred for 2 days at rt. The reaction was quenched with water (10 mL). The mixture was concentrated under reduced pressure to remove CH₃OH and then acidified to pH=4 with hydrochloric acid (2M). The mixture was extracted with EA (3×50 mL). The organic layers were combined, washed with brine (2×20 mL) and dried over anhydrous sodium sulfate. The combined organic layers were concentrated under reduced pressure to afford (1S,2R,3S,6R,7S,9R)-4-[(2S)-3,3-dimethyl-2-(2,2,2-trifluoroacetamido)butanoyl]-9-fluoro-4-azatricyclo[5.2.1.0\hat{ }{2,6}]decane-3-carboxylic acid (160 mg, crude) as a light yellow oil. LC-MS (ESI, m/z): 409 [M+H]⁺.

### Example 1

### Compound 1

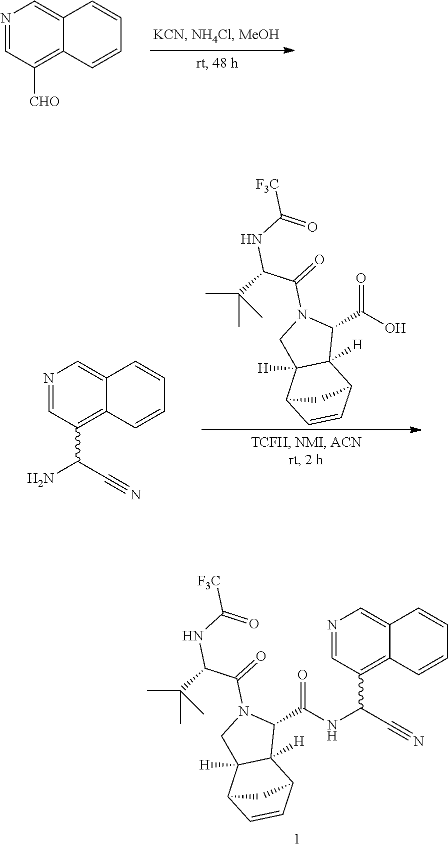

To a mixture isoquinoline-4-carbaldehyde (200 mg, 1.27 mmol) in methanol (4 mL) was added NH₄Cl (136 mg, 2.54 mmol). After stirring for 2 h at rt, KCN (124 mg, 1.91 mmol) was added. The mixture was stirred 48 h at rt. The mixture was filtered, and the filtrate was concentrated under reduced pressure to remove the CH₃OH. The residue was diluted with CH₂Cl₂ (5 mL) and filtered. The filtrate was concentrated under reduced pressure to provide 2-amino-2-(isoquinolin-4-yl)acetonitrile (200 mg, crude) as a brown oil. LC-MS (ESI, m/z): 184 [M+H]⁺.

To a mixture of (1S,3aR,4S,7R,7aS)-2-((S)-3,3-dimethyl-2-(2,2,2-trifluoroacetamido)butanoyl)-2,3,3a,4,7,7a-hexahydro-1H-4,7-methanoisoindole-1-carboxylic acid (212 mg, 0.546 mmol), 2-amino-2-(isoquinolin-4-yl)acetonitrile (100 mg, 0.546 mmol) and N,N,N,N-tetramethylchloroformamidinium hexafluorophosphate (TCFH) (168 mg, 0.601 mmol) in CH₃CN (3 mL) was added 1-methyl-1H-imidazole (224 mg, 2.73 mmol) at 0° C. The mixture was stirred for 2 h at rt. The reaction was quenched with water (30 mL). The mixture was extracted with ethyl acetate (EA) (3×80 mL). The organic layers were combined, washed with brine (2×30 mL) and dried over anhydrous sodium sulfate. The solids were removed by filtration and the filtrate was concentrated under reduced pressure to afford the crude product. The crude product was purified by prep-TLC (Mobile phase: EA:petroleum ether (PE)=2:1; Rf=0.5; detection: UV) to provide the crude product. The crude product was purified by prep-HPLC (Column: xSelect CSH Prep C18 OBD Column, 19×150 mm, 5 μm; Mobile Phase A: water (0.1% FA), Mobile Phase B: CH₃CN; Flow rate: 25 mL/min; Gradient: 37% B to 64% B in 10 min, 64% B; Wave Length: 254 nm; RT1(min): 8.65) to provide (1S,3aR,4S,7R,7aS)—N-(cyano(isoquinolin-4-yl)methyl)-2-((S)-3,3-dimethyl-2-(2,2,2-trifluoroacetamido)butanoyl)-2,3,3a,4,7,7a-hexahydro-1H-4,7-methanoisoindole-1-carboxamide (39.5 mg, 13%) as a white solid. ¹H NMR (400 MHz, 80° C., DMSO-d₆) δ 9.36-9.41 (m, 1H), 9.25-9.30 (m, 1H), 8.52-8.90 (m, 2H), 8.20-8.26 (m, 1H), 7.83-8.05 (m, 1H), 7.69-7.80 (m, 2H), 6.70-6.82 (m, 1H), 5.90-6.19 (m, 2H), 4.45-4.66 (m, 1H), 3.99-4.30 (m, 1H), 3.35-3.72 (m, 2H), 2.95-3.00 (m, 1H), 2.82-2.90 (m, 2H), 2.61-2.79 (m, 1H), 1.22-1.49 (m, 2H), 0.76-1.05 (m, 9H). LC-MS (ESI, m/z): 554 [M+H]⁺.

### Example 2

### Compound 2

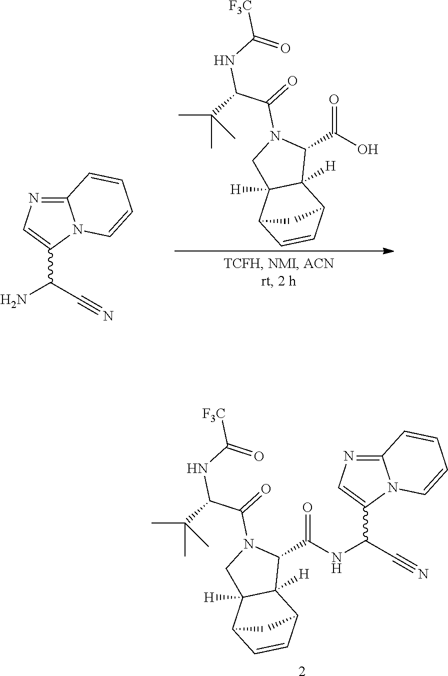

2-amino-2-(imidazo[1,2-a]pyridin-3-yl)acetonitrile was prepared according to the procedure to prepare 2-amino-2-(isoquinolin-4-yl)acetonitrile using imidazo[1,2-a]pyridine-3-carbaldehyde. LC-MS (ESI, m/z): 173 [M+H]⁺.

To a mixture of (1S,3aR,4S,7R,7aS)-2-((S)-3,3-dimethyl-2-(2,2,2-trifluoroacetamido)butanoyl)-2,3,3a,4,7,7a-hexahydro-1H-4,7-methanoisoindole-1-carboxylic acid (225 mg, 0.581 mmol), 2-amino-2-(imidazo[1,2-a]pyridin-3-yl)acetonitrile (100 mg, 0.581 mmol) and N,N-tetramethylchloroformamidinium hexafluorophosphate (TCFH) (179 mg, 0.639 mmol) in CH₃CN (5 mL) was added 1-methyl-1H-imidazole (238 mg, 2.91 mmol) at 0° C. The mixture was stirred for 2 h at rt. The reaction was quenched with water (50 mL). The mixture was extracted with EA (3×100 mL). The organic layers were combined, washed with brine (2×30 mL), dried over anhydrous sodium sulfate, filtered and concentrated under reduced pressure to afford crude product. The crude product was purified by TLC (Mobile phase: EA:PE=5:1; Rf=0.5; detection: UV) to provide the desired product. The crude product was purified by prep-HPLC (Column: xSelect CSH Prep C18 OBD Column, 19×150 mm, 5 μm; Mobile Phase A: Water (0.1% FA), Mobile Phase B: CH₃CN; Flow rate: 25 mL/min; Gradient: 24% B to 54% B in 7 min, 54% B; Wave Length: 254 nm; RT1(min): 6.4) to provide (1S,3aR,4S,7R,7aS)—N-(cyano(imidazo[1,2-a]pyridin-3-yl)methyl)-2-((S)-3,3-dimethyl-2-(2,2,2-trifluoroacetamido)butanoyl)-2,3,3a,4,7,7a-hexahydro-1H-4,7-methanoisoindole-1-carboxamide (80.3 mg, 25%) as a white solid. ¹H NMR (400 MHz, 80° C., DMSO-d₆) δ 9.10-9.26 (m, 1H), 8.54-8.86 (m, 1H), 8.11-8.43 (m, 1H), 7.63-7.82 (m, 2H), 7.31-7.45 (m, 1H), 6.85-7.05 (m, 1H), 6.64-6.71 (m, 1H), 5.90-6.20 (m, 2H), 4.50-4.55 (m, 1H), 3.99-4.22 (m, 1H), 3.56-3.68 (m, 1H), 3.42-3.52 (m, 1H), 2.85-3.01 (m, 3H), 2.66-2.78 (m, 1H), 1.20-1.44 (m, 2H), 0.86-1.10 (m, 9H). LC-MS (ESI, m/z): 543 [M+H]⁺.

### Example 3

### Compound 3

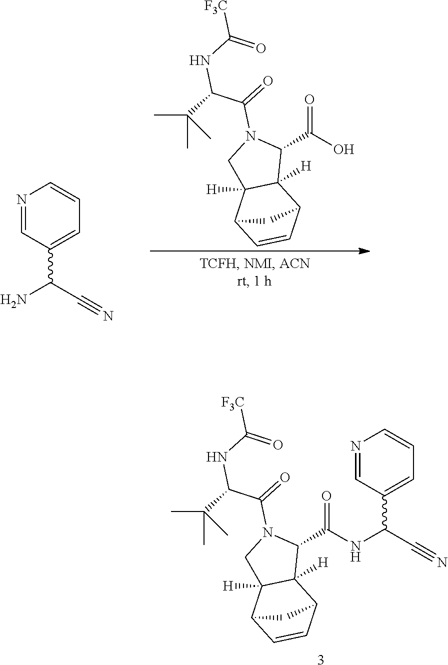

2-amino-2-(pyridin-3-yl)acetonitrile was prepared according to the procedure to prepare 2-amino-2-(isoquinolin-4-yl)acetonitrile using 3-pyridinecarboxaldehyde. LC-MS (ESI, m/z): 134 [M+H]⁺.

To a mixture of 2-amino-2-(pyridin-3-yl)acetonitrile (70.0 mg, 0.526 mmol), (1R,2S,3S,6R,7S)-4-[(2S)-3,3-dimethyl-2-(2,2,2-trifluoroacetamido)butanoyl]-4-azatricyclo[5.2.1.0\hat{ }{2,6}]dec-8-ene-3-carboxylic acid (204 mg, 0.526 mmol) and TCFH (192 mg, 0.684 mmol) in CH₃CN (3 mL) was added NMI (432 mg, 5.26 mmol). The mixture was stirred for 1 h at rt. The reaction was quenched with water (10 mL). The mixture was extracted with EA (3×10 mL). The organic layers were combined, washed with brine (2×10 mL), dried over anhydrous sodium sulfate, filtered and concentrated under reduced pressure to remove the solvent. The residue was purified by prep-HPLC (Column: xSelect CSH Prep C18 OBD Column, 19×250 mm, 5 μm; Mobile Phase A: Water (0.05% TFA), Mobile Phase B: CH₃CN; Flow rate: 25 mL/min; Gradient: 35% B to 65% B in 7 min, 65% B; Wave Length: 254 nm; RT(min): 6) to afford the crude product. The crude product was purified by prep-HPLC (Column: xSelect CSH Prep C18 OBD Column, 19×250 mm, 5 μm; Mobile Phase A: Water (0.1% FA), Mobile Phase B: CH₃CN; Flow rate: 25 mL/min; Gradient: 42% B to 72% B in 7 min, 72% B; Wave Length: 254 nm; RT(min): 6) to provide (1R,2S,3S,6R,7S)—N-[cyano(pyridin-3-yl)methyl]-4-[(2S)-3,3-dimethyl-2-(2,2,2-trifluoroacetamido)butanoyl]-4-azatricyclo[5.2.1.0\hat{ }{2,6}]dec-8-ene-3-carboxamide (33.5 mg, 12%) as a white solid. ¹H NMR (400 MHz, 80° C., DMSO-d₆) δ 9.28-9.35 (m, 1H), 8.98-9.06 (m, 1H), 8.62-8.70 (m, 1H), 8.55-8.61 (m, 1H), 7.95-8.03 (m, 1H), 7.40-7.50 (m, 1H), 6.10-6.30 (m, 1H), 6.02-6.08 (m, 1H), 5.95-6.01 (m, 1H), 4.45-4.55 (m, 1H), 4.10-4.20 (m, 1H), 3.60-3.72 (m, 1H), 3.40-3.50 (m, 1H), 3.00-3.10 (m, 1H), 2.85-2.99 (m, 2H), 2.65-2.80 (m, 1H), 1.30-1.45 (m, 2H), 0.85-1.05 (m, 9H). LC-MS (ESI, m/z): 504 [M+H]⁺.

### Example 4

### Compound 4

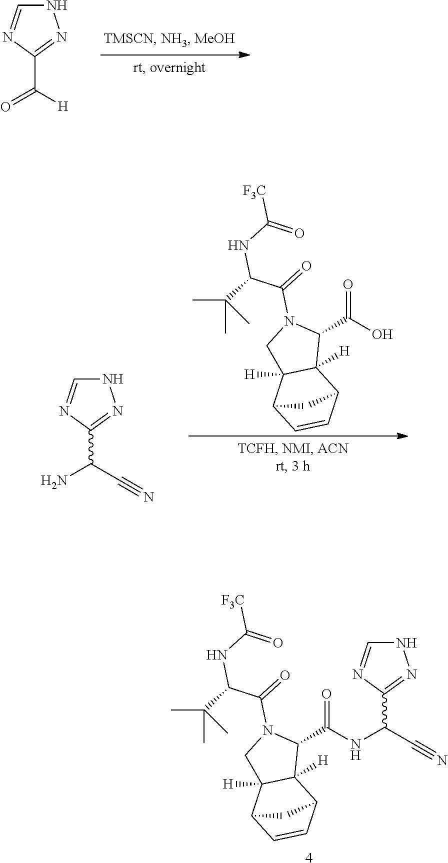

To a mixture of 1H-1,2,4-triazole-3-carbaldehyde (200 mg, 2.06 mmol, 1.0 eq.) in ammonia (5 mL, 7 M in CH₃OH) was added trimethylsilyl cyanide (286 mg, 2.88 mmol, 1.4 eq.) at 0° C. The mixture was stirred overnight at rt and then concentrated under reduced pressure to afford 2-amino-2-(1H-1,2,4-triazol-3-yl)acetonitrile (250 mg, crude) as a brown semi-solid. LC-MS (ESI, m/z): 124 [M+H]⁺.

To a mixture of 2-amino-2-(1H-1,2,4-triazol-3-yl)acetonitrile (250 mg, crude), (1R,2S,3S,6R,7S)-4-[(2S)-3,3-dimethyl-2-(2,2,2-trifluoroacetamido)butanoyl]-4-azatricyclo[5.2.1.0\hat{ }{2,6}]dec-8-ene-3-carboxylic acid (80.0 mg, 0.206 mmol), TCFH (75.0 mg, 0.268 mmol) in CH₃CN (5 mL) was added NMI (254 mg, 3.09 mmol). The mixture was stirred for 3 h at rt. The reaction was quenched with water (15 mL) and extracted with EA (3×15 mL). The organic layers were combined, washed with brine (2×15 mL), dried over anhydrous sodium sulfate, the solids were removed by filtration and the filtrate was concentrated under reduced pressure to remove the solvent. The residue was purified by TLC (Mobile phase: CH₃OH: CH₂Cl₂=1:12; Rf=0.5; detection: UV) to afford the crude product, that was further purified by prep-HPLC (Column: xSelect CSH Prep C18 OBD Column, 19×150 mm, 5 μm; Mobile Phase A: Water (0.1% FA), Mobile Phase B: CH₃CN; Flow rate: 25 mL/min; Gradient: 30% B to 53% B in 10 min, 53% B; Wave Length: 254 nm; RT1(min): 7.56) to provide (1R,2S,3S,6R,7S)—N-[cyano(1H-1,2,4-triazol-3-yl)methyl]-4-[(2S)-3,3-dimethyl-2-(2,2,2-trifluoroacetamido)butanoyl]-4-azatricyclo[5.2.1.0\hat{ }{2,6}]dec-8-ene-3-carboxamide (38.2 mg, 48%) as a white solid. ¹H NMR (400 MHz, 100° C., DMSO-d₆) δ 8.80-9.15 (m, 1H), 8.56-8.79 (m, 1H), 8.47 (s, 1H), 6.05-6.25 (m, 1H), 5.82-6.04 (m, 2H), 4.07-4.61 (m, 2H), 3.55-3.70 (m, 1H), 3.40-3.54 (m, 1H), 3.05-3.10 (m, 1H), 2.99-3.02 (m, 1H), 2.86-2.93 (m, 2H), 2.78-2.82 (m, 1H), 1.18-1.49 (m, 2H), 0.82-1.07 (m, 9H). LC-MS (ESI, m/z): 494 [M+H]⁺.

### Example 5

### Compound 5

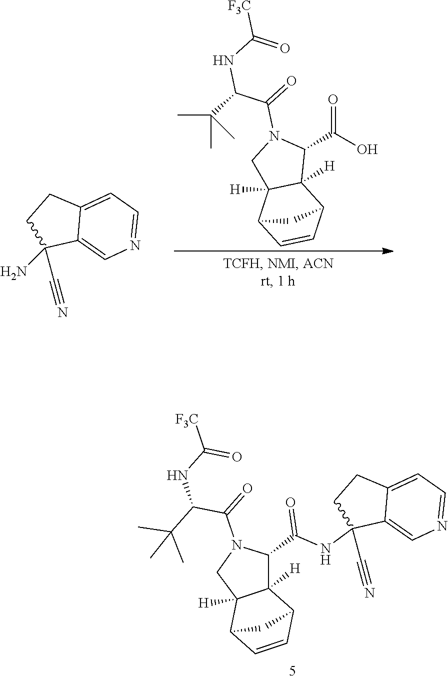

7-amino-5H,6H-cyclopenta[c]pyridine-7-carbonitrile was prepared according to the procedure to prepare 2-amino-2-(1H-1,2,4-triazol-3-yl)acetonitrile using 5H,6H-cyclopenta[c]pyridin-7-one hydrochloride. LC-MS (ESI, m/z): 160 [M+H]⁺.

To a mixture of 7-amino-5H,6H-cyclopenta[c]pyridine-7-carbonitrile (140 mg, crude), (1R,2S,3S,6R,7S)-4-[(2S)-3,3-dimethyl-2-(2,2,2-trifluoroacetamido)butanoyl]-4-azatricyclo[5.2.1.0\hat{ }{2,6}]dec-8-ene-3-carboxylic acid (120 mg, 0.308 mmol) and TCFH (112 mg, 0.400 mmol) in CH₃CN (3 mL) was added NMI (253 mg, 3.08 mmol). The mixture was stirred for 1 h at rt. The reaction was quenched with water (20 mL). The mixture was extracted with EA (3×20 mL). The organic layers were combined, washed with brine (2×20 mL), dried over anhydrous sodium sulfate, filtered and concentrated under reduced pressure to remove the solvent. The residue was purified by TLC (Mobile phase: CH₃OH:CH₂Cl₂=1:10; Rf=0.6; detection: UV) to afford the crude product. The crude product was purified by prep-HPLC (Column: xSelect CSH Prep C18 OBD Column, 19×250 mm, 5 μm; Mobile Phase A: Water (0.1% FA), Mobile Phase B: CH₃CN; Flow rate: 25 mL/min; Gradient: 34% B to 64% B in 7 min, 64% B; Wave Length: 220 nm; RT1(min): 6.8) to provide (1R,2S,3S,6R,7S)—N-{7-cyano-5H,6H-cyclopenta[c]pyridin-7-yl}-4-[(2S)-3,3-dimethyl-2-(2,2,2-trifluoroacetamido)butanoyl]-4-azatricyclo[5.2.1.0\hat{ }{2,6}]dec-8-ene-3-carboxamide (9.00 mg, 5%) as a pink solid. ¹H NMR (400 MHz, 80° C., DMSO-d₆) δ 8.80-8.95 (m, 1H), 8.68-8.79 (m, 2H), 8.40-8.65 (m, 1H), 7.35-7.50 (m, 1H), 5.90-6.20 (m, 2H), 4.42-4.55 (m, 1H), 4.05-4.25 (m, 1H), 3.62-3.75 (m, 1H), 3.40-3.50 (m, 1H), 2.80-3.05 (m, 5H), 2.55-2.79 (m, 3H), 1.32-1.46 (m, 2H), 0.83-1.05 (m, 9H). LC-MS (ESI, m/z): 530 [M+H]⁺.

### Example 6

### Compound 6

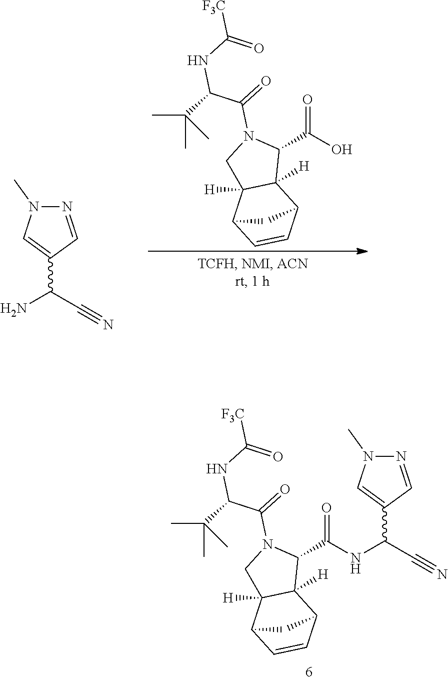

2-amino-2-(1-methylpyrazol-4-yl)acetonitrile was prepared according to the procedure to prepare 2-amino-2-(1H-1,2,4-triazol-3-yl)acetonitrile using 1-methylpyrazole-4-carbaldehyde. LC-MS (ESI, m/z): 137 [M+H]⁺.

To a mixture of 2-amino-2-(1-methylpyrazol-4-yl)acetonitrile (42.0 mg, 0.308 mmol), (1R,2S,3S,6R,7S)-4-[(2S)-3,3-dimethyl-2-(2,2,2-trifluoroacetamido)butanoyl]-4-azatricyclo[5.2.1.0\hat{ }{2,6}]dec-8-ene-3-carboxylic acid (120 mg, 0.308 mmol) and TCFH (113 mg, 0.400 mmol) in CH₃CN (2.5 mL) was added NMI (253 mg, 3.08 mmol). The mixture was stirred for 1 h at rt. The mixture was purified by C18 column with CH₃CN/Water (0.05% FA). The fraction was concentrated under reduced pressure to afford the crude product. The crude product was purified by prep-HPLC (Column: Xselect CSH F-Phenyl OBD column, 19×250 mm, 5 μm; Mobile Phase A: Water (0.1% FA), Mobile Phase B: CH₃CN; Flow rate: 25 mL/min; Gradient: 40% B to 70% B in 7 min, 70% B; Wave Length: 220 nm; RT1(min): 6.55) to provide (1R,2S,3S,6R,7S)—N-[cyano(1-methylpyrazol-4-yl)methyl]-4-[(2S)-3,3-dimethyl-2-(2,2,2-trifluoroacetamido)butanoyl]-4-azatricyclo[5.2.1.0\hat{ }{2,6}]dec-8-ene-3-carboxamide (43.8 mg, 27%) as a white solid. ¹H NMR (400 MHz, 80° C., DMSO-d₆) δ 8.88-9.00 (m, 1H), 8.75-8.87 (m, 1H), 7.75-7.90 (m, 1H), 7.42-7.52 (m, 1H), 5.92-6.20 (m, 2H), 5.85-5.91 (m, 1H), 4.43-4.56 (m, 1H), 4.05-4.20 (m, 1H), 3.75-3.92 (m, 3H), 3.68-3.73 (m, 1H), 3.35-3.50 (m, 1H), 3.15-3.25 (m, 1H), 2.83-3.00 (m, 2H), 2.65-2.82 (m, 1H), 1.32-1.45 (m, 2H), 0.85-1.10 (m, 9H). LC-MS (ESI, m/z): 529 [M+Na]⁺.

### Example 7

### Compound 7

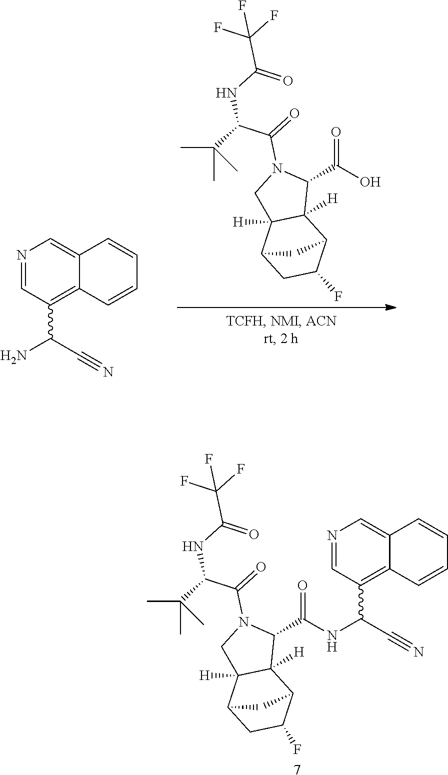

To a mixture of 2-amino-2-(isoquinolin-4-yl)acetonitrile (148 mg, 0.808 mmol), (1S,2R,3S,6R,7S,9R)-4-[(2S)-3,3-dimethyl-2-(2,2,2-trifluoroacetamido)butanoyl]-9-fluoro-4-azatricyclo[5.2.1.0\hat{ }{2,6}]decane-3-carboxylic acid (330 mg, 0.808 mmol) and TCFH (295 mg, 1.05 mmol) in CH₃CN (5 mL) was added NMI (663 mg, 8.08 mmol) at 0° C. The mixture was stirred for 2 h at rt. The crude product was purified by C18 column with CH₃CN/Water (0.05% FA). The fraction was concentrated under reduced pressure to afford the crude product. The crude product was purified by prep-HPLC (Column: Xselect CSH F-Phenyl OBD column, 19×250 mm, 5 μm; Mobile Phase A: Water (0.1% FA), Mobile Phase B: CH₃OH—HPLC; Flow rate: 25 mL/min; Gradient: 53% B to 75% B in 10 min, 75% B; Wave Length: 254 nm; RT1(min): 8.43) to provide (1S,2R,3S,6R,7S,9R)—N-[cyano(isoquinolin-4-yl)methyl]-4-[(2S)-3,3-dimethyl-2-(2,2,2-trifluoroacetamido)butanoyl]-9-fluoro-4-azatricyclo[5.2.1.0\hat{ }{2,6}]decane-3-carboxamide (77.0 mg, 16%) as an off-white solid. (10.6 mg). ¹H NMR (400 MHz, 60° C., DMSO-d₆) δ 9.37-9.48 (m, 1H), 8.68-8.89 (m, 1H), 8.21-8.32 (m, 1H), 7.77-8.09 (m, 3H), 6.67-6.81 (m, 1H), 4.56-4.86 (m, 2H), 4.21-4.51 (m, 1H), 3.67-3.81 (m, 2H), 2.58-2.66 (m, 1H), 2.44-2.52 (m, 1H), 2.29-2.42 (m, 1H), 1.83-2.02 (m, 1H), 1.47-1.71 (m, 2H), 1.21-1.45 (m, 2H), 0.88-1.09 (m, 9H). LC-MS (ESI, m/z): 574 [M+H]⁺.

### Example 8

### Compound 8

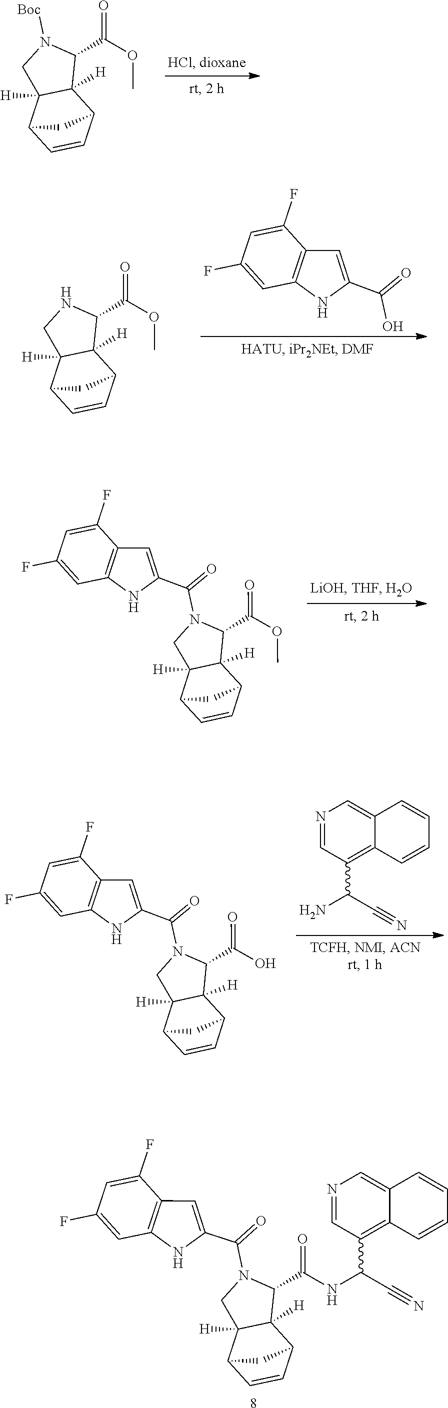

A mixture of 4-t-butyl 3-methyl (1R,2S,3S,6R,7S)-4-azatricyclo[5.2.1.0\hat{ }{2,6}]dec-8-ene-3,4-dicarboxylate (300 mg, 1.02 mmol) in HCl (10 mL, 4 M in dioxane) was stirred for 2 h at rt. The mixture was concentrated under reduced pressure to afford methyl (1R,2S,3S,6R,7S)-4-azatricyclo[5.2.1.0\hat{ }{2,6}]dec-8-ene-3-carboxylate (200 mg, crude) as a yellow solid. LC-MS (ESI, m/z): 194 [M+H]⁺.

To a mixture of methyl (1R,2S,3S,6R,7S)-4-azatricyclo[5.2.1.0\hat{ }{2,6}]dec-8-ene-3-carboxylate (200 mg, 1.02 mmol), 4,6-difluoro-1H-indole-2-carboxylic acid (201 mg, 1.02 mmol) and O-(7-azabenzotriazol-1-yl)-N,N-tetramethyluronium hexafluorophosphate (HATU) (465 mg, 1.22 mmol) in DMF (8 mL) was added N-ethyl-N-isopropylpropan-2-amine (791 mg, 6.11 mmol) at 0° C. The mixture was stirred for 1 h at rt. The crude product was purified by C18 column with CH₃CN/water (0.05% TFA). The fraction was concentrated under reduced pressure to afford methyl (1R,2S,3S,6R,7S)-4-(4,6-difluoro-1H-indole-2-carbonyl)-4-azatricyclo[5.2.1.0\hat{ }{2,6}]dec-8-ene-3-carboxylate (200 mg, crude) as an orange solid. LC-MS (ESI, m/z): 373 [M+H]⁺.

To a mixture of methyl (1R,2S,3S,6R,7S)-4-(4,6-difluoro-1H-indole-2-carbonyl)-4-azatricyclo[5.2.1.0\hat{ }{2,6}]dec-8-ene-3-carboxylate (200 mg, 0.537 mmol) in THF (3 mL) was added a solution of lithium hydroxide (39.0 mg, 1.63 mmol) in water (3 mL). The mixture was stirred for 2 h at rt. The mixture was diluted with water (10 mL) and adjusted to pH=6 with HCl (1 M). The mixture was extracted with EA (3×20 mL). The organic layers were combined, washed with brine (2×20 mL), dried over anhydrous sodium sulfate, filtered and concentrated under reduced pressure to afford (1R,2S,3S,6R,7S)-4-(4,6-difluoro-1H-indole-2-carbonyl)-4-azatricyclo[5.2.1.0\hat{ }{2,6}]dec-8-ene-3-carboxylic acid (180 mg, crude) as a yellow solid. LC-MS (ESI, m/z): 359 [M+H]⁺.

A mixture of isoquinoline-4-carbaldehyde (200 mg, 1.27 mmol) in NH₃ (5 mL, 7 M in CH₃OH) was stirred for 2 h at rt. TMSCN (252 mg, 2.54 mmol) was added at 0° C. The mixture was stirred overnight at rt and then concentrated under reduced pressure to afford 2-amino-2-(isoquinolin-4-yl)acetonitrile) (230 mg, crude) as a brown semi-solid. LC-MS (ESI, m/z): 184 [M+H]⁺.

To a mixture of 2-amino-2-(isoquinolin-4-yl)acetonitrile (82.0 mg, 0.448 mmol), (1R,2S,3S,6R,7S)-4-(4,6-difluoro-1H-indole-2-carbonyl)-4-azatricyclo[5.2.1.0\hat{ }{2,6}]dec-8-ene-3-carboxylic acid (160 mg, 0.448 mmol) and TCFH (163 mg, 0.582 mmol) in CH₃CN (3.5 mL) was added NMI (367 mg, 4.48 mmol). The mixture was stirred for 1 h at rt. The crude product was purified by C18 column with CH₃CN/Water (0.05% NH₄HCO₃). The fraction was concentrated under reduced pressure to afford the crude product. The crude product was purified by prep-HPLC Column: xBridge Prep Phenyl OBD Column, 19×250 mm, 5 μm; Mobile Phase A: Water (0.1% FA), Mobile Phase B: CH₃CN; Flow rate: 25 mL/min; Gradient: 45% B to 75% B in 10 min, 75% B; Wave Length: 220 nm; RT1(min): 7.32 to provide (1R,2S,3S,6R,7S)—N-[cyano(isoquinolin-4-yl)methyl]-4-(4,6-difluoro-1H-indole-2-carbonyl)-4-azatricyclo[5.2.1.0\hat{ }{2,6}]dec-8-ene-3-carboxamide (14.1 mg, 5%) as a light yellow solid. ¹H NMR (400 MHz, 100° C., DMSO-d₆) δ 11.35-11.68 (m, 1H), 9.15-9.48 (m, 2H), 8.65-8.80 (m, 1H), 7.42-8.30 (m, 4H), 6.60-7.18 (m, 4H), 5.95-6.20 (m, 2H), 4.30-4.50 (m, 1H), 3.75-4.10 (m, 1H), 3.55-3.74 (m, 1H), 3.10-3.20 (m, 1H), 2.65-2.95 (m, 3H), 1.30-1.50 (m, 2H). LC-MS (ESI, m/z): 524 [M+H]⁺.

### Example 9

### Compound 9

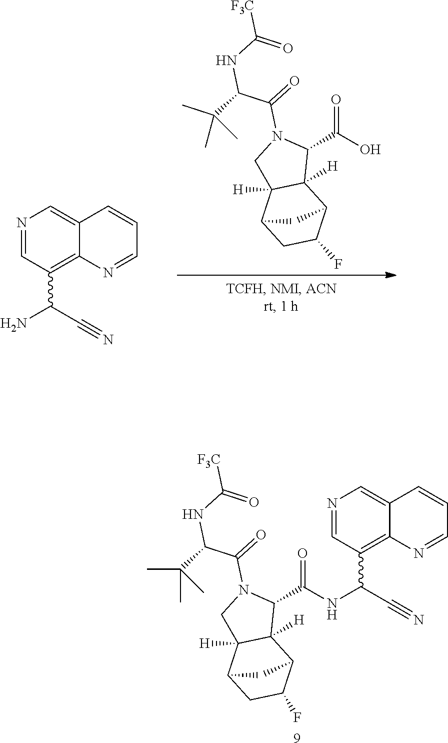

2-amino-2-(1,6-naphthyridin-8-yl)acetonitrile was prepared according to the procedure to prepare 2-amino-2-(1-methylpyrazol-4-yl)acetonitrile using 1,6-naphthyridine-8-carbaldehyde. LC-MS (ESI, m/z): 185 [M+H]⁺.

To a mixture of 2-amino-2-(1,6-naphthyridin-8-yl)acetonitrile (54.0 mg, 0.293 mmol), (1S,2R,3S,6R,7S,9R)-4-[(2S)-3,3-dimethyl-2-(2,2,2-trifluoroacetamido)butanoyl]-9-fluoro-4-azatricyclo[5.2.1.0\hat{ }{2,6}]decane-3-carboxylic acid (120 mg, 0.293 mmol) and TCFH (107 mg, 0.381 mmol) in CH₃CN (2 mL) was added NMI (241 mg, 2.93 mmol). The mixture was stirred for 1 h at rt, then purified by C18 column with CH₃CN/water (0.05% FA). The best fractions were concentrated under reduced pressure to afford the crude product (70 mg). The crude product was purified by prep-HPLC (Column: xSelect CSH Prep C18 OBD Column, 19×150 mm, 5 μm; Mobile Phase A: Water (0.1% FA), Mobile Phase B: CH₃CN; Flow rate: 25 mL/min; Gradient: 40% B to 70% B in 7 min, 70% B; Wave Length: 254 nm; RT1(min): 5.42) to provide (1S,2R,3S,6R,7S,9R)—N-[cyano(1,6-naphthyridin-8-yl)methyl]-4-[(2S)-3,3-dimethyl-2-(2,2,2-trifluoroacetamido)butanoyl]-9-fluoro-4-azatricyclo[5.2.1.0\hat{ }{2,6}]decane-3-carboxamide (25.6 mg, 15%) as a light brown solid. ¹H NMR (400 MHz, 80° C., DMSO-d₆) δ 9.42-9.56 (m, 1H), 9.11-9.32 (m, 2H), 8.85-9.10 (m, 2H), 8.60-8.75 (m, 1H), 7.70-7.85 (m, 1H), 6.72-6.88 (m, 1H), 4.70-4.85 (m, 1H), 4.50-4.69 (m, 1H), 4.20-4.49 (m, 1H), 3.65-3.80 (m, 1H), 3.45-3.64 (m, 1H), 2.50-2.78 (m, 3H), 2.20-2.40 (m, 1H), 1.85-2.05 (m, 1H), 1.55-1.75 (m, 1H), 1.22-1.54 (m, 2H), 0.72-1.08 (m, 9H). LC-MS (ESI, m/z): 575 [M+H]⁺.

### Example 10

### Compound 10

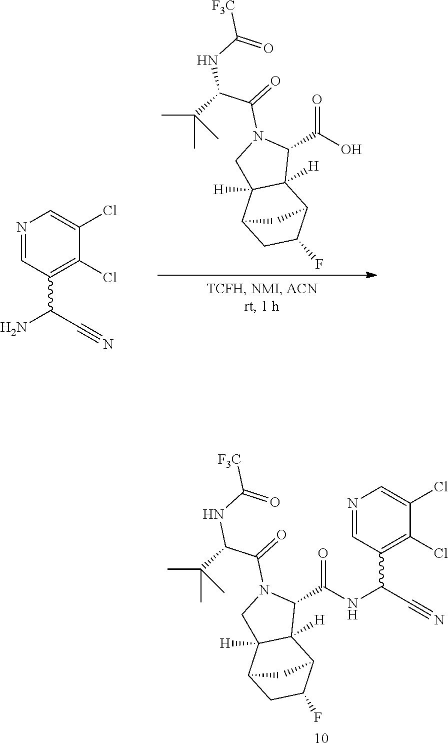

2-amino-2-(4,5-dichloropyridin-3-yl)acetonitrile was prepared according to the procedure to prepare 2-amino-2-(1-methylpyrazol-4-yl)acetonitrile using 4,5-dichloropyridine-3-carbaldehyde. LC-MS (ESI, m/z): 202 [M+H]⁺.

To a mixture of 2-amino-2-(4,5-dichloropyridin-3-yl)acetonitrile (50.0 mg, 0.247 mmol) and (1S,2R,3S,6R,7S,9R)-4-[(2S)-3,3-dimethyl-2-(2,2,2-trifluoroacetamido)butanoyl]-9-fluoro-4-azatricyclo[5.2.1.0\hat{ }{2,6}]decane-3-carboxylic acid (101 mg, 0.247 mmol) in CH₃CN (5 mL) was added NMI (203 mg, 2.47 mmol) and TCFH (90.0 mg, 0.321 mmol). The mixture was stirred for 1 h at rt. The mixture was purified by C18 column with CH₃CN/water (0.05% FA). The fraction was concentrated under reduced pressure to afford the crude product. The crude product was purified by prep-HPLC (Column: xBridge Prep Phenyl OBD Column, 19×250 mm, 5 μm; Mobile Phase A: Water (0.1% FA), Mobile Phase B: CH₃CN; Flow rate: 25 mL/min; Gradient: 45% B to 75% B in 7 min, 75% B; Wave Length: 254 nm; RT1(min): 6.78) to provide (1S,2R,3S,6R,7S,9R)—N-[cyano(4,5-dichloropyridin-3-yl)methyl]-4-[(2S)-3,3-dimethyl-2-(2,2,2-trifluoroacetamido)butanoyl]-9-fluoro-4-azatricyclo[5.2.1.0\hat{ }{2,6}]decane-3-carboxamide (12.8 mg, 8%) as a white solid. ¹H NMR (400 MHz, 80° C., DMSO-d₆) δ 8.91-9.95 (m, 2H), 8.25-8.90 (m, 2H), 5.91-6.49 (m, 1H), 4.50-4.70 (m, 2H), 4.20-4.49 (m, 1H), 3.47-3.98 (m, 2H), 2.51-2.88 (m, 3H), 2.14-2.37 (m, 1H), 1.78-1.98 (m, 1H), 1.57-1.77 (m, 1H), 1.18-1.76 (m, 2H), 0.81-1.17 (m, 9H). LC-MS (ESI, m/z): 592[M+H]⁺.

### Example 11

### Compound 11

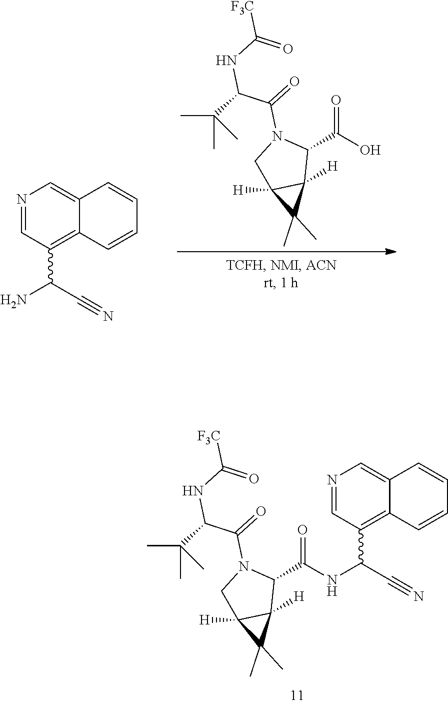 [SMILES: N#CC(N)c1cncc2ccccc12 ; CC(C)(C)[C@H](NC(=O)C(F)(F)F)C(=O)N1C[C@H]2[C@@H]([C@H]1C(=O)O)C2(C)C ; CC(C)(C)[C@H](NC(=O)C(F)(F)F)C(=O)N1C[C@H]2[C@@H]([C@H]1C(=O)NC(C#N)c1cncc3ccccc13)C2(C)C]

To a mixture of 2-amino-2-(isoquinolin-4-yl)acetonitrile (60.0 mg, 0.329 mmol), (1R,2S,5S)-3-[(2S)-3,3-dimethyl-2-(2,2,2-trifluoroacetamido)butanoyl]-6,6-dimethyl-3-azabicyclo[3.1.0]hexane-2-carboxylic acid (120 mg, 0.329 mmol) and TCFH (120 mg, 0.428 mmol) in CH₃CN (2.5 mL) was added NMI (270 mg, 3.29 mmol). The mixture was stirred for 1 h at rt. The crude product was purified by C18 column with CH₃CN/water (0.05% NH₄HCO₃). The fraction was concentrated under reduced pressure to afford the crude product. The crude product was purified by prep-HPLC (Column: xSelect CSH Prep C18 OBD Column, 19×250 mm, 5 μm; Mobile Phase A: Water (0.1% FA), Mobile Phase B: CH₃CN; Flow rate: 25 mL/min; Gradient: 43% B to 73% B in 7 min, 73% B; Wave Length: 220 nm; RT1(min): 6.87) to provide (1R,2S,5S)—N-[cyano(isoquinolin-4-yl)methyl]-3-[(2S)-3,3-dimethyl-2-(2,2,2-trifluoroacetamido)butanoyl]-6,6-dimethyl-3-azabicyclo[3.1.0]hexane-2-carboxamide (21.1 mg, 12%) as a light yellow solid. ¹H NMR (400 MHz, 100° C., DMSO-d₆) δ 9.35-9.45 (m, 1H), 9.15-9.34 (m, 1H), 8.60-8.85 (m, 2H), 8.15-8.28 (m, 1H), 7.95-8.08 (m, 1H), 7.70-7.94 (m, 2H), 6.65-6.80 (m, 1H), 4.40-4.50 (m, 1H), 4.20-4.38 (m, 1H), 3.85-4.00 (m, 1H), 3.65-3.78 (m, 1H), 1.45-1.60 (m, 1H), 1.10-1.40 (m, 1H), 0.95-1.08 (m, 12H), 0.72-0.94 (m, 3H). LC-MS (ESI, m/z): 530 [M+H]⁺.

### Example 12

### Compound 12

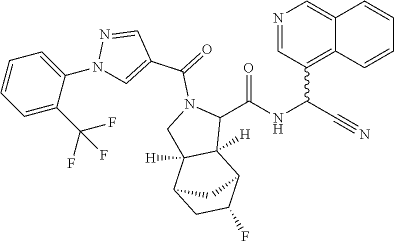 [SMILES: N#CC(NC(=O)C1[C@@H]2[C@H](CN1C(=O)c1cnn(-c3ccccc3C(F)(F)F)c1)[C@H]1C[C@@H]2[C@H](F)C1)c1cncc2ccccc12]

To a mixture of methyl (1S,2R,3S,6R,7S,9R)-9-fluoro-4-azatricyclo[5.2.1.0\hat{ }{2,6}]decane-3-carboxylate (136 mg, 0.638 mmol), 1-[2-(trifluoromethyl)phenyl]pyrazole-4-carboxylic acid (169 mg, 0.657 mmol) and HATU (250 mg, 0.657 mmol) in DCM (6 mL) and DMF (1.5 mL) was added N-methylmorphline (194 mg, 1.91 mmol) at 0° C. The mixture was stirred for 1 h at rt, and the reaction was quenched with water (20 mL). The mixture was extracted with EA (3×20 mL). The organic layers were combined, washed with brine (2×20 mL) and dried over anhydrous sodium sulfate. The solids were removed by filtration and the filtrate was concentrated under reduced pressure to afford the crude product. The crude product was chromatographed on a silica gel column with EA:PE (2:5) to provide methyl (1S,2R,3S,6R,7S,9R)-9-fluoro-4-{1-[2-(trifluoromethyl)phenyl]pyrazole-4-carbonyl}-4-azatricyclo[5.2.1.0\hat{ }{2,6}]decane-3-carboxylate (275 mg, 84%) as a light yellow oil. LC-MS (ESI, m/z): 452 [M+H]⁺.

To a mixture of methyl (1S,2R,3S,6R,7S,9R)-9-fluoro-4-{1-[2-(trifluoromethyl)phenyl]pyrazole-4-carbonyl}-4-azatricyclo[5.2.1.0\hat{ }{2,6}]decane-3-carboxylate (275 mg, 0.609 mmol) in THF (3 mL) was added a solution of LiOH (44.0 mg, 1.83 mmol) in water (3 mL). The mixture was stirred for 2 h at rt, then diluted with water (10 mL) and adjusted to pH=6 with HCl (1 M, aq.). The mixture was extracted with EA (3×10 mL). The organic layers were combined, washed with brine (2×10 mL), dried over anhydrous sodium sulfate, filtered and concentrated under reduced pressure to provide (1S,2R,3S,6R,7S,9R)-9-fluoro-4-{1-[2-(trifluoromethyl)phenyl]pyrazole-4-carbonyl}-4-azatricyclo[5.2.1.0\hat{ }{2,6}]decane-3-carboxylic acid (260 mg, 78%) as a light yellow oil. LC-MS (ESI, m/z): 438 [M+H]⁺.

To a mixture of (1S,2R,3S,6R,7S,9R)-9-fluoro-4-{1-[2-(trifluoromethyl)phenyl]pyrazole-4-carbonyl}-4-azatricyclo[5.2.1.0\hat{ }{2,6}]decane-3-carboxylic acid (100 mg, 0.229 mmol), 2-amino-2-(isoquinolin-4-yl)acetonitrile (42.0 mg, 0.229 mmol) and TCFH (84.0 mg, 0.298 mmol) in CH₃CN (2 mL) was added NMI (188 mg, 2.29 mmol). The mixture was stirred for 1 h at rt. The crude product was purified by C18 column with CH₃CN/water (0.05% FA), the fraction was concentrated under reduced pressure to afford the crude product. The crude product was purified by prep-HPLC (Column: Xselect CSH C18 OBD Column 30×150 mm, 5 μm; Mobile Phase A: water (0.1% FA), Mobile Phase B: ACN; Flow rate: 60 mL/min; Gradient: 43% B to 51% B in 8 min, 51% B; Wave Length: 254; 220 nm; RT1(min): 6.77) to provide (1S,2R,3S,6R,7S,9R)—N-[cyano(isoquinolin-4-yl)methyl]-9-fluoro-4-{1-[2-(trifluoromethyl)phenyl]pyrazole-4-carbonyl}-4-azatricyclo[5.2.1.0\hat{ }{2,6}]decane-3-carboxamide (13.6 mg, 9%) as alight yellow oil. ¹H NMR (400 MHz, 100° C., DMSO-d₆) δ 9.21-9.50 (m, 2H), 8.62-8.81 (m, 1H), 8.29-8.52 (m, 1H), 8.12-8.28 (m, 1H), 7.93-8.11 (m, 2H), 7.51-7.91 (m, 6H), 6.61-6.78 (m, 1H), 4.71-5.01 (m, 1H), 4.39-4.70 (m, 1H), 3.55-4.00 (m, 2H), 2.49-2.79 (m, 3H), 2.21-2.39 (m, 1H), 1.73-2.04 (m, 1H), 1.57-1.72 (m, 1H), 1.27-1.56 (m, 2H). LC-MS (ESI, m/z): 603 [M+H]⁺.

### Example 13

### Compound 13

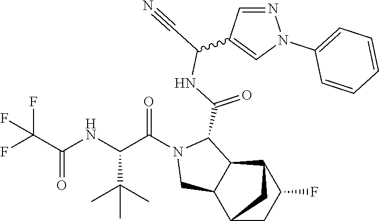 [SMILES: CC(C)(C)[C@H](NC(=O)C(F)(F)F)C(=O)N1C[C@@H]2[C@@H]3C[C@H]([C@@H]2[C@H]1C(=O)NC(C#N)c1cnn(-c2ccccc2)c1)[C@H](F)C3]

Compound 14 was prepared similarly as described for compound 4 using 1-phenylpyrazole-4-carbaldehyde in place of 1H-1,2,4-triazole-3-carbaldehyde. ¹H NMR (400 MHz, 80° C., DMSO-d₆) δ 8.80-9.30 (m, 2H), 8.45-8.79 (m, 1H), 7.70-7.90 (m, 3H), 7.41-7.55 (m, 2H), 7.30-7.40 (m, 1H), 5.65-6.15 (m, 1H), 4.55-4.86 (m, 2H), 4.21-4.54 (m, 1H), 3.66-3.90 (m, 1H), 3.50-3.65 (m, 1H), 2.55-2.90 (m, 3H), 2.20-2.40 (m, 1H), 1.90-2.05 (m, 1H), 1.60-1.75 (m, 1H), 1.32-1.50 (m, 1H), 1.20-1.31 (m, 1H), 0.80-1.10 (m, 9H). LC-MS (ESI, m/z): 589 [M+H]⁺.

To a mixture of DMF (8.87 g, 121 mmol) and phosphorus oxychloride (18.6 g, 121 mmol) was added 1-phenyl-1H-pyrazole (3.50 g, 24.3 mmol). The mixture was stirred for 4 h at 100° C. and the reaction quenched with ice-water (150 mL). The mixture was neutralized to pH=7 with sodium carbonate. The mixture was extracted with EA (3×60 mL). The organic layers were combined, washed with brine (2×60 mL) and dried over anhydrous sodium sulfate. The solids were removed by filtration. The filtrate was concentrated under reduced pressure to afford the crude product that was chromatographed on a silica gel column with EA:PE (1:7) to provide 1-phenylpyrazole-4-carbaldehyde (2.58 g) as a light yellow solid. LC-MS (ESI, m/z): 173 [M+H]⁺.

### Example 14

### Compound 14

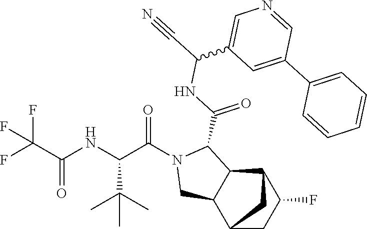 [SMILES: CC(C)(C)[C@H](NC(=O)C(F)(F)F)C(=O)N1C[C@@H]2[C@@H]3C[C@H]([C@@H]2[C@H]1C(=O)NC(C#N)c1cncc(-c2ccccc2)c1)[C@H](F)C3]

Compound 14 was prepared similarly as described for compound 4 using 5-phenylpyridine-3-carbaldehyde in place of 1H-1,2,4-triazole-3-carbaldehyde. ¹H NMR (400 MHz, 80° C., DMSO-d₆) δ 9.28-9.45 (m, 1H), 8.80-9.20 (m, 2H), 8.30-8.75 (m, 1H), 8.05-8.15 (m, 1H), 7.65-7.88 (m, 2H), 7.50-7.60 (m, 2H), 7.38-7.49 (m, 1H), 6.15-6.40 (m, 1H), 4.59-4.80 (m, 2H), 4.30-4.58 (m, 1H), 3.71-3.88 (m, 1H), 3.50-3.70 (m, 1H), 2.55-2.74 (m, 3H), 2.24-2.50 (m, 1H), 1.90-2.01 (m, 1H), 1.60-1.75 (m, 1H), 1.15-1.55 (m, 2H), 0.88-1.10 (m, 9H). LC-MS (ESI, m/z): 600 [M+H]⁺.

To a mixture of 5-bromopyridine-3-carbaldehyde (2.00 g, 10.8 mmol), Pd(PPh₃)₄ (621 mg, 0.538 mmol) in sodium carbonate (25 mL, 2M, aq.) and toluene (27 mL) was added phenyl boronic acid (1.97 g, 16.1 mmol) under nitrogen. The mixture was stirred for 3 h at 100° C. under nitrogen and then diluted with water (6 mL). The mixture was extracted with EA (3×150 mL). The organic layers were combined, washed with brine (2×100 mL) and dried over anhydrous sodium sulfate. The solids were removed by filtration. The filtrate was concentrated under reduced pressure to afford crude product that was chromatographed on a silica gel column with EA:PE (1:1) to provide 5-phenylpyridine-3-carbaldehyde (2 g, crude, no further purification was done). ¹H NMR (400 MHz, CDCl₃) δ 10.22 (s, 1H), 9.10-9.30 (m, 2H), 8.25-8.45 (m, 1H), 7.45-7.65 (m, 5H). LC-MS (ESI, m/z): 184 [M+H]⁺.

### Example 15

### Compound 15

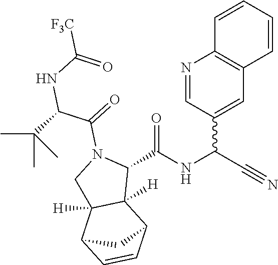 [SMILES: CC(C)(C)[C@H](NC(=O)C(F)(F)F)C(=O)N1C[C@H]2[C@@H]([C@H]1C(=O)NC(C#N)c1cnc3ccccc3c1)[C@H]1C=C[C@@H]2C1]

Compound 15 was prepared similarly as described for compound 4 using quinoline-3-carbaldehyde in place of 1H-1,2,4-triazole-3-carbaldehyde. ¹H NMR (500 MHz, 364K, DMSO-d₆) δ 9.08-9.28 (m, 1H), 8.93 (dd, 1H), 8.53-8.74 (m, 1H), 8.42 (m, 1H), 8.00-8.11 (m, 2H), 7.80 (m, 1H), 7.65 (m, 1H), 6.31-6.45 (dd, 1H), 5.97-6.21 (m, 2H), 4.27-4.52 (m, 1H), 4.12-4.24 (m, 1H), 3.66 (m, 1H), 3.44-3.50 (m, 1H), 3.01-3.08 (m, 1H), 2.86-2.97 (m, 2H), 2.72-2.85 (m, 1H), 1.33-1.45 (m, 2H), 0.86-0.99 (m, 9H). LC-MS (ESI, m/z): 554 [M+H]⁺.

### Example 16

### Compound 16

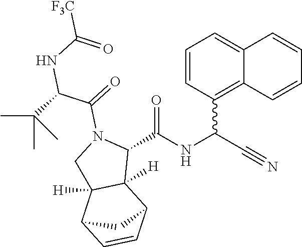 [SMILES: CC(C)(C)[C@H](NC(=O)C(F)(F)F)C(=O)N1C[C@H]2[C@@H]([C@H]1C(=O)NC(C#N)c1cccc3ccccc13)[C@H]1C=C[C@@H]2C1]

Compound 16 was prepared similarly as described for compound 2 using 2-amino-2-(naphthalen-1-yl)acetonitrile in place of 2-amino-2-(imidazo[1,2-a]pyridin-3-yl)acetonitrile. ¹H NMR (500 MHz, 363K, DMSO-d₆) δ 9.02-9.21 (m, 1H), 8.67 (br. s., 1H), 7.99 (m, 2H), 7.93 (m, 1H), 7.83 (m, 1H), 7.49-7.69 (m, 3H), 6.69 (m, 1H), 5.88-6.20 (m, 2H), 4.24-4.55 (m, 1H), 4.08-4.20 (m, 1H), 3.68 (m, 1H), 3.46 (m, 1H), 3.01-3.07 (m, 1H), 2.84-2.95 (m, 2H), 2.59-2.83 (m, 1H), 1.19-1.45 (m, 2H), 0.81-1.09 (m, 9H). LC-MS (ESI, m/z): 553 [M+H]⁺.

### Example 17

### Compound 17

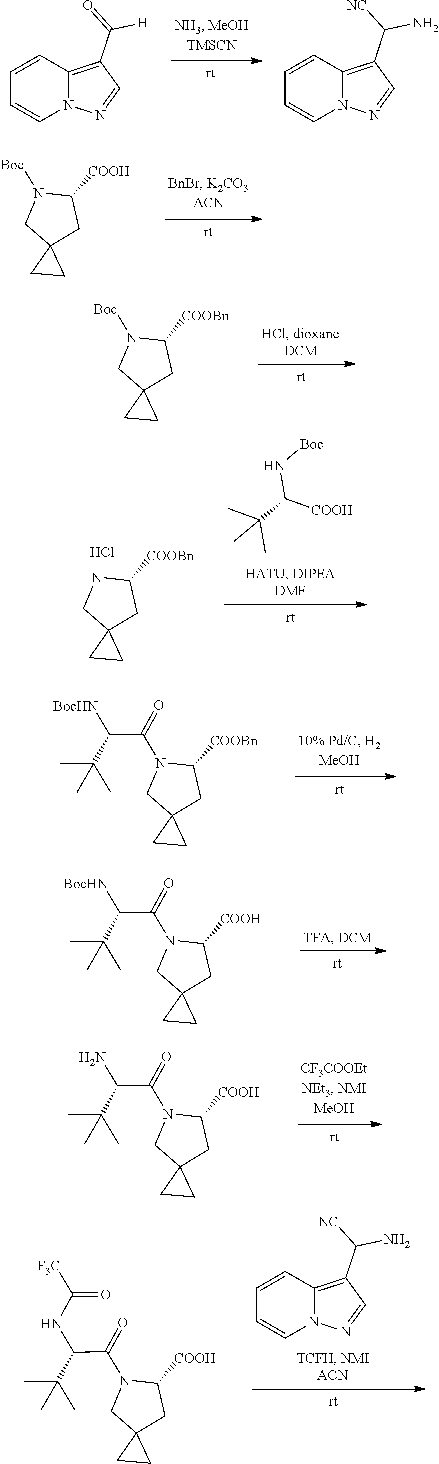

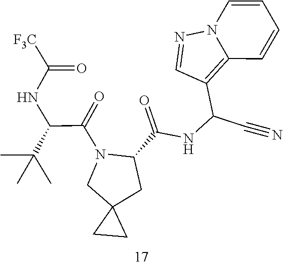 [SMILES: CC(C)(C)[C@H](NC(=O)C(F)(F)F)C(=O)N1CC2(CC2)C[C@H]1C(=O)NC(C#N)c1cnn2ccccc12]

A solution of pyrazolo[1,5-a]pyridine-3-carbaldehyde (200 mg, 1.36 mmol) in 7M ammonia in methanol (2 mL) was stirred at rt for 1 h. TMSCN (0.20 mL, 1.64 mmol) was added and the mixture was stirred at rt for 16 h. The mixture was concentrated under reduced pressure to afford 2-amino-2-(pyrazolo[1,5-a]pyridin-3-yl)acetonitrile (200 mg, 85%) as a brown oil.

To a solution of (S)-5-(t-butoxycarbonyl)-5-azaspiro[2.4]heptane-6-carboxylic acid (500 mg, 2.07 mmol) in ACN (10 mL) was added K₂CO₃ (571 mg, 4.14 mmol). After stirring for 15 min, benzyl bromide (0.28 mL, 2.49 mmol) was added, and the mixture was stirred at rt for 16 h. The mixture was filtered through celite and the filtrate was concentrated under reduced pressure. The crude product was purified by flash chromatography on silica gel using a gradient of EA (15 to 30%) in PE to afford 6-benzyl 5-(tert-butyl) (S)-5-azaspiro[2.4]heptane-5,6-dicarboxylate (600 mg, 87%) as a colorless oil.

To a solution of 6-benzyl 5-(t-butyl) (S)-5-azaspiro[2.4]heptane-5,6-dicarboxylate (600 mg, 1.81 mmol) in DCM (6 mL) cooled at 0° C. was added 4M HCL in dioxane (1.8 mL, 7.25 mmol). The mixture was stirred at rt for 1 h and then concentrated under reduced pressure to afford quantitatively benzyl (S)-5-azaspiro[2.4]heptane-6-carboxylate hydrochloride as an off-white solid.

To a solution of benzyl (S)-5-azaspiro[2.4]heptane-6-carboxylate hydrochloride (600 mg, 2.26 mmol) in DMF (6 mL) cooled at 0° C. were added (S)-2-((t-butoxycarbonyl)amino)-3,3-dimethylbutanoic acid (625 mg, 2.71 mmol), HATU (1.02 g, 2.71 mmol) and DIPEA (0.98 mL, 5.64 mmol). The mixture was stirred at rt for 5 h. The mixture was diluted with water (10 mL) and extracted with EA (2×15 mL). The organic phases were combined, washed with brine (2×10 mL), dried over Na₂SO₄, filtered and concentrated under reduced pressure. The residue was purified by flash chromatography on silica gel using a gradient of EA (15 to 30%) in PE to afford benzyl (S)-5-((S)-2-((t-butoxycarbonyl)amino)-3,3-dimethylbutanoyl)-5-azaspiro[2.4]heptane-6-carboxylate (800 mg, 95%) as a colorless oil.

A mixture of benzyl (S)-5-((S)-2-((t-butoxycarbonyl)amino)-3,3-dimethylbutanoyl)-5-azaspiro[2.4]heptane-6-carboxylate (800 mg, 1.80 mmol) and 10% Pd/C (100 mg) in MeOH (8 mL) was stirred for 2 h under hydrogen atmosphere. The mixture was filtered through celite and the filtrate was concentrated under reduced pressure to afford (S)-5-((S)-2-((tert-butoxycarbonyl)amino)-3,3-dimethylbutanoyl)-5-azaspiro[2.4]heptane-6-carboxylic acid (600 mg, 90%) as a white solid.

To a solution of (S)-5-((S)-2-((t-butoxycarbonyl)amino)-3,3-dimethylbutanoyl)-5-azaspiro[2.4]heptane-6-carboxylic acid (600 mg, 1.69 mmol) in DCM (6 mL) cooled at 0° C. was added TFA (0.6 mL, 8.47 mmol) and the mixture was stirred at rt for 5 h. The mixture was concentrated under reduced pressure to afford quantitatively (S)-5-((S)-2-amino-3,3-dimethylbutanoyl)-5-azaspiro[2.4]heptane-6-carboxylic acid under its trifluoroacetic acid salt form as a yellow oil.

To a solution of (S)-5-((S)-2-amino-3,3-dimethylbutanoyl)-5-azaspiro[2.4]heptane-6-carboxylic acid trifluoroacetic acid salt (600 mg, 2.36 mmol) in MeOH (10 mL) were added ethyl 2,2,2-trifluoroacetate (1.41 mL, 11.8 mmol), NEt₃ (1.65 mL, 11.8 mmol) and NMI (0.37 mL, 4.72 mmol). The mixture was stirred at rt for 16 h and then concentrated under reduced pressure. The residue was acidified with 1N HCl until pH=6 and extracted with EA (2×15 mL). The organic phases were combined, dried over Na₂SO₄. The solids were removed by filtration and the filtrate was concentrated under reduced pressure to afford (S)-5-((S)-3,3-dimethyl-2-(2,2,2-trifluoroacetamido)butanoyl)-5-azaspiro[2.4]heptane-6-carboxylic acid (400 mg, 67%) as an off-white solid.

To a solution of (S)-5-((S)-3,3-dimethyl-2-(2,2,2-trifluoroacetamido)butanoyl)-5-azaspiro[2.4]heptane-6-carboxylic acid (200 mg, 0.571 mmol) in ACN (2 mL) were added 2-amino-2-(pyrazolo[1,5-a]pyridin-3-yl)acetonitrile (117 mg, 0.685 mmol), TCFH (191 mg, 0.685 mmol) and NMI (0.45 mL, 5.71 mmol). The mixture was stirred at rt for 2 h. The mixture was diluted with water (5 mL) and extracted with EA (2×10 mL). The organic phases were combined, washed with brine (10 mL) and dried over Na₂SO₄. The solids were removed by filtration and the filtrate was concentrated under reduced pressure. The residue was purified by flash chromatography on C18 using a gradient of ACN (20 to 40%) in 0.1% FA in H₂O to afford (6S)—N-(cyano(pyrazolo[1,5-a]pyridin-3-yl)methyl)-5-((S)-3,3-dimethyl-2-(2,2,2-trifluoroacetamido)butanoyl)-5-azaspiro[2.4]heptane-6-carboxamide (90 mg, 31%) as a white solid. ¹H NMR (500 MHz, 369K, DMSO-d₆) δ 8.81 (m, 1H), 8.51-8.70 (m, 2H), 8.06 (m, 1H), 7.75 (m, 1H), 7.24-7.34 (m, 1H), 6.94 (m, 1H), 6.33 (m, 1H), 4.36-4.61 (m, 2H), 3.37-3.71 (m, 2H), 1.95-2.11 (m, 1H), 1.76-1.95 (m, 1H), 0.93-1.05 (m, 9H), 0.46-0.71 (m, 4H). LC-MS (ESI, m/z): 505 [M+H]⁺.

### Example 18

### Compound 18

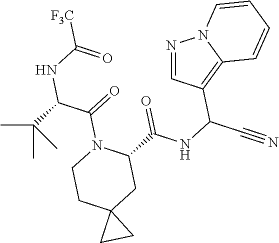 [SMILES: CC(C)(C)[C@H](NC(=O)C(F)(F)F)C(=O)N1CCC2(CC2)C[C@H]1C(=O)NC(C#N)c1cnn2ccccc12]

Compound 18 was prepared similarly as described for Compound 17 using (S)-6-(t-butoxycarbonyl)-6-azaspiro[2.5]octane-5-carboxylic acid in place of (S)-5-(t-butoxycarbonyl)-5-azaspiro[2.4]heptane-6-carboxylic acid. ¹H N MR (500 MHz, 363K, DMSO-d₆) δ 8.55-8.82 (m, 3H), 8.06 (m, 1H), 7.68-7.81 (m, 1H), 7.30 (m, 1H), 6.95 (m, 1H), 6.26-6.38 (m, 1H), 4.81-5.21 (m, 2H), 3.51-4.10 (m, 2H), 2.41-2.56 (m, 1H), 1.87-2.06 (m, 1H), 1.46-1.80 (m, 2H), 0.92-1.10 (m, 9H), 0.06-0.45 (m, 4H). LC-MS (ESI, m/z): 519 [M+H]⁺.

### Example 19

### Compound 19

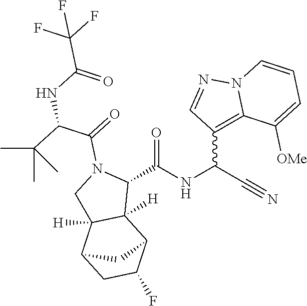 [SMILES: COc1cccn2ncc(C(C#N)NC(=O)[C@@H]3[C@@H]4[C@H](CN3C(=O)[C@@H](NC(=O)C(F)(F)F)C(C)(C)C)[C@H]3C[C@@H]4[C@H](F)C3)c12]

Compound 19 was prepared similarly as described for Compound 7 using 2-amino-2-(4-methoxypyrazolo[1,5-a]pyridin-3-yl)acetonitrile in place of 2-amino-2-(isoquinolin-4-yl)acetonitrile. ¹H NMR (500 MHz, 362K, DMSO-d₆) δ 8.69-8.81 (m, 2H), 8.25 (m, 1H), 8.05 (m, 1H), 6.87 (m, 1H), 6.72 (m, 1H), 6.30 (m, 1H), 4.71 (m, 1H), 4.63 (m, 1H), 4.30-4.47 (m, 1H), 3.95 (m, 3H), 3.74 (m, 1H), 3.56-3.66 (m, 1H), 2.50-2.67 (m, 3H), 2.32 (m, 1H), 1.93 (m, 1H), 1.64 (m, 1H), 1.27-1.52 (m, 2H), 0.88-1.06 (m, 9H). LCMS (ESI, m/z): 593 [M+H]⁺.

2-Amino-2-(4-methoxypyrazolo[1,5-a]pyridin-3-yl)acetonitrile: To a solution of 4-methoxypyrazolo[1,5-a]pyridine (350 mg, 2.36 mmol) in dry DMF (4 mL) cooled at 0° C. was added POCl₃ (0.663 mL, 7.09 mmol). The mixture was stirred at rt for 2 h. After cooling to 0° C., the mixture was basified with 6N NaOH until pH=10 and extracted with Et₂O (3×20 mL). The organic phases were combined, washed with water (2×10 mL) and concentrated under reduced pressure to afford 4-methoxypyrazolo[1,5-a]pyridine-3-carbaldehyde (300 mg, 72%) as a brown solid.

A solution of 4-methoxypyrazolo[1,5-a]pyridine-3-carbaldehyde (100 mg, 0.568 mmol) in 7M NH₃ in methanol (2 mL) was heated at 35° C. for 10 min in a sealed tube. TMSCN (0.056 mL, 0.62 mmol) was added, and the mixture was stirred at rt for 16 h. The mixture was concentrated under reduced pressure to afford 2-amino-2-(4-methoxypyrazolo[1,5-a]pyridin-3-yl)acetonitrile (110 mg, 96%) as a brown oil.

### Example 20

### Compound 20

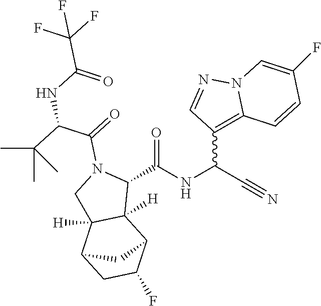 [SMILES: CC(C)(C)[C@H](NC(=O)C(F)(F)F)C(=O)N1C[C@@H]2[C@H]3C[C@@H]([C@@H]2[C@H]1C(=O)NC(C#N)c1cnn2cc(F)ccc12)[C@H](F)C3]

Compound 20 was prepared similarly as described for Compound 7 using 2-amino-2-(6-fluoropyrazolo[1,5-a]pyridin-3-yl)acetonitrile in place of 2-amino-2-(isoquinolin-4-yl)acetonitrile. ¹H NMR (500 MHz, 364K, DMSO-d₆) δ 9.04 (m, 1H), 8.86-8.97 (m, 1H), 8.70-8.83 (m, 1H), 8.06-8.22 (m, 1H), 7.75-7.88 (m, 1H), 7.33-7.49 (m, 1H), 6.24-6.40 (m, 1H), 4.55-4.79 (m, 2H), 4.27-4.50 (m, 1H), 3.72 (m, 1H), 3.46-3.68 (m, 1H), 2.51-2.71 (m, 3H), 2.31 (m, 1H), 1.94 (m, 1H), 1.58-1.71 (m, 1H), 1.23-1.54 (m, 2H), 0.89-1.02 (m, 9H). LCMS (ESI, m/z): 581 [M+H]⁺.

2-Amino-2-(6-fluoropyrazolo[1,5-a]pyridin-3-yl)acetonitrile: a solution of ethyl 6-fluoropyrazolo[1,5-a]pyridine-3-carboxylate (450 mg, 2.15 mmol) in 40% H₂SO₄ (4.5 mL) was heated at 100° C. for 3 h. After cooling to 0° C., the mixture was basified with 6N NaOH until pH=10 and extracted with Et₂O (2×30 mL). The organic phases were combined, dried over Na₂SO₄, filtered and concentrated under reduced pressure. The residue was purified by flash chromatography on silica gel using a gradient of EA (15 to 30%) in PE to afford 6-fluoropyrazolo[1,5-a]pyridine (170 mg, 58%) as an off-white solid.

To a solution of 6-fluoropyrazolo[1,5-a]pyridine (170 mg, 1.47 mmol) in dry DMF (2 mL) cooled at 0° C. was added POCl₃ (0.350 mL, 3.75 mmol). The mixture was stirred at rt for 3 h. After cooling to 0° C., the mixture was basified with 6N NaOH until pH=10 and extracted with Et₂O (2×10 mL). The organic phases were combined, washed with water (2×10 mL) and concentrated under reduced pressure to afford 6-fluoropyrazolo[1,5-a]pyridine-3-carbaldehyde (170 mg, 83%) as an off-white solid.

A solution of 6-fluoropyrazolo[1,5-a]pyridine-3-carbaldehyde (170 mg, 1.03 mmol) in 7M ammonia in methanol (3 mL) was stirred at rt for 10 min. TMSCN (0.155 mL, 1.24 mmol) was added and the mixture was stirred at rt for 16 h. The mixture was concentrated under reduced pressure to afford quantitatively 2-amino-2-(6-fluoropyrazolo[1,5-a]pyridin-3-yl)acetonitrile as a brown oil.

### Example 21

### Compound 21

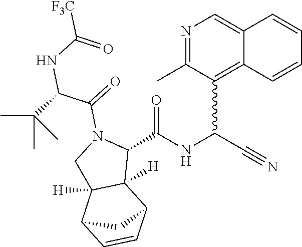 [SMILES: Cc1ncc2ccccc2c1C(C#N)NC(=O)[C@@H]1[C@@H]2[C@H](CN1C(=O)[C@@H](NC(=O)C(F)(F)F)C(C)(C)C)[C@@H]1C=C[C@H]2C1]

Compound 21 was prepared similarly as described for Compound 4 using 3-methylisoquinoline-4-carbaldehyde in place of 1H-1,2,4-triazole-3-carbaldehyde. ¹H NMR (500 MHz, 365K, DMSO-d₆) δ 9.20-9.35 (m, 1H), 8.88-9.08 (m, 1H), 8.43-8.70 (m, 1H), 8.27 (m, 1H), 8.14 (m, 1H), 7.85 (m, 1H), 7.67 (m, 1H), 6.60 (m, 1H), 5.87-6.21 (m, 2H), 4.37-4.56 (m, 1H), 4.08-4.31 (m, 1H), 3.62 (m, 1H), 3.45 (m, 1H), 2.74-2.89 (m, 5H), 1.14-1.59 (m, 4H), 0.69-1.06 (m, 9H). LCMS (ESI, m/z): 568 [M+H]⁺.

3-Methylisoquinoline-4-carbaldehyde: To a solution of 4-bromo-3-methylisoquinoline (1.5 g, 6.76 mmol) in THF (30 mL) cooled to −78° C. was added a 1.6M BuLi solution in hexane (5.1 mL, 8.16 mmol). The mixture was stirred at −78° C. for 1 h. DMF (1 mL, 13.5 mmol) was added and the mixture was allowed to warm to rt over 1 h. The reaction was quenched by the addition of sat. NH₄C₁. The mixture was extracted with EA (2×20 mL). The organic phases were combined, dried over Na₂SO₄, filtered and concentrated under reduced pressure. The residue was purified by flash chromatography on silica gel using a gradient of EA (50 to 70%) in PE to afford 3-methylisoquinoline-4-carbaldehyde (550 mg, 47%) as a brown oil. ¹H NMR (400 MHz, CDCl₃) δ 10.93 (s, 1H), 9.30 (s, 1H), 9.02 (dd, 1H), 7.98 (d, 1H), 7.83 (m, 1H), 7.63 (m, 1H), 3.04 (s, 3H).

### Example 22

### Compounds 22-30

The following compounds were prepared according to analogous methods used to prepare the previously described compounds.

| | | LC-MS | |
|---|---|---|---|
| Entry | Structure | (ESI, m/z) | NMR |
| 22 | 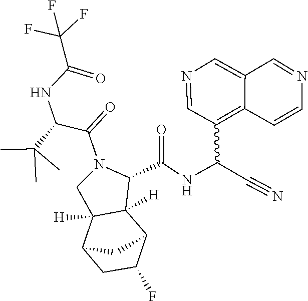 [SMILES: CC(C)(C)[C@H](NC(=O)C(F)(F)F)C(=O)N1C[C@@H]2[C@H]3C[C@@H]([C@@H]2[C@H]1C(=O)NC(C#N)c1cncc2cnccc12)[C@H](F)C3] | 575 [M + H]⁺ | ¹H NMR (400 MHz, 80° C., DMSO-d₆) δ 9.55 − 9.68 (m, 2H), 9.30 − 9.53 (m, 1H), 8.98 − 9.18 (m, 1H), 8.66 − 8.97 (m, 2H), 7.75 − 7.95 (m, 1H), 6.65 − 6.85 (m, 1H), 4.50 − 4.75 (m, 2H), 4.22 − 4.49 (m, 1H), 3.50 − 3.80 (m, 2H), 2.53 − 2.70 (m, 2H), 2.15 − 2.48 (m, 2H), 1.85 − 2.05 (m, 1H), 1.20 − 1.70 (m, 3H), 0.86 − 1.08 (m, 9H). |
| 23 | 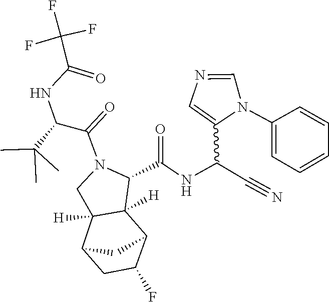 [SMILES: CC(C)(C)[C@H](NC(=O)C(F)(F)F)C(=O)N1C[C@@H]2[C@H]3C[C@@H]([C@@H]2[C@H]1C(=O)NC(C#N)c1cncn1-c1ccccc1)[C@H](F)C3] | 589 [M + H]⁺ | ¹H NMR (400 MHz, 80° C., DMSO-d₆) δ 8.86 − 9.25 (m, 2H), 8.20 − 8.42 (m, 1H), 7.40 − 7.70 (m, 6H), 5.60 − 6.29 (m, 1H), 4.50 − 4.80 (m, 1H), 4.15 − 4.50 (m, 2H), 3.40 − 3.75 (m, 2H), 2.55 − 2.65 (m, 1H), 2.32 − 2.45 (m, 1H), 2.20 − 2.32 (m, 1H), 1.80 − 2.00 (m, 1H), 1.55 − 1.70 (m, 1H), 1.40 − 1.55 (m, 1H), 1.10 − 1.40 (m, 2H), 0.75 − 1.10 (m, 9H). |
| 24 | 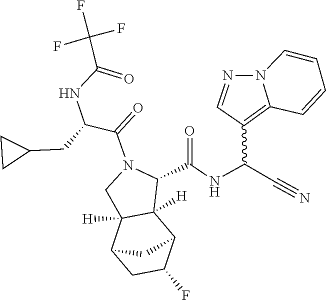 [SMILES: N#CC(NC(=O)[C@@H]1[C@@H]2[C@H](CN1C(=O)[C@H](CC1CC1)NC(=O)C(F)(F)F)[C@H]1C[C@@H]2[C@H](F)C1)c1cnn2ccccc12] | 561 [M + H]⁺ | ¹H NMR (400 MHz, 100° C., DMSO-d₆) δ 9.16 − 9.44 (m, 1H), 8.88 − 9.06 (m, 1H), 8.53 − 8.72 (m, 1H), 7.92 − 8.08 (m, 1H), 7.60 − 7.80 (m, 1H), 7.18 − 7.37 (m, 1H), 6.80 − 7.04 (m, 1H), 6.08 − 6.31 (m, 1H), 4.45 − 4.68 (m, 2H), 4.29 − 4.44 (m, 1H), 3.44 − 3.78 (m, 2H), 2.48 − 2.77 (m, 3H), 2.19 − 2.38 (m, 1H), 1.97 − 2.18 (m, 1H), 1.13 − 1.67 (m, 5H), 0.62 − 0.88 (m, 1H), 0.28 − 0.47 (m, 2H), −0.90 − 0.18 (m, 2H). |
| 25 | 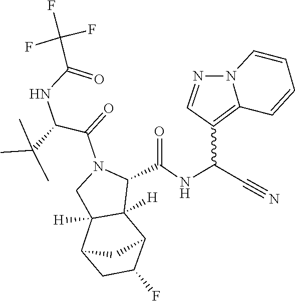 [SMILES: CC(C)(C)[C@H](NC(=O)C(F)(F)F)C(=O)N1C[C@@H]2[C@H]3C[C@@H]([C@@H]2[C@H]1C(=O)NC(C#N)c1cnn2ccccc12)[C@H](F)C3] | 563 [M + H]⁺ | ¹H NMR (400 MHz, 100° C., DMSO-d₆) δ 8.98 − 9.18 (m, 1H), 8.76 − 8.97 (m, 1H), 8.59 − 8.75 (m, 1H), 8.00 − 8.19 (m, 1H), 7.64 − 7.88 (m, 1H), 7.22 − 7.42 (m, 1H), 6.88 − 7.07 (m, 1H), 6.16 − 6.44 (m, 1H), 4.51 − 4.88 (m, 2H), 4.21 − 4.50 (m, 1H), 3.57 − 3.83 (m, 2H), 2.54 − 2.80 (m, 3H), 2.26 − 2.38 (m, 1H), 1.82 − 2.04 (m, 1H), 1.58 − 1.84 (m, 1H), 1.23 − 1.57 (m, 2H), 1.01 − 1.09 (m, 4H), 0.90 − 1.00 (m, 5H). |
| 26 | 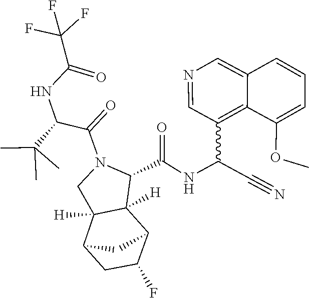 [SMILES: COc1cccc2cncc(C(C#N)NC(=O)[C@@H]3[C@@H]4[C@H](CN3C(=O)[C@@H](NC(=O)C(F)(F)F)C(C)(C)C)[C@H]3C[C@@H]4[C@H](F)C3)c12] | 604 [M + H]⁺ | ¹H NMR (400 MHz, 80° C., DMSO-d₆) δ 9.20 − 9.40 (m, 1H), 8.90 − 9.19 (m, 2H), 8.56 − 8.70 (m, 1H), 7.70 − 7.81 (m, 1H), 7.58 − 7.69 (m, 1H), 7.35 − 7.50 (m, 1H), 6.80 − 7.02 (m, 1H), 4.74 (s, 1H), 4.60 − 4.69 (m, 1H), 4.20 − 4.59 (m, 1H), 3.90 − 4.10 (m, 3H), 3.70 − 3.85 (m, 1H), 3.48 − 3.69 (m, 1H), 2.60 − 2.70 (m, 2H), 2.20 − 2.38 (m, 1H), 1.90 − 2.05 (m, 1H), 1.55 − 1.70 (m, 1H), 1.45 − 1.54 (m, 1H), 1.32 − 1.44 (m, 1H), 1.20 − 1.31 (m, 1H), 0.75 − 1.10 (m, 9H). |
| 27 | 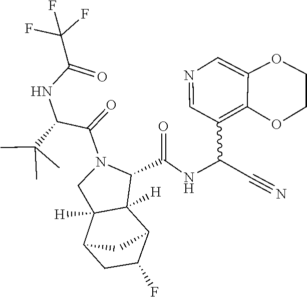 [SMILES: CC(C)(C)[C@H](NC(=O)C(F)(F)F)C(=O)N1C[C@@H]2[C@H]3C[C@@H]([C@@H]2[C@H]1C(=O)NC(C#N)c1cncc2c1OCCO2)[C@H](F)C3] | 604 [M + Na]⁺ | ¹H NMR (400 MHz, 80° C. DMSO-d₆) δ 8.91- 9.21 (m, 2H), 8.05 − 8.41 (m, 2H), 5.91 − 6.15 (m, 1H), 4.61 − 4.85 (m, 3H), 4.50 − 4.61 (m, 2H), 4.31 − 4.45 (m, 2H), 3.71 − 3.28 (m, 1H), 3.51 − 3.68 (m, 1H), 2.53 − 2.71 (m, 3H), 2.21 − 2.41 (m, 1H), 1.85 − 2.05 (m, 1H), 1.61 − 1.72(m, 1H), 1.22 − 1.58 (m, 2H), 0.81 − 1.11 (m, 9H) |
| 28 | 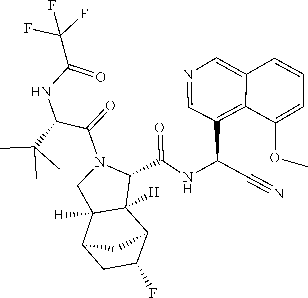 [SMILES: COc1cccc2cncc([C@@H](C#N)NC(=O)[C@@H]3[C@@H]4[C@H](CN3C(=O)[C@@H](NC(=O)C(F)(F)F)C(C)(C)C)[C@H]3C[C@@H]4[C@H](F)C3)c12] | 604 [M + H]⁺ | ¹H NMR (400 MHz, 80° C., DMSO-d₆) δ 9.26 − 9.32 (m, 1H), 8.89 − 9.04 (m, 2H), 8.60 − 8.70 (m, 1H), 7.71 − 7.78 (m, 1H), 7.63 − 7.70 (m, 1H), 7.31 − 7.39 (m, 1H), 6.85 − 6.99 (m, 1H), 4.66 − 4.77 (m, 1H), 4.57 − 4.64 (m, 1H), 4.20 − 4.46 (m, 1H), 3.96 − 4.03 (m, 3H), 3.68 − 3.77 (m, 1H), 3.50 − 3.64 (m, 1H), 2.57 − 2.69 (m, 3H), 2.25 − 2.36 (m, 1H), 1.84 − 2.00 (m, 1H), 1.57 − 1.66 (m, 1H), 1.44 − 1.51 (m, 1H), 1.22 − 1.40 (m, 1H), 0.84 − 0.88 (m, 9H) |
| 29 | 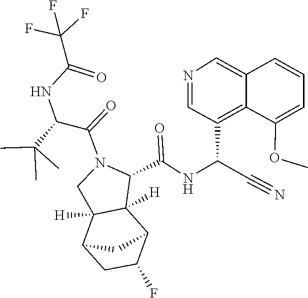 [SMILES: COc1cccc2cncc([C@H](C#N)NC(=O)[C@@H]3[C@@H]4[C@H](CN3C(=O)[C@@H](NC(=O)C(F)(F)F)C(C)(C)C)[C@H]3C[C@@H]4[C@H](F)C3)c12] | 604 [M + H]⁺ | ¹H NMR (400 MHz, 80° C., DMSO-d₆) δ 9.23 − 9.32 (m, 1H), 8.96 − 9.10 (m, 2H), 8.59 − 8.66 (m, 1H), 7.71 − 7.79 (m, 1H), 7.63 − 7.70 (m, 1H), 7.32 − 7.40 (m, 1H), 6.85 − 6.93 (m, 1H), 4.69 − 4.79 (m, 1H), 4.59 − 4.67 (m, 1H), 4.26 − 4.47 (m, 1H), 3.92 − 4.01 (m, 3H), 3.66 − 3.76 (m, 1H), 3.54 − 3.64 (m, 1H), 2.49 − 2.60 (m, 2H), 2.21 − 2.31 (m, 1H), 1.84 − 2.04 (m, 1H), 1.53 − 1.62 (m, 1H), 1.26 − 1.39 (m, 2H), 1.21 − 1.25 (m, 1H), 0.94 − 1.03 (m, 9H). |
| 30 | 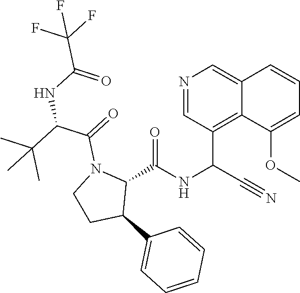 [SMILES: COc1cccc2cncc(C(C#N)NC(=O)[C@@H]3[C@@H](c4ccccc4)CCN3C(=O)[C@@H](NC(=O)C(F)(F)F)C(C)(C)C)c12] | 596 [M + H]⁺ | ¹H NMR (400 MHz, 80° C., DMSO-d₆) δ 9.20 − 9.40 (m, 1H), 8.80 − 9.10 (m, 2H), 8.40 − 8.70 (m, 1H), 7.70 − 7.90 (m, 1H), 7.60 − 7.69 (m, 1H), 7.20 − 7.45 (m, 4H), 7.10 − 7.19 (m, 2H), 6.90 − 7.09 (m, 1H), 4.50 − 4.70 (m, 1H), 4.40 − 4.49 (m, 1H), 3.80 − 4.10 (m, 4H), 3.65 − 3.79 (m, 1H), 3.30 − 3.60 (m, 1H), 2.30 − 2.50 (m, 1H), 1.90 − 2.20 (m, 1H), 0.70 − 1.10 (m, 9H). |

### Example 23

### Compound 31

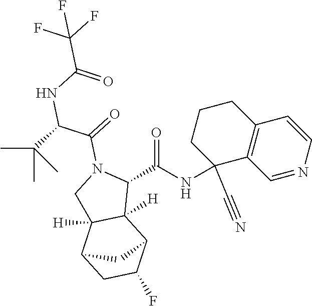 [SMILES: CC(C)(C)[C@H](NC(=O)C(F)(F)F)C(=O)N1C[C@@H]2[C@H]3C[C@@H]([C@@H]2[C@H]1C(=O)NC1(C#N)CCCc2ccncc21)[C@H](F)C3]

Compound 31 was prepared similarly as described for Compound 7 using 8-amino-5,6,7,8-tetrahydroisoquinoline-8-carbonitrile in place of 2-amino-2-(isoquinolin-4-yl)acetonitrile.

A mixture of 6,7-dihydroisoquinolin-8(5H)-one (200 mg, 1.36 mmol) and ammonia (25 mL, 7M in MeOH) was stirred for 2d at rt. Trimethylsilyl cyanide (189 mg, 1.90 mmol) was added to the above solution slowly at 0° C. The mixture was stirred for 2d at rt and then concentrated under reduced pressure to afford 8-amino-5,6,7,8-tetrahydroisoquinoline-8-carbonitrile (200 mg, crude) as a yellow solid. LC-MS (ESI, m/z): 174 [M+H]⁺.

### Example 24

### Compound 32

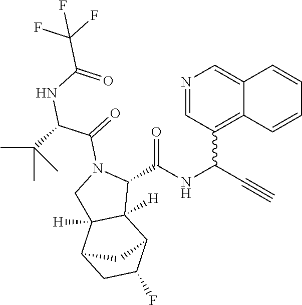 [SMILES: C#CC(NC(=O)[C@@H]1[C@@H]2[C@H](CN1C(=O)[C@@H](NC(=O)C(F)(F)F)C(C)(C)C)[C@H]1C[C@@H]2[C@H](F)C1)c1cncc2ccccc12]

To a mixture of isoquinoline-4-carbaldehyde (500 mg, 3.18 mmol) and tert-butanesulfinamide (771 mg, 6.36 mmol) in THF (7 mL) was added titanium tetraisopropanolate (1.81 g, 6.36 mmol) under nitrogen. The mixture was stirred for 2 h at 65° C. under nitrogen and the reaction was quenched with water (15 mL). The mixture was extracted with EtOAc (3×20 mL). The organic layers were combined, washed with brine (2×20 mL) and dried over anhydrous sodium sulfate. The solids were removed by filtration and the filtrate was concentrated under reduced pressure to afford the crude product. The crude product was purified on a silica gel column with EA:PE (22:88) to provide N-(isoquinolin-4-ylmethylene)-2-methylpropane-2-sulfinamide (700 mg, 84%) as a white solid. ¹H NMR (400 MHz, DMSO-d₆) δ 9.52 (s, 1H), 9.10-9.12 (m, 1H), 9.05 (s, 1H), 8.97 (s, 1H), 8.30-8.32 (m, 1H), 8.00-8.04 (m, 1H), 7.82-7.86 (m, 1H), 1.26 (s, 9H). LC-MS (ESI, m/z): 261 [M+H]⁺.

A mixture of N-(isoquinolin-4-ylmethylene)-2-methylpropane-2-sulfinamide (650 mg, 2.49 mmol) was added bromo(ethynyl)magnesium (25 mL, 12.4 mmol, 0.5M in THF) at 0° C. The mixture was stirred overnight at rt under nitrogen and the reaction was quenched with ammonium chloride solution (35 mL). The mixture was extracted with EA (3×40 mL). The organic layers were combined, washed with brine (2×40 mL) and dried over anhydrous sodium sulfate. The solids were removed by filtration. The filtrate was concentrated under reduced pressure to afford crude product, that was chromatographed on a silica gel column with MeOH:DCM (3:97) to provide N-(1-(isoquinolin-4-yl)prop-2-yn-1-yl)-2-methylpropane-2-sulfinamide (460 mg, 64%) as a yellow solid. ¹H NMR (400 MHz, DMSO-d₆) δ 9.31 (s, 1H), 8.76 (s, 1H), 8.14-8.34 (m, 2H), 7.81-7.90 (m, 1H), 7.67-7.79 (m, 1H), 6.23-6.38 (m, 1H), 5.78-5.95 (m, 1H), 3.58-3.76 (m, 1H), 0.83-1.27 (m, 9H). LC-MS (ESI, m/z): 287 [M+H]⁺.

A mixture of N-(1-(isoquinolin-4-yl)prop-2-yn-1-yl)-2-methylpropane-2-sulfinamide (100 mg, 0.349 mmol) in HCl (2 mL, 4M in MeOH) was stirred for 1 h at rt. The mixture was concentrated under reduced pressure to afford 1-(isoquinolin-4-yl)prop-2-yn-1-amine (64.0 mg, crude) as a yellow solid. LC-MS (ESI, m/z): 183 [M+H]⁺.

To a mixture of 1-(isoquinolin-4-yl)prop-2-yn-1-amine (64.0 mg, 0.351 mmol), (1S,2R,3S,6R,7S,9R)-4-[(2S)-3,3-dimethyl-2-(2,2,2-trifluoroacetamido)butanoyl]-9-fluoro-4-azatricyclo[5.2.1.0\hat{ }{2,6}]decane-3-carboxylic acid (143 mg, 0.351 mmol) and o-(7-azabenzotriazol-1-yl)-N,N,N′,N′-tetramethyluronium hexafluorophosphate (160 mg, 0.421 mmol) in DMF (2.5 mL) was added N-ethyl-N-isopropylpropan-2-amine (272 mg, 2.10 mmol) at 0° C. The mixture was stirred for 1 h at rt and the reaction was quenched with water (6 mL). The mixture was extracted with EA (3×10 mL). The organic layers were combined, washed with brine (2×10 mL) and dried over anhydrous sodium sulfate. The solids were removed by filtration and the filtrate was concentrated under reduced pressure to afford the crude product. The crude product was purified by prep-HPLC (Column: Xselect CSH Prep C18, 30×150 mm, 5 μm; Mobile Phase A: Water (0.1% FA), Mobile Phase B: ACN; Flow rate: 60 mL/min; Gradient: 35% B to 65% B in 10 min; Wave Length: 220 nm; RT1(min): 8.68) to provide (1S,3aR,4S,6R,7S,7aR)-2-((S)-3,3-dimethyl-2-(2,2,2-trifluoroacetamido)butanoyl)-6-fluoro-N-(1-(isoquinolin-4-yl)prop-2-yn-1-yl)octahydro-1H-4,7-methanoisoindole-1-carboxamide (110 mg, 54%) as a white solid. ¹H NMR (400 MHz, 80° C., DMSO-d₆) δ 8.33-8.51 (m, 1H), 7.93-8.10 (m, 1H), 7.28-7.46 (m, 2H), 6.99-7.17 (m, 1H), 6.84-6.98 (m, 1H), 5.62-5.83 (m, 1H), 3.93-4.06 (m, 1H), 3.84-3.91 (m, 1H), 3.53-3.72 (m, 1H), 3.00-3.11 (m, 1H), 2.88-2.99 (m, 1H), 2.14-2.26 (m, 1H), 1.89-2.06 (m, 1H), 1.80-1.87 (m, 1H), 1.64-1.78 (m, 1H), 1.51-1.63 (m, 1H), 1.06-1.21 (m, 1H), 0.88-1.05 (m, 1H), 0.69-0.79 (m, 1H), 0.50-0.63 (m, 1H), −0.01-0.33 (m, 9H). LC-MS (ESI, m/z): 573 [M+H]⁺.

### LC-MS Methods

| Compound No. | Rt (min) | [M + H]⁺ or [M − H]⁻ | LCMS Method |
|---|---|---|---|
| 1 | 0.982 | [M + H]⁺ = 554 | B |
| 2 | 1.120 | [M + H]⁺ = 543 | D |
| 3 | 0.853 | [M + H]⁺ = 504 | B |
| 4 | 0.846 | [M + H]⁺ = 494 | B |
| 5 | 0.768 | [M + H]⁺ = 530 | B |
| 6 | 1.457 | [M + Na]⁺ = 529 | C |
| 7 | 1.452 | [M + H]⁺ = 574 | D |
| 8 | 1.525 | [M + H]⁺ = 524 | C |
| 9 | 0.897 | [M + H]⁺ = 575 | B |
| 10 | 1.596 | [M + H]⁺ = 592 | D |
| 11 | 1.481 | [M + H]⁺ = 530 | C |
| 12 | 1.032 | [M + H]⁺ = 603 | F |
| 13 | 1.467 | [M + H]⁺ = 589 | E |
| 14 | 5.087 | [M + H]⁺ = 600 | G |
| 15 | 2.290 | [M + H]⁺ = 554 | A |
| 16 | 2.417 | [M + H]⁺ = 553 | A |
| 17 | 2.181 | [M + H]⁺ = 505 | A |
| 18 | 2.236 | [M + H]⁺ = 519 | A |
| 19 | 2.303 | [M + H]⁺ = 593 | A |
| 20 | 2.302 | [M + H]⁺ = 581 | A |
| 21 | 2.304 | [M + H]⁺ = 568 | A |
| 22 | 1.490, 1.523, | [M + H]⁺ = 575 | L |
| | 1.562, 1.589 | | |
| 23 | 0.752, 0.780 | [M + H]⁺ = 589 | M |
| 24 | 0.899 | [M + Na]⁺ = 583 | M |
| 25 | 1.565, 1.603, | [M + Na]⁺ = 585 | N |
| | 1.637 | | |
| 26 | 1.538, 1.572, | [M + H]⁺ = 604 | K |
| | 1.602 | | |
| 27 | 1.265, 1.296, | [M + Na]⁺ = 604 | H |
| | 1.319, 1.335 | | |
| 28 | 1.361, 1.397, | [M + H]⁺ = 604 | N |
| | 1.425, 1.437, | | |
| | 1.456 | | |
| 29 | 1.329, 1.370, | [M+Na]⁺ = 626 | N |
| | 1.409, 1.435, | | |
| | 1.457 | | |
| 30 | 0.873 | [M + Na]⁺ = 596 | M |
| 31 | 1.312, 1.325 | [M + Na]⁺ = 564 | J |
| 32 | 0.739, 0.767, | [M + H]⁺ = 573 | M |
| | 0.772 | | |

Final compounds can be obtained in some cases as a mixture with a corresponding stereoisomer. Retention times of the main isomers are depicted in the table above.

### Description of LC-MS Methods

| | | | | | Flow | Col | Run |
|---|---|---|---|---|---|---|---|
| LC | | | | | mL/ | T | Time |
| Method | Instrument | Column | Mobile Phase | Gradient | min | (° C.) | (min) |
| A | Agilent | Acquity | A: 0.1% FA | 98% A held | 0.6 | 70 | 3.8 |
| | 6150 SQ- | UPLC BEH | in Water, | for 0.2 min, | | | |
| | Agilent | C18 (1.7 μm, | B: 0.1% FA in | to 2% A in | | | |
| | 1290 | 2.1*50 mm) | Acetonitrile | 1.3 min, held | | | |
| | Infinity | | | for 1.8 min, | | | |
| | | | | to 98% A in | | | |
| | | | | 0.1 min, held | | | |
| | | | | for 0.4 min | | | |
| B | Shimadzu | HALO | A: Water/ | From 95% A | 1.5 | 40 | 1.85 |
| | LCMS- | 2.0 μm C18 | 0.1% FA | to 0% A in | | | |
| | 2020 | 90A (2.0 μm, | B: Acetonitrile/ | 1.09 min, | | | |
| | | 3.0*30 mm) | 0.1% FA | held for 0.6 | | | |
| | | | | min, to 95% | | | |
| | | | | A in 0.05 | | | |
| | | | | min, held for | | | |
| | | | | 0.10 min | | | |
| C | Shimadzu | HALO | A: Water/ | From 95% A | 1.5 | 40 | 3 |
| | LCMS- | 2.0 μm C18 | 0.1% FA | to 30% A in | | | |
| | 2020 | 90A | B: ACN/ | 1.69 min, to | | | |
| | | (2.0 μm, | 0.1% FA | 0% A in 0.6 | | | |
| | | 3.0*30 mm) | | min, held for | | | |
| | | | | 0.5 min, to | | | |
| | | | | 95% A in | | | |
| | | | | 0.03 min, | | | |
| | | | | held for 0.17 | | | |
| | | | | min | | | |
| D | Shimadzu | HALO | A: Water/ | From 95% A | 1.5 | 40 | 3 |
| | LCMS- | 2.0 μm C18 | 0.1% FA | to 5% A in | | | |
| | 2020 | 90A | B: ACN/ | 2.09min, held | | | |
| | | (2.0 μm, | 0.1% FA | for 0.6 min, | | | |
| | | 3.0*30 mm) | | to 95% A in | | | |
| | | | | 0.05 min, | | | |
| | | | | held for 0.25 | | | |
| | | | | min | | | |
| E | Shimadzu | HALO | A: Water/ | From 70% A | 1.2 | 40 | 3 |
| | LCMS- | (2.0 μm, | 0.1% FA | to 30% A in | | | |
| | 2020 | 3.0*30 mm) | B: Acetonitrile/ | 1.69 min, | | | |
| | | | 0.1% FA | held for | | | |
| | | | | 0.6min, to | | | |
| | | | | 100% A in | | | |
| | | | | 0.5 min, to | | | |
| | | | | 95% A in | | | |
| | | | | 0.03 min, | | | |
| | | | | held for 0.17 | | | |
| | | | | min | | | |
| F | Shimadzu | Shim-pack | A: Water − | From 90% A | 1.5 | 40 | 2 |
| | LCMS- | Scepter | 5 mM | to 5% A in | | | |
| | 2020 | C18-120 | NH₄HCO₃ | 1.19 min, | | | |
| | | (3.0 μm, | B: Acetonitrile | held for 0.6 | | | |
| | | 3.0*33 mm) | | min, to 90% | | | |
| | | | | A in 0.02 | | | |
| | | | | min, held for | | | |
| | | | | 0.18 min | | | |
| G | Shimadzu | HALO | A: Water/ | From 50% A | 1.2 | 40 | 8 |
| | LCMS- | 2.0 μm C18 | 0.05% TFA | to 50% A in | | | |
| | 2020 | 90A (2.7 μm, | B: MeOH | 0 min, held | | | |
| | | 100*4.6 | | for 6 min, | | | |
| | | mm) | | From 95% A | | | |
| | | | | to 5% A in 0 | | | |
| | | | | min, held for | | | |
| | | | | 2 min | | | |
| H | Shimadzu | HALO 90 A | A: Water/ | From 95% A | 1.5 | 40 | 3 |
| | LCMS- | C18 | 0.05% TFA | to 40% A in | | | |
| | 2020 | (2.0 μm, | B: CH₃CN/ | 1.69 min, to | | | |
| | | 3.0*30 mm) | 0.05% TFA | 5% A in 0.6 | | | |
| | | | | min, held for | | | |
| | | | | 0.5 min, to | | | |
| | | | | 95% A in | | | |
| | | | | 0.03 min, | | | |
| | | | | held for 0.17 | | | |
| | | | | min | | | |
| J | Shimadzu | HALO C18 | A: Water + | From 95% A | 1.5 | 40 | 3 |
| | LCMS- | (2.0 μm, | 0.05% TFA | to 40% A in | | | |
| | 2020 | 3.0*30 mm) | B: CH₃CN + | 1.69 min, to | | | |
| | | | 0.05% TFA | 5% A in 0.6 | | | |
| | | | | min, held for | | | |
| | | | | 0.5 min, to | | | |
| | | | | 95% A in | | | |
| | | | | 0.03 min, | | | |
| | | | | held for 0.17 | | | |
| | | | | min | | | |
| K | Shimadzu | HALO | A: Water/ | From 80% A | 1.2 | 40 | 3 |
| | LCMS- | (2.0 μm, | 0.1% FA | to 40% A in | | | |
| | 2020 | 3.0*30 mm) | B: CH₃CN/ | 1.69 min, to | | | |
| | | | 0.1% FA | 0% A in 0.6 | | | |
| | | | | min, held for | | | |
| | | | | 0.5 min, to | | | |
| | | | | 95% A in | | | |
| | | | | 0.03 min, | | | |
| | | | | held for 0.17 | | | |
| | | | | min | | | |
| L | Shimadzu | HALO | A: Water/ | From 95% A | 1.2 | 40 | 3 |
| | LCMS- | 2.0 μm C18 | 0.1% FA | to 40% A in | | | |
| | 2020 | 90A (2.0 μm, | B: CH₃CN/ | 1.69 min, to | | | |
| | | 3.0*30 mm) | 0.1%FA | 0% A in 0.6 | | | |
| | | | | min, held for | | | |
| | | | | 0.5 min, to | | | |
| | | | | 95% A in | | | |
| | | | | 0.03 min, | | | |
| | | | | held for 0.17 | | | |
| | | | | min | | | |
| M | Shimadzu | HALO C18 | A: Water + | From 95% A | 1.5 | 40 | 2 |
| | LCMS- | (2.0 μm, | 0.05% TFA | to 0% A in | | | |
| | 2020 | 3.0*30 mm) | B: CH₃CN + | 1.19 min, | | | |
| | | | 0.05% TFA | held for 0.6 | | | |
| | | | | min, to 95% | | | |
| | | | | A in 0.02 | | | |
| | | | | min, held for | | | |
| | | | | 0.18 min | | | |
| N | Shimadzu | HALO | A: Water/ | From 80% A | 1.5 | 40 | 3 |
| | LCMS- | (2.0 μm, | 0.1% TFA | to 40% A in | | | |
| | 2020 | 3.0*30 mm) | B: CH₃CN/ | 1.69 min, to | | | |
| | | | 0.1% TFA | 5% A in 0.6 | | | |
| | | | | min, held for | | | |
| | | | | 0.5 min, to | | | |
| | | | | 95% A in | | | |
| | | | | 0.03 min, | | | |
| | | | | held for 0.17 | | | |
| | | | | min | | | |

### Example A

### SARS-Cov-2 3CLpro

Protease assays were performed in 384-well low volume polypropylene microtiter plates at ambient temperature. For the duplex assay, 3CLpro was added using a Multidrop Combi (Thermo Scientific; Waltham, MA) and preincubated for 30 mins with small molecules. The reactions were initiated by the addition of the two peptide substrates. The reactions were incubated for 30 mins and quenched by the addition of 0.5% formic acid (final) with subsequent neutralization using 1% sodium bicarbonate (final). Internal standard peptides were added in 20 mM Hepes pH 8.0 for quantitation of the protease products. For SAMDI-MS analysis, 2 μL of each reaction mixture was transferred using a 384-channel automated liquid handler to SAMDI biochip arrays functionalized with a neutravidin-presenting self-assembled monolayer. The SAMDI arrays were incubated for 1 h in a humidified chamber to allow the specific immobilization of the biotinylated peptide substrates, cleaved products and internal standards. The samples were purified by washing the SAMDI arrays with deionized ultrafiltered water and dried with compressed air. A matrix comprising alpha-cyano cinnamic acid in 80% acetonitrile:20% aqueous ammonium citrate was applied in an automated format by dispensing 50 nL to each spot in the array. SAMDI-MS was performed using reflector-positive mode on an AB Sciex TOF-TOF 5800 System (AB Sciex, Framingham, MA) with 400 shots/spot analyzed in a random raster sampling. For data analysis, area under the curves (peaks) (AUCs) for the product and internal standard were calculated using the TOF/TOF Series Explorer (AB Sciex), and the amount of product formed was calculated using the equation (AUC product/AUC internal standard). The amount of product generated was calculated using the ratio of product area under the curve (AUC) divided by the AUC of the internal standard. Negative controls were pre-quenched with 0.5% formic acid final. Assay robustness was determined by Z-Factor. The IC₅₀s were determined by fitting the curves using a four-parameter equation in Graphpad Prism 8.

Table 1 indicates related IC₅₀ values for the tested compounds where ‘A’ indicates an EC₅₀<20 nM, ‘B’ indicates an IC₅₀ of ≥20 nM and <200 nM, ‘C’ indicates an IC₅₀≥200 nM and <2000 nM, ‘D’ indicates an IC₅₀≥2000 nM and <20000 nM and ‘E’ indicates an IC₅₀≥20000 nM and <100000 nM. As shown by the data in Table 1, compounds described herein (including pharmaceutically acceptable salts thereof) can effectively inhibit and be used to treat a coronavirus and rhinovirus.

TABLE 1

| Compound | SARS-Cov-2 |
|---|---|
| 1 | A |
| 2 | A |
| 3 | A |
| 4 | C |
| 5 | C |
| 6 | B |
| 7 | A |
| 8 | B |
| 9 | A |
| 10 | A |
| 11 | A |
| 12 | B |
| 13 | C |
| 14 | A |
| 15 | A |
| 16 | C |
| 17 | B |
| 18 | A |
| 19 | A |
| 20 | A |
| 21 | D |
| 22 | A |
| 23 | B* |
| 24 | A |
| 25 | A |
| 26 | A |
| 27 | B |
| 28 | A |
| 29 | B |
| 30 | B |
| 31 | D⁺ |
| 32 | B |

*activity of >500 nM
⁺activity of >2000 nM

### Example B

### Coronavirus Assay

### OC43 Coronavirus Assay in HeLa Cells

The human beta-coronavirus OC43 was purchased from ATCC (Manassas, VA) and propagated using HCT-8 human colorectal epithelial cells (ATCC). HeLa human cervical epithelial cells (ATCC) were used as susceptible host cell lines and were cultured using EMEM media, supplemented with 10% fetal bovine serum (FBS), 1% (v/v) penicillin/streptomycin (P/S), 1% (v/v) HEPES and 1% (v/v) cellgro Glutagro™ supplement (all Corning, Manassas, VA) at 37° C. For the OC43 antiviral assay, 1.5×10⁴ HeLa cells per well were plated in 100 μL complete media in white 96-well plates with clear bottoms at 37° C. for up to 24 h to facilitate attachment and allow cells to recover from seeding stresses. Next day, the cell culture medium was removed. Serially diluted compounds in 100 μL assay media (EMEM, 2% FBS, 1% P/S, 1% cellgro Glutagro™ supplement, 1% HEPES) were added to the cells and incubated for 4 h at 37° C. in a humidified 5% CO₂ incubator. 100 μL of OC43 virus stock was diluted to a concentration known to produce optimal cytopathic effect, inducing 80-90% reduction in cell viability. 96-well plates were incubated for 6 days at 33° C.; each plate contains uninfected control wells as well as virus-infected wells that were not treated with compound. Cytotoxicity plates without the addition of OC43 virus were carried out in parallel. At the end of the incubation period, 100 μL cell culture supernatant was replaced with 100 μL cell-titer-glo reagent (Promega, Madison, WI) and incubated for at least 10 min at rt prior to measuring luminescence. Luminescence was measured on a Perkin Elmer (Waltham, MA) Envision plate reader. Antiviral % inhibition was calculated as follows: [(Compound treated cells infected sample)−(no compound infected control)]/[(Uninfected control)−(no compound infected control)]*100; Using GraphPad (San Diego, CA) prism software version 8.3.1, the antiviral dose-response plot was generated as a sigmoidal fit, log(inhibitor) vs response-variable slope (four parameters) model and the EC₅₀ was calculated which is the predicted compound concentration corresponding to a 50% inhibition of the viral cytopathic effect.

Table 2 indicates related EC₅₀ and CC₅₀ values for the tested compounds ‘A’ indicates an EC₅₀<100 nM, ‘B’ indicates an EC₅₀ of ≥100 nM and <1000 nM, ‘C’ indicates an EC₅₀≥1000 nM and <10000 nM and ‘D’ indicates an EC₅₀≥10000 nM and <100000 nM. For CC₅₀, the values are reported in micromolar (M), ‘A’ indicates a CC₅₀≥10000 nM and ‘B’ indicates a CC₅₀≥1 μM and <10 μM.

TABLE 2

| Compound | EC₅₀ | CC₅₀ |
|---|---|---|
| 1 | A | A |
| 2 | B | A |
| 3 | B | A |
| 4 | C | A |
| 5 | C | A |
| 6 | C | A |
| 7 | A | A |
| 8 | B | B |
| 9 | A | A |
| 10 | B | A |
| 11 | A | A |
| 14 | D | A |
| 15 | B | A |
| 17 | B | A |
| 18 | B | A |
| 19 | B | A |
| 20 | B | A |
| 22 | C | A |
| 24 | B | A |
| 25 | B | A |
| 26 | A | A |
| 27 | C | A |
| 28 | A | A |
| 29 | B | A |
| 30 | C | A |
| 31 | C | A |

### B.1.1.7 Infection Model in A549-Dual_ACE2_TMPRSS2 Cells

The A549-dual_ACE2_TMPRSS2 cells (InvivoGen Cat #a549-cov2r) were propagated in the growth medium which was prepared by supplementing DMEM (gibco cat no 41965-039) with 10% v/v heat-inactivated FCS and 10 μg/mL blasticidin (InvivoGen ant-bl-05), 100 μg/mL hygromycin (InvivoGen ant-hg-1), 0.5 μg/mL puromycin (InvivoGen ant-pr-1) and 100 μg/mL zeocin (InvivoGen ant-zn-05) in a humidified 5% CO₂ incubator at 37° C. The assay medium was prepared by supplementing DMEM (gibco cat no 41965-039) with 2% v/v heat-inactivated FCS.

The virus isolate used is from the B.1.1.7 lineage (derived from hCoV-19/Belgium/rega-12211513/2020; EPI_ISL_791333,2020-12-21; see Abdelnabi et al., “Comparing infectivity and virulence of emerging SARS-CoV-2 variants in Syrian hamsters” EBioMedicine (2021) June; 68:103403. doi: 10.1016/j.ebiom.2021.103403).

For antiviral testing, cells were seeded in 96-well plates (Falcon) at a density of 15,000 cells per well in assay medium. After overnight growth, cells were treated with the indicated compound concentrations and infected with a MOI of 0.001 TCID50/cell (final volume 200 μL/well in assay medium). On day 4 p.i. differences in cell viability caused by virus-induced CPE or by compound-specific side effects are analyzed using MTS as described previously (PMID: 22575574).

For toxicity testing, the same experimental setup was used except that assay medium without virus was added to the cells and that an additional control of well without cells was added to the plate.

Table 3 indicates related EC₅₀ and CC₅₀ values for the tested compounds ‘A’ indicates an EC₅₀<100 nM, ‘B’ indicates an EC₅₀ of ≥100 nM and <1000 nM, ‘C’ indicates an EC₅₀≥1000 nM. A “C” indicates that the compound has activity. For CC₅₀, the values are reported in micromolar (M). ‘A’ indicates a CC₅₀≥1000 nM. ‘B’ indicates a CC₅₀≥100 nM and <1000 nM.

TABLE 3

| | A549- | A549- |
|---|---|---|
| | dual_ACE2_TMPRSS2 | dual_ACE2_TMPRSS2 |
| Compound | (EC₅₀) | (CC₅₀) |
| 1 | A | A |
| 2 | B | A |
| 3 | B | A |
| 5 | C | A |
| 6 | B | A |
| 7 | A | A |
| 8 | B | A |
| 9 | A | A |
| 10 | C | A |
| 11 | A | A |
| 12 | C | A |
| 13 | C | A |
| 14 | B | A |
| 15 | B | A |
| 16 | C | A |
| 17 | B | A |
| 18 | B | A |
| 19 | A | A |
| 20 | A | A |
| 21 | C | A |
| 22 | B | A |
| 23 | C | A |
| 24 | B | A |
| 25 | B | A |
| 26 | A | A |
| 27 | C | A |
| 28 | A | A |
| 29 | A | A |
| 30 | C | A |
| 31 | C | A |

Tables 1, 2 and 3 demonstrate that compounds described herein (including pharmaceutically acceptable salts thereof) can effectively inhibit and treat a coronavirus.

### Example C

### Picornavirus & Norovirus Assays

Compounds of Formula (I), including pharmaceutically acceptable salts thereof, are tested following a protocol similar to the protocol described in one of the following articles: Kim et al., Journal of Virology (2012) 86(21):11754-11762, Zhang et al, JACS (2020) (https://dx.doi.org/10.1021/acs.jmedchem.9b01828), and U.S. Pat. No. 9,603,864.

The protocols of Kim et al., and Zhang et al., can be used to test for activity against a picornavirus and norovirus.

### Example D

For the cathepsin L assay, 10 pM of human cathepsin L (R&D Systems; Minneapolis, MN) was preincubated for 30 mins with test compounds. Reactions were initiated by the addition of a peptide substrate Z-FR-AMC (final concentration 2 μM, Anaspec; Fremont, CA). Fluorescence was measured at 2-minute intervals for 30 mins using a 355/460 excitation/emission filter module on an Envision plate reader (Perkin Elmer; Waltham, MA). The IC₅₀ values were calculated for each assay by fitting the curves using a four-parameter equation in GraphPad Prism.

Table 4 indicates related IC₅₀ values for the tested compounds where ‘A’ indicates an IC₅₀≥10000 nM, ‘B’ indicates an IC₅₀ of ≥1000 nM and <10000 nM, ‘C’ indicates an IC₅₀; ≥100 nM and <1000 nM, ‘D’ indicates an IC₅₀<100 nM.

TABLE 4

| Compound | Cathepsin L IC₅₀ |
|---|---|
| 1 | A |
| 2 | A |
| 3 | A |

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the present disclosure.

1. A compound of Formula (I), or a pharmaceutically acceptable salt thereof, having the structure: 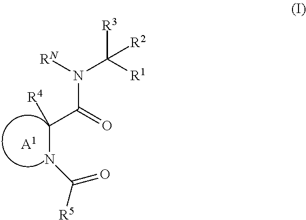
wherein:
R^{N} is hydrogen, deuterium or an unsubstituted or a substituted C₁₋₆ alkyl;
Ring A¹ is an unsubstituted or a substituted 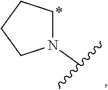 [SMILES: *N1CCCC1]
an unsubstituted or a substituted 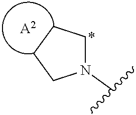
an unsubstituted or a substituted 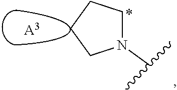
an unsubstituted or a substituted 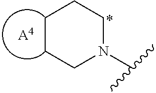
or an unsubstituted or a substituted 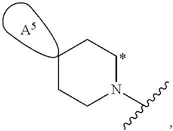
wherein the N is the nitrogen of Ring A¹ shown in Formula (I) and the carbon indicated with an asterisk is the carbon to which R⁴ is connected;
Ring A² and Ring A⁴ are an unsubstituted or a substituted 3- to 10-membered ring system that optionally includes 1 to 3 heteroatoms selected from the group consisting of O, S and N, and wherein Ring A² and Ring A⁴ are optionally substituted with one or more moieties independently selected from the group consisting of ═O, ═CH₂, deuterium, halogen, hydroxy, an unsubstituted C₁₋₄ alkyl, an unsubstituted C₁₋₄ haloalkyl, an unsubstituted C₂₋₄ alkenyl and an unsubstituted or a substituted C₃₋₆ monocyclic cycloalkyl;
Ring A³ and Ring A⁵ are an unsubstituted or a substituted monocyclic C₃₋₆ cycloalkyl;
R¹ is selected from the group consisting of cyano, an unsubstituted or a substituted C₂₋₅ alkynyl, an unsubstituted or a substituted acyl, an unsubstituted or a substituted ketoamide, —CH(OH)—(S(═O)₂—OH), —CH(OH)—(S(═O)₂—O—), —CH(OH)((P═O)(OR⁶)₂) and —C(═O)CH₂—O—((P═O)(OR⁷)₂);
each R⁶ and each R⁷ are independently hydrogen, an unsubstituted C₁₋₆ alkyl, an unsubstituted C₂₋₆ alkenyl, an unsubstituted C₁₋₆ haloalkyl, an unsubstituted or a substituted aryl or an unsubstituted or a substituted aryl(C₁₋₄ alkyl);
R² is hydrogen, deuterium, halogen or an unsubstituted C₁₋₄ alkyl; and
R³ is an unsubstituted or a substituted monocyclic nitrogen-containing heteroaryl, an unsubstituted or a substituted bicyclic nitrogen-containing heteroaryl, an unsubstituted or a substituted bicyclic nitrogen-containing heterocyclyl or an unsubstituted or a substituted naphthyl; or
R² and R³ are taken together along with the carbon to which they are attached to form an unsubstituted or a substituted bicyclic, nitrogen-containing heterocyclyl;
R⁴ is hydrogen, deuterium or halogen;
R⁵ is 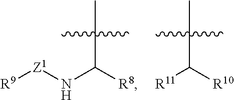
or R¹²;
Z¹ is —C(═O)— or —S(═O)₂—;
R⁸ and R¹⁰ are independently selected from the group consisting of an unsubstituted or a substituted C₁₋₆ alkyl, an unsubstituted or a substituted C₂₋₆ alkenyl, an unsubstituted or a substituted C₂₋₆ alkynyl, an unsubstituted or a substituted monocyclic C₃₋₆ cycloalkyl, an unsubstituted or a substituted bicyclic C₅₋₈ cycloalkyl, an unsubstituted or a substituted monocyclic 4- to 6-membered heterocyclyl and an unsubstituted monocyclic C₃₋₆ cycloalkyl(CH₂)—,
wherein when the C₁₋₆ alkyl is substituted, the C₁₋₆ alkyl is substituted 1, 2, 3 or 4 times with a substituent independently selected from the group consisting of halogen, cyano, an unsubstituted or a substituted monocyclic C₃₋₆ cycloalkyl, an unsubstituted C₁₋₄ alkoxy and an unsubstituted C₁₋₄ haloalkoxy, or the C₁₋₆ alkyl is substituted 1 to 13 times with deuterium;
wherein when the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl are substituted, the C₂₋₆ alkenyl, the C₂₋₆ alkynyl, the monocyclic C₃₋₆ cycloalkyl, the bicyclic C₅₋₈ cycloalkyl and the monocyclic 4- to 6-membered heterocyclyl are substituted 1, 2, 3 or 4 times with a substituent independently selected from the group consisting of halogen, an unsubstituted C₁₋₄ alkyl, an unsubstituted C₂₋₄ alkenyl, an unsubstituted C₂₋₄ alkynyl, an unsubstituted C₁₋₄ haloalkyl, an unsubstituted or a substituted monocyclic C₃₋₆ cycloalkyl and an unsubstituted C₁₋₄ alkoxy; and
R⁹ is selected from the group consisting of an unsubstituted or a substituted C₁₋₆ alkyl, an unsubstituted or a substituted C₁₋₆ haloalkyl, an unsubstituted or a substituted monocyclic C₃₋₆ cycloalkyl, an unsubstituted or a substituted bicyclic C₅₋₆ cycloalkyl, an unsubstituted or a substituted monocyclic heteroaryl, an unsubstituted or a substituted monocyclic heterocyclyl, an unsubstituted or a substituted alkoxy and —NR¹⁷R¹⁸, wherein the substituted C₁₋₆ alkyl is substituted 1 or 2 times with an unsubstituted C₁₋₄ alkoxy, wherein the substituted monocyclic C₃₋₆ cycloalkyl is substituted 1, 2, 3 or 4 times with a substituent independently selected from the group consisting of halogen, an unsubstituted C₁₋₄ alkyl, an unsubstituted C₂₋₄ alkenyl, an unsubstituted C₁₋₄ alkoxy, an unsubstituted C₁₋₄ haloalkyl and an unsubstituted monocyclic C₃₋₆ cycloalkyl, and wherein the substituted C₁₋₆ haloalkyl is substituted 1 or 2 times with an unsubstituted C₁₋₄ alkoxy;
R¹¹ is an unsubstituted or a substituted monocyclic 4- to 6-membered heterocyclyl, —(NH)ₘ-(an unsubstituted or a substituted 5- to 6-membered monocyclic heteroaryl), —O-(an unsubstituted or a substituted C₁₋₆ alkyl), —O-(an unsubstituted or a substituted C₃₋₈ cycloalkyl) or —O—(C₁₋₄ alkyl)-(an unsubstituted or a substituted C₃₋₈ cycloalkyl), wherein m is 0 or 1;
R¹² is an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl), an unsubstituted or a substituted heteroaryl(alkyl), an unsubstituted or a substituted heterocyclyl(alkyl), an unsubstituted or a substituted C-carboxy, —OR¹³, —NR¹⁴R¹⁵ or —C(═O)—NR^{16A}R^{16B};
R¹³ is an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl);
R¹⁴ and R¹⁵ are independently selected from the group consisting of hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted aryl, an unsubstituted or a substituted heteroaryl, an unsubstituted or a substituted 3- to 8-membered monocyclic heterocyclyl, an unsubstituted or a substituted aryl(alkyl) or an unsubstituted or a substituted heteroaryl(alkyl); or
R¹⁴ and R¹⁵ are taken together with the nitrogen to which R¹⁴ and R¹⁵ are attached to form an optionally substituted 3- to 8-membered heterocyclyl;
R^{16A} is hydrogen or an unsubstituted C₁₋₃ alkyl;
R^{16B} is an unsubstituted or a substituted aryl, an unsubstituted or a substituted heteroaryl or an unsubstituted or a substituted 3- to 8-membered monocyclic heterocyclyl; and
R¹⁷ and R¹⁸ are independently selected from the group consisting of hydrogen, an unsubstituted or a substituted C₁₋₈ alkyl, an unsubstituted or a substituted C₂₋₈ alkenyl, an unsubstituted or a substituted C₂₋₈ alkynyl, an unsubstituted or a substituted C₃₋₈ cycloalkyl, an unsubstituted or a substituted 3-8 membered heterocyclyl, an unsubstituted or a substituted aryl, an unsubstituted or a substituted heteroaryl, an unsubstituted or a substituted aryl(alkyl) and an unsubstituted or a substituted heteroaryl(alkyl); or
R¹⁷ and R¹⁸ are taken together along with the nitrogen to which they are connected to form an unsubstituted or a substituted 3-8 membered heterocyclyl; and
provided that when R¹² is 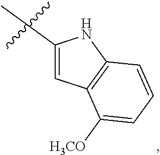 [SMILES: *c1cc2c(OC)cccc2[nH]1]
then R² and R³ cannot be taken together along with the carbon to which they are attached to form an unsubstituted 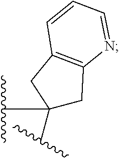 [SMILES: *C1(*)Cc2cccnc2C1]
and
provided that Ring A¹ cannot be an unsubstituted or a substituted

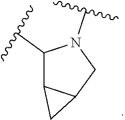 [SMILES: *C1C2CC2CN1*]

2. The compound of claim 1, wherein R³ is an unsubstituted or a substituted monocyclic nitrogen-containing heteroaryl, an unsubstituted or a substituted bicyclic nitrogen-containing heteroaryl or is an unsubstituted or a substituted bicyclic nitrogen-containing heterocyclyl.

3.-5. (canceled)

6. The compound of claim 1, wherein R³ is selected from the group consisting of: 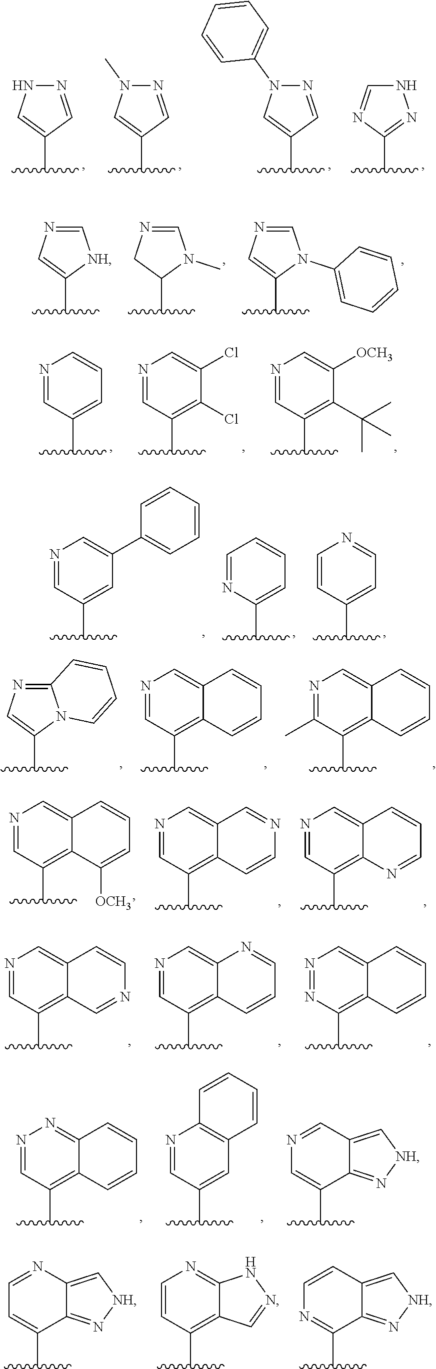 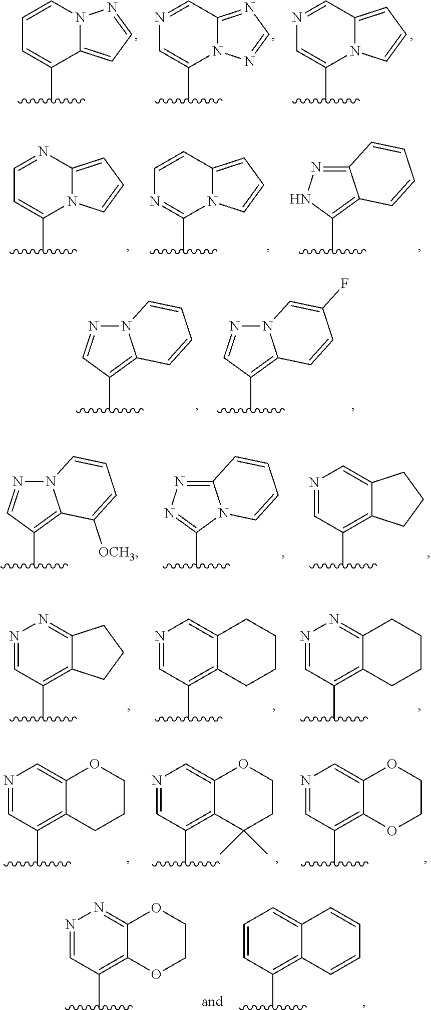
and wherein each of the moieties is unsubstituted or substituted.

7. The compound of claim 1, wherein R² is hydrogen; R⁴ is hydrogen; and R^{N} is hydrogen.

8.-13. (canceled)

14. The compound of claim 1, wherein Ring A¹ is an unsubstituted or a substituted 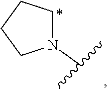 [SMILES: *N1CCCC1]
an unsubstituted or a substituted 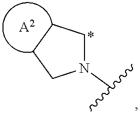
an unsubstituted or a substituted 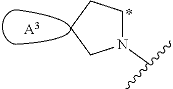
or an unsubstituted or a substituted

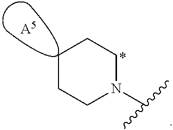

15.-22. (canceled)

23. The compound of claim 14, wherein 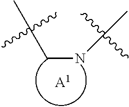
is selected from the group consisting of: 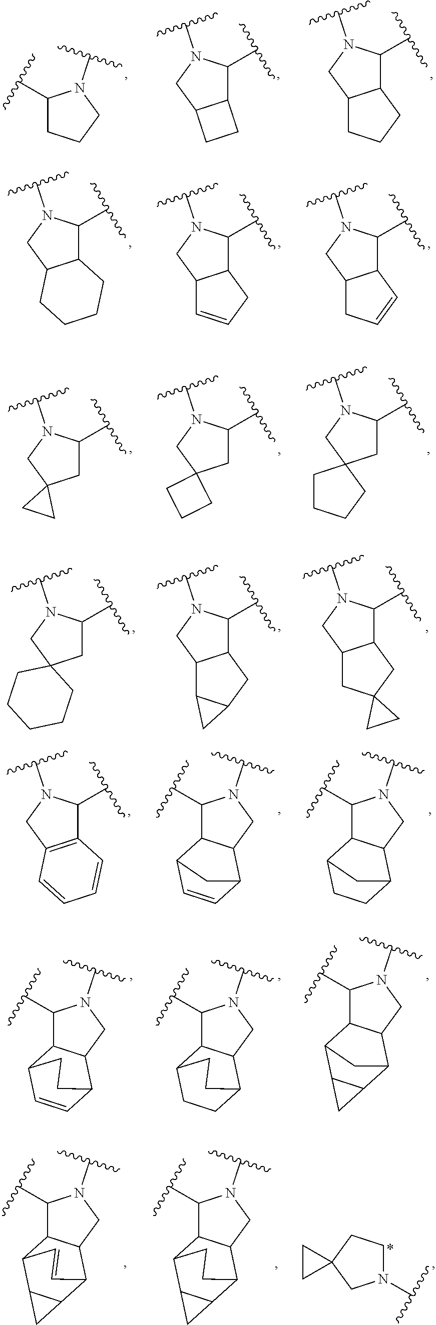 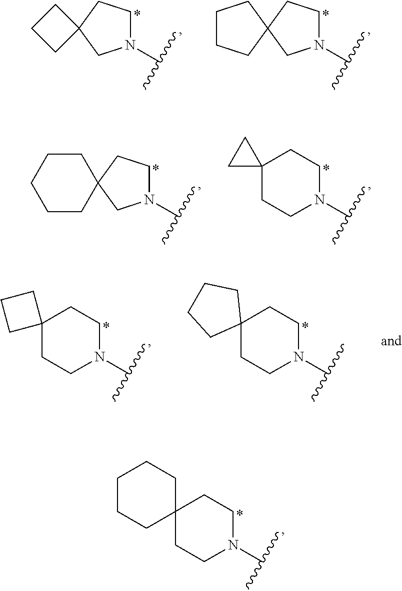
and wherein each is unsubstituted or substituted.

24. (canceled)

25. (canceled)

26. The compound of claim 23, wherein 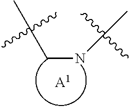
is substituted with one or more moieties independently selected from the group consisting of ═O, ═CH₂, deuterium, halogen, hydroxy, an unsubstituted C₁₋₄ alkyl, an unsubstituted C₁₋₄ haloalkyl, an unsubstituted C₂₋₄ alkenyl, an unsubstituted or a substituted C₃₋₆ monocyclic cycloalkyl and an substituted or a substituted phenyl.

27.-33. (canceled)

34. The compound of claim 1, wherein R¹ is cyano or an unsubstituted C₂₋₅ alkynyl.

35. (canceled)

36. (canceled)

37. The compound of claim 1, wherein R⁵ is 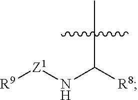
and Z¹ is —C(═O)—.

38. The compound of claim 37, wherein R⁸ is an unsubstituted or a substituted C₁₋₆ alkyl, an unsubstituted monocyclic C₃₋₆ cycloalkyl or an unsubstituted monocyclic C₃₋₆ cycloalkyl(CH₂)—.

39.-52. (canceled)

53. The compound of claim 37, wherein R⁹ is an unsubstituted C₁₋₆ haloalkyl or an unsubstituted or a substituted alkoxy.

54.-61. (canceled)

62. The compound of claim 1, wherein R⁵ is 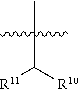
or R¹².

63.-86. (canceled)

87. The compound of claim 62, wherein R¹² is an unsubstituted or a substituted heteroaryl.

88. The compound of claim 87, wherein R¹² is 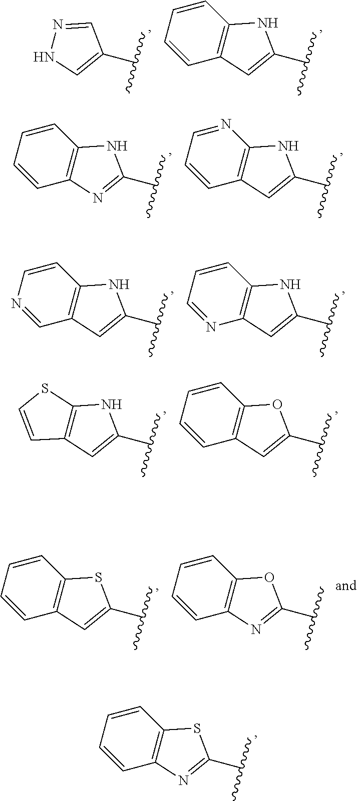
wherein each is unsubstituted or substituted.

89.-97. (canceled)

98. The compound of claim 1, wherein R⁵ is selected from the group consisting of:

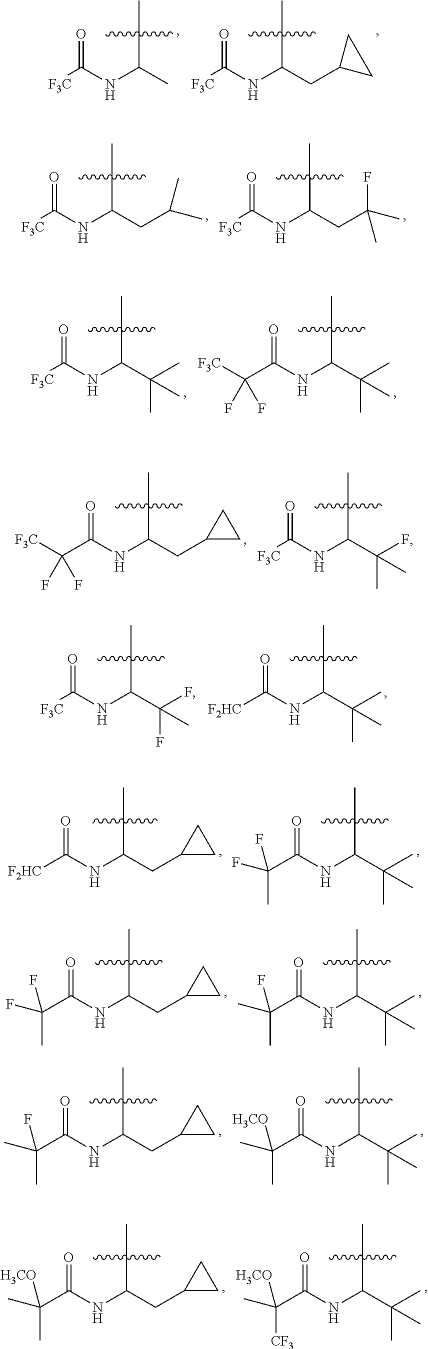

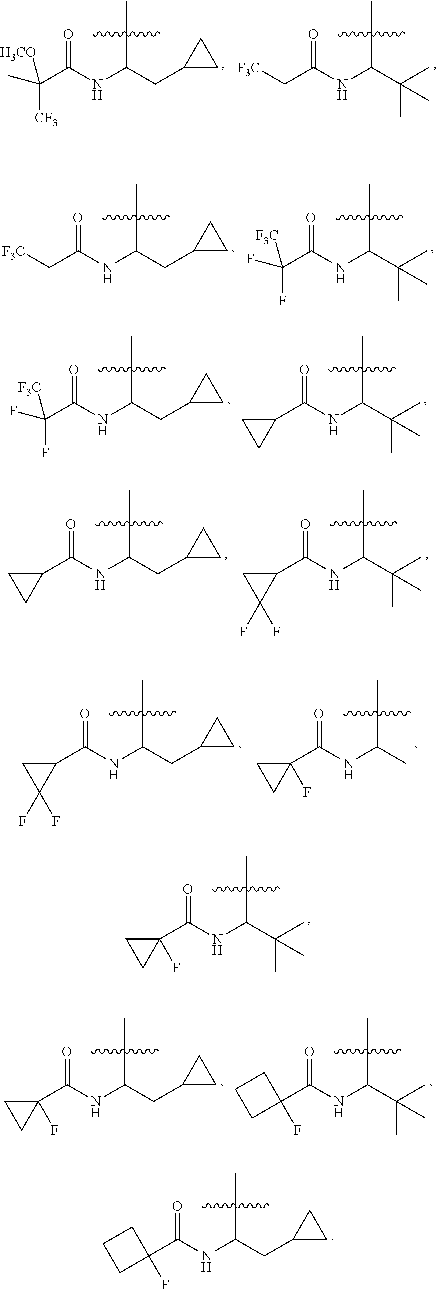

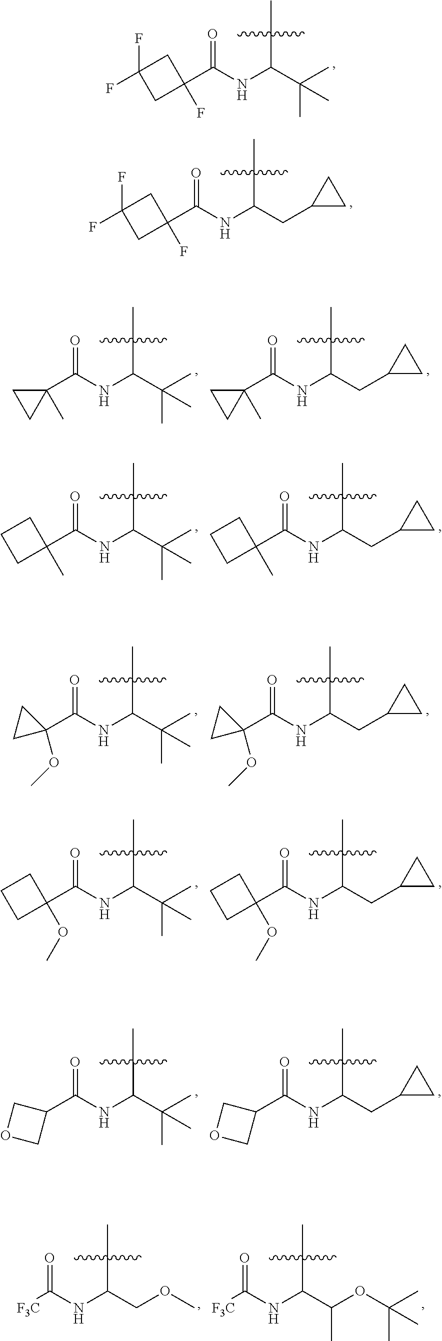

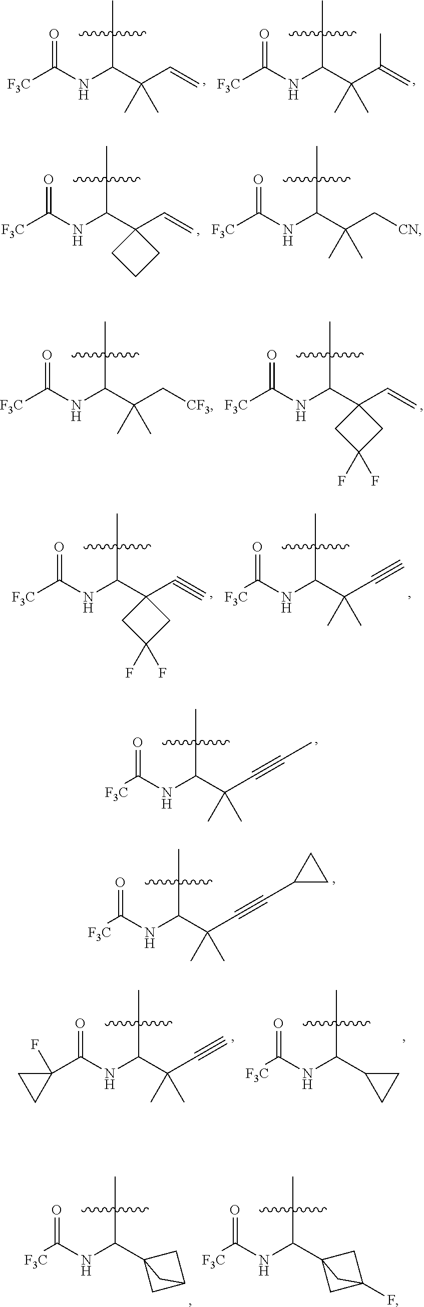

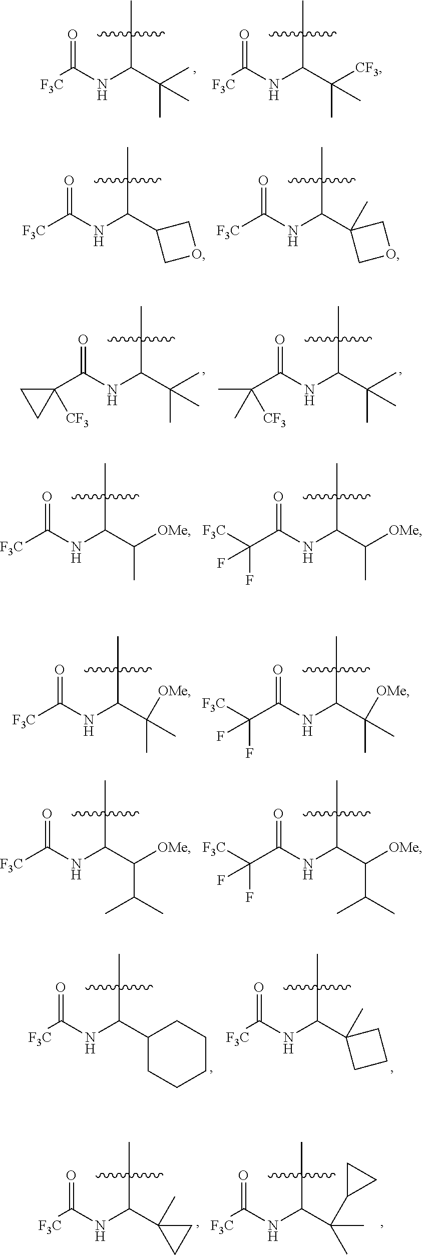

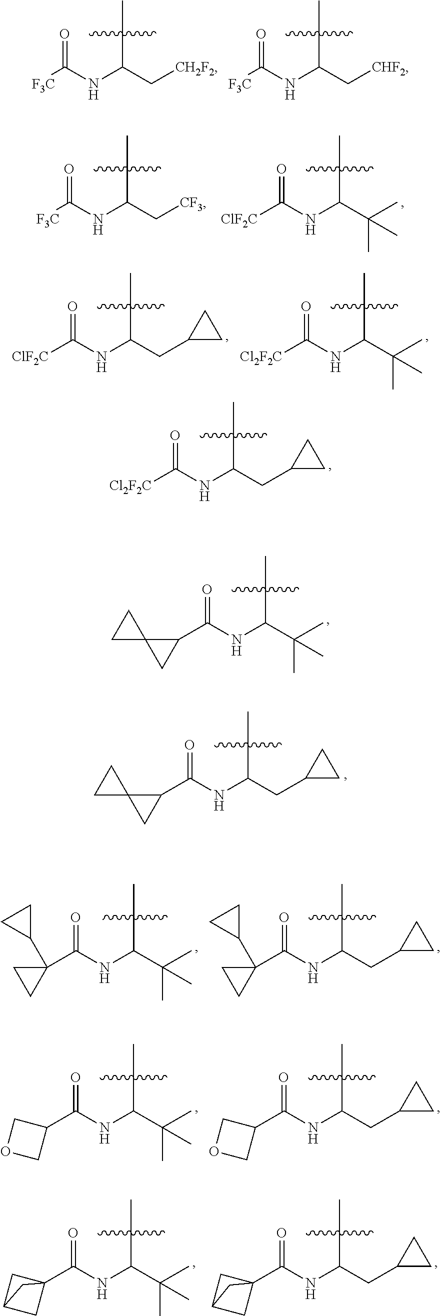

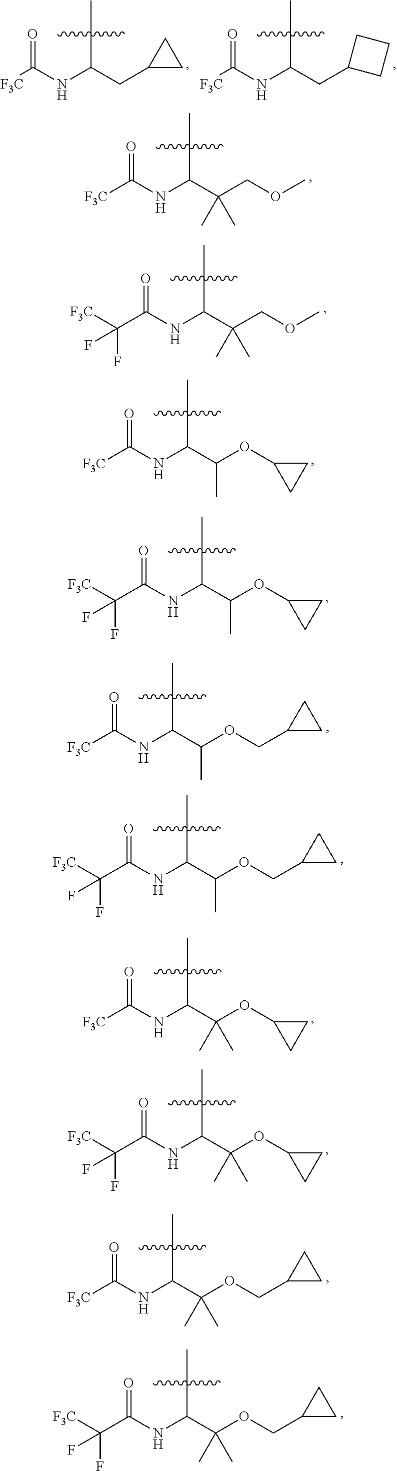

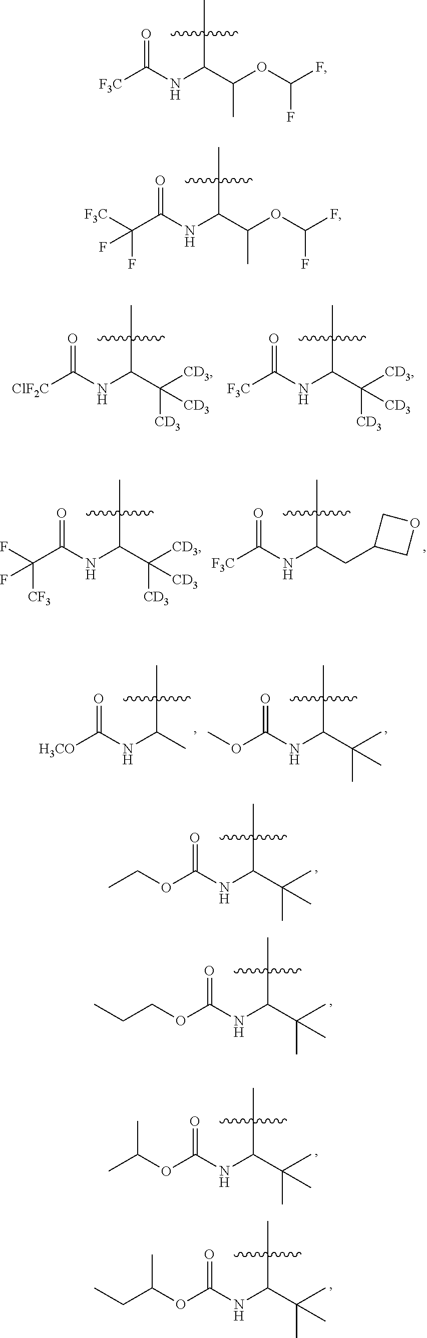

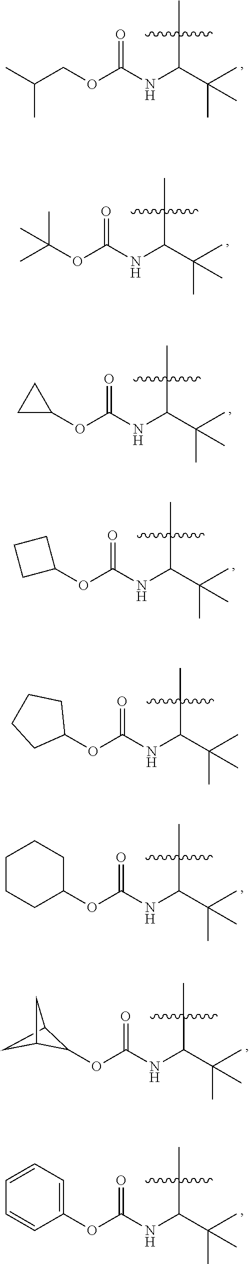

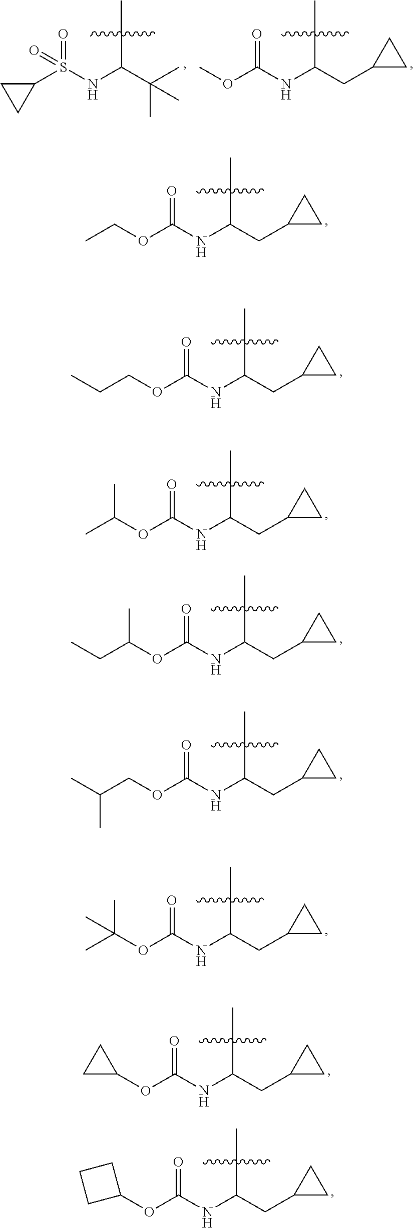

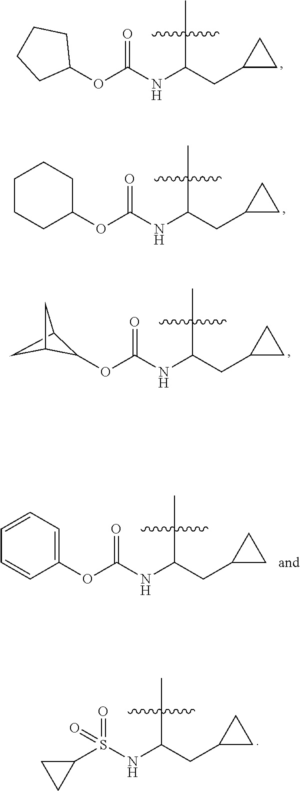

99.-106. (canceled)

107. The compound of claim 1, wherein the compound is selected from the group consisting of: 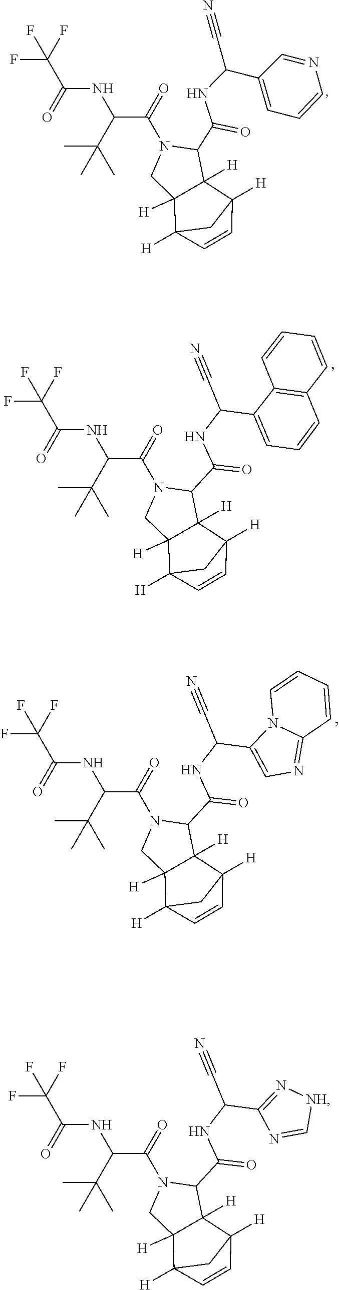 [SMILES: CC(C)(C)C(NC(=O)C(F)(F)F)C(=O)N1CC2C3C=CC(C3)C2C1C(=O)NC(C#N)c1cccnc1 ; CC(C)(C)C(NC(=O)C(F)(F)F)C(=O)N1CC2C3C=CC(C3)C2C1C(=O)NC(C#N)c1cccc2ccccc12 ; CC(C)(C)C(NC(=O)C(F)(F)F)C(=O)N1CC2C3C=CC(C3)C2C1C(=O)NC(C#N)c1cnc2ccccn12 ; CC(C)(C)C(NC(=O)C(F)(F)F)C(=O)N1CC2C3C=CC(C3)C2C1C(=O)NC(C#N)c1nc[nH]n1] 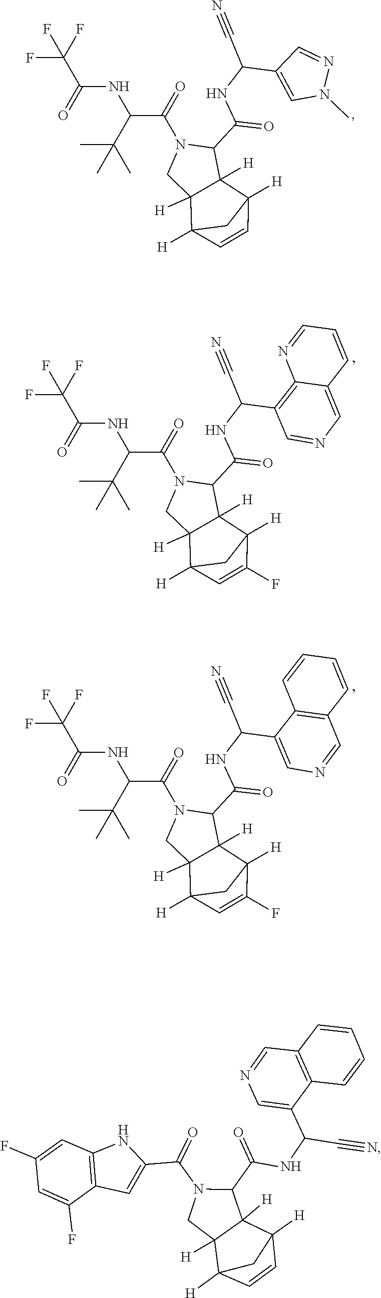 [SMILES: Cn1cc(C(C#N)NC(=O)C2C3C4C=CC(C4)C3CN2C(=O)C(NC(=O)C(F)(F)F)C(C)(C)C)cn1 ; CC(C)(C)C(NC(=O)C(F)(F)F)C(=O)N1CC2C3C=C(F)C(C3)C2C1C(=O)NC(C#N)c1cncc2cccnc12 ; CC(C)(C)C(NC(=O)C(F)(F)F)C(=O)N1CC2C3C=C(F)C(C3)C2C1C(=O)NC(C#N)c1cncc2ccccc12 ; N#CC(NC(=O)C1C2C3C=CC(C3)C2CN1C(=O)c1cc2c(F)cc(F)cc2[nH]1)c1cncc2ccccc12] 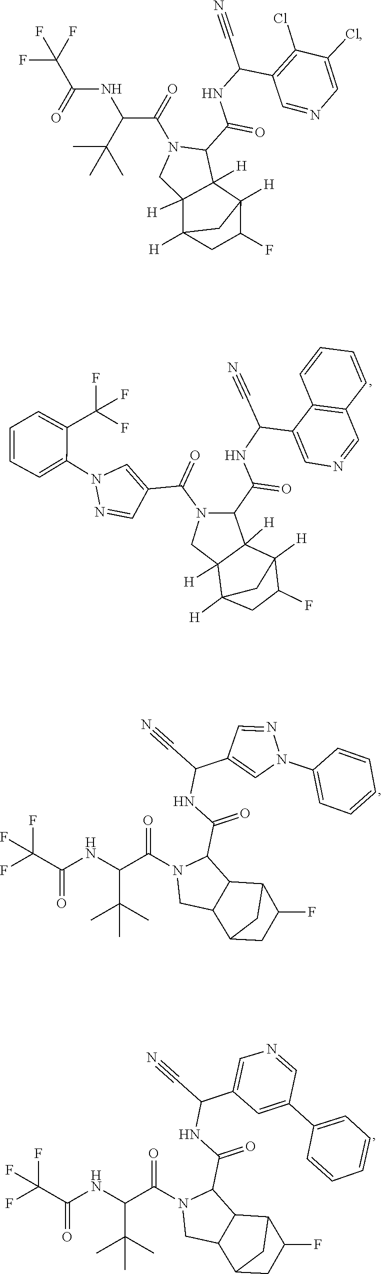 [SMILES: CC(C)(C)C(NC(=O)C(F)(F)F)C(=O)N1CC2C3CC(F)C(C3)C2C1C(=O)NC(C#N)c1cncc(Cl)c1Cl ; N#CC(NC(=O)C1C2C3CC(CC3F)C2CN1C(=O)c1cnn(-c2ccccc2C(F)(F)F)c1)c1cncc2ccccc12 ; CC(C)(C)C(NC(=O)C(F)(F)F)C(=O)N1CC2C3CC(F)C(C3)C2C1C(=O)NC(C#N)c1cnn(-c2ccccc2)c1 ; CC(C)(C)C(NC(=O)C(F)(F)F)C(=O)N1CC2C3CC(F)C(C3)C2C1C(=O)NC(C#N)c1cncc(-c2ccccc2)c1] 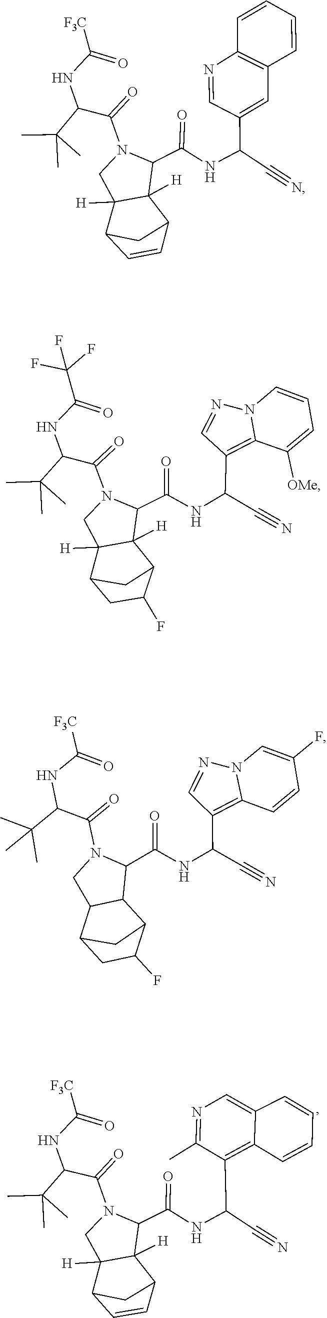 [SMILES: CC(C)(C)C(NC(=O)C(F)(F)F)C(=O)N1CC2C3C=CC(C3)C2C1C(=O)NC(C#N)c1cnc2ccccc2c1 ; COc1cccn2ncc(C(C#N)NC(=O)C3C4C5CC(CC5F)C4CN3C(=O)C(NC(=O)C(F)(F)F)C(C)(C)C)c12 ; CC(C)(C)C(NC(=O)C(F)(F)F)C(=O)N1CC2C3CC(F)C(C3)C2C1C(=O)NC(C#N)c1cnn2cc(F)ccc12 ; Cc1ncc2ccccc2c1C(C#N)NC(=O)C1C2C3C=CC(C3)C2CN1C(=O)C(NC(=O)C(F)(F)F)C(C)(C)C] 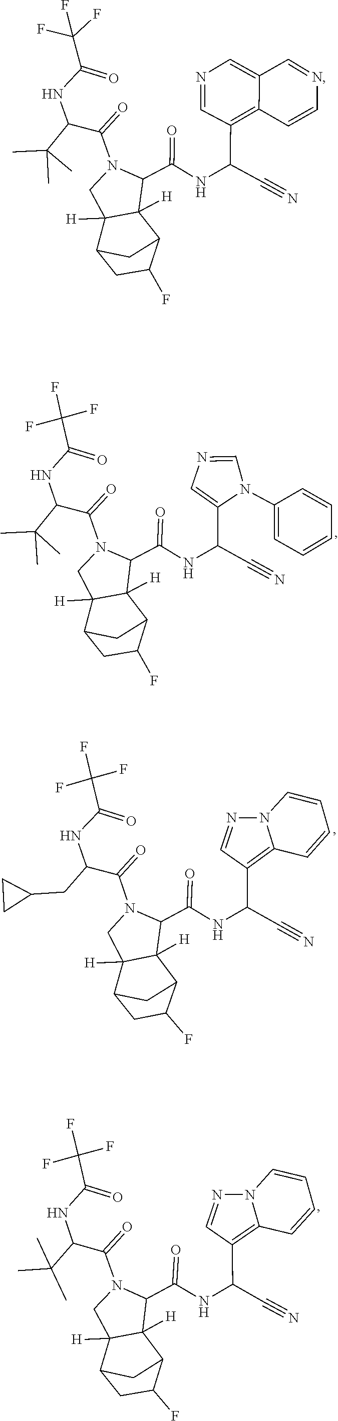 [SMILES: CC(C)(C)C(NC(=O)C(F)(F)F)C(=O)N1CC2C3CC(F)C(C3)C2C1C(=O)NC(C#N)c1cncc2cnccc12 ; CC(C)(C)C(NC(=O)C(F)(F)F)C(=O)N1CC2C3CC(F)C(C3)C2C1C(=O)NC(C#N)c1cncn1-c1ccccc1 ; N#CC(NC(=O)C1C2C3CC(CC3F)C2CN1C(=O)C(CC1CC1)NC(=O)C(F)(F)F)c1cnn2ccccc12 ; CC(C)(C)C(NC(=O)C(F)(F)F)C(=O)N1CC2C3CC(F)C(C3)C2C1C(=O)NC(C#N)c1cnn2ccccc12] 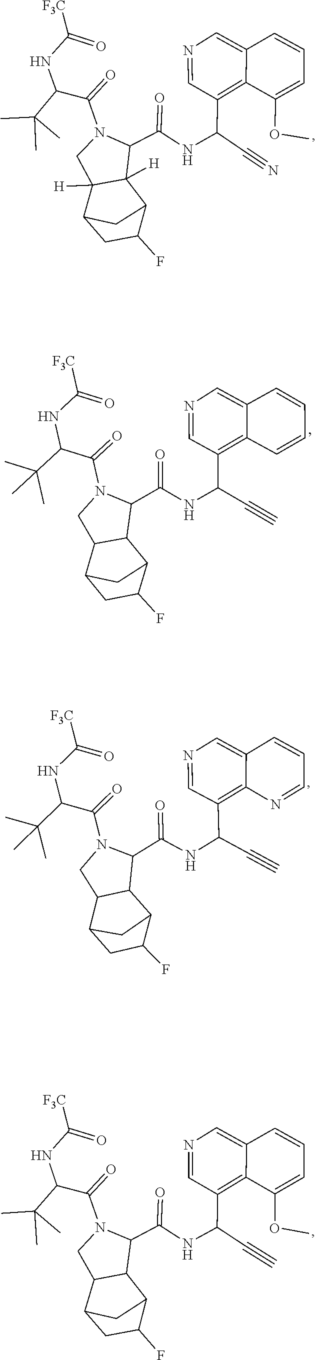 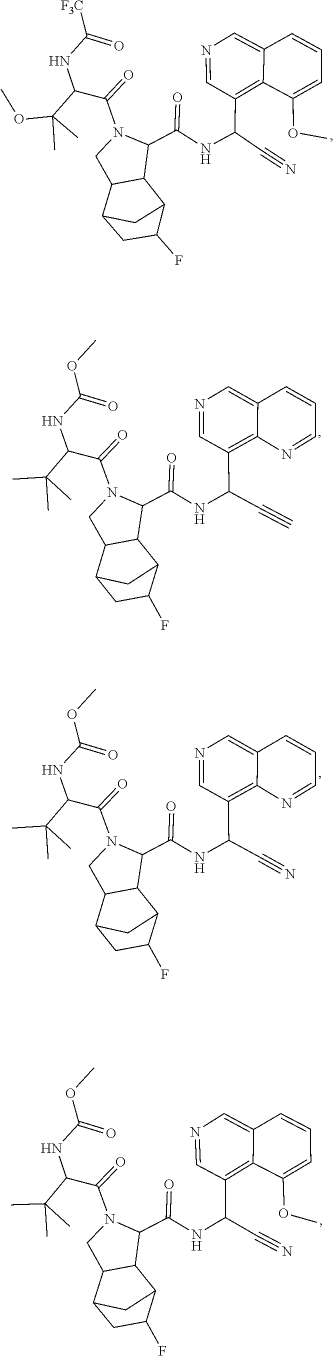 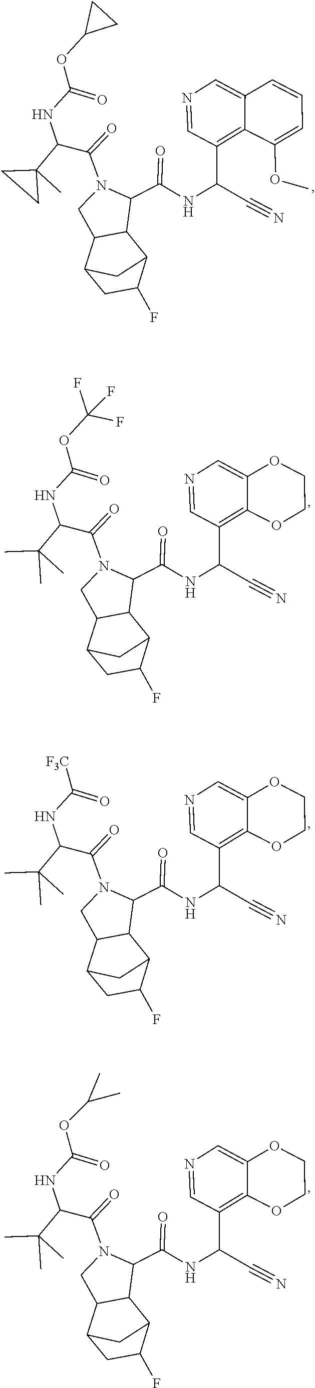 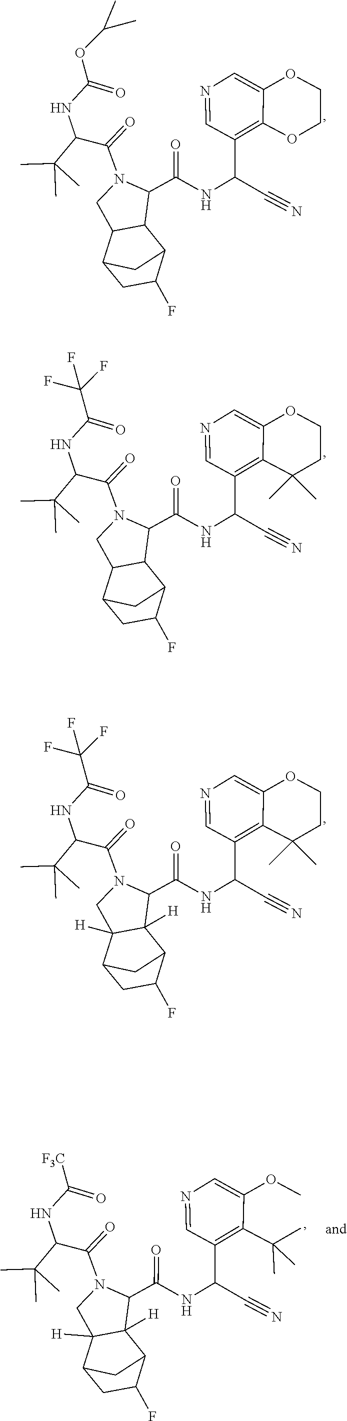 [SMILES: CC(C)OC(=O)NC(C(=O)N1CC2C3CC(F)C(C3)C2C1C(=O)NC(C#N)c1cncc2c1OCCO2)C(C)(C)C ; CC1(C)CCOc2cncc(C(C#N)NC(=O)C3C4C5CC(CC5F)C4CN3C(=O)C(NC(=O)C(F)(F)F)C(C)(C)C)c21 ; CC1(C)CCOc2cncc(C(C#N)NC(=O)C3C4C5CC(CC5F)C4CN3C(=O)C(NC(=O)C(F)(F)F)C(C)(C)C)c21 ; COc1cncc(C(C#N)NC(=O)C2C3C4CC(CC4F)C3CN2C(=O)C(NC(=O)C(F)(F)F)C(C)(C)C)c1C(C)(C)C] 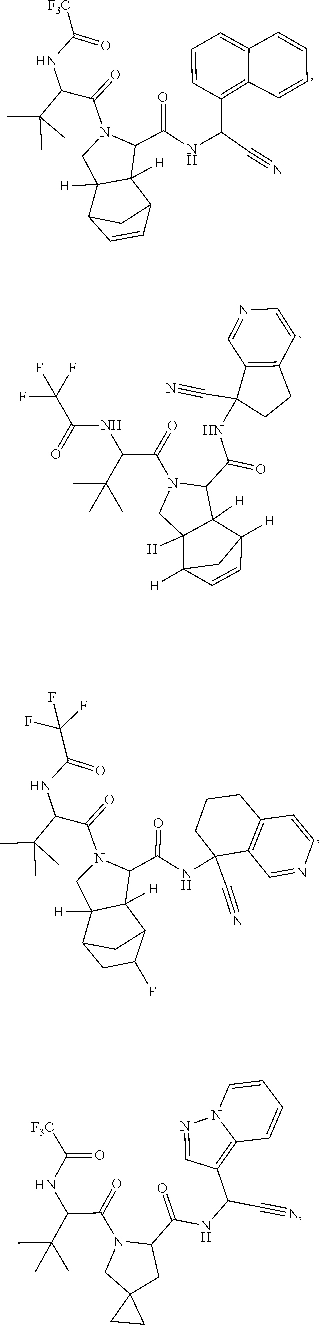 [SMILES: CC(C)(C)C(NC(=O)C(F)(F)F)C(=O)N1CC2C3C=CC(C3)C2C1C(=O)NC(C#N)c1cccc2ccccc12 ; CC(C)(C)C(NC(=O)C(F)(F)F)C(=O)N1CC2C3C=CC(C3)C2C1C(=O)NC1(C#N)CCc2ccncc21 ; CC(C)(C)C(NC(=O)C(F)(F)F)C(=O)N1CC2C3CC(F)C(C3)C2C1C(=O)NC1(C#N)CCCc2ccncc21 ; CC(C)(C)C(NC(=O)C(F)(F)F)C(=O)N1CC2(CC2)CC1C(=O)NC(C#N)c1cnn2ccccc12] 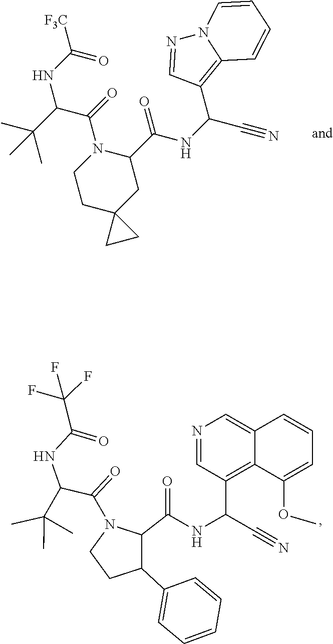 [SMILES: CC(C)(C)C(NC(=O)C(F)(F)F)C(=O)N1CCC2(CC2)CC1C(=O)NC(C#N)c1cnn2ccccc12 ; COc1cccc2cncc(C(C#N)NC(=O)C3C(c4ccccc4)CCN3C(=O)C(NC(=O)C(F)(F)F)C(C)(C)C)c12]
or a pharmaceutically acceptable salt of any of the foregoing.

108. (canceled)

109. The compound of claim 1, wherein the compound is selected from the group consisting of: 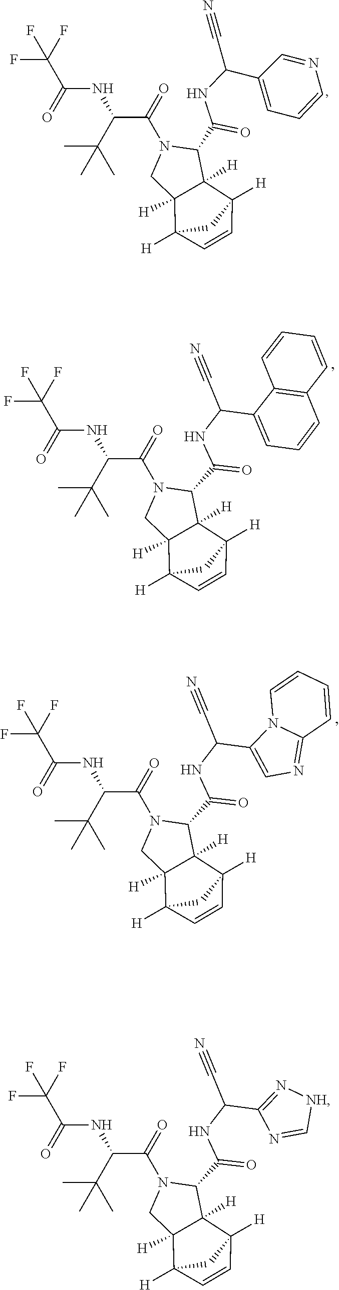 [SMILES: CC(C)(C)[C@H](NC(=O)C(F)(F)F)C(=O)N1C[C@H]2[C@@H]([C@H]1C(=O)NC(C#N)c1cccnc1)[C@H]1C=C[C@@H]2C1 ; CC(C)(C)[C@H](NC(=O)C(F)(F)F)C(=O)N1C[C@H]2[C@@H]([C@H]1C(=O)NC(C#N)c1cccc3ccccc13)[C@H]1C=C[C@@H]2C1 ; CC(C)(C)[C@H](NC(=O)C(F)(F)F)C(=O)N1C[C@H]2[C@@H]([C@H]1C(=O)NC(C#N)c1cnc3ccccn13)[C@H]1C=C[C@@H]2C1 ; CC(C)(C)[C@H](NC(=O)C(F)(F)F)C(=O)N1C[C@H]2[C@@H]([C@H]1C(=O)NC(C#N)c1nc[nH]n1)[C@H]1C=C[C@@H]2C1] 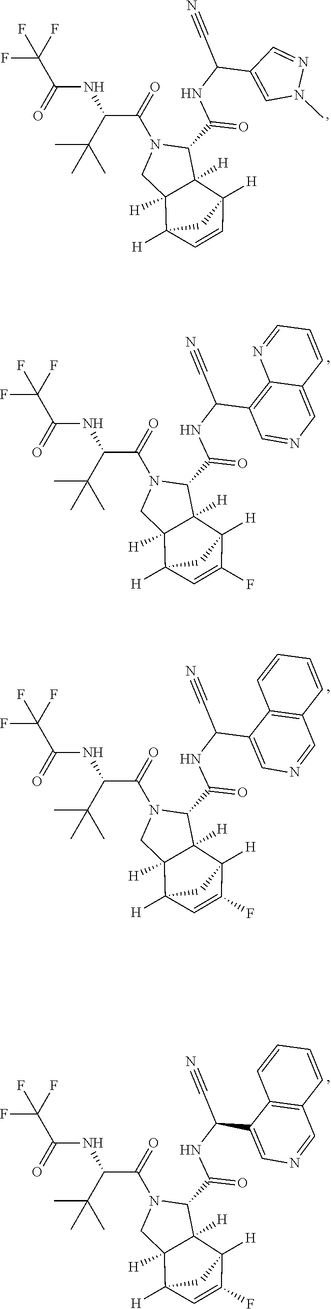 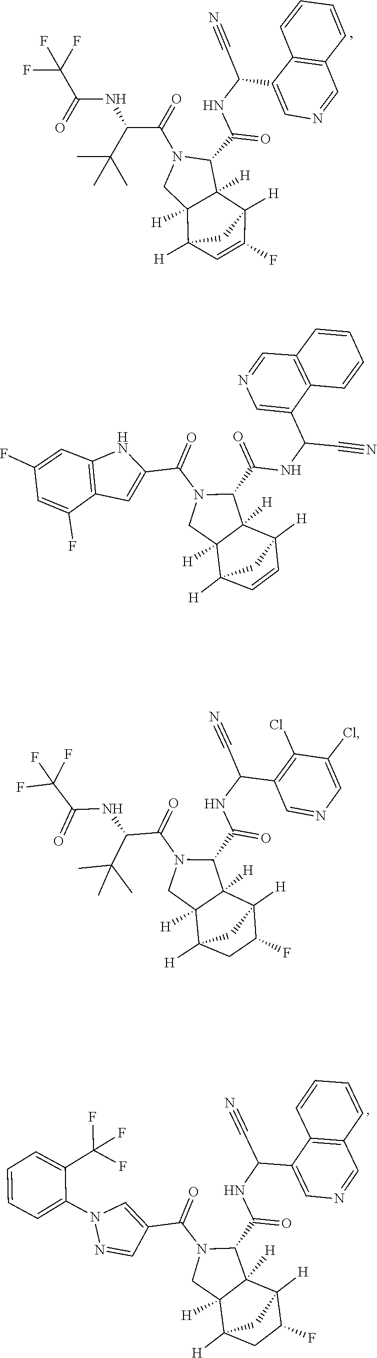 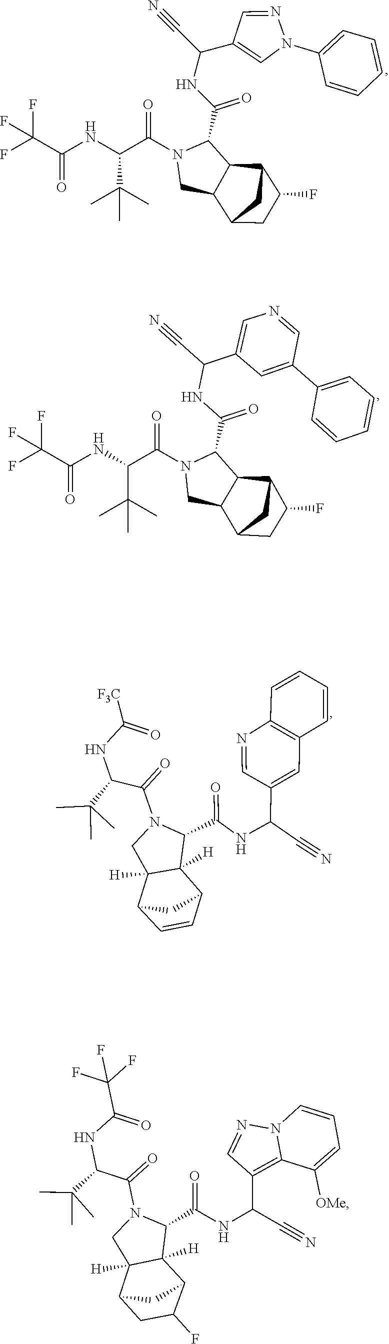 [SMILES: CC(C)(C)[C@H](NC(=O)C(F)(F)F)C(=O)N1C[C@@H]2[C@@H]3C[C@H]([C@@H]2[C@H]1C(=O)NC(C#N)c1cnn(-c2ccccc2)c1)[C@H](F)C3 ; CC(C)(C)[C@H](NC(=O)C(F)(F)F)C(=O)N1C[C@@H]2[C@@H]3C[C@H]([C@@H]2[C@H]1C(=O)NC(C#N)c1cncc(-c2ccccc2)c1)[C@H](F)C3 ; CC(C)(C)[C@H](NC(=O)C(F)(F)F)C(=O)N1C[C@H]2[C@@H]([C@H]1C(=O)NC(C#N)c1cnc3ccccc3c1)[C@H]1C=C[C@@H]2C1 ; COc1cccn2ncc(C(C#N)NC(=O)[C@@H]3[C@@H]4[C@H](CN3C(=O)[C@@H](NC(=O)C(F)(F)F)C(C)(C)C)[C@@H]3CC(F)[C@H]4C3)c12] 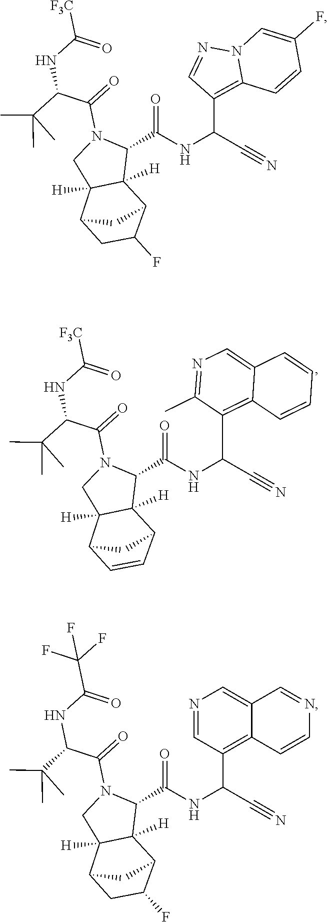 [SMILES: CC(C)(C)[C@H](NC(=O)C(F)(F)F)C(=O)N1C[C@@H]2[C@@H]3CC(F)[C@@H](C3)[C@@H]2[C@H]1C(=O)NC(C#N)c1cnn2cc(F)ccc12 ; Cc1ncc2ccccc2c1C(C#N)NC(=O)[C@@H]1[C@@H]2[C@H](CN1C(=O)[C@@H](NC(=O)C(F)(F)F)C(C)(C)C)[C@@H]1C=C[C@H]2C1 ; CC(C)(C)[C@H](NC(=O)C(F)(F)F)C(=O)N1C[C@@H]2[C@H]3C[C@@H]([C@@H]2[C@H]1C(=O)NC(C#N)c1cncc2cnccc12)[C@H](F)C3] 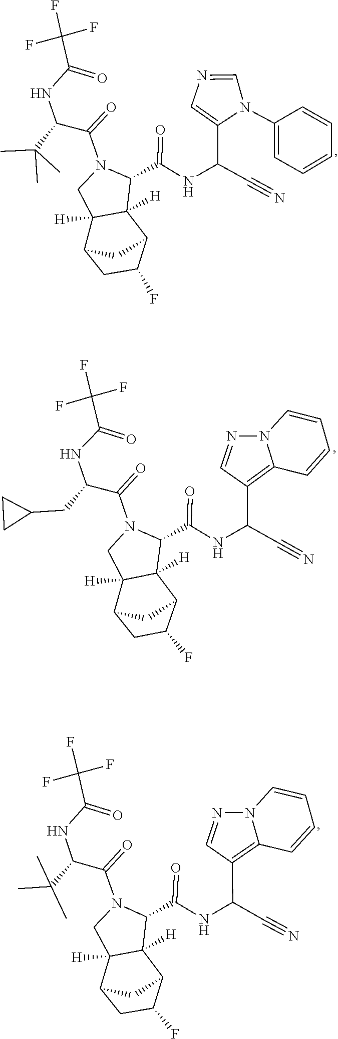 [SMILES: CC(C)(C)[C@H](NC(=O)C(F)(F)F)C(=O)N1C[C@@H]2[C@H]3C[C@@H]([C@@H]2[C@H]1C(=O)NC(C#N)c1cncn1-c1ccccc1)[C@H](F)C3 ; N#CC(NC(=O)[C@@H]1[C@@H]2[C@H](CN1C(=O)[C@H](CC1CC1)NC(=O)C(F)(F)F)[C@H]1C[C@@H]2[C@H](F)C1)c1cnn2ccccc12 ; CC(C)(C)[C@H](NC(=O)C(F)(F)F)C(=O)N1C[C@@H]2[C@H]3C[C@@H]([C@@H]2[C@H]1C(=O)NC(C#N)c1cnn2ccccc12)[C@H](F)C3] 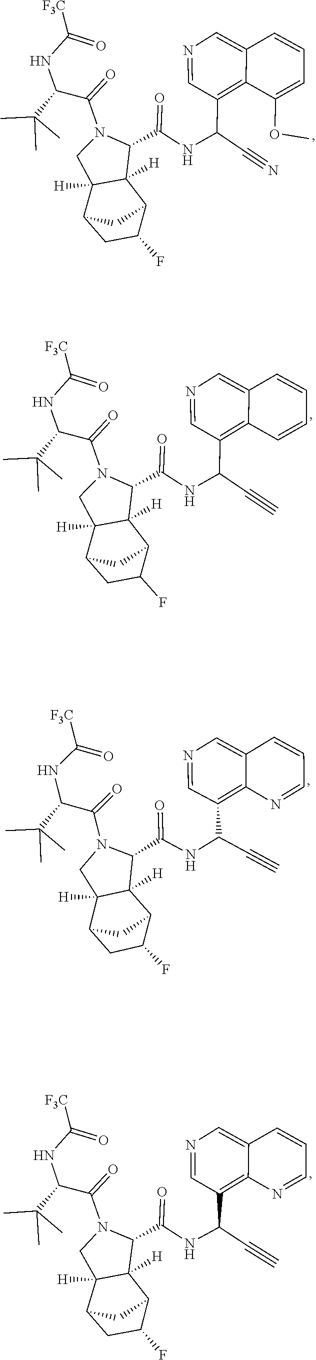 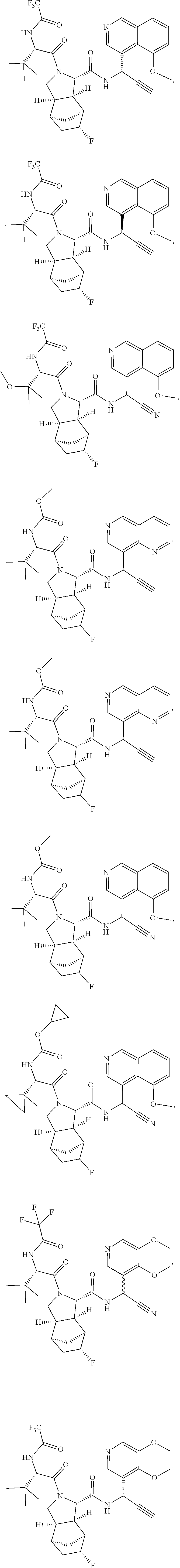 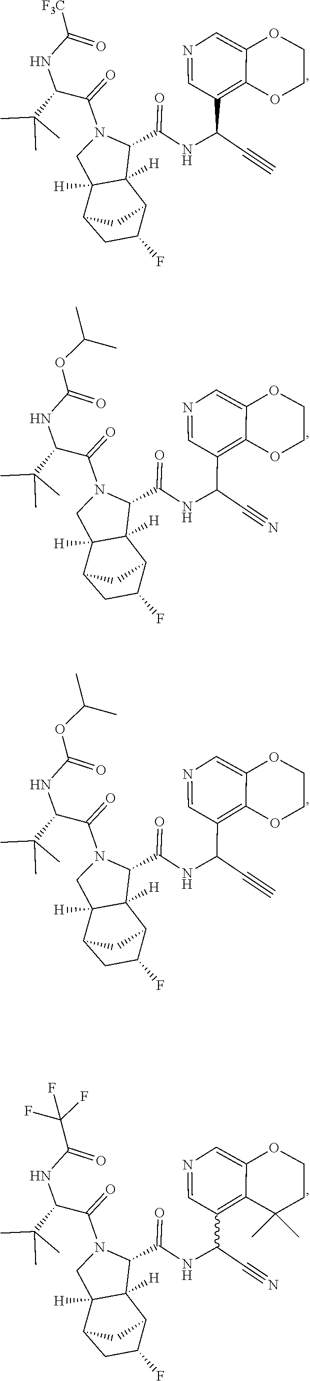 [SMILES: C#C[C@@H](NC(=O)[C@@H]1[C@@H]2[C@H](CN1C(=O)[C@@H](NC(=O)C(F)(F)F)C(C)(C)C)[C@H]1C[C@@H]2[C@H](F)C1)c1cncc2c1OCCO2 ; CC(C)OC(=O)N[C@H](C(=O)N1C[C@@H]2[C@H]3C[C@@H]([C@@H]2[C@H]1C(=O)NC(C#N)c1cncc2c1OCCO2)[C@H](F)C3)C(C)(C)C ; C#CC(NC(=O)[C@@H]1[C@@H]2[C@H](CN1C(=O)[C@@H](NC(=O)OC(C)C)C(C)(C)C)[C@H]1C[C@@H]2[C@H](F)C1)c1cncc2c1OCCO2 ; CC1(C)CCOc2cncc(C(C#N)NC(=O)[C@@H]3[C@@H]4[C@H](CN3C(=O)[C@@H](NC(=O)C(F)(F)F)C(C)(C)C)[C@H]3C[C@@H]4[C@H](F)C3)c21] 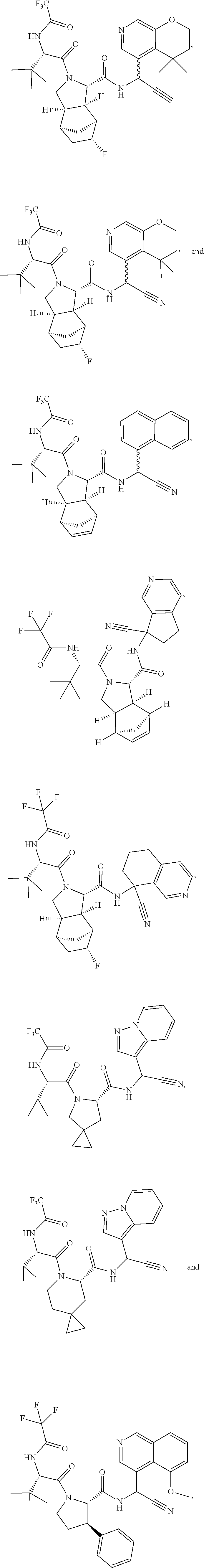
or a pharmaceutically acceptable salt of any of the foregoing.

110. (canceled)

111. A pharmaceutical composition comprising an effective amount of a compound of claim 1, or a pharmaceutically acceptable salt thereof, and excipient.

112.-127. (canceled)

128. A method for treating a coronavirus infection in a subject comprising administering to the subject in need thereof an effective amount of a compound of claim 1, or a pharmaceutically acceptable salt thereof.

129. The method of claim 128, further comprising administering an additional agent selected from the group consisting of an ACE inhibitor, an anticoagulant, an anti-inflammatory, an ARB, an ASO, a Covid-19 convalescent plasma, an entry inhibitor, an H₂ pump antagonist, an H-conducting channel, an HIV protease inhibitor, an HMG-CoA reductase inhibitor, an immune globulin, an immunosuppressant, an immunotherapeutic agent, a neuraminidase inhibitor, a nucleoside inhibitor, a nucleoside analog inhibitor, a polymerase inhibitor, a protease inhibitor, an siRNA, a statin, a tissue plasminogen activator, an antibiotic, an antimicrobial and a vaccine.

130.-144. (canceled)

145. A method for inhibiting a coronavirus protease comprising contacting a cell infected with a coronavirus with an effective amount of a compound of claim 1, or a pharmaceutically acceptable salt thereof, wherein the compound of claim 1, or a pharmaceutically acceptable salt thereof, selectively inhibits the coronavirus protease compared to a host protease.

146. (canceled)

147. (canceled)

148. (canceled)
